(19) United States
(12) Patent Application Publication
(10) Pub. No.: US 2025/0129054 A1
(43) Pub. Date: Apr. 24, 2025

(54) PRODRUGS OF 3,4-METHYLENEDIOXY-N-METHCATHINONE AND USES THEREOF

(71) Applicant: Terran Biosciences Inc., Miami Beach, FL (US)

(72) Inventors: Matthew DUNCTON, Las Vegas, NV (US); Samuel CLARK, Miami, FL (US)

(21) Appl. No.: 18/728,575

(22) PCT Filed: Jan. 13, 2023
(86) PCT No.: PCT/US2023/060669

§ 371 Date: Jul. 12, 2024

Related U.S. Application Data
(60) Provisional application No. 63/299,860, filed on Jan. 14, 2022.

(51) Int. Cl. C07D 405/04; A61K 31/36; A61K 31/423; A61K 31/4545; A61K 31/497; A61K 45/06; A61P 25/00; A61P 25/22; C07D 317/58; C07D 405/14; C07D 413/04

(52) U.S. Cl. CPC C07D 405/04; A61K 31/36; A61K 31/423; A61K 31/4545; A61K 31/497; A61K 45/06; A61P 25/00; A61P 25/22; C07D 317/58; C07D 405/14; C07D 413/04

(57) ABSTRACT

The present disclosure relates to compounds Formula (I): methods for making the compounds and methods for their use.

### CROSS-REFERENCE TO RELATED APPLICATIONS

This application claims the benefit of U.S. Provisional Application No. 63/299,860, filed Jan. 14, 2022, which is incorporated herein by reference in its entirety.

### FIELD OF THE INVENTION

The present disclosure relates to analogs of 3,4-methylenedioxy-N-methcathinone (methylone), including prodrugs thereof. The disclosure further relates to the use of such compounds to treat brain and neurological disorders.

### BACKGROUND OF THE INVENTION

Major depressive disorder and related neuropsychiatric diseases are among the leading causes of disability worldwide. Despite recent advances, there remains a need for new therapeutics to support treatment of debilitating neuropsychiatric diseases.

Psychedelics have been shown to have therapeutic benefits. Recently, psychedelic compounds have received renewed interest for the treatment of depression and other disorders. For example, the Food and Drug Administration (FDA) recently approved the dissociative anesthetic ketamine for treatment-resistant depression, making it the first mechanistically distinct medicine to be introduced to psychiatry in nearly thirty years. Ketamine is a member of a class of compounds known as psychoplastogens. Psychoplastogens promote neuronal growth through a mechanism involving the activation of AMPA receptors, the tropomyosin receptor kinase B (TrkB), and the mammalian target of rapamycin (mTOR). As pyramidal neurons in the PFC exhibit top-down control over areas of the brain controlling motivation, fear, and reward, these effects support clinical development of psychoplastogenic compounds for their antidepressant, anxiolytic, and anti-addictive effects properties.

Methylone (3,4-methylenedioxy-N-methylcathinone) is a synthetic analog of the psychedelic phenethylamine class of compounds.

### BRIEF SUMMARY OF THE INVENTION

Disclosed herein are compounds of Formula (I):

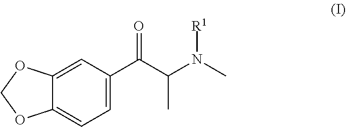

or a pharmaceutically acceptable salt thereof, wherein:
R¹ is —C(O)OR³, —C(O)R⁴, —CH(R⁵)OR⁶, —C(O)OCH(R⁵)OC(O)R⁴, —C(O)OCH(R⁵)OC(O)OR⁴, —C(O)OCH(R⁵)OC(O)NHR⁴, —CH(R⁵)NHC(O)R⁶, —CH(R⁵)C(O)R⁶, —S(O)₂R⁷, —S(O)₂OR⁷, —P(O)OR⁸[N(R⁹)R¹⁰], —C(O)N(R⁹)R¹⁰, —P(O)OR¹¹(OR¹²), —CH(R⁴)OP(O)OR⁸[N(R⁹)R¹⁰], or —CH(R⁴)OP(O)OR¹¹(OR¹²);
each of R³, R⁴, R⁶, R⁷, and R¹ is independently alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
R⁵ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
or R³ is

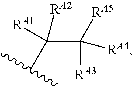

wherein each of R^{A1}, R^{A2}, R^{A3}, and R^{A4} is independently hydrogen or alkyl; and R^{A5} is heteroalkyl, heterocycloalkyl, heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶;
or R⁴ is

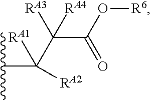

wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; and R⁶ is hydrogen, alkyl, cycloalkyl, aryl, or heteroaryl;
or R⁴ is

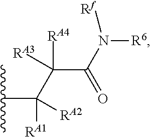

wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; R^{f} is hydrogen or alkyl; and R⁶ is hydrogen, alkyl, cycloalkyl, aryl, or heteroaryl;
each of R⁹ and R¹⁰ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
each of R¹¹ and R¹² is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), wherein alkyl, heteroalkyl, cycloalkyl, heterocycloalkyl, aryl, or heteroaryl is unsubstituted or substituted with alkyl, aryl, halogen, —OR¹³, —NR(R¹⁸)R¹⁹, —C(O)R¹⁴, —OC(O)R¹⁵, —OC(O)OR¹⁶, —NHC(O)OR¹⁶, or —OC(O)N(R¹⁸)R¹⁹;
each of R¹³, R¹⁴, R¹⁵, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰ and R²¹ is independently hydrogen, alkyl, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and
each R^{B} is independently halogen, amino, cyano, hydroxyl, alkyl, heteroalkyl, cycloalkyl, heterocycloalkyl, aryl, heteroaryl, arylalkyl, —C(O)CH₃, —C(O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

Also disclosed herein are methods for making and using compounds of Formula (I).

The foregoing and other objects, features, and advantages of the invention will become more apparent from the following detailed description.

### BRIEF DESCRIPTION OF THE DRAWINGS

FIG. 1-A shows mean concentration-time profiles of methylone following IV dosing of methylone (1 mg/kg) to male Sprague Dawley (SD) rats.

FIG. 1-B shows mean concentration-time profiles of methylone following oral dosing of methylone (10 mg/Kg) to male Sprague Dawley (SD) rats.

FIG. 2 shows mean concentration-time profiles of methylone following oral dosing of methylone acetamide prodrug (10 mg/kg) to male SD rats.

FIG. 3 shows mean concentration-time profiles of methylone following oral dosing of methylone trimethyl lock prodrug (10 mg/kg) to male SD rats.

FIG. 4 shows mean concentration-time profiles of methylone following oral dosing of methylone methyl pivalate prodrug (10 mg/kg) to male SD rats.

FIG. 5 shows mean concentration-time profiles of methylone following oral dosing of methylone THP methyl ester prodrug (10 mg/kg) to male SD rats.

FIG. 6 shows mean concentration-time profiles of methylone following oral dosing of methylone t-butyl methyl glutarate prodrug (10 mg/kg) to male SD rats.

FIG. 7 shows mean concentration-time profiles of methylone following oral dosing of methylone methyl hemi-glutarate prodrug (10 mg/kg) to male SD rats.

FIG. 8 shows mean concentration-time profiles of methylone following oral dosing of methylone oxazolone prodrug (10 mg/kg) to male SD rats.

FIG. 9 shows mean concentration-time profiles of methylone following oral dosing of methylone THP 1-ethyl ester prodrug (10 mg/kg) to male SD rats.

FIG. 10 shows mean concentration-time profiles of methylone following oral dosing of methylone methyl hemi-adipate prodrug (10 mg/kg) to male SD rats.

FIG. 11 illustrates the percentage of time spent in the open arms after racemic methylone compared to vehicle and chlordiazepoxide control on the elevated zero maze.

FIG. 12 illustrates the percentage of time spent in the open arms after S-methylone compared to vehicle and chlordiazepoxide control on the elevated zero maze.

FIG. 13 illustrates the percentage of time spent in the open arms after R-methylone compared to vehicle and chlordiazepoxide control on the elevated zero maze.

FIG. 14 illustrates the frequency of SAPs after racemic methylone compared to vehicle and chlordiazepoxide control on the elevated zero maze.

FIG. 15 illustrates the frequency of SAPs after S-methylone compared to vehicle and chlordiazepoxide control on the elevated zero maze.

FIG. 16 illustrates the frequency of SAPs after R-methylone compared to vehicle and chlordiazepoxide control on the elevated zero maze.

FIG. 17 illustrates a logarithmic graph showing the concentration of racemic methylone, R-methylone, and S-methylone in plasma and brain measured at the end of the zero maze study.

FIG. 18 illustrates a graph showing the concentration of racemic methylone, R-methylone, and S-methylone in plasma and brain measured at the end of the zero maze study.

### DETAILED DESCRIPTION

Described herein, in certain embodiments, are compositions and methods relating to synthesis of analogs of 3,4-methylenedioxy-N-methcathinone (methylone). In one embodiment, the analogs described herein are prodrugs, that is, compounds that are converted to methylone under physiologic conditions. Methylone contains a chiral center the two enantiomers of methylone are the (R)- and (S)-enantiomers. It is also possible that a prodrug of an individual enantiomer of methyone may have advantages over the other enantiomer or the racemic mixture.

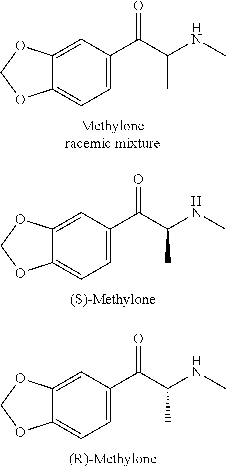

### Compounds of the Disclosure

In one aspect, the present disclosure provides a compound of Formula (I):

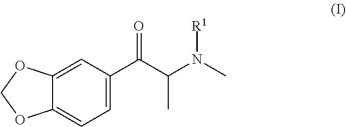

or a pharmaceutically acceptable salt thereof, wherein:
R¹ is —C(O)OR³, —C(O)R⁴, —CH(R⁵)OR⁶, —C(O)OCH(R⁵)OC(O)R⁴, —C(O)OCH(R⁵)OC(O)OR⁴, —C(O)OCH(R⁵)OC(O)NHR⁴, —CH(R⁵)NHC(O)R⁶, —CH(R⁵)C(O)R⁶, —S(O)₂R⁷, —S(O)₂OR⁷, —P(O)OR⁸[N(R⁹)R¹⁰], —C(O)N(R⁹)R¹⁰, —P(O)OR¹¹(OR¹²), —CH(R⁴)OP(O)OR⁸[N(R⁹)R¹⁰], or —CH(R⁴)OP(O)OR¹¹(OR¹²);
each of R³, R⁴, R⁶, R⁷, and R¹ is independently alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
R⁵ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
or R³ is

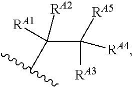

wherein each of R^{A1}, R^{A2}, R^{A3}, and R^{A4} is independently hydrogen or alkyl; and R^{A5} is heteroalkyl, heterocycloalkyl, heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶;
or R⁴ is

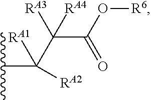

wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; and R⁶ is hydrogen, alkyl, cycloalkyl, aryl, or heteroaryl;
or R⁴ is

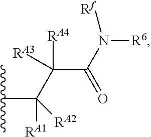

wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; R^{f} is hydrogen or alkyl; and R⁶ is hydrogen, alkyl, cycloalkyl, aryl, or heteroaryl;
each of R⁹ and R¹⁰ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
each of R¹¹ and R¹² is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R^{1′})R¹⁹, or —OP(O)OR²⁰(OR²¹), wherein alkyl, heteroalkyl, cycloalkyl, heterocycloalkyl, aryl, or heteroaryl is unsubstituted or substituted with alkyl, aryl, halogen, —OR¹³, —NR(R¹⁸)R¹⁹, —C(O)R¹⁴, —OC(O)R¹⁵, —OC(O)OR¹⁶, —NHC(O)OR¹⁶, or —OC(O)N(R¹⁸)R¹⁹;
each of R¹³, R¹⁴, R¹⁵, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰ and R²¹ is independently hydrogen, alkyl, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and
each R^{B} is independently hydrogen, halogen, amino, cyano, hydroxyl, alkyl, heteroalkyl, cycloalkyl, heterocycloalkyl, aryl, heteroaryl, arylalkyl, —C(O)CH₃, —C(O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

In some embodiments, each of R³, R⁴, R⁶, R⁷, and R¹ is independently C₁-C₁₀ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}. In some embodiments, each of R³, R⁴, R⁶, R⁷, and R¹ is independently C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}.

In some embodiments, R⁵ is hydrogen, C₁-C₁₀ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}. In some embodiments, R⁵ is hydrogen, C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}.

In some embodiments, or R³ is

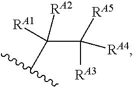

wherein each of R^{A1}, R^{A2}, R^{A3}, and R^{A4} is independently hydrogen or C₁-C₁₀ alkyl; and R^{A5} is C₃-C₆ heteroalkyl, 3- to 6-membered heterocycloalkyl, monocyclic heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶.

In some embodiments, or R⁴ is

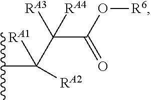

wherein each of R^{A1} and R^{A2} is independently hydrogen, C₁-C₁₀ alkyl, C₃-C₆ cycloalkyl, phenyl, or monocyclic heteroaryl, or R^{A1} and R^{A2} together with the atom to which they are attached form a C₃-C₆ cycloalkyl ring; each of R^{A3} and R^{A4} is independently hydrogen, C₁-C₁₀ alkyl, C₃-C₆ cycloalkyl, phenyl, or monocyclic heteroaryl, or R^{A3} and R^{A4} together with the atom to which they are attached form a C₃-C₆ cycloalkyl ring; and R⁶ is hydrogen, C₁-C₁₀ alkyl, C₃-C₆ cycloalkyl, phenyl, or monocyclic heteroaryl.

In some embodiments, or R⁴ is

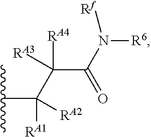

wherein each of R^{A1} and R^{A2} is independently hydrogen, C₁-C₁₀ alkyl, C₃-C₆ cycloalkyl, phenyl, or monocyclic heteroaryl, or R^{A1} and R^{A2} together with the atom to which they are attached form a C₃-C₆ cycloalkyl ring; each of R^{A3} and R^{A4} is independently hydrogen, C₁-C₁₀ alkyl, C₃-C₆ cycloalkyl, phenyl, or monocyclic heteroaryl, or R^{A3} and R^{A4} together with the atom to which they are attached form a C₃-C₆ cycloalkyl ring; R^{f} is hydrogen or C₁-C₁₀ alkyl; and R⁶ is hydrogen, C₁-C₁₀ alkyl, C₃-C₆ cycloalkyl, phenyl, or monocyclic heteroaryl.

In some embodiments, each of R⁹ and R¹⁰ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R⁹ and R¹⁰ together with the atom to which they are attached form a 3- to 6-membered heterocycloalkyl ring or a heteroaryl ring that is unsubstituted or substituted with one to five R^{A}. In some embodiments, each of R⁹ and R¹⁰ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R⁹ and R¹⁰ together with the atom to which they are attached form a 3- to 6-membered heterocycloalkyl ring or a heteroaryl ring that is unsubstituted or substituted with one to three R^{A}.

In some embodiments, each of R¹¹ and R¹² is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R¹¹ and R¹² together with the atoms to which they are attached form a 3- to 6-membered heterocycloalkyl ring that is unsubstituted or substituted with one to five R^{A}. In some embodiments, each of R¹¹ and R¹² is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R¹¹ and R¹² together with the atoms to which they are attached form a 3- to 6-membered heterocycloalkyl ring that is unsubstituted or substituted with one to three R^{A}.

In some embodiments, each R^{A} is independently C₁-C₁₀ alkyl, C₃-C₆ heteroalkyl, C₃-C₈ cycloalkyl, 3- to 6-membered heterocycloalkyl, phenyl, monocyclic heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR¹³, —NR(R¹⁸)R¹⁹, —C(O)R¹⁴, —OC(O)R¹⁵, —OC(O)OR¹⁶, —OC(O)N(R¹⁸)R¹⁹, or —OP(O)OR²⁰(OR²¹). In some embodiments, each R^{A} is independently C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R^{1′})R¹⁹, —OC(O)N(R^{1′})R¹⁹, or —OP(O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR¹³, —NR(R¹⁸)R¹⁹, —C(O)R¹⁴, —OC(O)R¹⁵, —OC(O)OR¹⁶, —OC(O)N(R^{1′})R¹⁹, or —OP(O)OR²⁰(OR²¹).

In some embodiments, each of R¹³, R¹⁴, R¹⁵, R¹⁶, or R¹⁷ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, and heteroaryl is unsubstituted or substituted with one to five R^{B}. In some embodiments, each of R¹³, R¹⁴, R¹⁵, R¹⁶, or R¹⁷ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, and heteroaryl is unsubstituted or substituted with one to three R^{B}.

In some embodiments, each of R¹⁸ and R¹⁹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}; or R¹⁸ and R¹⁹ together with the atom to which they are attached form a 3- to 6-membered heterocycloalkyl ring or heteroaryl ring, each of which is unsubstituted or substituted with one to five R^{B}. In some embodiments, each of R¹⁸ and R¹⁹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}; or R¹⁸ and R¹⁹ together with the atom to which they are attached form a 3- to 6-membered heterocycloalkyl ring or heteroaryl ring, each of which is unsubstituted or substituted with one to three R^{B}.

In some embodiments, each of R²⁰ and R²¹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}, or R²⁰ and R²¹ together with the atoms to which they are attached form a 3- to 6-membered heterocycloalkyl ring that is unsubstituted or substituted with one to five R^{B}. In some embodiments, each of R²⁰ and R²¹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}, or R²⁰ and R²¹ together with the atoms to which they are attached form a 3- to 6-membered heterocycloalkyl ring that is unsubstituted or substituted with one to three R^{B}.

In some embodiments, each R^{B} is independently halogen, amino, cyano, hydroxyl, C₁-C₁₀ alkyl, C₃-C₆ heteroalkyl, C₃-C₈ cycloalkyl, 3- to 6-membered heterocycloalkyl, phenyl, monocyclic heteroaryl, benzyl, —C(O)CH₃, —C(O)Ph, or (monocyclic heteroaryl)-C₁-C₄ alkyl wherein cycloalkyl, heterocycloalkyl, phenyl, or heteroaryl is unsubstituted or substituted with one to five halogen, amino, cyano, hydroxyl, C₁-C₆ alkyl, C₁-C₆ acetyl, or benzoyl. In some embodiments, each R^{B} is independently halogen, amino, cyano, hydroxyl, C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, benzyl, —C(O)CH₃, —C(O)Ph, or (5- or 6-membered monocyclic heteroaryl)-CH₂—, wherein cycloalkyl, heterocycloalkyl, phenyl, or heteroaryl is unsubstituted or substituted with one to three halogen, amino, cyano, hydroxyl, C₁-C₆ alkyl, C₁-C₆ acetyl, or benzoyl.

In some embodiments, compounds of Formula (I) have Formula (Ia),

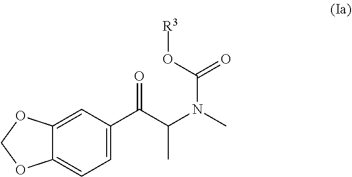

or a pharmaceutically acceptable salt thereof, wherein R³ is alkyl, alkenyl, haloalkyl, heteroalkyl, cycloalkyl, heterocycloalkyl, aryl, or heteroaryl, each of which is substituted or unsubstituted.

In some embodiments of Formulas (I) and (Ia), R³ is alkyl that is substituted. In some embodiments is a compound of Formula (I), wherein R³ is alkyl substituted with heteroalkyl, heterocycloalkyl, or heteroaryl, wherein each of heteroalkyl, heterocycloalkyl, and heteroaryl is unsubstituted or substituted. In some embodiments of Formula (Ia) R³ is alkyl that is unsubstituted. In some embodiments of Formula (Ia), R³ is heteroalkyl. In some embodiments of Formula (Ia), R³ is heteroalkyl that is unsubstituted. In some embodiments of Formula (I), R³ is ethyl.

In some embodiments of Formula (I), R¹ is —C(O)OR³, wherein R³ is alkyl. In some embodiments of Formula (I), R¹ is —C(O)OR³, wherein R³ is alkyl substituted with heterocycloalkyl. In some embodiments of Formula (I), R¹ is —C(O)OR³, wherein R³ is alkyl substituted with —N(R¹³)C(O)OR¹⁴. In some embodiments of Formula (I), R¹³ is hydrogen or alkyl. In some embodiments of Formula (I), R¹⁴ is alkyl, aryl, or heteroaryl.

In some embodiments of compounds of Formula (Ia,) R³ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl. In some embodiments is a compound of Formula (Ia), wherein R³ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formulas (I) and (Ia), wherein the compound is:

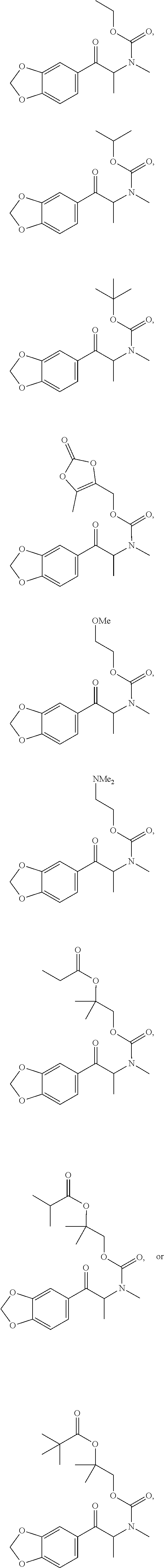

or a pharmaceutically acceptable salt thereof.

In some embodiments is a compound of Formulas (I) and (Ia), wherein the compound is:

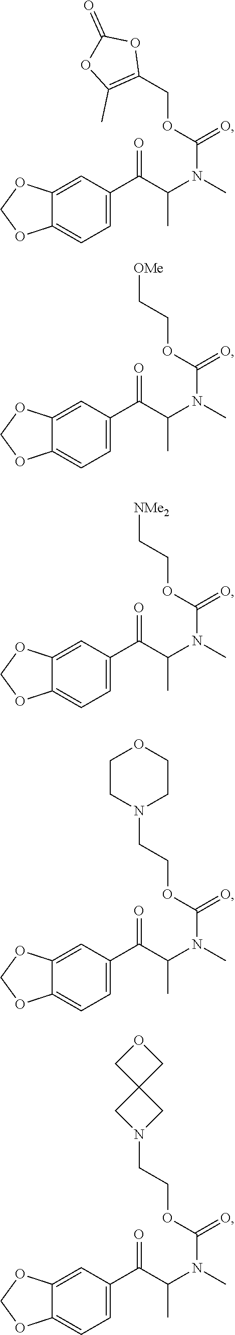 [SMILES: Cc1oc(=O)oc1COC(=O)N(C)C(C)C(=O)c1ccc2c(c1)OCO2 ; COCCOC(=O)N(C)C(C)C(=O)c1ccc2c(c1)OCO2 ; CC(C(=O)c1ccc2c(c1)OCO2)N(C)C(=O)OCCN(C)C ; CC(C(=O)c1ccc2c(c1)OCO2)N(C)C(=O)OCCN1CCOCC1 ; CC(C(=O)c1ccc2c(c1)OCO2)N(C)C(=O)OCCN1CC2(COC2)C1]

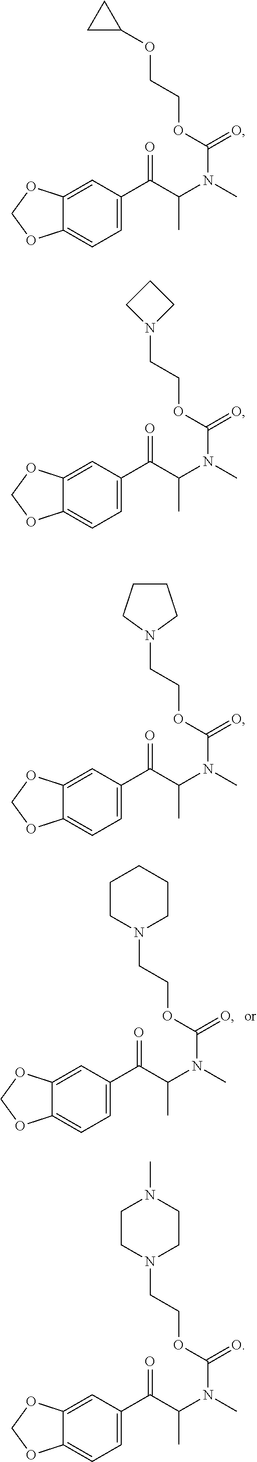 [SMILES: CC(C(=O)c1ccc2c(c1)OCO2)N(C)C(=O)OCCOC1CC1 ; CC(C(=O)c1ccc2c(c1)OCO2)N(C)C(=O)OCCN1CCC1 ; CC(C(=O)c1ccc2c(c1)OCO2)N(C)C(=O)OCCN1CCCC1 ; CC(C(=O)c1ccc2c(c1)OCO2)N(C)C(=O)OCCN1CCCCC1 ; CC(C(=O)c1ccc2c(c1)OCO2)N(C)C(=O)OCCN1CCN(C)CC1]

or a pharmaceutically acceptable salt thereof.

In some embodiments is a compound of Formulas (I) and (Ia), the compound is:

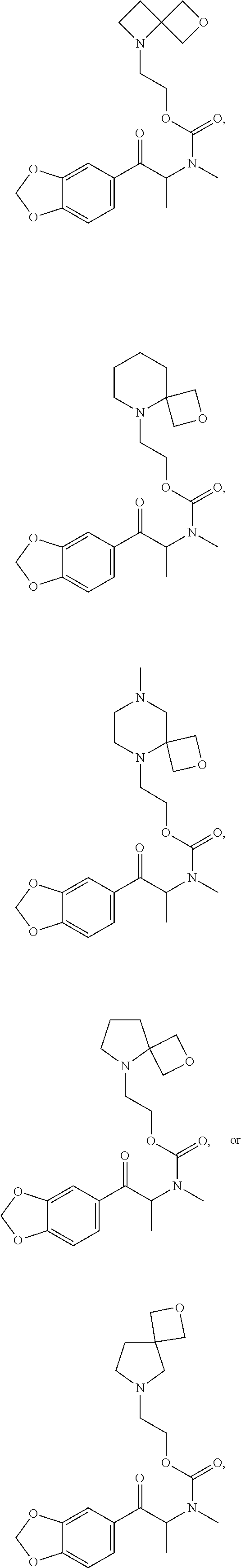 [SMILES: CC(C(=O)c1ccc2c(c1)OCO2)N(C)C(=O)OCCN1CCC12COC2 ; CC(C(=O)c1ccc2c(c1)OCO2)N(C)C(=O)OCCN1CCCCC12COC2 ; CC(C(=O)c1ccc2c(c1)OCO2)N(C)C(=O)OCCN1CCN(C)CC12COC2 ; CC(C(=O)c1ccc2c(c1)OCO2)N(C)C(=O)OCCN1CCCC12COC2 ; "or" ; CC(C(=O)c1ccc2c(c1)OCO2)N(C)C(=O)OCCN1CCC2(COC2)C1]

or a pharmaceutically acceptable salt thereof.

In some embodiments is a compound of Formulas (I) and (Ia), R³ is cycloalkyl that is substituted or unsubstituted. In some embodiments is a compound of Formula (I), wherein R³ is cycloalkyl that is substituted. In some embodiments is a compound of Formula (I), wherein R³ is cycloalkyl that is substituted with heteroalkyl, heterocycloalkyl, or amino. In some embodiments is a compound of Formulas (I) and (Ia), R¹ is cycloalkyl that is substituted with amino, aminoalkyl, or a nitrogen-containing heterocycle.

In some embodiments is a compound of Formulas (I) and (Ia), compounds have the structure of Formula (Ia-1), or a pharmaceutically acceptable salt thereof:

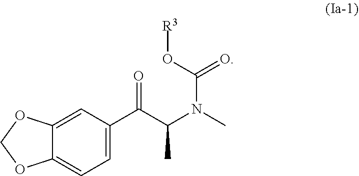

In some embodiments of Formulas (I), (Ia) and (Ia-1), wherein the compound is:

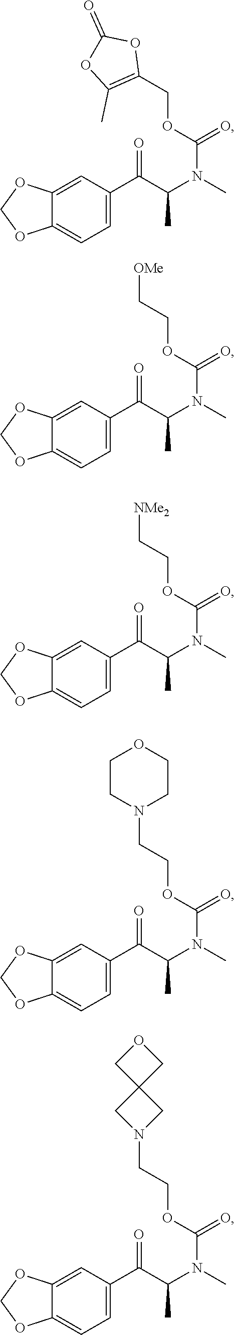 [SMILES: Cc1oc(=O)oc1COC(=O)N(C)[C@@H](C)C(=O)c1ccc2c(c1)OCO2 ; COCCOC(=O)N(C)[C@@H](C)C(=O)c1ccc2c(c1)OCO2 ; C[C@@H](C(=O)c1ccc2c(c1)OCO2)N(C)C(=O)OCCN(C)C ; C[C@@H](C(=O)c1ccc2c(c1)OCO2)N(C)C(=O)OCCN1CCOCC1 ; C[C@@H](C(=O)c1ccc2c(c1)OCO2)N(C)C(=O)OCCN1CC2(COC2)C1]

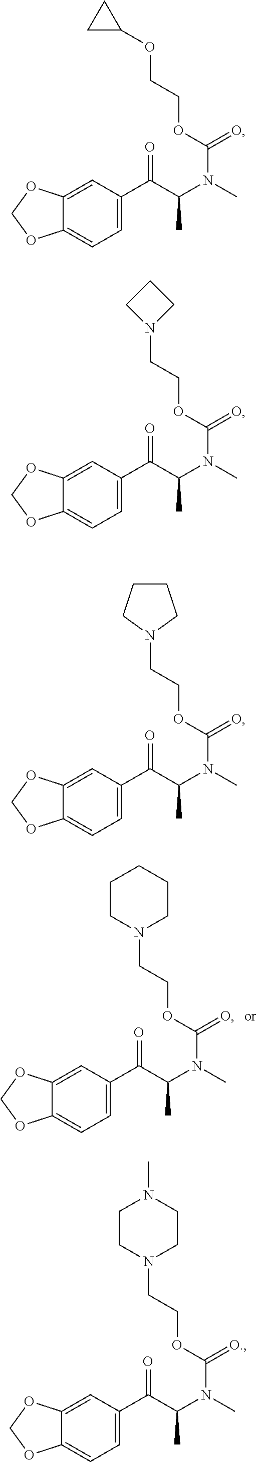 [SMILES: C[C@@H](C(=O)c1ccc2c(c1)OCO2)N(C)C(=O)OCCOC1CC1 ; C[C@@H](C(=O)c1ccc2c(c1)OCO2)N(C)C(=O)OCCN1CCC1 ; C[C@@H](C(=O)c1ccc2c(c1)OCO2)N(C)C(=O)OCCN1CCCC1 ; C[C@@H](C(=O)c1ccc2c(c1)OCO2)N(C)C(=O)OCCN1CCCCC1 ; C[C@@H](C(=O)c1ccc2c(c1)OCO2)N(C)C(=O)OCCN1CCN(C)CC1]

or a pharmaceutically acceptable salt thereof.

In some embodiments is a compound of Formulas (I), (Ia) and (Ia-1), the compound is:

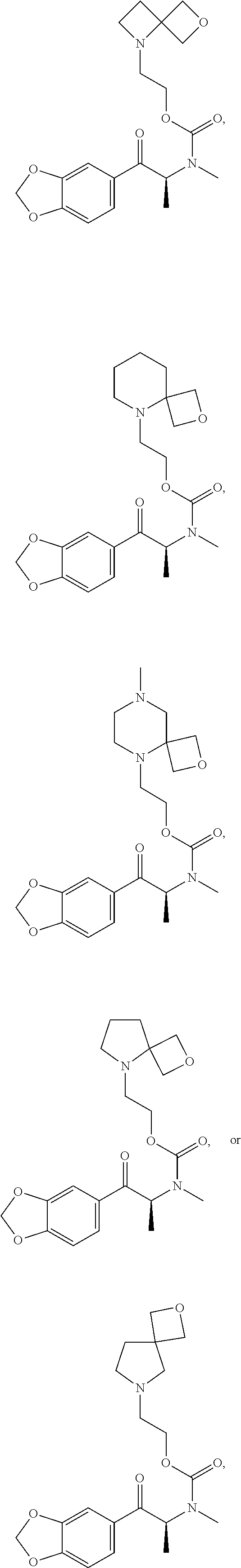 [SMILES: C[C@@H](C(=O)c1ccc2c(c1)OCO2)N(C)C(=O)OCCN1CCC12COC2 ; C[C@@H](C(=O)c1ccc2c(c1)OCO2)N(C)C(=O)OCCN1CCCCC12COC2 ; C[C@@H](C(=O)c1ccc2c(c1)OCO2)N(C)C(=O)OCCN1CCN(C)CC12COC2 ; C[C@@H](C(=O)c1ccc2c(c1)OCO2)N(C)C(=O)OCCN1CCCC12COC2 ; "or" ; C[C@@H](C(=O)c1ccc2c(c1)OCO2)N(C)C(=O)OCCN1CCC2(COC2)C1]

or a pharmaceutically acceptable salt thereof.

In some embodiments of Formulas (I) and (Ia) compounds having the structure of Formula (Ia-2), or a pharmaceutically acceptable salt thereof, are provided herein:

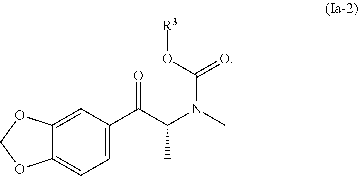

In some embodiments herein is provided a compound of Formulas (I), (Ia) and (Ia-2), wherein the compound is:

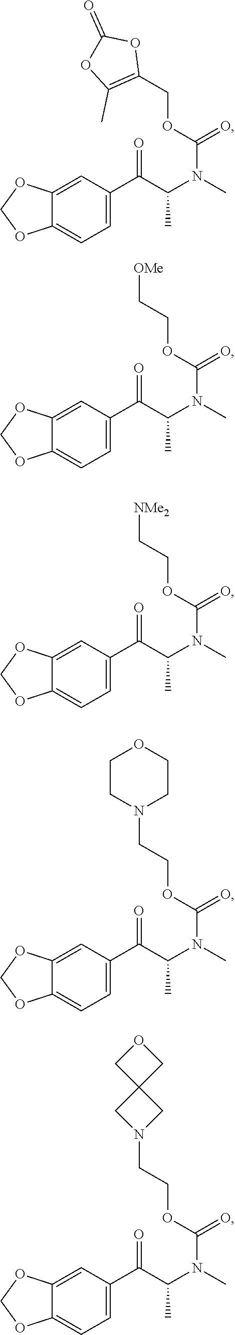 [SMILES: Cc1oc(=O)oc1COC(=O)N(C)[C@H](C)C(=O)c1ccc2c(c1)OCO2 ; COCCOC(=O)N(C)[C@H](C)C(=O)c1ccc2c(c1)OCO2 ; C[C@H](C(=O)c1ccc2c(c1)OCO2)N(C)C(=O)OCCN(C)C ; C[C@H](C(=O)c1ccc2c(c1)OCO2)N(C)C(=O)OCCN1CCOCC1 ; C[C@H](C(=O)c1ccc2c(c1)OCO2)N(C)C(=O)OCCN1CC2(COC2)C1]

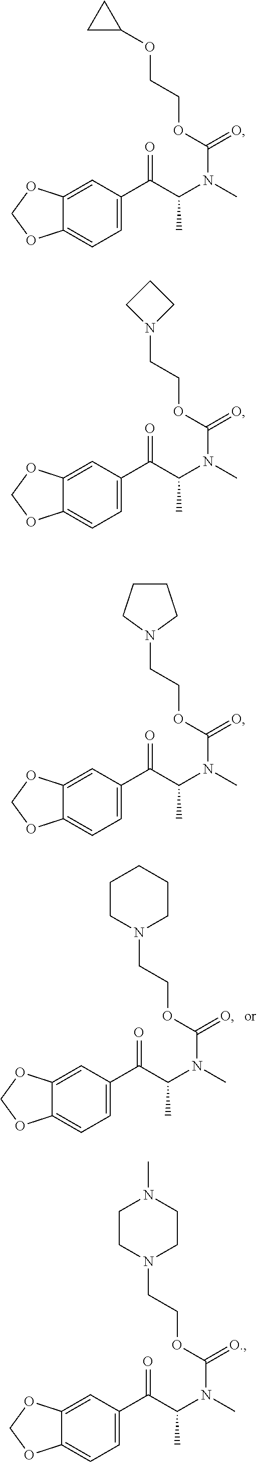 [SMILES: C[C@H](C(=O)c1ccc2c(c1)OCO2)N(C)C(=O)OCCOC1CC1 ; C[C@H](C(=O)c1ccc2c(c1)OCO2)N(C)C(=O)OCCN1CCC1 ; C[C@H](C(=O)c1ccc2c(c1)OCO2)N(C)C(=O)OCCN1CCCC1 ; C[C@H](C(=O)c1ccc2c(c1)OCO2)N(C)C(=O)OCCN1CCCCC1 ; C[C@H](C(=O)c1ccc2c(c1)OCO2)N(C)C(=O)OCCN1CCN(C)CC1]

or a pharmaceutically acceptable salt thereof.

In some embodiments disclosed herein is a compound of Formulas (I), (Ia) and (Ia-2), wherein the compound is:

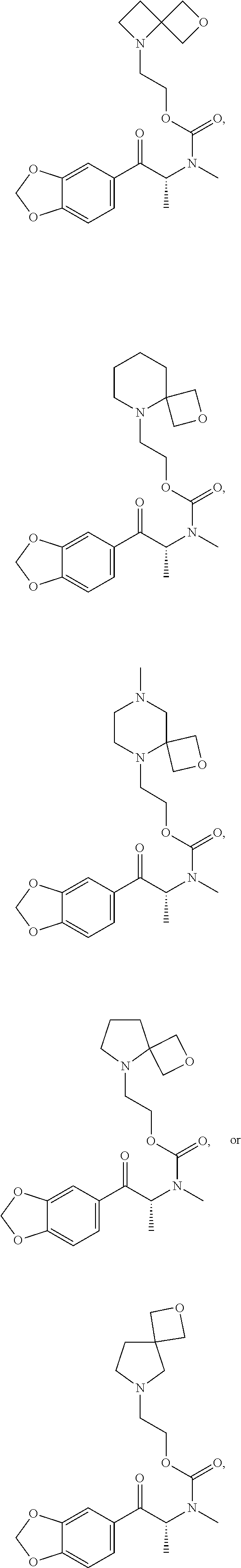 [SMILES: C[C@H](C(=O)c1ccc2c(c1)OCO2)N(C)C(=O)OCCN1CCC12COC2 ; C[C@H](C(=O)c1ccc2c(c1)OCO2)N(C)C(=O)OCCN1CCCCC12COC2 ; C[C@H](C(=O)c1ccc2c(c1)OCO2)N(C)C(=O)OCCN1CCN(C)CC12COC2 ; C[C@H](C(=O)c1ccc2c(c1)OCO2)N(C)C(=O)OCCN1CCCC12COC2 ; "or" ; C[C@H](C(=O)c1ccc2c(c1)OCO2)N(C)C(=O)OCCN1CCC2(COC2)C1]

or a pharmaceutically acceptable salt thereof.

In some embodiments of compounds of Formula (I), (Ia), (Ia-1) or (Ia-2), if R³ is unsubstituted alkyl, then R³ is not tert-butyl.

In some embodiments is a compound of Formula (I) and (Ia) having the structure of Formula (Ia1), or a pharmaceutically acceptable salt thereof:

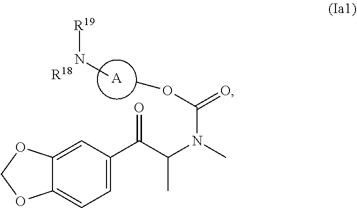

wherein

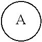

is cycloalkyl or heterocycloalkyl, and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In some embodiments is a compound of Formula (I), (Ia) and (Ia1) having the structure of Formula (Ia1-1), or a pharmaceutically acceptable salt thereof:

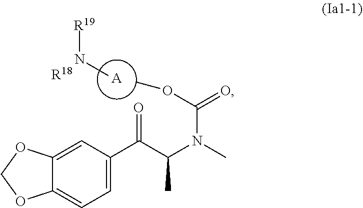

wherein

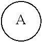

is cycloalkyl or heterocycloalkyl, and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In some embodiments is a compound of Formula (Ia) and (Ia1) having the structure of Formula (Ia1-2), or a pharmaceutically acceptable salt thereof:

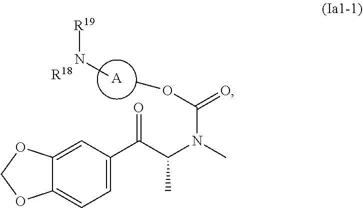

wherein

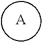

is cycloalkyl or heterocycloalkyl, and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In some embodiments is a compound of Formulas (I), (Ia) and (Ia1) having the structure of Formula (Ia2), or a pharmaceutically acceptable salt thereof:

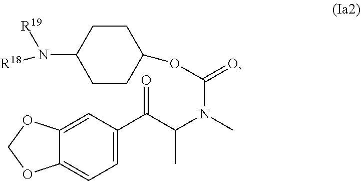

and each of each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In some embodiments is a compound of Formulas (I), (Ia), (Ia1) and (Ia2) having the structure of Formula (Ia2-1), or a pharmaceutically acceptable salt thereof:

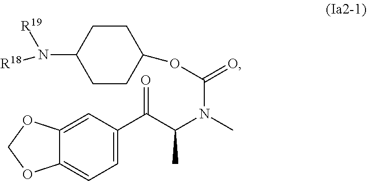

and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In some embodiments is a compound of Formulas (I), (Ia), (Ia1) and (Ia2)having the structure of Formula (Ia2-2), or a pharmaceutically acceptable salt thereof:

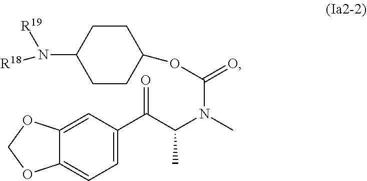

and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In some embodiments is a compound of Formulas (I), (Ia), (Ia1) and (Ia2), wherein the compound is:

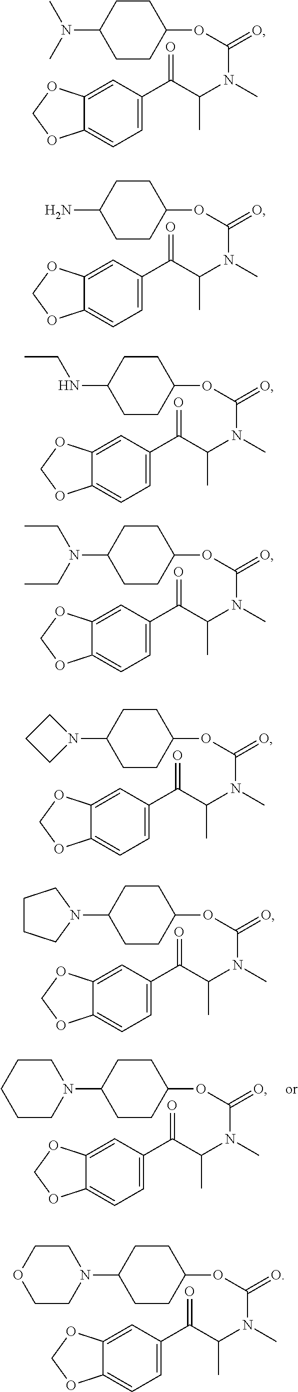

or a pharmaceutically acceptable salt thereof.

In another aspect, the present disclosure provides a compound of Formula (Ia3):

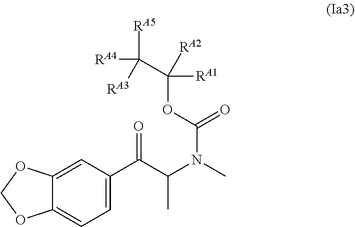

or a pharmaceutically acceptable salt thereof, wherein:
each of R^{A1}, R^{A2}, R^{A3}, and R^{A4} is independently hydrogen or alkyl, and
R^{A5} is heteroalkyl, heterocycloalkyl, heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶.

In some embodiments of Formula (Ia3) one of R^{A1}, R^{A2}, R^{A3}, and R^{A4} is alkyl, and each of R^{A1}, R^{A2}, R^{A3}, and R^{A4} that is not alkyl is hydrogen. In some of Formula (Ia3), two of R^{A1}, R^{A2}, R^{A3}, and R^{A4} are alkyl, and each of R^{A1}, R^{A2}, R^{A3}, and R^{A4} that is not alkyl is hydrogen. In some embodiments of Formula (Ia3), each of R^{A1}, R^{A2}, R^{A3}, and R^{A4} is hydrogen. In some embodiments of Formula (Ia3), R^{A3}, and R^{A4} together with the atom to which they are attached form a cycloalkyl ring, and R^{A1} and R^{A2} are each hydrogen.

In some embodiments is a compound of Formula (Ia3) R^{A5} is C(O)OR¹³, and R¹³ is selected from methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, and 6-pyrimidyl. In some embodiments is a compound of Formula (Ia3), wherein each of R^{A1}, R^{A2}, R^{A3}, and R^{A4} is hydrogen.

In some embodiments is a compound of Formula (Ia3), wherein the compound is:

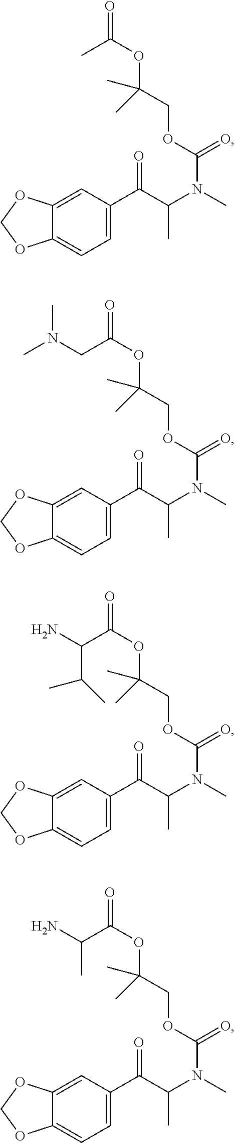 [SMILES: CC(=O)OC(C)(C)COC(=O)N(C)C(C)C(=O)c1ccc2c(c1)OCO2 ; CC(C(=O)c1ccc2c(c1)OCO2)N(C)C(=O)OCC(C)(C)OC(=O)CN(C)C ; CC(C)C(N)C(=O)OC(C)(C)COC(=O)N(C)C(C)C(=O)c1ccc2c(c1)OCO2 ; CC(N)C(=O)OC(C)(C)COC(=O)N(C)C(C)C(=O)c1ccc2c(c1)OCO2]

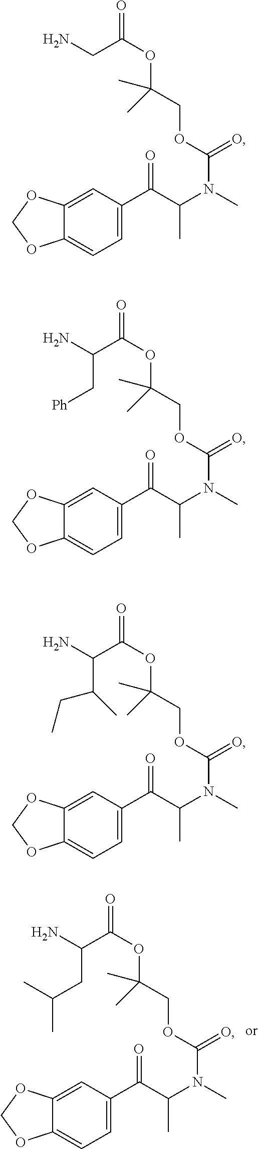

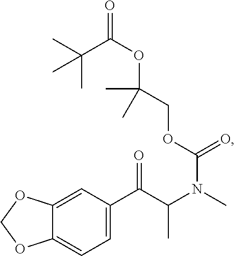 [SMILES: CC(C(=O)c1ccc2c(c1)OCO2)N(C)C(=O)OCC(C)(C)OC(=O)C(C)(C)C]

or a pharmaceutically acceptable salt thereof.

In some embodiments is a compound of Formula (Ia3), wherein the compound is:

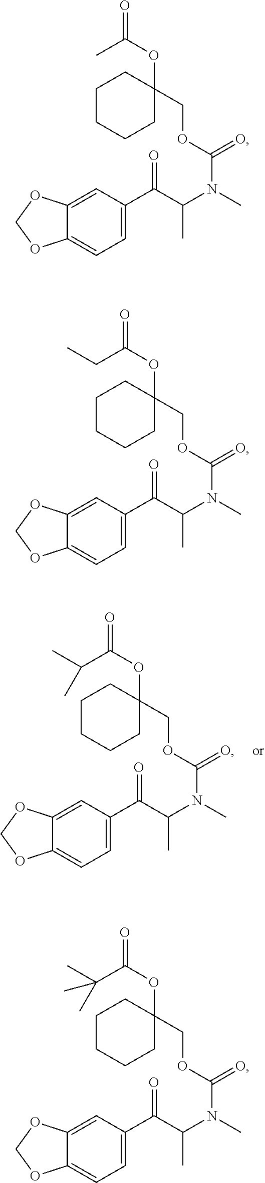 [SMILES: CC(=O)OC1(COC(=O)N(C)C(C)C(=O)c2ccc3c(c2)OCO3)CCCCC1 ; CCC(=O)OC1(COC(=O)N(C)C(C)C(=O)c2ccc3c(c2)OCO3)CCCCC1 ; CC(C)C(=O)OC1(COC(=O)N(C)C(C)C(=O)c2ccc3c(c2)OCO3)CCCCC1 ; CC(C(=O)c1ccc2c(c1)OCO2)N(C)C(=O)OCC1(OC(=O)C(C)(C)C)CCCCC1]

or a pharmaceutically acceptable salt thereof.

In some embodiments is a compound of Formula (Ia3), wherein the compound is:

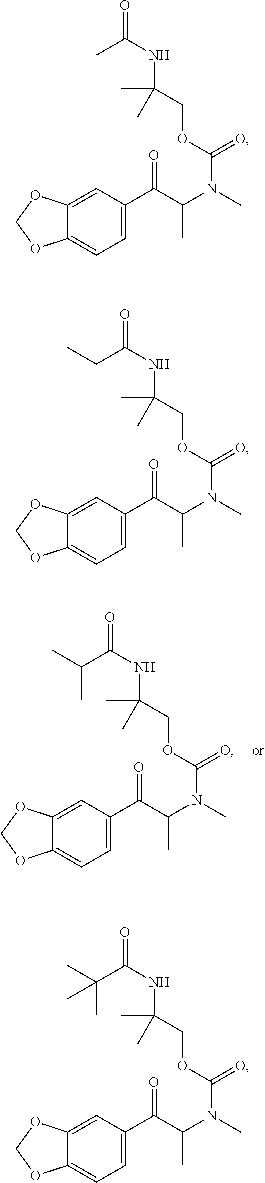 [SMILES: CC(=O)NC(C)(C)COC(=O)N(C)C(C)C(=O)c1ccc2c(c1)OCO2 ; CCC(=O)NC(C)(C)COC(=O)N(C)C(C)C(=O)c1ccc2c(c1)OCO2 ; CC(C)C(=O)NC(C)(C)COC(=O)N(C)C(C)C(=O)c1ccc2c(c1)OCO2 ; CC(C(=O)c1ccc2c(c1)OCO2)N(C)C(=O)OCC(C)(C)NC(=O)C(C)(C)C]

or a pharmaceutically acceptable salt thereof.

In some embodiments is a compound of Formula (Ia3) having the structure of Formula (Ia3-1), or a pharmaceutically acceptable salt thereof:

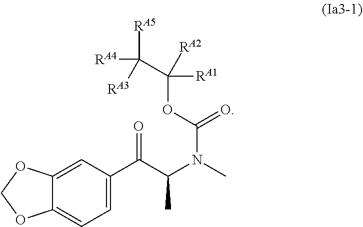

In some embodiments is a compound of Formula (Ia3) having the structure of Formula (Ia3-2), or a pharmaceutically acceptable salt thereof:

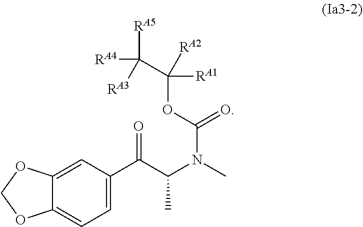

In another aspect, the present disclosure provides a compound of Formula (Ib):

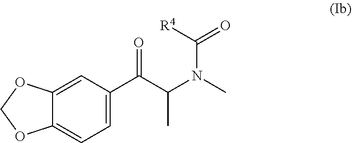

or a pharmaceutically acceptable salt thereof, wherein R⁴ is alkyl, alkenyl, haloalkyl, heteroalkyl, cycloalkyl, heterocycloalkyl, aryl, or heteroaryl, each of which is substituted or unsubstituted, or R⁴ and the carbonyl atom to which R⁴ is attached form an amino acid residue.

In some embodiments of Formula (Ib), R⁴ is alkyl. In some embodiments of a compound of Formula (Ib), R⁴ is CH₂CF₃. In some embodiments of a compound of Formula (Ib), R⁴ is unsubstituted alkyl. In some embodiments of Formula (Ib), R⁴ is methyl, ethyl, n-propyl, isopropyl, n-butyl, tert-butyl, 3-methyl-1-butyl, n-pentyl, n-hexyl, n-heptyl, n-octyl, or n-nonyl. In some embodiments a compound of Formula (Ib) is one wherein R⁴ is cycloalkyl. In some embodiments of compound of Formula (Ib), R⁴ is unsubstituted cycloalkyl. In some embodiments of compounds of Formula (Ib), R⁴ is cyclopropyl, cyclobutyl, cyclopentyl, cyclohexyl, or cycloheptyl. In some embodiments of a compound of Formula (Ib), the compound is one wherein R⁴ is aryl. In some embodiments of Formula (Ib), wherein R⁴ is substituted or unsubstituted phenyl. In other embodiments of Formula (Ib), R⁴ is heteroaryl and in certain such some embodiments of Formula (Ib), R⁴ is 2-pyridyl, 3-pyridyl, 4-pyridyl, 2-pyrimidyl, 3-pyrimidyl, or 6-pyrimidyl.

In some embodiments of Formula (Ib), R⁴ is R^{A} substituted with —OR¹³, —N(R¹⁸)R¹⁹, or —C(O)OR¹³, such as wherein R⁴ is alkyl, substituted with —OR¹³, —N(R¹⁸)R¹⁹, or —C(O)OR¹³. In some embodiments of Formula (Ib), R⁴ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Ib) R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(O)CH₃. In one embodiment of Formula (Ib) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Ib), R⁴ is heteroalkyl. In some embodiments of Formulas (I) and (Ib), R⁴ is CH₂CH₂OMe or CH₂CH₂SO₂Me. In some embodiments of Formulas (I) and (Ib), R⁴ is —(CH₂)ₙCO₂H, wherein n is 1, 2, 3, 4, 5, or 6. In some embodiments of Formulas (I) and (Ib), R⁴ is —(CH₂)ₙCO₂R¹³, wherein n is 1, 2, 3, 4, 5, or 6. In some embodiments of Formulas (I) and (Ib), R⁴ is —(CH₂)ₙCO₂R¹³, wherein R¹³ is alkyl. In some embodiments of Formulas (I) and (Ib), R⁴ is —(CH₂)ₙCO₂R¹³, wherein R¹³ is unsubstituted alkyl. In some embodiments of Formulas (I) and (Ib), R⁴ is —(CH₂)ₛCO₂R¹³, wherein R¹³ is methyl, ethyl, n-propyl, isopropyl, n-butyl, tert-butyl, or —CH(Et)₂.

In some embodiments of Formulas (I) and (Ib), R⁴ is —(CH₂)ₙOR¹³, wherein n is 1, 2, 3, 4, 5, 6 or 7. In some embodiments of Formulas (I) and (Ib), R⁴ is —(CH₂)ₙOR¹³, wherein R¹³ is alkyl. In some embodiments of Formulas (I) and (Ib), R⁴ is —(CH₂)ₙOR¹³, wherein R¹ is unsubstituted alkyl. In some embodiments of Formulas (I) and (Ib), R⁴ is —(CH₂)ₛOR¹³, wherein R¹³ is methyl, ethyl, n-propyl, isopropyl, n-butyl, tert-butyl, or —CH(Et)₂.

In some embodiments is a compound of Formula (Ib), the compound is:

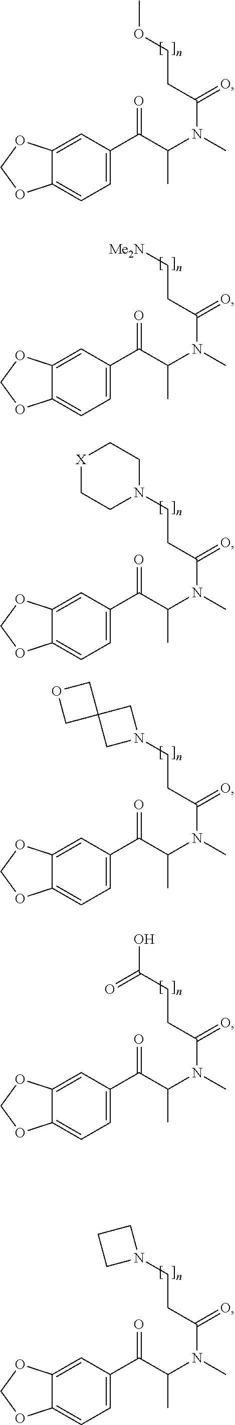

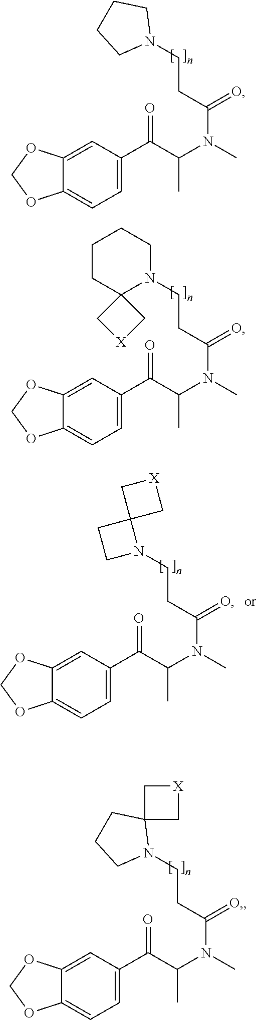

or a pharmaceutically acceptable salt thereof, wherein each n is independently 1, 2, 3, 4, 5, or 6; and each X is independently —O—, —S—, —S(O)—, —S(O)₂—, —NH—, or —NR^{B}, wherein R^{B} is selected from alkyl, heteroalkyl, —C(O)CH₃ and —C(O)Ph, each of which is substituted or unsubstituted.

In certain embodiments of compounds of Formulas (I) and (Ib), R⁴ is

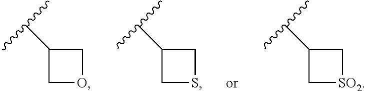 [SMILES: *C1COC1 ; *C1CSC1 ; *C1CS(=O)(=O)C1]

In some embodiments of Formula (Ib), R⁴ is

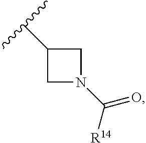

and in certain such embodiments of a compound of Formula (Ib), wherein R⁴ is

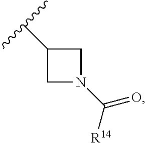

R¹⁴ is alkyl, cycloalkyl, or aryl, such as compounds wherein R¹⁴ is methyl, ethyl, n-propyl, isopropyl, or CH₂CH₂OMe. In some embodiments of Formula (Ib), wherein R⁴ is

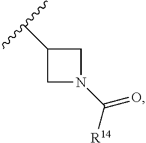

R¹⁴ is phenyl.

In some embodiments of Formulas (I) and (Ib), the compound is:

[symbol]

In some embodiments of compounds of Formula (Ib), R⁴ is

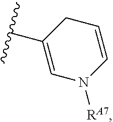

wherein R^{A7} is hydrogen or alkyl. In some embodiments of such compounds of Formula (Ib), R⁴ is

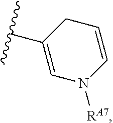

wherein R^{A7} is hydrogen. In some embodiments of Formula (Ib), R⁴ is

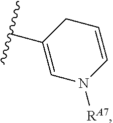

wherein R^{A7} is alkyl. In some embodiments of Formula (Ib), R⁴ is

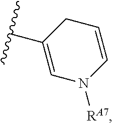

wherein R^{A7} is unsubstituted alkyl. In some embodiments of Formula (Ib), R⁴ is

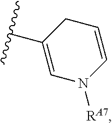

and R^{A7} is methyl, ethyl, n-propyl, isopropyl, or n-butyl.

In some embodiments of Formulas (I) and (Ib), R⁴ is —(CH₂)ₙN(R¹⁸)R¹⁹, wherein n is 1, 2, 3, 4, 5, 6 or 7. In some embodiments of Formulas (I) and (Ib), R⁴ is

—(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring, and in certain such embodiments the heterocycloalkyl ring is substituted with one or more R^{B}, such as wherein R^{B} is selected from alkyl, heteroalkyl, —C(O)CH₃ and —C(O)Ph.

In another aspect, the present disclosure provides a compound of Formula (Ib), wherein the compound is:

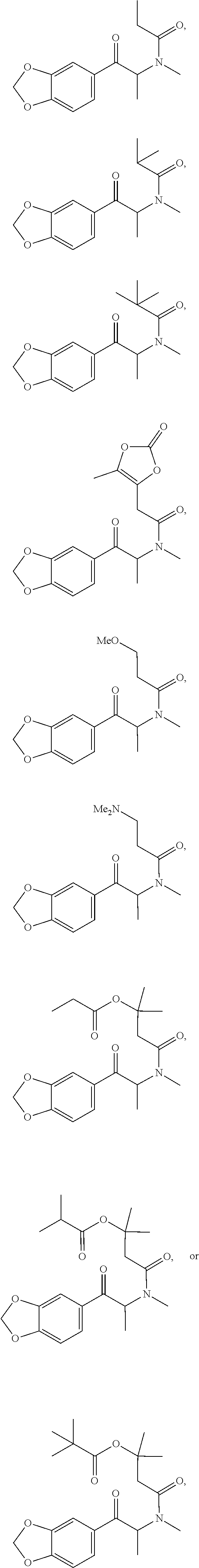

or a pharmaceutically acceptable salt thereof.

In another aspect, the present disclosure provides a compound of Formula (Ib), wherein the compound is:

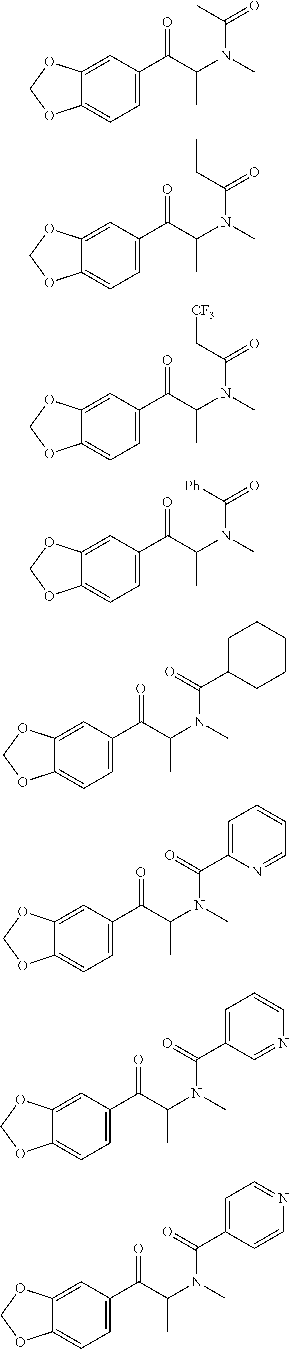

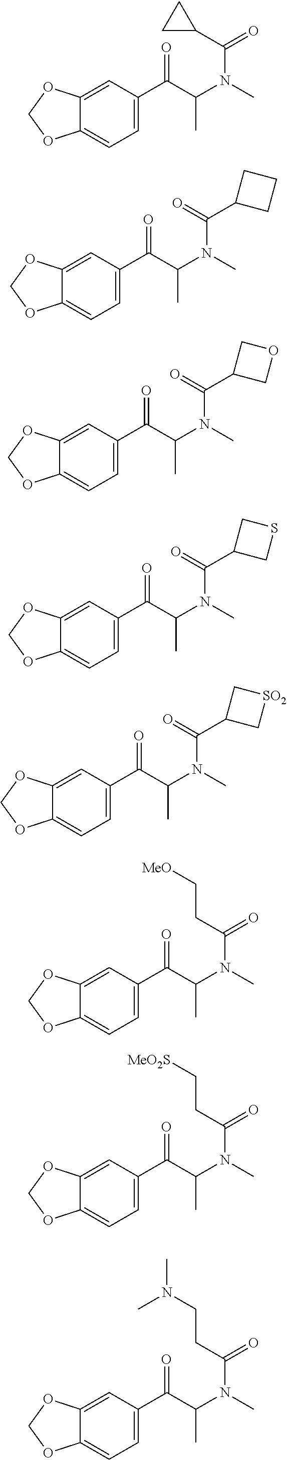

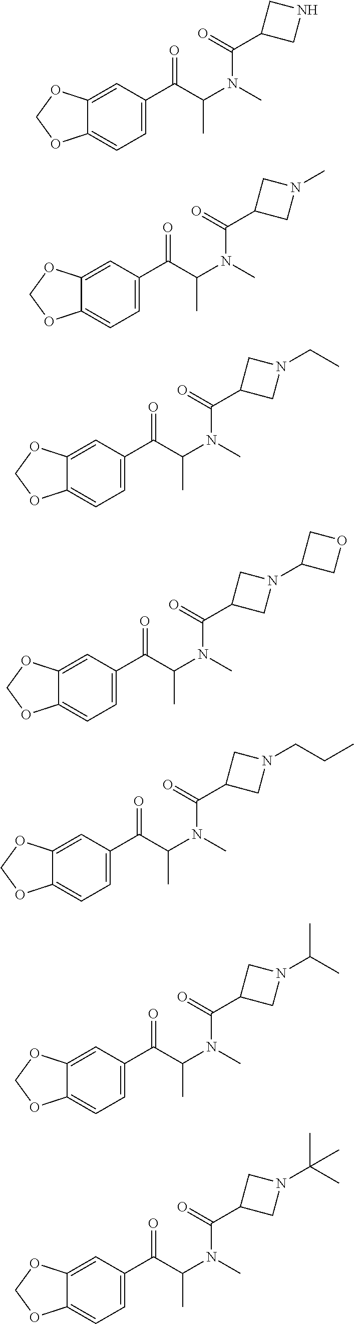

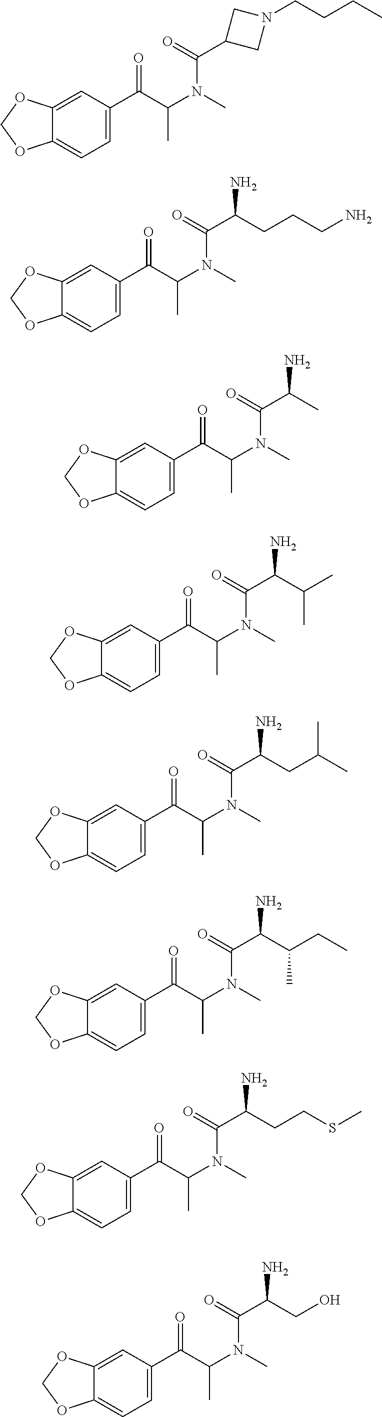

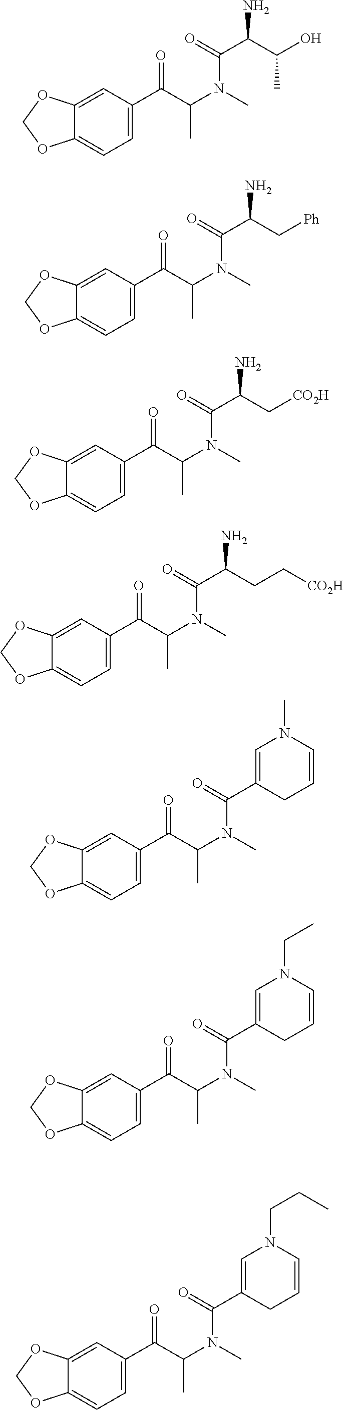

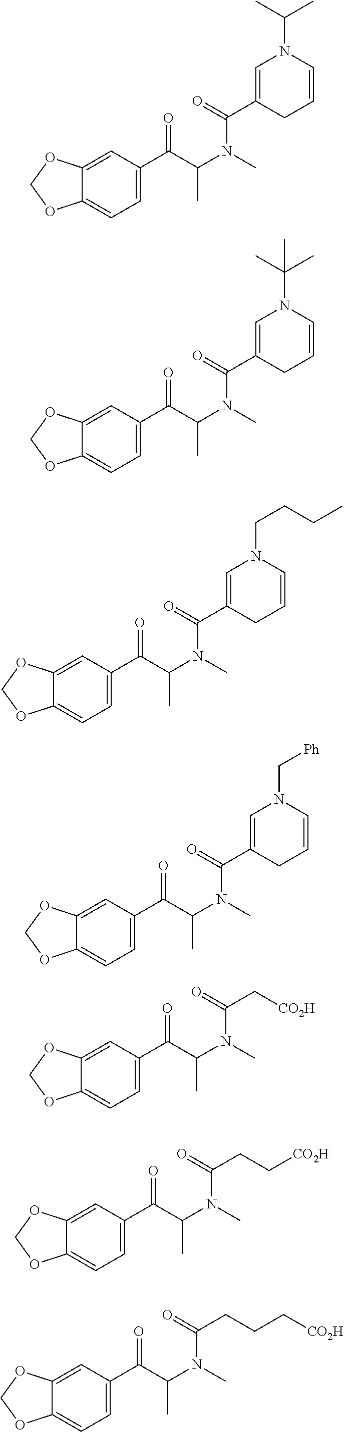

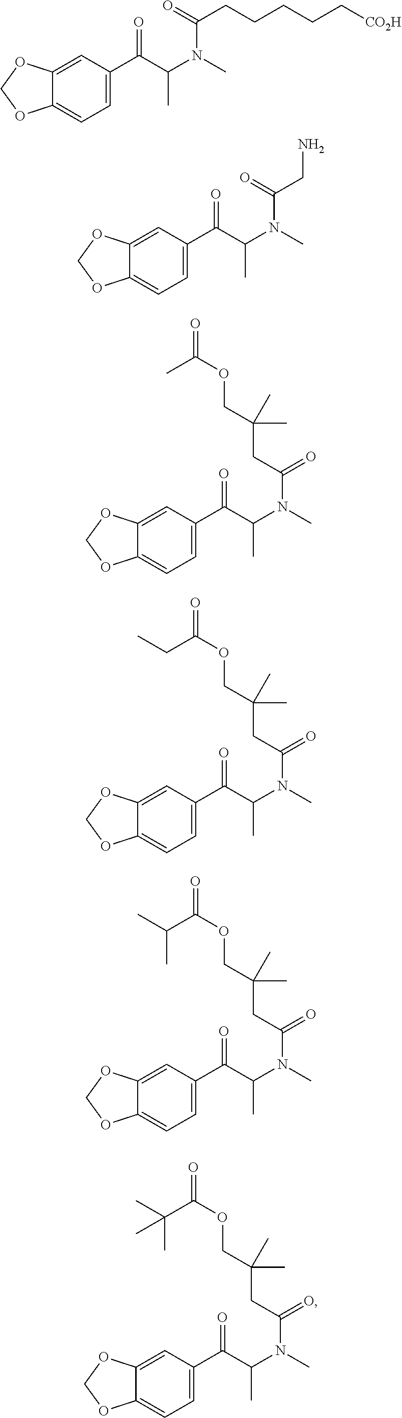

or a pharmaceutically acceptable salt thereof.

In some embodiments is a compound of Formula (Ib) having the structure of Formula (Ib-1), or a pharmaceutically acceptable salt thereof:

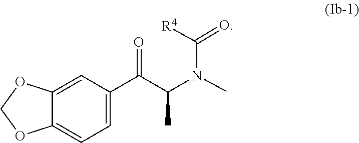

In some embodiments is a compound of Formulas (Ib) and (Ib-1), wherein the compound is:

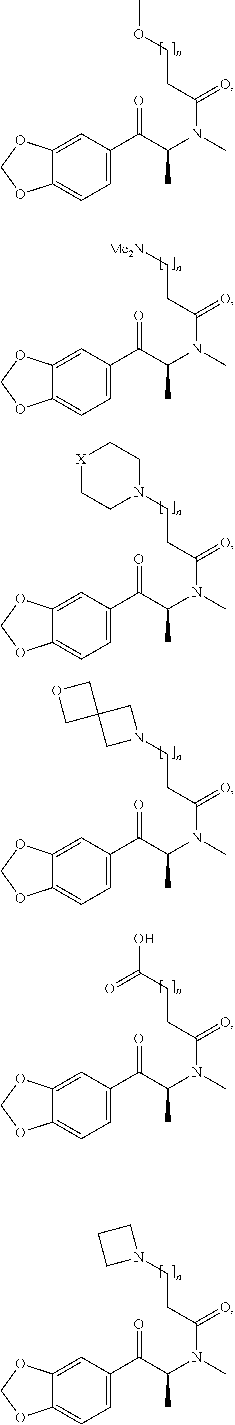

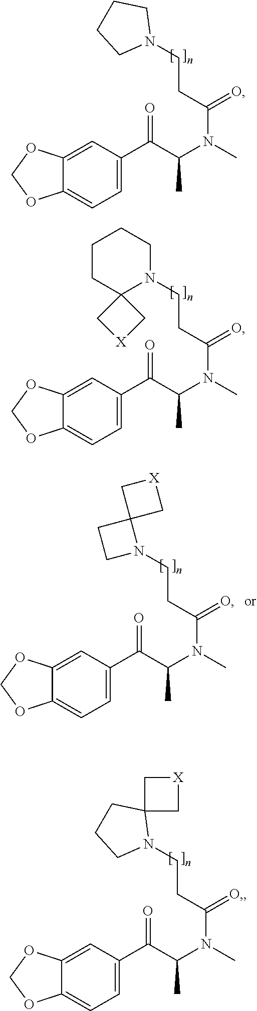

or a pharmaceutically acceptable salt thereof, wherein each n is independently 1, 2, 3, 4, 5, or 6; and each X is independently —O—, —S—, —S(O)—, —S(O)₂—, —NH—, or NR^{B}, wherein R^{B} is selected from alkyl, heteroalkyl, —C(O)CH₃ and —C(O)Ph, each of which is substituted or unsubstituted.

In some embodiments of Formulas (Ib) and (Ib-1), wherein the compound is:

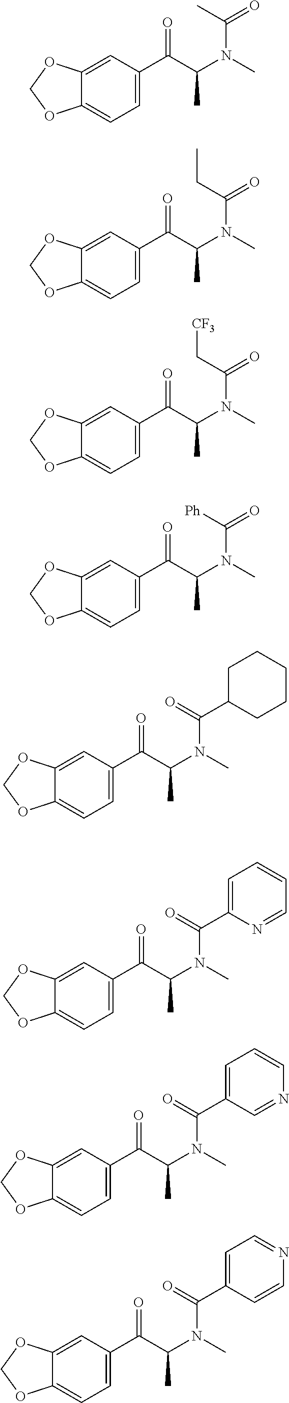

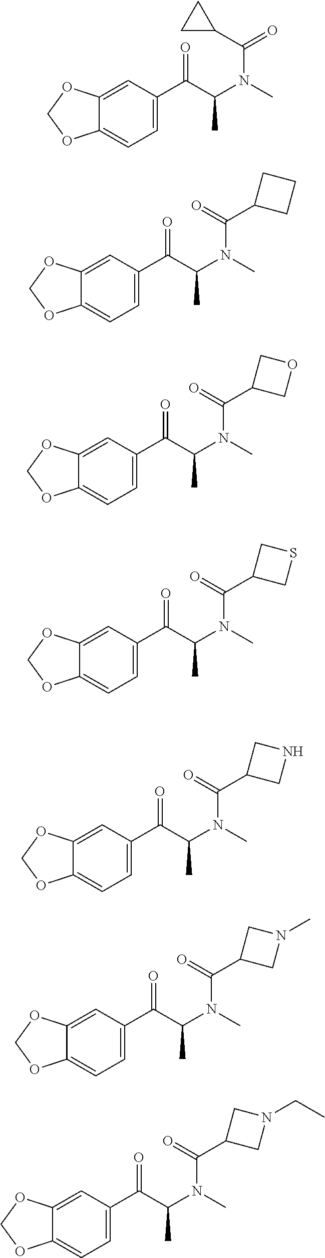

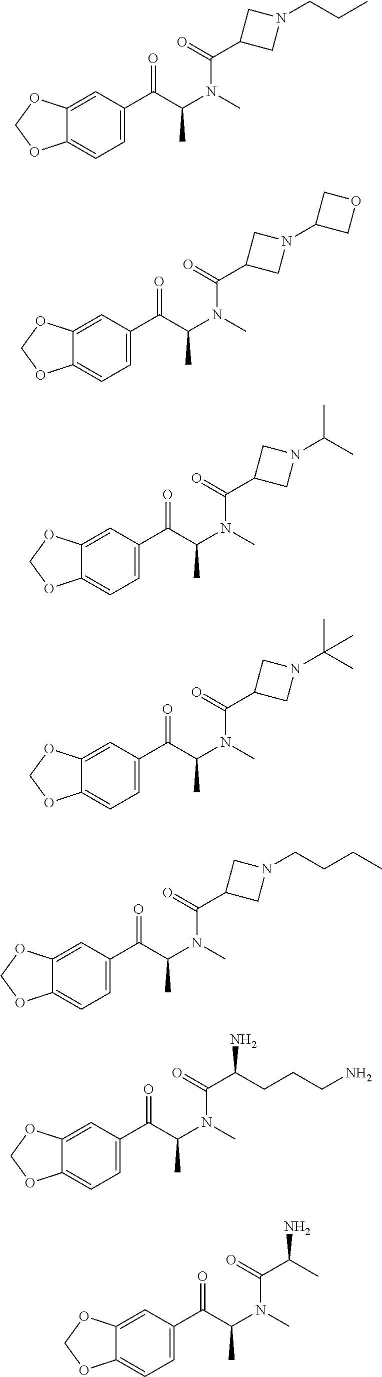

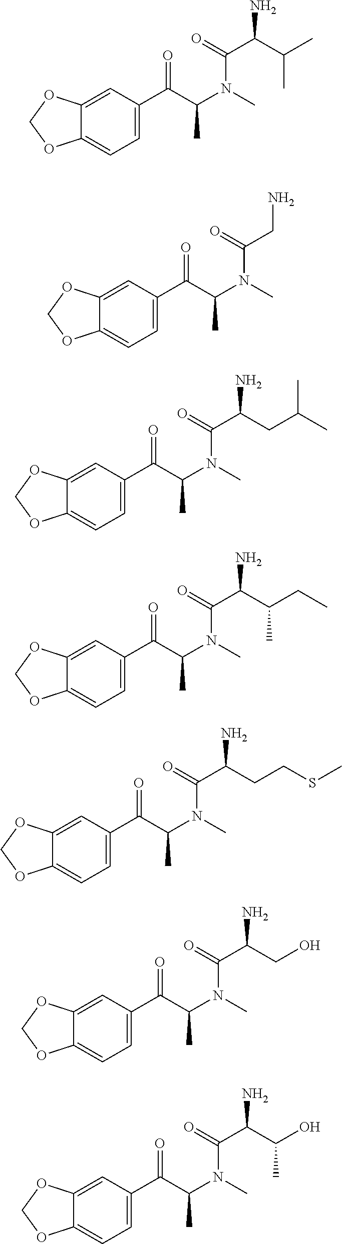

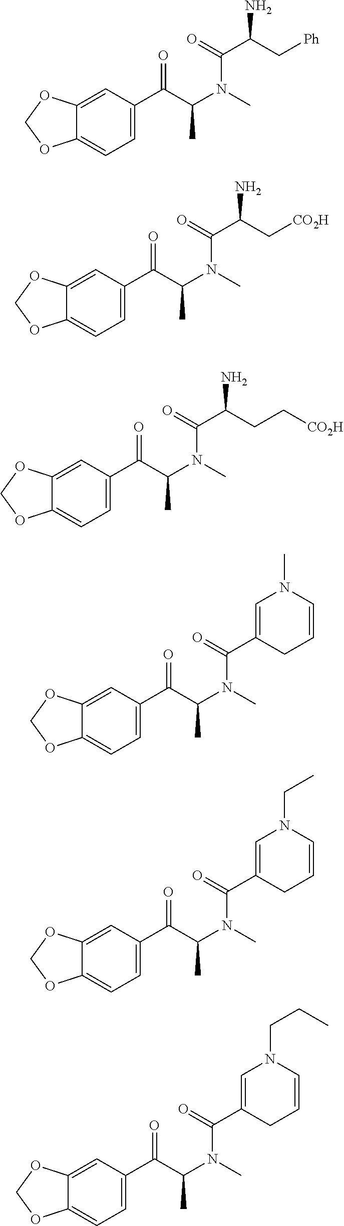 [SMILES: C[C@@H](C(=O)c1ccc2c(c1)OCO2)N(C)C(=O)[C@@H](N)Cc1ccccc1 ; C[C@@H](C(=O)c1ccc2c(c1)OCO2)N(C)C(=O)[C@@H](N)CC(=O)O ; C[C@@H](C(=O)c1ccc2c(c1)OCO2)N(C)C(=O)[C@@H](N)CCC(=O)O ; C[C@@H](C(=O)c1ccc2c(c1)OCO2)N(C)C(=O)C1=CN(C)C=CC1 ; CCN1C=CCC(C(=O)N(C)[C@@H](C)C(=O)c2ccc3c(c2)OCO3)=C1 ; CCCN1C=CCC(C(=O)N(C)[C@@H](C)C(=O)c2ccc3c(c2)OCO3)=C1]

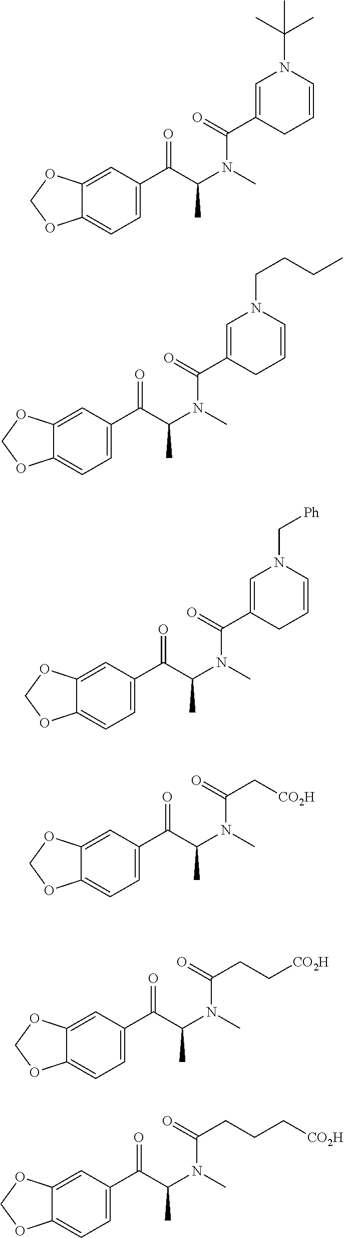 [SMILES: C[C@@H](C(=O)c1ccc2c(c1)OCO2)N(C)C(=O)C1=CN(C(C)(C)C)C=CC1 ; CCCCN1C=CCC(C(=O)N(C)[C@@H](C)C(=O)c2ccc3c(c2)OCO3)=C1 ; C[C@@H](C(=O)c1ccc2c(c1)OCO2)N(C)C(=O)C1=CN(Cc2ccccc2)C=CC1 ; C[C@@H](C(=O)c1ccc2c(c1)OCO2)N(C)C(=O)CC(=O)O ; C[C@@H](C(=O)c1ccc2c(c1)OCO2)N(C)C(=O)CCC(=O)O ; C[C@@H](C(=O)c1ccc2c(c1)OCO2)N(C)C(=O)CCCC(=O)O]

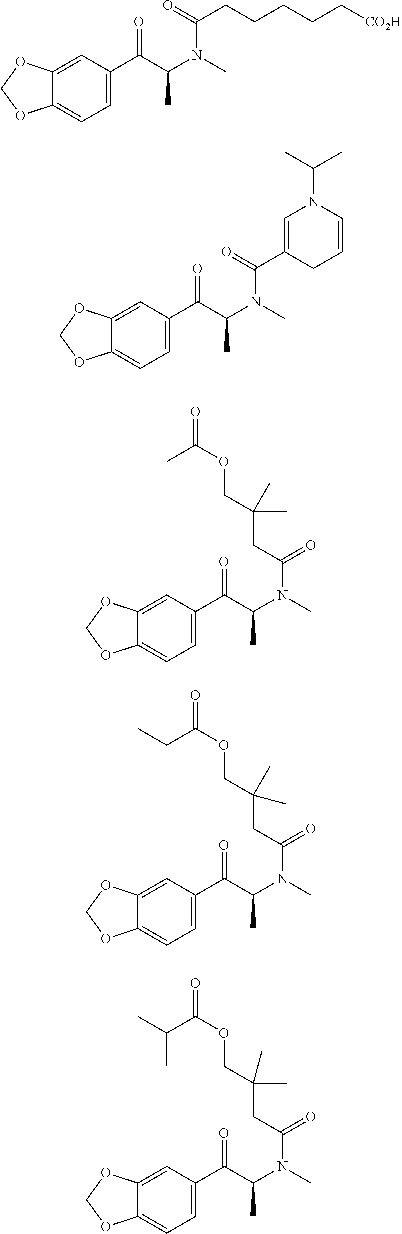

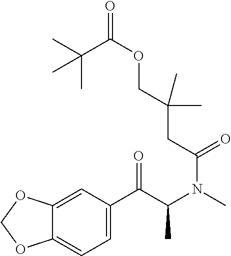 [SMILES: C[C@@H](C(=O)c1ccc2c(c1)OCO2)N(C)C(=O)CC(C)(C)COC(=O)C(C)(C)C]

or a pharmaceutically acceptable salt thereof,

In some embodiments is a compound of Formula (Ib) having the structure of Formula (Ib-2), or a pharmaceutically acceptable salt thereof:

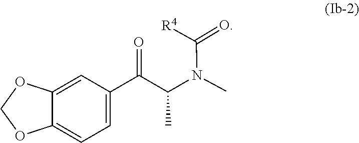

In some embodiments of Formulas (Ib) and (Ib-2, wherein the compound is:

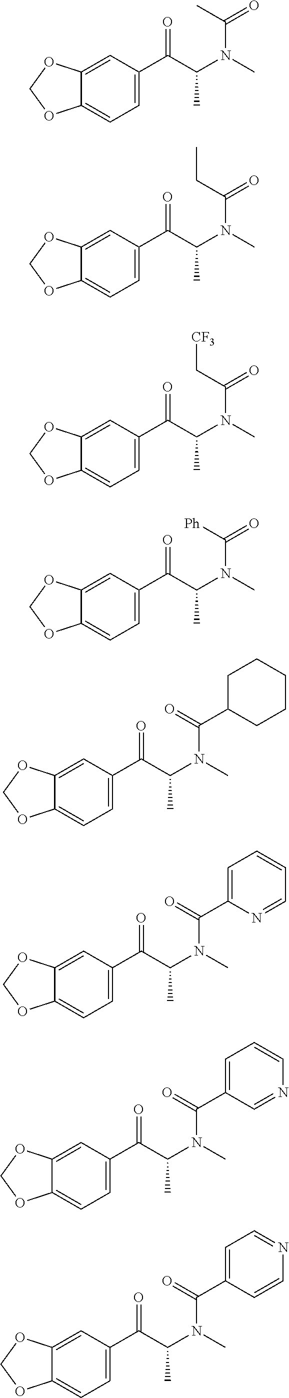

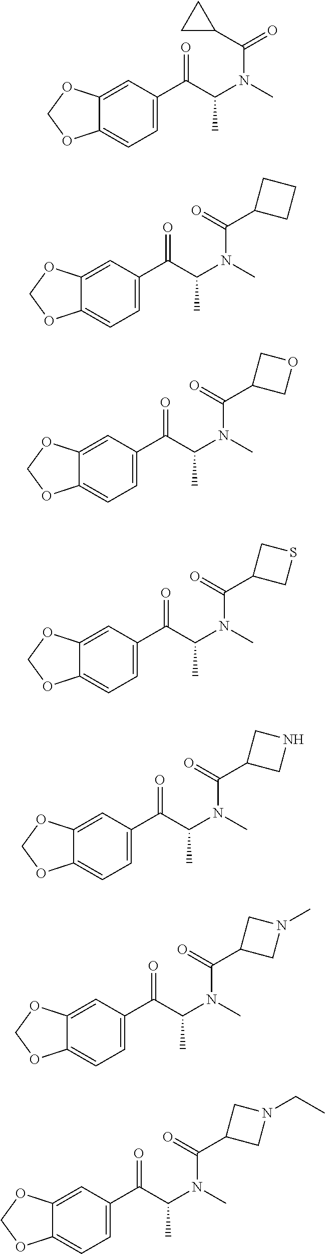

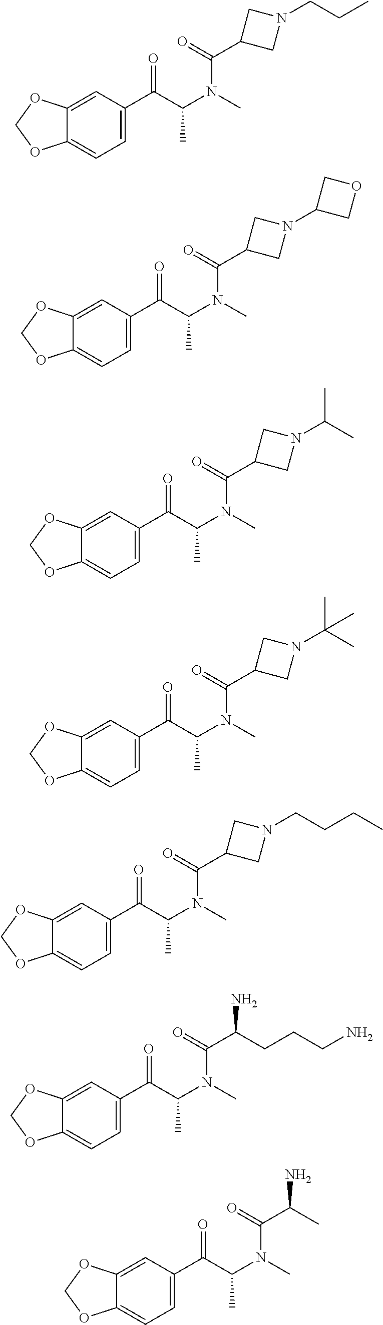

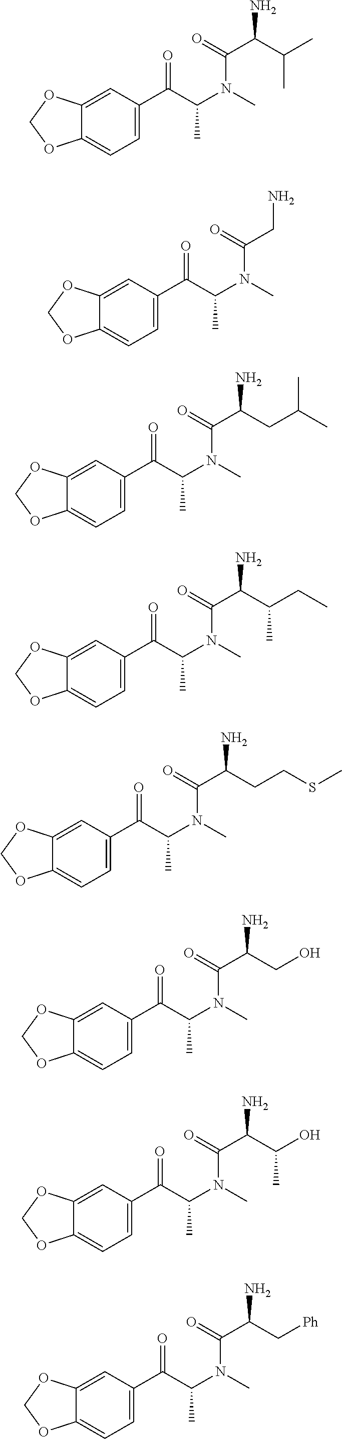

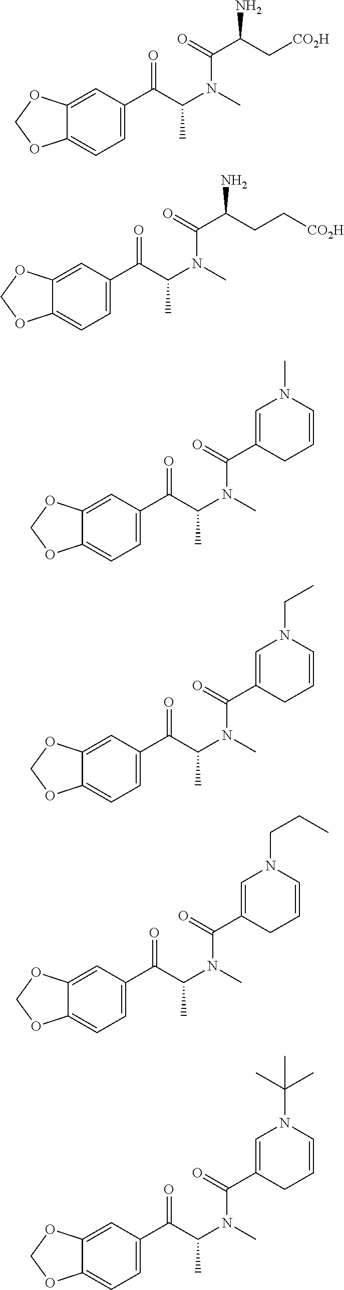 [SMILES: C[C@H](C(=O)c1ccc2c(c1)OCO2)N(C)C(=O)[C@@H](N)CC(=O)O ; C[C@H](C(=O)c1ccc2c(c1)OCO2)N(C)C(=O)[C@@H](N)CCC(=O)O ; C[C@H](C(=O)c1ccc2c(c1)OCO2)N(C)C(=O)C1=CN(C)C=CC1 ; CCN1C=CCC(C(=O)N(C)[C@H](C)C(=O)c2ccc3c(c2)OCO3)=C1 ; CCCN1C=CCC(C(=O)N(C)[C@H](C)C(=O)c2ccc3c(c2)OCO3)=C1 ; C[C@H](C(=O)c1ccc2c(c1)OCO2)N(C)C(=O)C1=CN(C(C)(C)C)C=CC1]

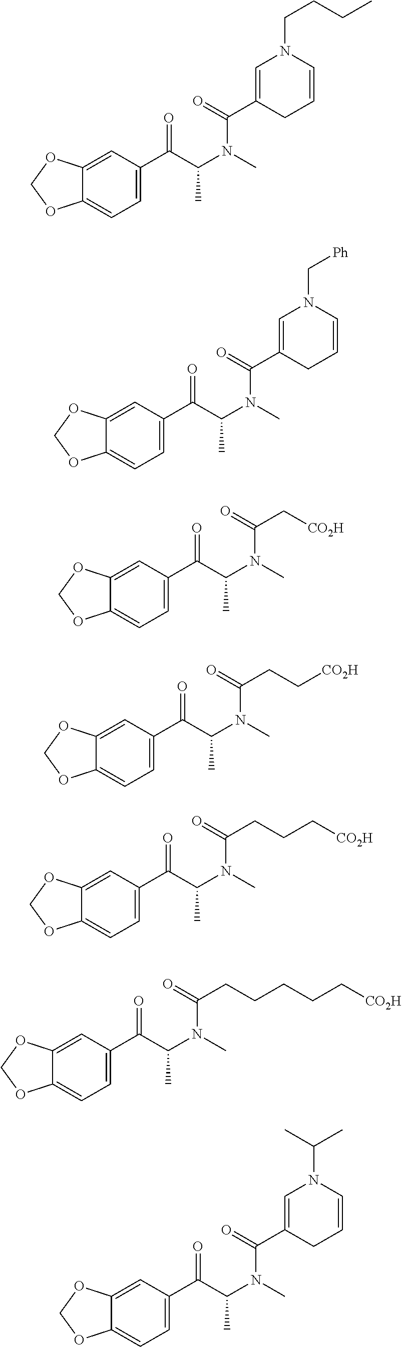

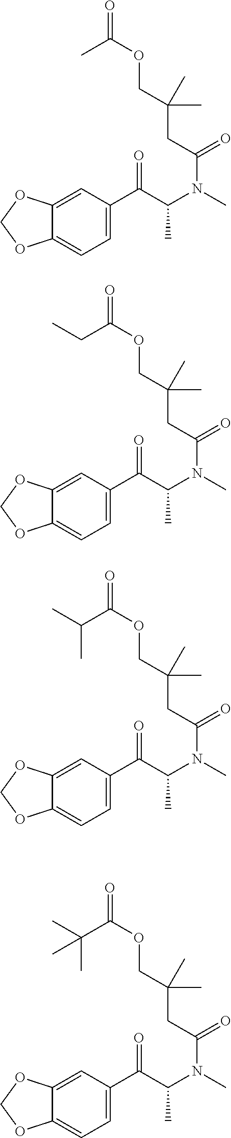

or a pharmaceutically acceptable salt thereof.

In some embodiments is a compound of Formulas (Ib) and (Ib-2), wherein the compound is:

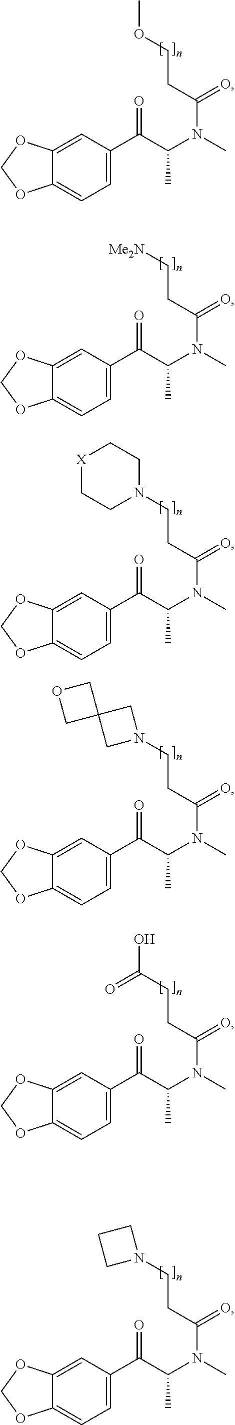

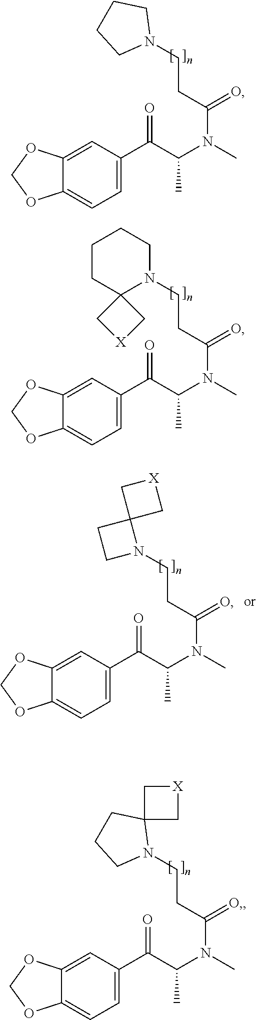

or a pharmaceutically acceptable salt thereof, wherein each n is independently 1, 2, 3, 4, 5, or 6; and each X is independently —O—, —S—, —S(O)—, —S(O)₂—, —NH—, or NR^{B}, wherein R^{B} is selected from alkyl, heteroalkyl, —C(O)CH₃ and —C(O)Ph, each of which is substituted or unsubstituted.

In some embodiments of Formulas (I), (Ib), (Ib-1) and (Ib-2), R⁴ is —CH(R^{A1})NH₂, wherein R^{A1} is hydrogen, alkyl, heteroalkyl, or an amino acid side chain. In one such embodiment of Formulas (I), (Ib), (Ib-1) and (Ib-2), R⁴ is —CH(R^{A1})NH₂, and R^{A1} is an amino acid side chain, the amino acid side chain is formed from an α-amino acid side chain, such as one of the naturally occurring amino acid side chains, such as an amino acid selected from alanine, serine, tryptophan, aspartic acid, glutamic acid and the like. By way of illustration and with reference to of Formulas (I), (Ib), (Ib-1) and (Ib-2), when R⁴ is formed from alanine, R^{A1} is methyl. In some embodiments of Formulas (I) and (Ib), R⁴ is —CH(R^{A1})NH₂, wherein R^{A1} is an amino acid side chain. In some embodiments of Formulas (I) and (Ib), R⁴ is —CH(R^{A1})NH₂, wherein R^{A1} is methyl, ethyl, n-propyl, isopropyl, tert-butyl, CH(Me)Et, CH₂CH(Me)₂, or CH₂CH₂SMe. In some embodiments of Formulas (I) and (Ib), wherein R⁴ is —CH(R^{A1})NH₂, wherein R^{A1} is benzyl.

In one embodiment of Formulas (I) and (Ib), R² is an amino acid residue, for example, in some embodiments of Formula (Ib), R⁴, together with the carbonyl to which it is attached, is an amino acid residue. Examples of compounds according to Formulas (I) and (Ib), wherein R² is an amino acid residue can be represented by Formula (Ib1):

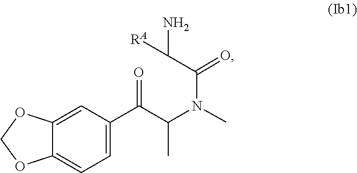

wherein the amino acid moiety Formula (Ib1) may be the (R) or the (S) configuration at the α-carbon as illustrated below:

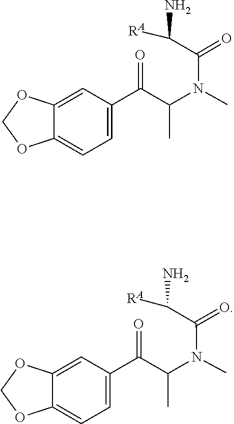

With reference to Formulas (Ib-1) and (Ib-2), compounds of Formula (Ib1) have two stereocenters, each of which can be in the (R) or the (S) configuration.

In another aspect, the present disclosure provides compounds of Formulas (I) and (Ib) having Formula (Ib2), or a pharmaceutically acceptable salt thereof:

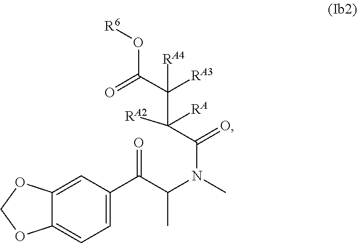

or a pharmaceutically acceptable salt thereof, wherein:
each of R^{A1} and R^{A2} is independently hydrogen, alkyl, cycloalkyl, aryl or heteroaryl, or R^{A1} and R^{A2} together with the atom to which they are attached form a cycloalkyl ring;
each of R^{A3} and R^{A4} is independently hydrogen, alkyl, cycloalkyl, aryl or heteroaryl, or R^{A3} and R^{A4} together with the atom to which they are attached form a cycloalkyl ring; and R⁶ is hydrogen, alkyl, cycloalkyl, aryl, or heteroaryl.

In some embodiments of compounds of Formulas (Ib) and (Ib2), each of R^{A1}, R^{A2}, R^{A3}, and R^{A4} is hydrogen. In some embodiments of a compound of Formulas (Ib) and (Ib2), each of R^{A1}, R^{A2}, R^{A3}, and R^{A4} is hydrogen or unsubstituted alkyl. In some embodiments of Formulas (Ib) and (Ib2), each of R^{A1}, R^{A2}, R^{A3}, and R^{A4} is hydrogen. In some embodiments of Formulas (Ib) and (Ib2), R⁶ is alkyl. In some embodiments of Formulas (Ib) and (Ib2), R⁶ is unsubstituted alkyl. In some embodiments of Formulas (Ib) and (Ib2), R⁶ is cycloalkyl. In some embodiments of Formulas (Ib) and (Ib2), R⁶ is methyl, ethyl, n-propyl, isopropyl, n-butyl, tert-butyl, 3-methyl-1-butyl, isopentyl, n-pentyl, n-hexyl, cyclopropyl, cyclobutyl, cyclopentyl, cyclohexyl, or cycloheptyl. In some embodiments of Formulas (Ib) and (Ib2), R⁶ is phenyl. In some embodiments of Formulas (Ib) and (Ib2), R⁶ is 4-nitrophenyl. In some embodiments of Formulas (Ib) and (Ib2), R⁶ is benzyl. In some embodiments of Formulas (Ib) and (Ib2), R⁶ is heteroaryl. In some embodiments of Formulas (Ib) and (Ib2), R⁶ is heteroaryl, such as 2-pyridyl, 3-pyridyl, 4-pyridyl, 2-pyrimidyl, or 4-pyrimidyl.

In some embodiments is a compound of Formula (Ib2), wherein the compound is:

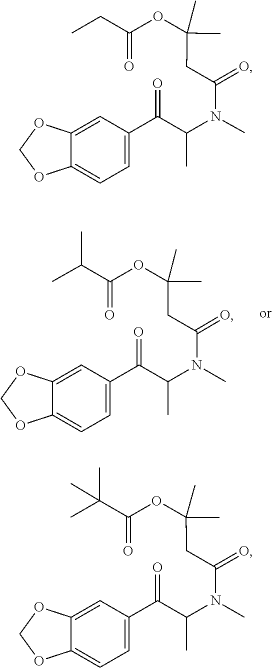

or a pharmaceutically acceptable salt thereof.

In some embodiments is a compound of Formulas (Ib) and (Ib2) having the structure of Formula (Ib2-1), or a pharmaceutically acceptable salt thereof:

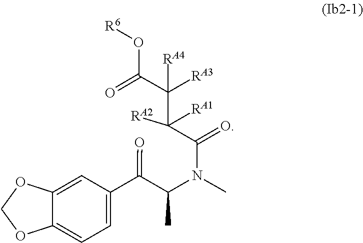

In some embodiments is a compound of Formulas (Ib) and (Ib2) having the structure of Formula (Ib2-2), or a pharmaceutically acceptable salt thereof:

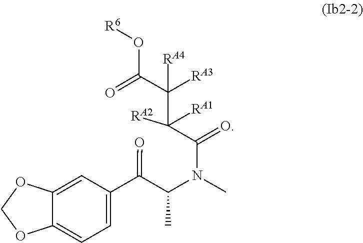

In another aspect, the present disclosure provides a compound of Formula (Ib) having Formula (Ib3):

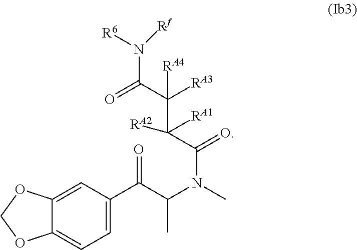

or a pharmaceutically acceptable salt thereof, wherein:
each of R^{A} and R^{A2} is independently hydrogen, alkyl, cycloalkyl, aryl or heteroaryl, or R^{A1} and R^{A2} together with the atom to which they are attached form a cycloalkyl ring;
each of R^{A3} and R^{A4} is independently hydrogen, alkyl, cycloalkyl, aryl or heteroaryl, or R^{A3} and R^{A4} together with the atom to which they are attached form a cycloalkyl ring; R^{f} is hydrogen or alkyl; and R⁶ is hydrogen, alkyl, cycloalkyl, aryl, or heteroaryl.

In some embodiments of compounds of Formulas (Ib) and (Ib3), each of R^{A1}, R^{A2}, R^{A3}, and R^{A4} is hydrogen. In some embodiments of a compound of Formulas (Ib) and (Ib3), each of R^{A1}, R^{A2}, R^{A3}, and R^{A4} is hydrogen or unsubstituted alkyl. In some embodiments of Formulas (Ib) and (Ib3), each of R^{A1}, R^{A2}, R^{A3}, and R^{A4} is hydrogen. In some embodiments of Formulas (Ib) and (Ib3), R⁶ is alkyl. In some embodiments of Formulas (Ib) and (Ib3), R⁶ is unsubstituted alkyl. In some embodiments of Formulas (Ib) and (Ib3), R⁶ is cycloalkyl. In some embodiments of Formulas (Ib) and (Ib3), R⁶ is methyl, ethyl, n-propyl, isopropyl, n-butyl, tert-butyl, 3-methyl-1-butyl, isopentyl, n-pentyl, n-hexyl, cyclopropyl, cyclobutyl, cyclopentyl, cyclohexyl, or cycloheptyl. In some embodiments of Formulas (Ib) and (Ib3), R⁶ is phenyl. In some embodiments of Formulas (Ib) and (Ib3), R⁶ is 4-nitrophenyl. In some embodiments of Formulas (Ib) and (Ib3), R⁶ is benzyl. In some embodiments of Formulas (Ib) and (Ib3), R⁶ is heteroaryl. In some embodiments of Formulas (Ib) and (Ib3), R⁶ is heteroaryl, such as 2-pyridyl, 3-pyridyl, 4-pyridyl, 2-pyrimidyl, or 4-pyrimidyl. In some embodiments of Formulas (Ib) and (Ib3), R^{f} is hydrogen. In some embodiments of Formulas (Ib) and (Ib3), R^{f} is alkyl, such as methyl.

In some embodiments is a compound of Formula (Ib3) having the structure of Formula (Ib3-1), or a pharmaceutically acceptable salt thereof:

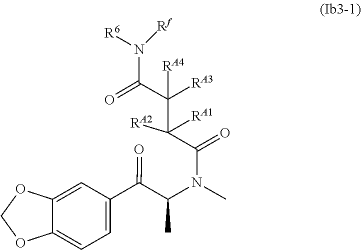

In some embodiments is a compound of Formula (Ib3) having the structure of Formula (Ib3-2), or a pharmaceutically acceptable salt thereof:

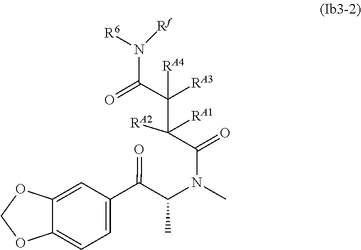

In another aspect, the present disclosure provides a compound of Formula (Ic):

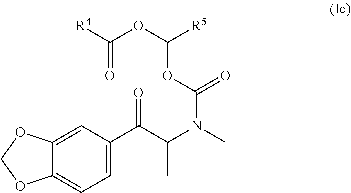

or a pharmaceutically acceptable salt thereof, wherein
R⁴ is alkyl, alkenyl, haloalkyl, cycloalkyl, heterocycloalkyl, aryl, heteroaryl, or heteroalkyl. Such compounds of Formula (Ic) may be referred to as N-acyloxyalkoxy prodrugs.

In some embodiments compounds of Formula (I) and (Ic), R⁵ is hydrogen, alkyl, or cycloalkyl; and R⁴ is alkyl, cycloalkyl, heteroalkyl, heterocycloalkyl, aryl, or heteroaryl. In some embodiments of Formulas (I) and (Ic), R⁵ is hydrogen or alkyl. In some embodiments of Formulas (I) and (Ic), R⁵ is hydrogen or unsubstituted alkyl. In some embodiments of Formulas (I) and (Ic), R⁵ is hydrogen. In some embodiments of Formulas (I) and (Ic), R⁴ is alkyl, cycloalkyl, heterocycloalkyl, aryl, or heteroaryl. In some embodiments of Formulas (I) and (Ic), R⁴ is alkyl. In some embodiments of Formulas (I) and (Ic), R⁴ is heteroalkyl. In some embodiments of Formulas (I) and (Ic), R⁴ is unsubstituted alkyl, unsubstituted cycloalkyl, unsubstituted heterocycloalkyl, unsubstituted aryl, or unsubstituted heteroaryl. In some embodiments of Formulas (I) and (Ic), R⁴ is alkyl. In some embodiments of Formulas (I) and (Ic), R⁴ is heteroalkyl. In some embodiments of Formulas (I) and (Ic), R⁴ is heterocycloalkyl substituted with arylalkyl. In some embodiments of Formulas (I) and (Ic), R⁵ is methyl, isopropyl, tert-butyl, or —CH(Et)₂.

In some embodiments of compounds of Formula (Ic), R⁴ is heteroalkyl. In some embodiments of Formula (Ic), R⁴ is heterocycloalkyl. In some embodiments of Formula (Ic), R⁴ is heteroalkyl or R⁴ is heterocycloalkyl.

In some embodiments of Formula (Ic), R⁴ is alkyl. In some embodiments of a compound of Formula (Ic), R⁴ is CH₂CF₃. In some embodiments of a compound of Formula (Ic), R⁴ is unsubstituted alkyl. In some embodiments of Formula (Ic), R⁴ is methyl, ethyl, n-propyl, isopropyl, n-butyl, tert-butyl, 3-methyl-1-butyl. In some embodiments a compound of Formula (Ic) is one wherein R⁴ is cycloalkyl. In some embodiments of compound of Formula (Ic), R⁴ is unsubstituted cycloalkyl.

In some embodiments of compounds of Formula (Ic), R⁴ is cyclopropyl, cyclobutyl, cyclopentyl, cyclohexyl, or cycloheptyl. In some embodiments of a compound of Formula (Ic), the compound is one wherein R⁴ is aryl. In some embodiments of Formula (Ic), wherein R⁴ is substituted or unsubstituted phenyl. In other embodiments of Formula (Ic), R⁴ is heteroaryl and in certain such some embodiments of Formula (Ic), R⁴ is 2-pyridyl, 3-pyridyl, 4-pyridyl, 2-pyrimidyl, 3-pyrimidyl, or 6-pyrimidyl.

In some embodiments of Formula (Ic), R⁴ is

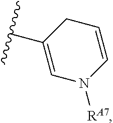

and R^{A7} is benzyl.

In some embodiments of Formula (Ic), R⁴ is R^{A} substituted with —OR¹³, —N(R¹⁸)R¹⁹, or —C(O)OR¹³, such as wherein R⁴ is alkyl, substituted with —OR¹³, —N(R¹⁸)R¹⁹, or —C(O)OR¹³. In some embodiments of Formula (Ic), R⁴ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Ic), R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(O)CH₃. In one embodiment of Formula (Ic) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Ic), R⁴ is heteroalkyl. In some embodiments of Formulas (I) and (Ic), R⁴ is CH₂CH₂OMe or CH₂CH₂SO₂Me. In some embodiments of Formulas (I) and (Ic), R⁴ is —(CH₂)ₙCO₂H, wherein n is 1, 2, 3, 4, 5, or 6. In some embodiments of Formulas (I) and (Ic), R⁴ is —(CH₂)ₙCO₂R¹³, wherein n is 1, 2, 3, 4, 5, or 6. In some embodiments of Formulas (I) and (Ic), R⁴ is —(CH₂)ₙCO₂R¹³, wherein R¹³ is alkyl. In some embodiments of Formulas (I) and (Ic), R⁴ is —(CH₂)ₙCO₂R¹³, wherein R¹³ is unsubstituted alkyl. In some embodiments of Formulas (I) and (Ic), R⁴ is —(CH₂)ₛCO₂R¹³, wherein R¹³ is methyl, ethyl, n-propyl, isopropyl, n-butyl, tert-butyl, or —CH(Et)₂.

In some embodiments of Formulas (I) and (Ic), R⁴ is —(CH₂)ₙOR¹³, wherein n is 1, 2, 3, 4, 5, 6 or 7. In some embodiments of Formulas (I) and (Ic), R⁴ is —(CH₂)ₙOR¹³, wherein R¹³ is alkyl. In some embodiments of Formulas (I) and (Ic), R⁴ is —(CH₂)ₙOR¹³, wherein R¹³ is unsubstituted alkyl. In some embodiments of Formulas (I) and (Ic), R⁴ is —(CH₂)ₛOR¹³, wherein R¹³ is methyl, ethyl, n-propyl, isopropyl, n-butyl, tert-butyl, or —CH(Et)₂.

In some embodiments of Formulas (I) and (Ic), R⁴ is —CH(R^{A1})NH₂, wherein R^{A1} is hydrogen, alkyl, heteroalkyl, or an amino acid side chain. In such embodiments of Formulas (I) and (Ic), R² is an amino acid residue, for example, with reference to Formula (Ic), R⁴, together with the carbonyl to which it is attached, in some embodiments, is an amino acid residue, that is R⁴ is —CH(R^{A1})NH₂. In one such embodiment of Formulas (I) and (Ic), wherein R⁴ is —CH(R^{A1})NH₂, and R^{A1} is an amino acid side chain, the amino acid side chain is formed from an α-amino acid side chain, such as one of the naturally occurring amino acid side chains, such as an amino acid selected from alanine, serine, tryptophan, aspartic acid, glutamic acid and the like. By way of illustration, when R^{A1} is formed from alanine, R^{A1} is methyl. In some embodiments of Formulas (I) and (Ic), R⁴ is —CH(R^{A1})NH₂, wherein R^{A1} is an amino acid side chain. In some embodiments of Formulas (I) and (Ic), R⁴ is —CH(R^{A1})NH₂, wherein R^{A1} is methyl, ethyl, n-propyl, isopropyl, tert-butyl, CH(Me)Et, CH₂CH(Me)₂, or CH₂CH₂SMe. In some embodiments of Formulas (I) and (Ic), wherein R⁴ is —CH(R^{A1})NH₂, wherein R^{A1} is benzyl.

Certain embodiments of disclosed compounds described above wherein R⁴ is together with the carbonyl to which it is attached, an amino acid residue, are represented by Formula (Ic1):

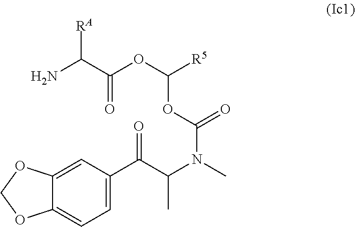

or a pharmaceutically acceptable salt thereof, wherein R^{A} is hydrogen, substituted or unsubstituted alkyl, substituted or unsubstituted heteroalkyl, or an amino acid side chain; and R⁵ is alkyl that is substituted or unsubstituted, or hydrogen. In some embodiments of a compound of Formulas (Ic) and (Ic1) the compound is:

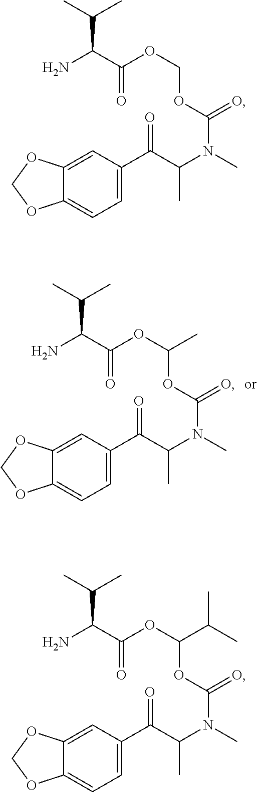 [SMILES: CC(C)[C@H](N)C(=O)OCOC(=O)N(C)C(C)C(=O)c1ccc2c(c1)OCO2 ; CC(OC(=O)[C@@H](N)C(C)C)OC(=O)N(C)C(C)C(=O)c1ccc2c(c1)OCO2 ; CC(C)C(OC(=O)[C@@H](N)C(C)C)OC(=O)N(C)C(C)C(=O)c1ccc2c(c1)OCO2]

or a pharmaceutically acceptable salt thereof.

In some embodiments is a compound of Formula (Ic), wherein the compound is:

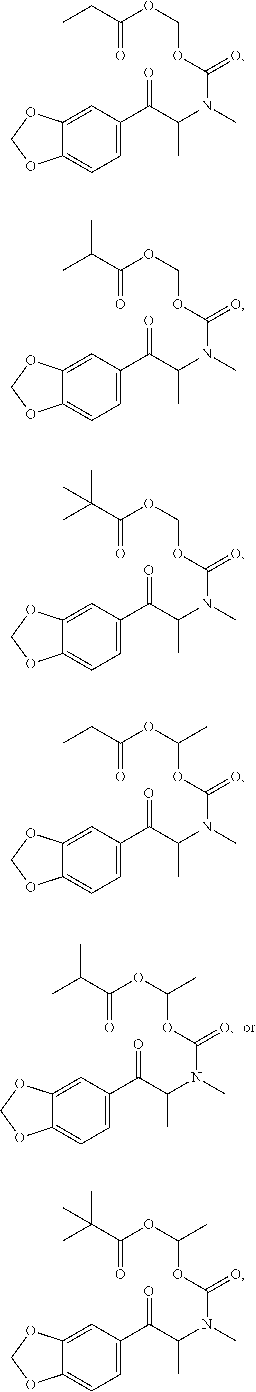 [SMILES: CCC(=O)OCOC(=O)N(C)C(C)C(=O)c1ccc2c(c1)OCO2 ; CC(C)C(=O)OCOC(=O)N(C)C(C)C(=O)c1ccc2c(c1)OCO2 ; CC(C(=O)c1ccc2c(c1)OCO2)N(C)C(=O)OCOC(=O)C(C)(C)C ; CCC(=O)OC(C)OC(=O)N(C)C(C)C(=O)c1ccc2c(c1)OCO2 ; CC(OC(=O)C(C)C)OC(=O)N(C)C(C)C(=O)c1ccc2c(c1)OCO2 ; CC(OC(=O)N(C)C(C)C(=O)c1ccc2c(c1)OCO2)OC(=O)C(C)(C)C]

a pharmaceutically acceptable salt thereof.

In some embodiments is a compound of Formula (Ic), wherein the compound is:

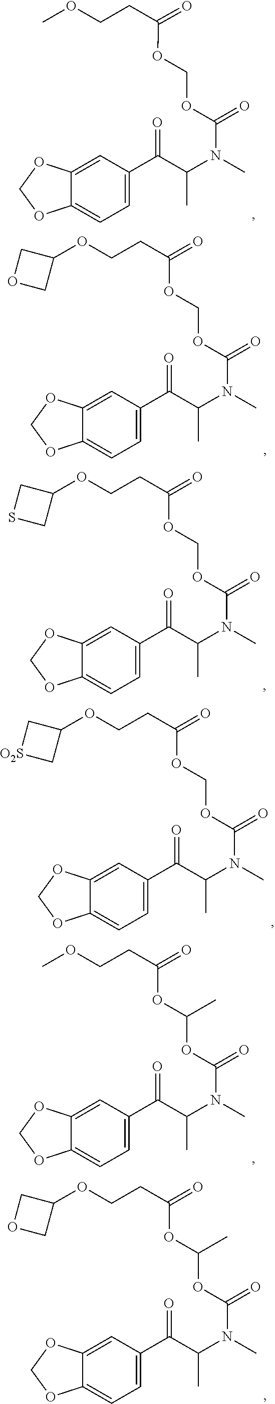

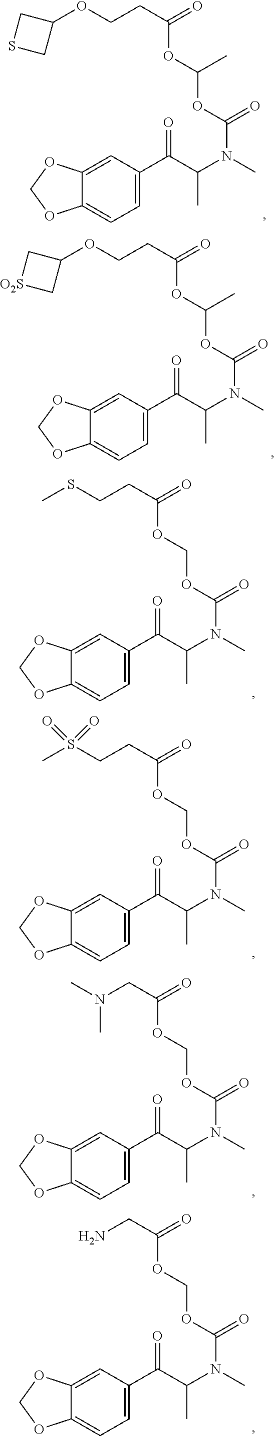

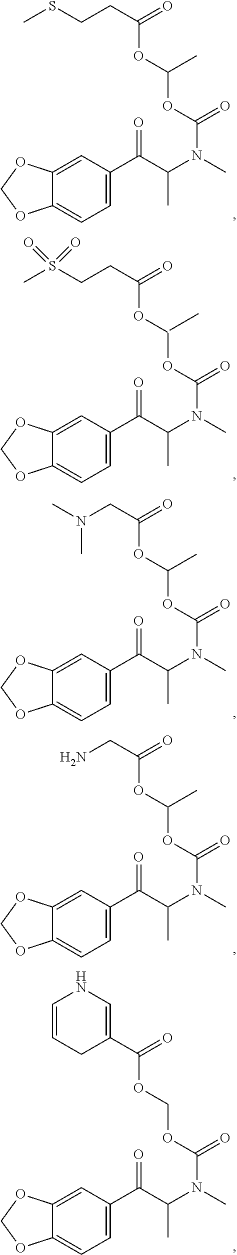

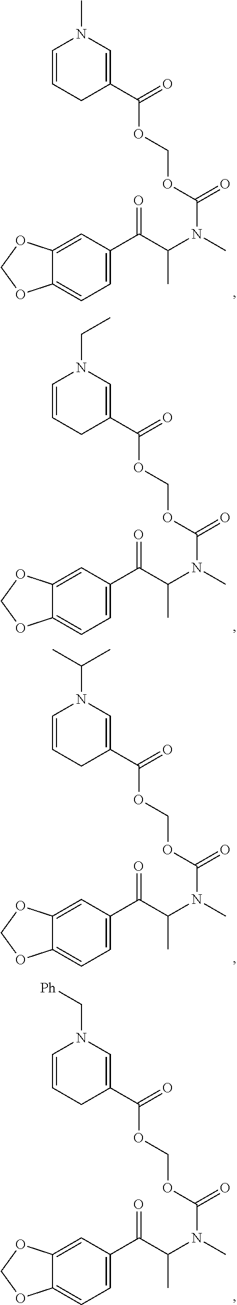

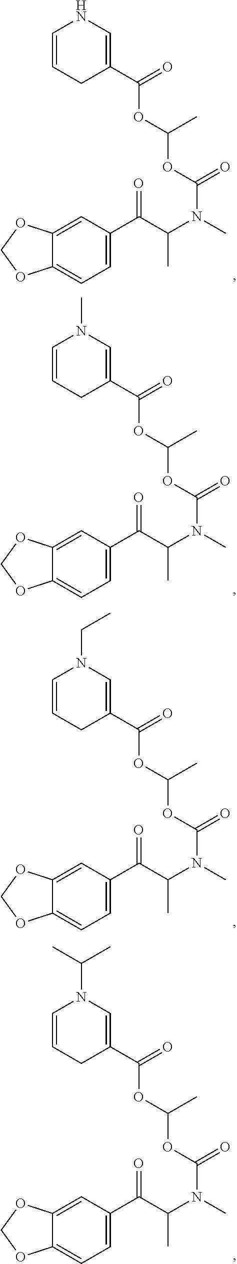

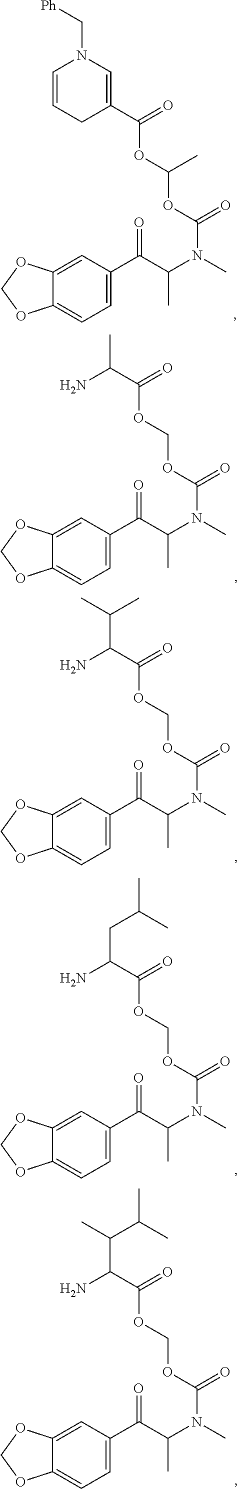

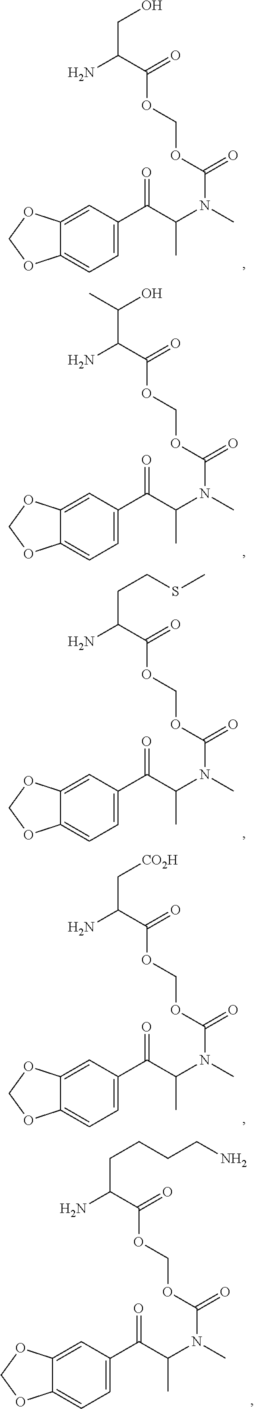

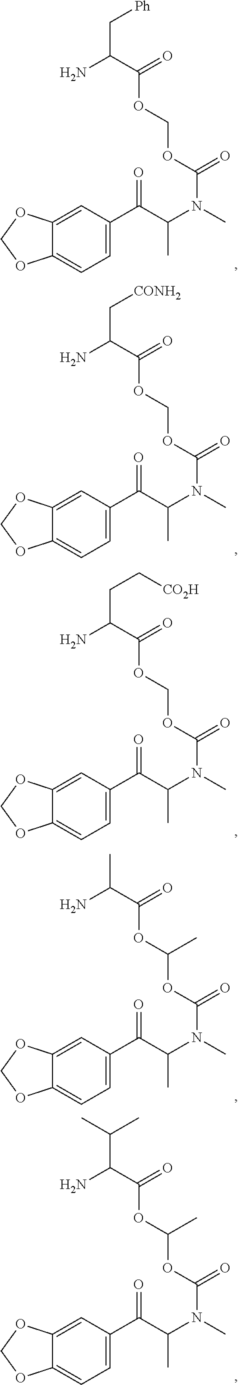

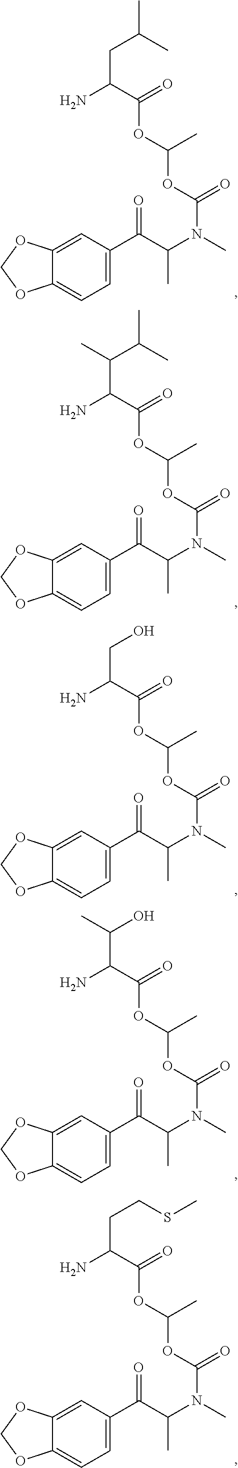

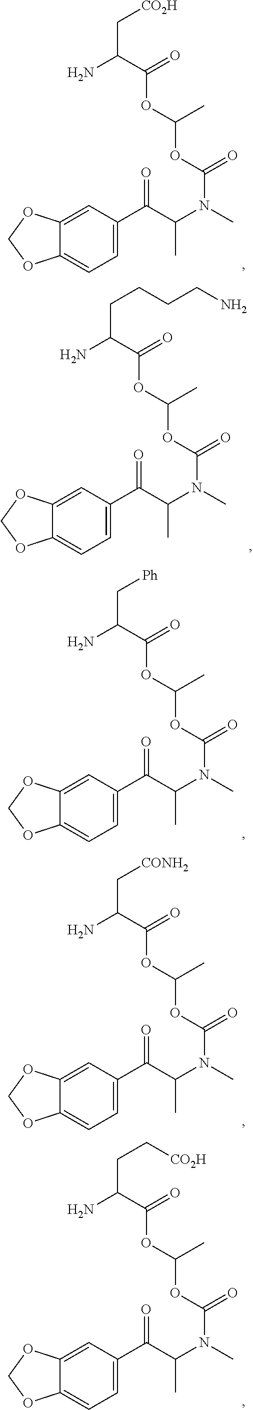

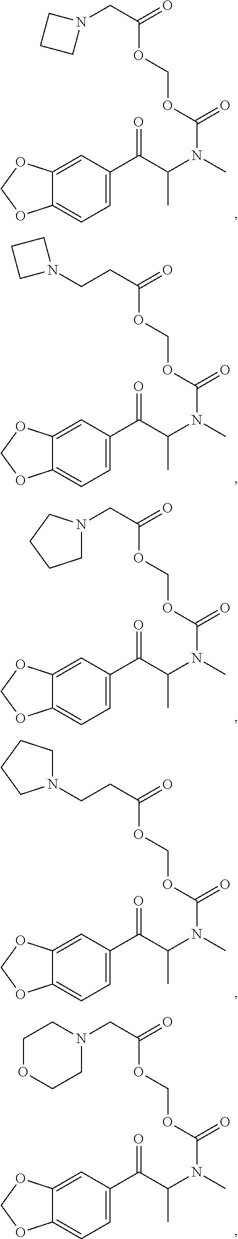

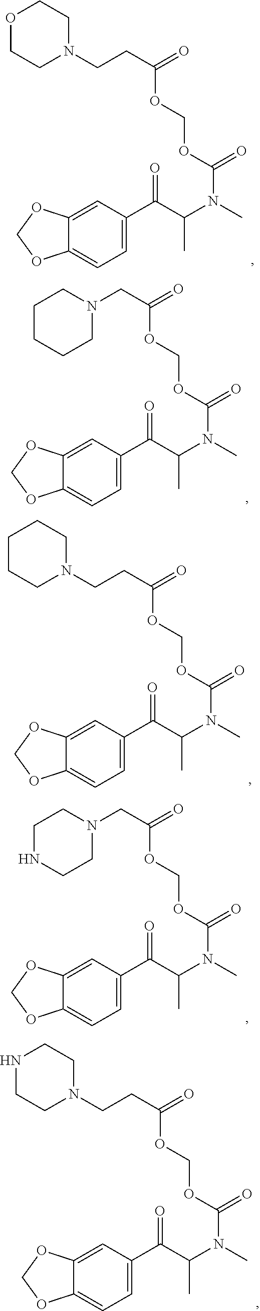

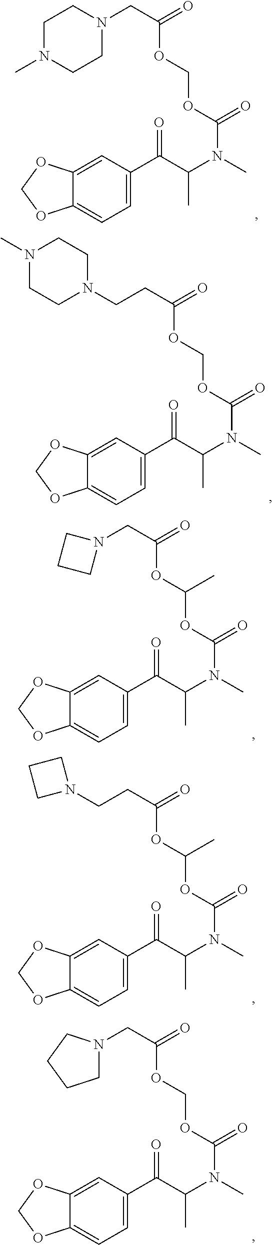

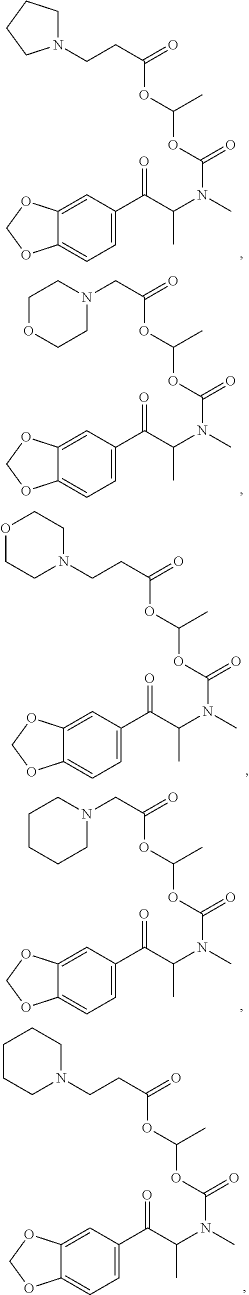

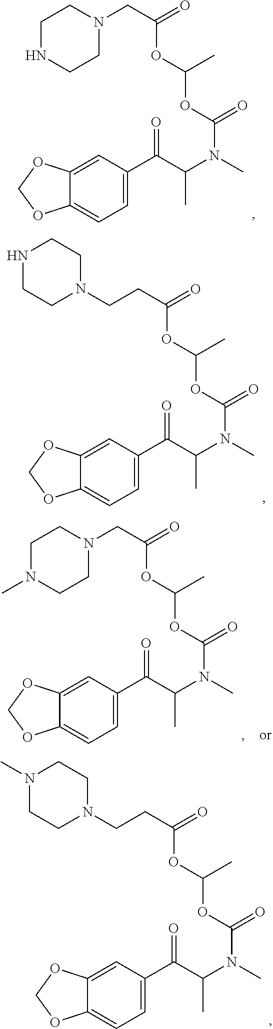

or a pharmaceutically acceptable salt thereof.

In some embodiments of Formulas (I) and (Ic), R⁴ is —(CH₂)ₙOR¹³, wherein n is 1, 2, 3, 4, 5, 6 or 7. In some embodiments of Formulas (I) and (Ic), R⁴ is —(CH₂)ₙOR¹³, wherein R¹³ is alkyl. In some embodiments of Formulas (I) and (Ic), R⁴ is —(CH₂)ₙOR¹³, wherein R¹³ is unsubstituted alkyl. In some embodiments of Formulas (I) and (Ic), R⁴ is —(CH₂)ₛOR¹³, wherein R¹³ is methyl, ethyl, n-propyl, isopropyl, n-butyl, tert-butyl, or —CH(Et)₂. In certain such embodiments of Formula (Ic), the compounds have a Ic2):

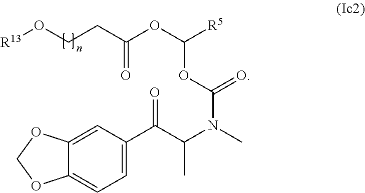

In particular embodiments of Formula (Ic2), R¹³ is alkyl, heteroalkyl, cycloalkyl, or heterocycloalkyl, each of which is substituted or unsubstituted; R² is alkyl that is substituted or unsubstituted, or hydrogen; and n is 1, 2, 3, 4, 5, or 6.

In some embodiments is a compound of Formula (Ic2), wherein R¹³ is methyl, ethyl, isopropyl, n-propyl, tert-butyl, n-butyl, n-pentyl, iso-amyl, cyclopropyl, cyclobutyl, cyclopentyl, or cyclohexyl. In some embodiments is a compound of Formula (Ic2), wherein R¹³ is methyl.

In some embodiments of Formulas (I) and (Ic), R⁴ is —(CH₂)ₙN(R¹⁸)R¹⁹, wherein n is 1, 2, 3, 4, 5, 6 or 7. In some embodiments of Formulas (I) and (Ic), R⁴ is —(CH₂)ₙ—N(R¹⁸)R¹⁹, wherein R¹⁸ and R¹⁹ independently are selected from hydrogen and alkyl, or together with the atom to which they are attached form a heterocycloalkyl ring, such as an azetidinyl, pyrrolidinyl, morpholinyl, piperazinyl or piperidinyl ring.

In some embodiments of a compound of Formula (Ic), compounds having the structure of Formula (Ic3), or a pharmaceutically acceptable salt thereof, are disclosed herein:

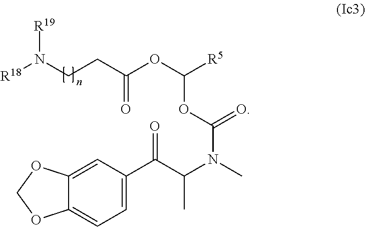

In some embodiments of Formulas (I) (Ic) and (Ic3), each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring that is substituted or unsubstituted; R⁵ is alkyl that is substituted or unsubstituted, or hydrogen; and n is 1, 2, 3, 4, 5, or 6.

In some embodiments is a compound of Formula (Ic3), wherein each of R¹⁸ and R¹⁹ independently is hydrogen, methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, or —CH₂cPr.

In some embodiments is a compound of Formula (Ic3), wherein RX and R^{Y} together with the atom to which they are attached form an azetidine ring, a piperidine ring, piperazine ring, a morpholine ring, or a pyrrolidine ring, each of which is substituted or unsubstituted.

In some embodiments is a compound of Formulas (Ic) and (Ic3) having the structure of Formula (Ic4), or a pharmaceutically acceptable salt thereof:

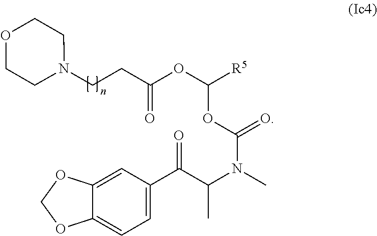

In some embodiments is a compound of Formula (Ic) and (Ic3) having the structure of Formula (Ic5):

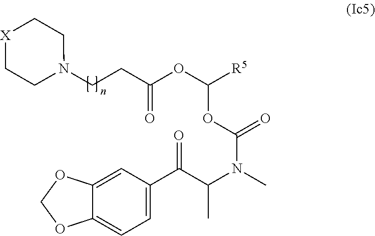

or a pharmaceutically acceptable salt thereof, wherein X is —NH—, or NR^{B}; R^{B} is alkyl, —C(O)CH₃, heteroalkyl, or cycloalkyl, wherein alkyl, heteroalkyl, or cycloalkyl is substituted or unsubstituted; R⁵ is alkyl that is substituted or unsubstituted, or hydrogen; and n is 1, 2, 3, 4, 5, or 6.

In some embodiments is a compound of Formula (Ic5), wherein R^{B} is methyl, ethyl, n-propyl, isopropyl, or —CH(Et)₂. In some embodiments is a compound of Formula (Ic5), wherein X is —NH—.

In some embodiments of Formula (Ic), compounds having the structure of Formula (Ic-1), or a pharmaceutically acceptable salt thereof, are provided:

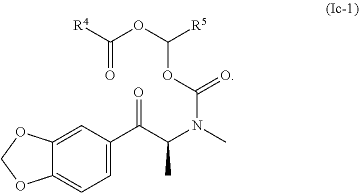

In some embodiments of compounds of Formulas (Ic), (Ic2) and (Ic-1), the compounds have the structure of Formula (Ic2-1), or a pharmaceutically acceptable salt thereof:

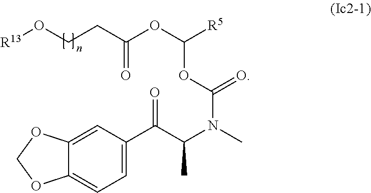

In some embodiments of compounds of Formula (Ic), (Ic3), and (Ic-1), the compounds have the structure of Formula (Ic3-1), or a pharmaceutically acceptable salt thereof:

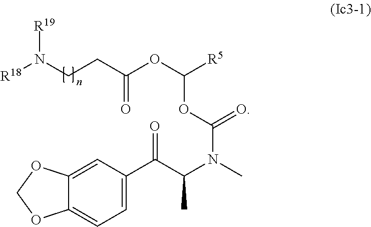

In some embodiments of compounds of Formulas (Ic), (Ic3), (Ic-1), and (Ic3-1) have the structure of Formula (Ic4-1), or a pharmaceutically acceptable salt thereof:

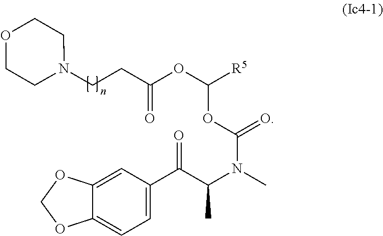

In some embodiments is a compound of Formulas (Ic), (Ic3, (Ic-1), and (Ic3-1) having the structure of Formula (Ic5-1):

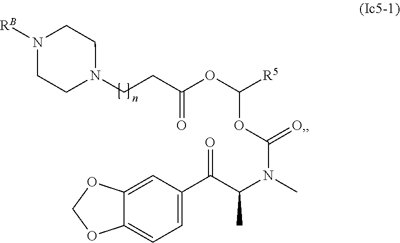

or a pharmaceutically acceptable salt thereof, wherein R^{B} is selected from hydrogen, alkyl, arylalkyl and —C(O)CH₃.

In some embodiments is a compound of Formulas (I), (Ic), and (Ic1) having the structure of Formula (Ic1-1), or a pharmaceutically acceptable salt thereof:

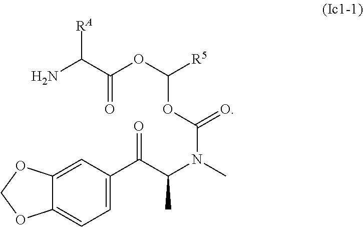

In some embodiments is a compound of Formula (Ic) having the structure of Formula (Ic-2), or a pharmaceutically acceptable salt thereof:

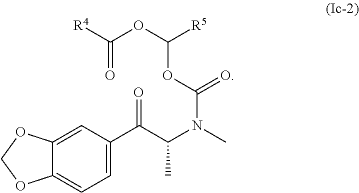

In some embodiments is a compound of Formula (Ic) and (Ic-2) having the structure of Formula (Ic2-2), or a pharmaceutically acceptable salt thereof:

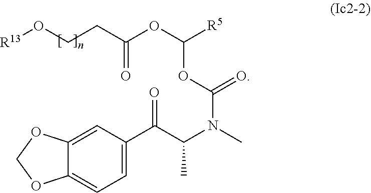

In some embodiments is a compound of Formula (Ic), (Ic3), and (Ic-2), having the structure of Formula (Ic2-2), or a pharmaceutically acceptable salt thereof:

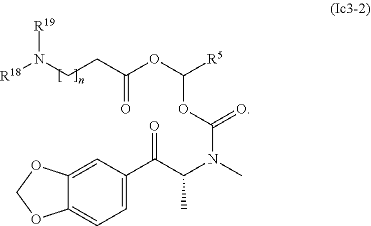

In some embodiments is a compound of Formula (Ic), (Ic3), (Ic-2), and (Ic4) having the structure of Formula (Ic4-2), or a pharmaceutically acceptable salt thereof:

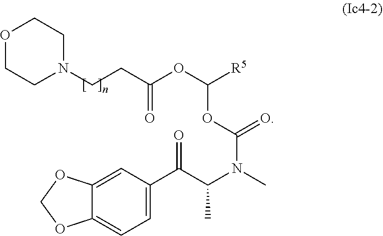

In some embodiments is a compound of Formula (Ic), (Ic3), (Ic-2), and (Ic5) having the structure of Formula (Ic4-2), or a pharmaceutically acceptable salt thereof:

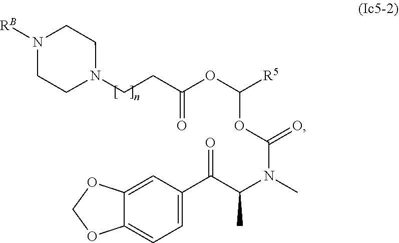

wherein R^{B} is selected from alkyl, arylalkyl and —C(O)CH₃.

In some embodiments is a compound of Formulas (I), (Ic), and (Ic1) having the structure of Formula (Ic1-2), or a pharmaceutically acceptable salt thereof:

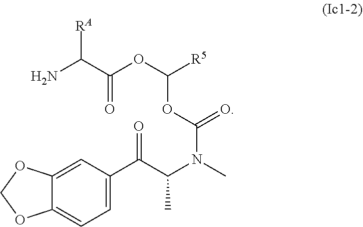

In some embodiments is a compound of Formulas (Ic), (Ic1), (Ic2), (Ic3), (Ic4), (Ic5), (Ic-1), (Ic1-1), (Ic2-1), (Ic3-1), (Ic4-1), (Ic5-1), (Ic-2), (Ic1-2), (Ic2-2), (Ic3-2), (Ic4-2), and/or (Ic5-2), wherein R⁵ is hydrogen. In some embodiments is a compound of Formulas (Ic), (Ic1), (Ic2), (Ic3), (Ic4), (Ic5), (Ic-1), (Ic1-1), (Ic2-1), (Ic3-1), (Ic4-1), (Ic5-1), (Ic-2), (Ic1-2), (Ic2-2), (Ic3-2), (Ic4-2), and/or (Ic5-2), wherein R⁵ is methyl, ethyl, n-propyl, isopropyl, or —CH(Et)₂.

In some embodiments is a compound of Formula (Ic2), (Ic3), (Ic4), (Ic5) (Ic2-1), (Ic3-1), (Ic4-1), (Ic5-1), (Ic2-2), (Ic3-2), (Ic4-2), and/or (Ic5-2), wherein n is 1.

In another aspect, the present disclosure provides a compound of Formula (Id), or a pharmaceutically acceptable salt thereof:

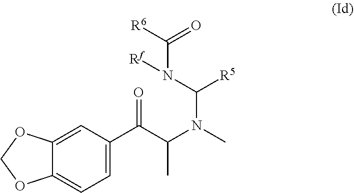

wherein R⁶ is alkyl, alkenyl, heteroalkyl, cycloalkyl, heterocycloalkyl, aryl, or heteroaryl, each of which is substituted or unsubstituted. In one embodiment R⁶ and the carbonyl to which R⁶ is attached form an amino acid residue; and each of R^{f} and R⁵ independently is alkyl that is substituted or unsubstituted, or hydrogen.

In some embodiments is a compound of Formula (Id), wherein R⁶ together which the carbonyl to which R⁶ is attached form an amino acid residue.

In some embodiments is a compound of Formula (Id), wherein R⁶ is alkyl or heteroalkyl that is substituted or unsubstituted. In some embodiments is a compound of Formula (Id), wherein R⁶ is alkyl that is substituted. In some embodiments is a compound of Formula (VI), wherein R⁶ is alkyl that is substituted with heterocycloalkyl that is substituted or unsubstituted.

In some embodiments of Formula (I) compounds have Formula (Id), wherein R⁵ is unsubstituted alkyl. In some embodiments of Formula (Id), R⁵ is hydrogen, methyl, ethyl, or isopropyl. In some embodiments of Formula (Id), In some embodiments of Formula (Id), R⁵ is unsubstituted alkyl, and R⁶ is hydrogen, methyl, ethyl, n-propyl, isopropyl, n-butyl, or benzyl.

In some embodiments of Formulas (I) and (Id), R⁵ is hydrogen, and R⁶ is alkyl. In some embodiments of Formulas (I) and (Id), R⁵ is alkyl, and R⁶ is alkyl. In some embodiments of Formulas (I) and (Id), R⁵ is hydrogen, and R⁶ is unsubstituted alkyl. In some embodiments of Formulas (I) and (Id), R⁵ is unsubstituted alkyl, and R⁶ is unsubstituted alkyl. In some embodiments of Formulas (I) and (Id), R⁶ is methyl, ethyl, isopropyl, tert-butyl, or cyclopropyl. In some embodiments of Formula (I) and (Id), R⁵ is hydrogen, and R⁶ is methyl, ethyl, isopropyl, tert-butyl, or cyclopropyl. In some embodiments of Formulas (I) and (Id), R⁵ is hydrogen, and R⁶ is tert-butyl. In some embodiments of Formulas (I) and (Id), R¹ is C₁₋₆ alkyl, such as methyl, R⁵ is hydrogen, and R⁴ is tert-butyl. In some embodiments of Formulas (I) and (Id), R¹ is C₁₋₆ alkyl, R⁵ is hydrogen, and R⁴ is tert-butyl.

In some embodiments of Formulas (I) and (Id), R⁶ is alkyl. In some embodiments of Formulas (I) and (Id), R⁶ is unsubstituted alkyl. In some embodiments of Formulas (I) and (Id), R⁶ is cycloalkyl. In some embodiments of Formulas (I) and (Id), R⁶ is methyl, ethyl, n-propyl, tert-butyl, 3-methyl-1-butyl, n-pentyl, n-hexyl, cyclopropyl, cyclobutyl, cyclopentyl, cyclohexyl, or cycloheptyl. In some embodiments of Formulas (I) and (Id), R⁶ is phenyl. In some embodiments of Formulas (I) and (Id), R⁶ is 4-nitrophenyl. In some embodiments of Formulas (I) and (Id), R⁶ is benzyl. In some embodiments of Formulas (I) and (Id), R⁶ is heteroaryl. In some embodiments of Formulas (I) and (Id), R⁶ is heteroaryl, such as 2-pyridyl, 3-pyridyl, 4-pyridyl, 2-pyrimidyl, or 4-pyrimidyl.

In some embodiments of Formulas (I) and (Id), R⁶ is heteroalkyl. In some embodiments of Formulas (I) and (Id), R⁶ is CH₂CH₂OMe or CH₂CH₂SO₂Me. In some embodiments of Formulas (I) and (Id), R⁶ is —(CH₂)ₙCO₂H, wherein n is 1, 2, 3, 4, 5, or 6. In some embodiments of Formulas (I) and (Id), R⁶ is —(CH₂)ₙCO₂R¹³, wherein n is 1, 2, 3, 4, 5, or 6. In some embodiments of Formulas (I) and (Id), R⁶ is —(CH₂)ₙCO₂R¹³, wherein R¹³ is alkyl. In some embodiments of Formulas (I) and (Id), R⁴ is —(CH₂)ₙCO₂R¹³, wherein R¹³ is unsubstituted alkyl. In some embodiments of Formulas (I) and (Id), R⁴ is —(CH₂)ₛCO₂R¹³, wherein R¹³ is methyl, ethyl, n-propyl, isopropyl, n-butyl, tert-butyl, or —CH(Et)₂.

In some embodiments is a compound of Formulas (I) and (Id), wherein the compound is:

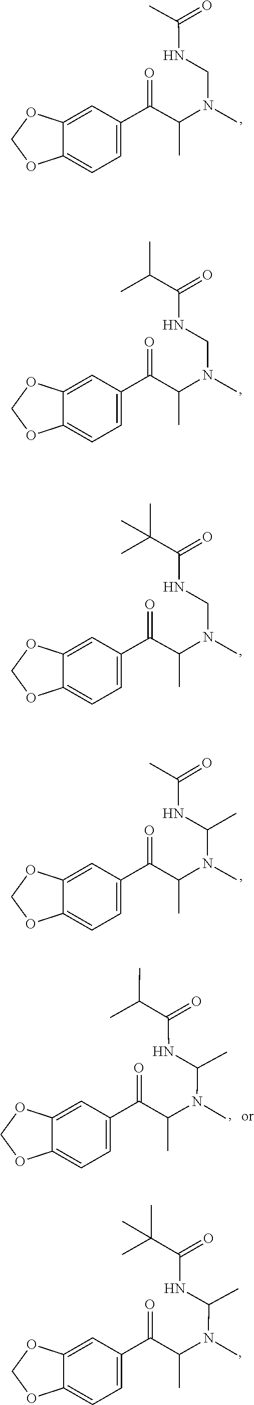 [SMILES: CC(=O)NCN(C)C(C)C(=O)c1ccc2c(c1)OCO2 ; CC(C)C(=O)NCN(C)C(C)C(=O)c1ccc2c(c1)OCO2 ; CC(C(=O)c1ccc2c(c1)OCO2)N(C)CNC(=O)C(C)(C)C ; CC(=O)NC(C)N(C)C(C)C(=O)c1ccc2c(c1)OCO2 ; CC(C)C(=O)NC(C)N(C)C(C)C(=O)c1ccc2c(c1)OCO2 ; CC(NC(=O)C(C)(C)C)N(C)C(C)C(=O)c1ccc2c(c1)OCO2]

or a pharmaceutically acceptable salt thereof.

In some embodiments is a compound of Formulas (I) and (Id), wherein the compound is:

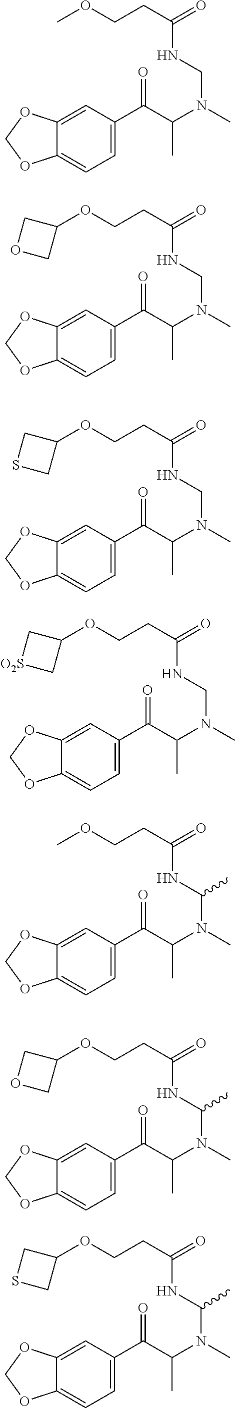

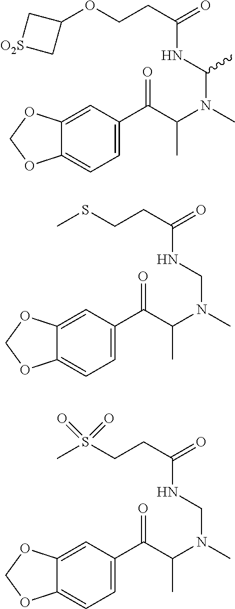

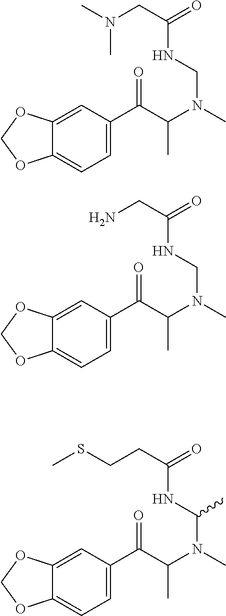 [SMILES: CC(C(=O)c1ccc2c(c1)OCO2)N(C)CNC(=O)CN(C)C ; CC(C(=O)c1ccc2c(c1)OCO2)N(C)CNC(=O)CN ; CSCCC(=O)NC(C)N(C)C(C)C(=O)c1ccc2c(c1)OCO2]

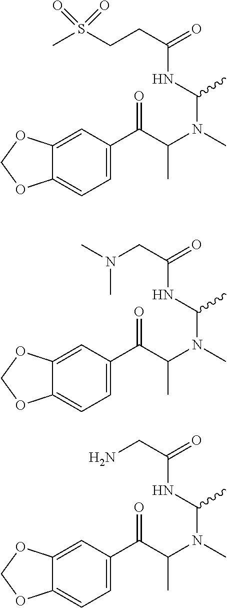

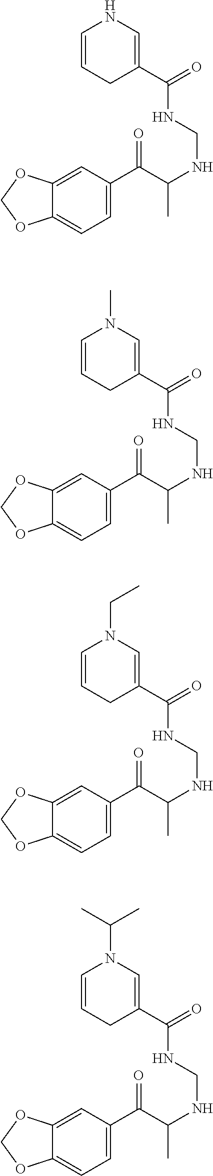 [SMILES: CC(NCNC(=O)C1=CNC=CC1)C(=O)c1ccc2c(c1)OCO2 ; CC(NCNC(=O)C1=CN(C)C=CC1)C(=O)c1ccc2c(c1)OCO2 ; CCN1C=CCC(C(=O)NCNC(C)C(=O)c2ccc3c(c2)OCO3)=C1 ; CC(NCNC(=O)C1=CN(C(C)C)C=CC1)C(=O)c1ccc2c(c1)OCO2]

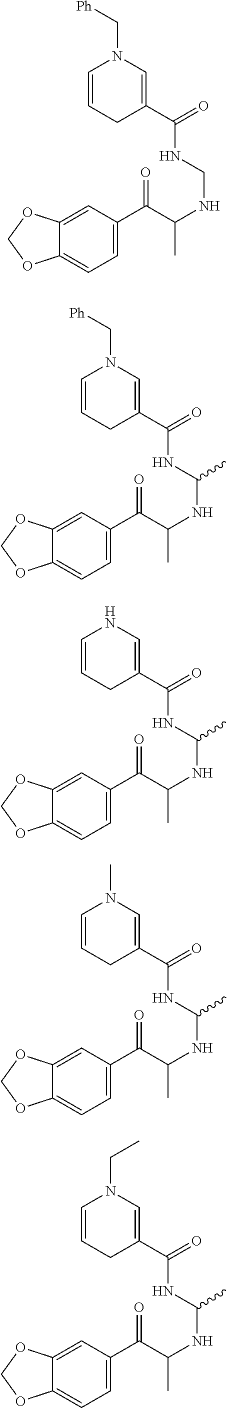

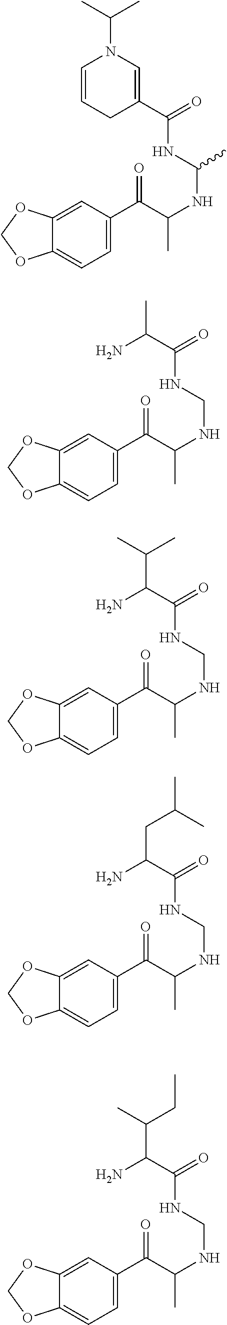

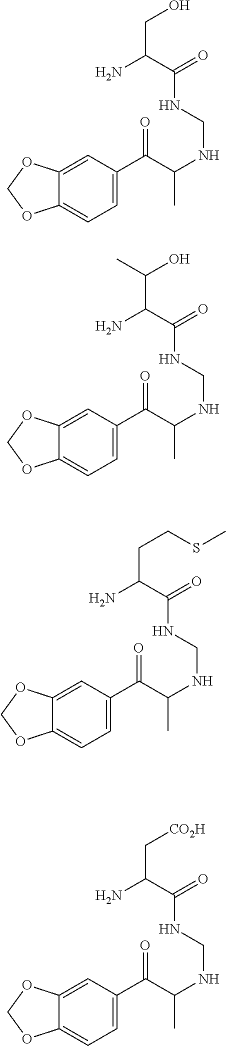 [SMILES: CC(NCNC(=O)C(N)CO)C(=O)c1ccc2c(c1)OCO2 ; CC(NCNC(=O)C(N)C(C)O)C(=O)c1ccc2c(c1)OCO2 ; CSCCC(N)C(=O)NCNC(C)C(=O)c1ccc2c(c1)OCO2 ; CC(NCNC(=O)C(N)CC(=O)O)C(=O)c1ccc2c(c1)OCO2]

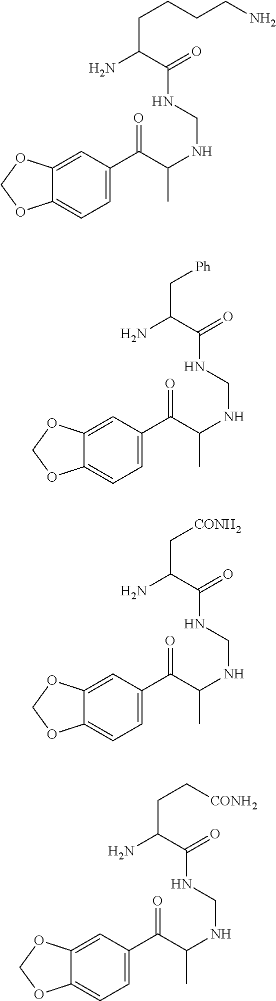 [SMILES: CC(NCNC(=O)C(N)CCCCN)C(=O)c1ccc2c(c1)OCO2 ; CC(NCNC(=O)C(N)Cc1ccccc1)C(=O)c1ccc2c(c1)OCO2 ; CC(NCNC(=O)C(N)CC(N)=O)C(=O)c1ccc2c(c1)OCO2 ; CC(NCNC(=O)C(N)CCC(N)=O)C(=O)c1ccc2c(c1)OCO2]

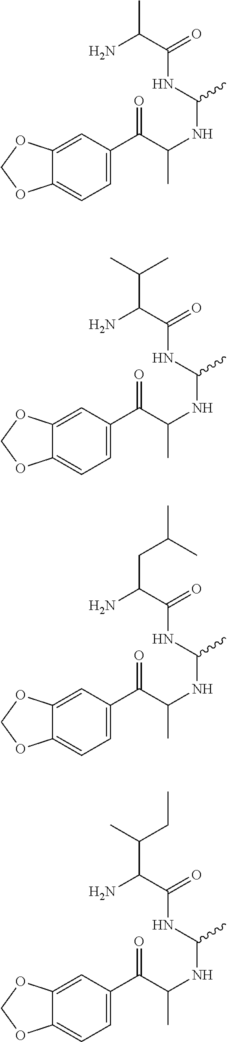

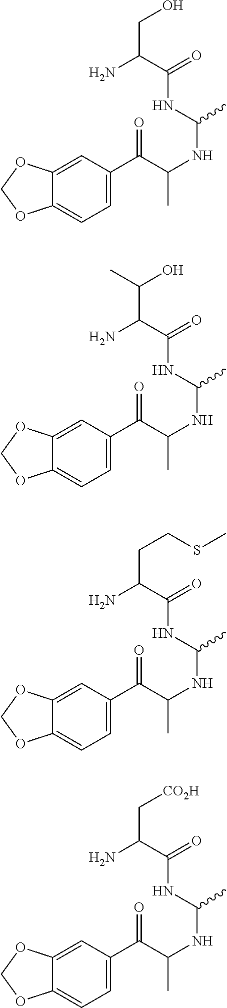

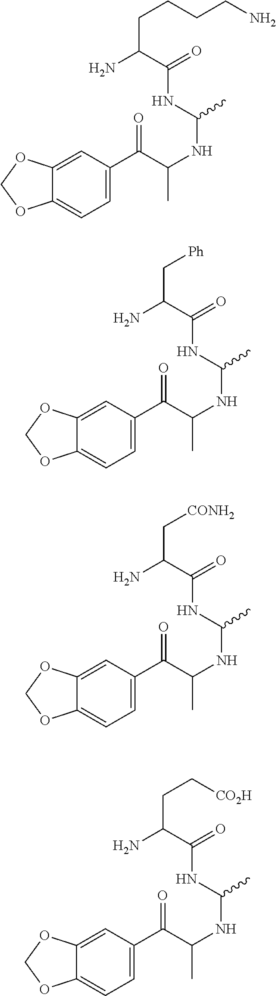

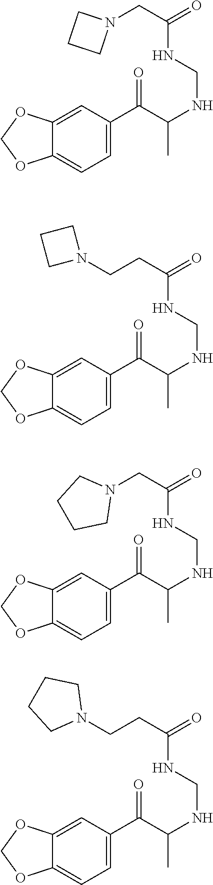

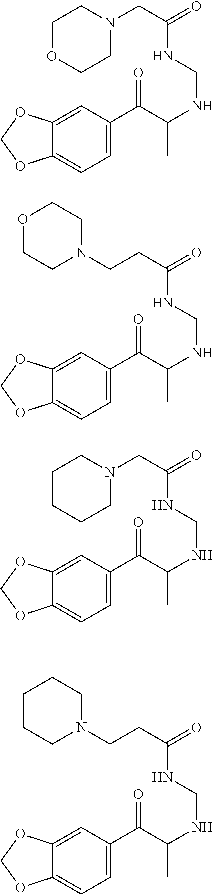

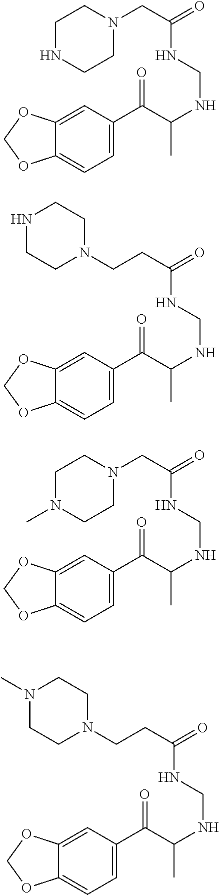

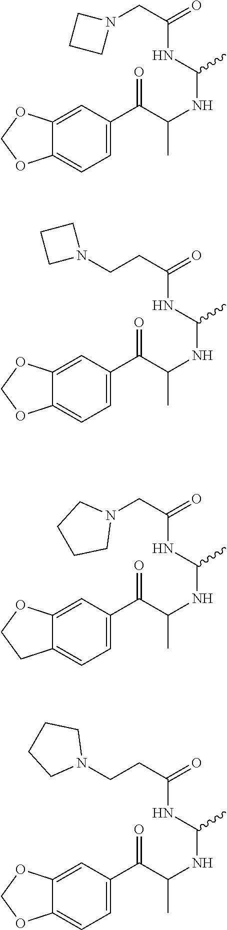

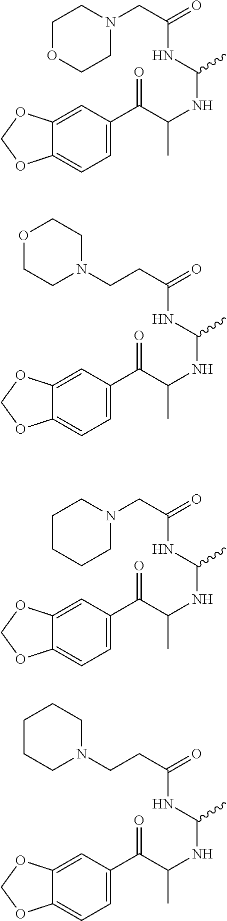

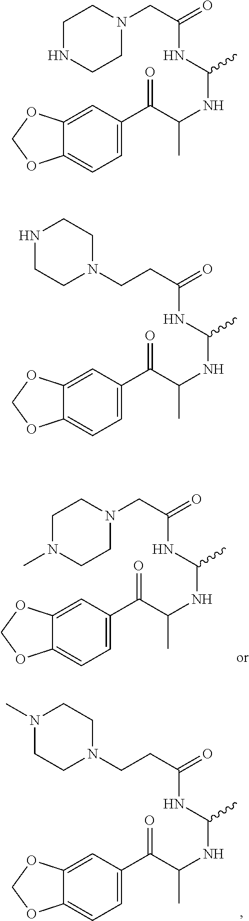

or a pharmaceutically acceptable salt thereof.

In some embodiments is a compound of Formulas (I) and (Id) having the structure of Formula (Id1):

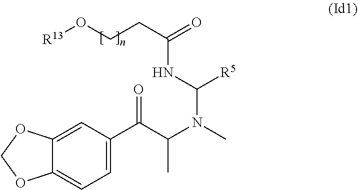

or a pharmaceutically acceptable salt thereof, wherein R¹³ is alkyl, heteroalkyl, cycloalkyl, or heterocycloalkyl, each of which is substituted or unsubstituted; R⁵ is hydrogen or alkyl that is substituted or unsubstituted; and n is 1, 2, 3, 4, 5, or 6.

In some embodiments is a compound of Formulas (Id) and (Id1), wherein R¹³ is methyl, ethyl, isopropyl, n-propyl, tert-butyl, n-butyl, n-pentyl, iso-amyl, cyclopropyl, cyclobutyl, cyclopentyl, or cyclohexyl. In some embodiments is a compound of Formula (Id1), wherein R¹³ is methyl.

In some embodiments is a compound of Formulas (Id) and (Id2) having the structure of Formula (VII-2):

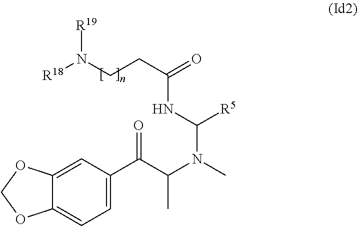

or a pharmaceutically acceptable salt thereof, wherein each of R¹⁸ and R¹⁹ is independently hydrogen, alkyl, heteroalkyl, cycloalkyl, or heterocycloalkyl, wherein alkyl, heteroalkyl, cycloalkyl, or heterocycloalkyl are substituted or unsubstituted; or R¹⁸ and R¹⁹ together with the atom to which they are attached form a heterocycloalkyl ring that is substituted or unsubstituted; R⁵ is alkyl that is substituted or unsubstituted, or hydrogen; and n is 1, 2, 3, 4, 5, or 6.

In some embodiments is a compound of Formula (Id2), wherein each of R¹⁸ and R¹⁹ is independently hydrogen methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, or —CH₂cPr.

In some embodiments is a compound of Formula (Id2), wherein R¹⁸ and R¹⁹ together with the atom to which they are attached form an azetidine ring, piperidine ring, piperazine ring, a morpholine ring, or a pyrrolidine ring, each of which is substituted or unsubstituted.

In some embodiments is a compound of Formula (Id) and (Id2) having the structure of Formula (Id3), or a pharmaceutically acceptable salt thereof:

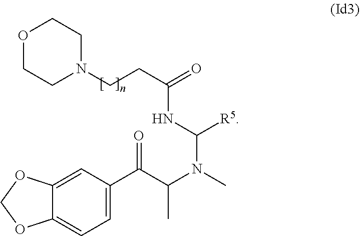

In some embodiments is a compound of Formula (Id) and(Id2) having the structure of Formula (Id4):

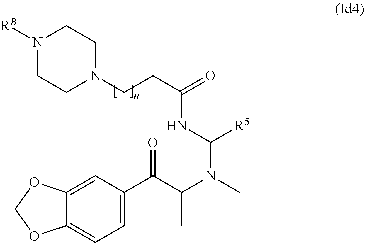

or a pharmaceutically acceptable salt thereof, wherein R^{B} is hydrogen, —C(O)CH₃, —C(O)Ph, alkyl, heteroalkyl, or cycloalkyl, wherein alkyl, heteroalkyl, and cycloalkyl are substituted or unsubstituted; R⁵ is alkyl that is substituted or unsubstituted, or hydrogen; and n is 1, 2, 3, 4, 5, or 6.

In some embodiments is a compound of Formula (Id4), wherein R^{B} is methyl, ethyl, n-propyl, isopropyl, or —CH(Et)₂. In some embodiments is a compound of Formula (Id4), wherein R^{B} is hydrogen.

In some embodiments is a compound of Formula (Id) having the structure of Formula (Id5)):

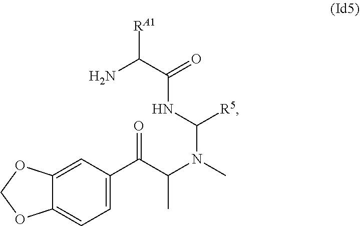

or a pharmaceutically acceptable salt thereof, wherein R^{A1} is hydrogen, alkyl or an amino acid side chain; is an amino acid side chain formed from an α-amino acid side chain, such as one of the naturally occurring amino acid side chains, such as an amino acid selected from alanine, serine, tryptophan, aspartic acid, glutamic acid and the like. The carbon to which R^{A1} is attached is chiral (unless R^{A1} is hydrogen, as in glycine), and this carbon atom can have either the (R) or (S) configuration. By way of illustration, when R^{A1} is formed from alanine, R^{A1} is methyl. In some embodiments is a compound of Formula (Id5), wherein R^{A1} is methyl, isopropyl, —CH(Me)Et, —CH₂CH(Me)₂, or —CH₂Ph.

In some embodiments is a compound of Formula (Id) and (Id5), wherein the compound is:

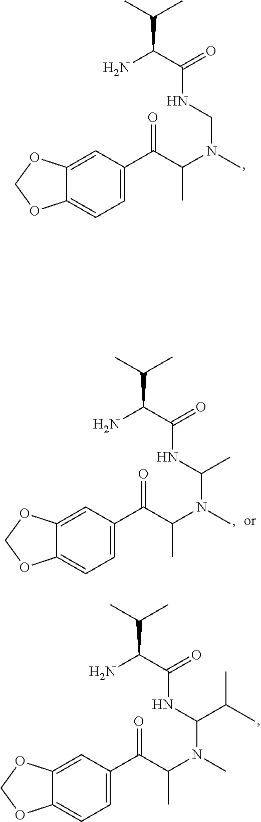

or a pharmaceutically acceptable salt thereof.

In some embodiments is a compound of Formula (Id) having the structure of Formula (Id-1), or a pharmaceutically acceptable salt thereof:

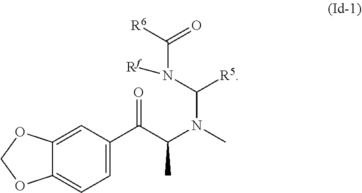

In some embodiments is a compound of Formula (Id) and (Id-1) having the structure of Formula (Id1-1), or a pharmaceutically acceptable salt thereof:

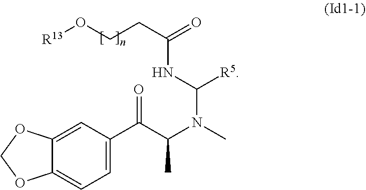

In some embodiments is a compound of Formula (Id) and (Id-1) having the structure of Formula (Id2-1), or a pharmaceutically acceptable salt thereof:

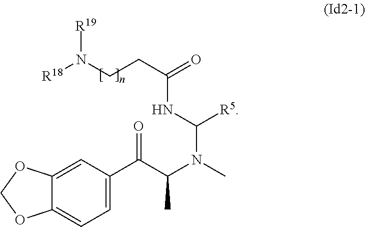

In some embodiments is a compound of Formula (Id), (Id2), (Id-1), and (Id2-1) having the structure of Formula (Id3-1), or a pharmaceutically acceptable salt thereof:

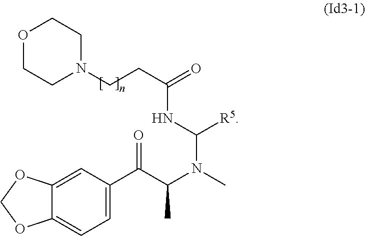

In some embodiments is a compound of Formula (Id), (Id2), (Id2-1), and (Id4) having the structure of Formula (Id4-1), or a pharmaceutically acceptable salt thereof:

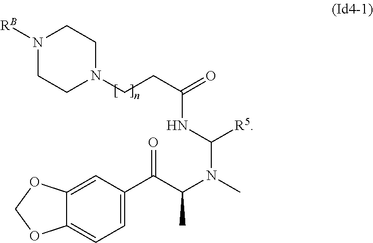

In some embodiments is a compound of Formula (Id), (Id-1), and (Id5) having the structure of Formula (Id5-1), or a pharmaceutically acceptable salt thereof:

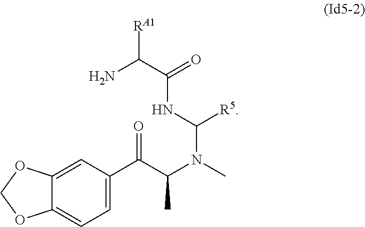

In some embodiments is a compound of Formula (Id) having the structure of Formula (Id-2), or a pharmaceutically acceptable salt thereof:

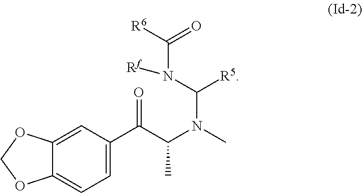

In some embodiments is a compound of Formula (Id) and (Id-2) having the structure of Formula (Id1-2), or a pharmaceutically acceptable salt thereof:

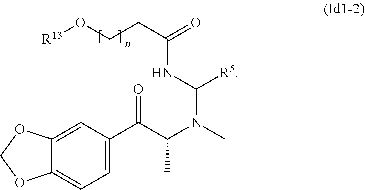

In some embodiments is a compound of Formula (Id) and (Id-2) having the structure of Formula (Id2-2), or a pharmaceutically acceptable salt thereof:

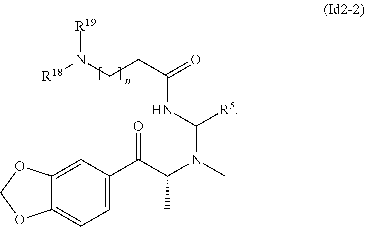

In some embodiments is a compound of Formula (Id), (Id2), (Id-2), and (Id2-2) having the structure of Formula (Id3-2), or a pharmaceutically acceptable salt thereof:

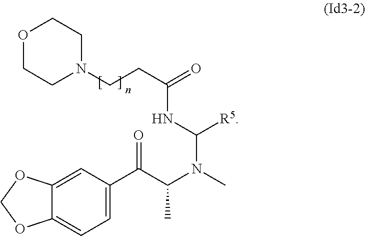

In some embodiments is a compound of Formula (Id), (Id2), (Id2-2), and (Id4) having the structure of Formula (Id4-2), or a pharmaceutically acceptable salt thereof:

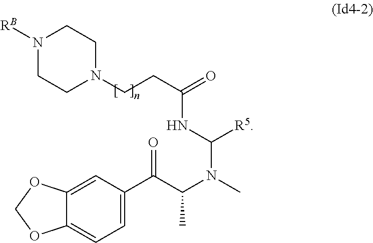

In some embodiments is a compound of Formula (Id), (Id-2), and (Id5) having the structure of Formula (Id5-2), or a pharmaceutically acceptable salt thereof:

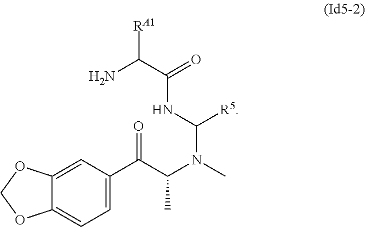

In some embodiments is a compound of Formulas (Id), (Id1), (Id2), (Id3), (Id4), (Id5), (Id-1), (Id1-1), (Id2-1), (Id3-1), (Id4-1), (Id5-1) (Id-2), (Id1-2), (Id2-2), (Id3-2), (Id4-2), and/or (Id5-2), wherein R⁵ is hydrogen. In some embodiments is a compound of Formulas (Id), (Id1), (Id2), (Id3), (Id4), (Id5), (Id-1), (Id1-1), (Id2-1), (Id3-1), (Id4-1), (Id5-1) (Id-2), (Id1-2), (Id2-2), (Id3-2), (Id4-2), and/or (Id5-2), wherein R⁵ is methyl, ethyl, n-propyl, isopropyl, or —CH(Et)₂.

In some embodiments is a compound of Formulas (Id1), (Id2), (Id3), (Id4), (Id1-1), (Id2-1), (Id3-1), (Id4-1), (Id1-2), (Id2-2), (Id3-2) and/or (Id4-2), wherein n is 1.

In another aspect, the present disclosure provides a compound of Formula (Ie):

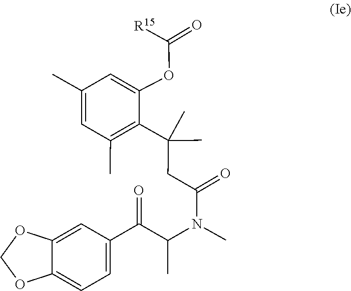

or a pharmaceutically acceptable salt thereof, wherein R¹⁵ is alkyl, alkenyl, haloalkyl, heteroalkyl, cycloalkyl, heterocycloalkyl, aryl, or heteroaryl, each of which is substituted or unsubstituted. In one embodiment of Formula (Ie), R¹⁵ and the carbonyl to which R¹⁵ is attached form an amino acid residue.

In some embodiments is a compound of Formulas (I) and (Ie), wherein R¹⁵ is alkyl or heteroalkyl that is substituted or unsubstituted. In some embodiments is a compound of Formula (Ie), wherein R¹⁵ is alkyl that is substituted. In some embodiments is a compound of Formula (Ie), wherein R¹⁵ is alkyl that is substituted with heterocycloalkyl that is substituted or unsubstituted. In some embodiments of Formulas (I) and (Ie), R¹⁵ is alkyl, such as methyl.

In some embodiments is a compound of Formula (Ie), wherein R¹⁵ is heteroalkyl that is substituted. In some embodiments is a compound of Formula (Ie), wherein R¹⁵ is heteroalkyl that is substituted with cycloalkyl or heterocycloalkyl, wherein cycloalkyl or heterocycloalkyl are substituted or unsubstituted.

In some embodiments is a compound of Formula (Ie), wherein R¹⁵ is heterocycloalkyl that is substituted or unsubstituted. In some embodiments is a compound of Formula (Ie), wherein R⁵ is heterocycloalkyl that is substituted with alkyl.

In some embodiments is a compound of Formula (Ie), wherein the compound is:

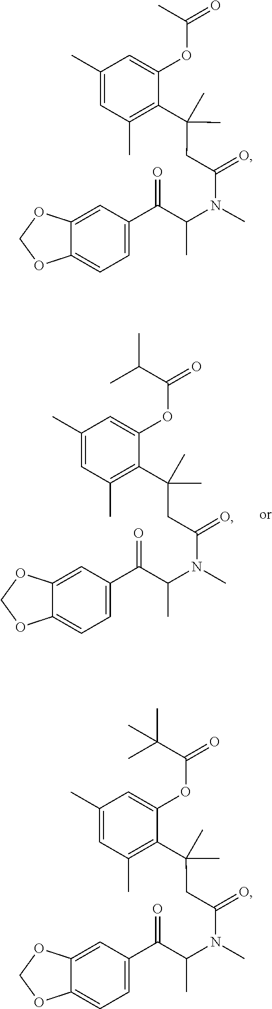 [SMILES: CC(=O)Oc1cc(C)cc(C)c1C(C)(C)CC(=O)N(C)C(C)C(=O)c1ccc2c(c1)OCO2 ; Cc1cc(C)c(C(C)(C)CC(=O)N(C)C(C)C(=O)c2ccc3c(c2)OCO3)c(OC(=O)C(C)C)c1 ; Cc1cc(C)c(C(C)(C)CC(=O)N(C)C(C)C(=O)c2ccc3c(c2)OCO3)c(OC(=O)C(C)(C)C)c1]

or a pharmaceutically acceptable salt thereof.

In some embodiments is a compound of Formula (Ie), wherein R¹⁵ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, —CH₂CH₂OMe, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (Ie) having the structure of Formula (Ie-1), or a pharmaceutically acceptable salt thereof:

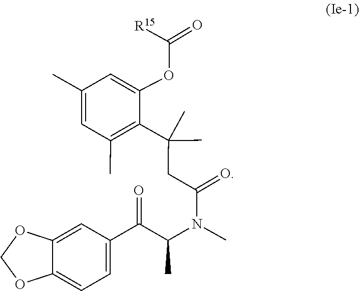

In some embodiments is a compound of Formula (Ie) having the structure of Formula (Ie-2), or a pharmaceutically acceptable salt thereof:

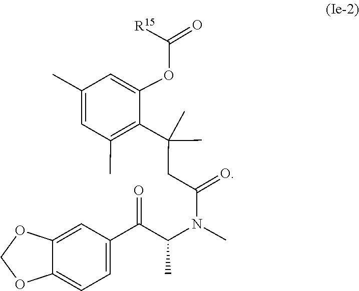

In another aspect, the present disclosure provides a compound of Formula (If):

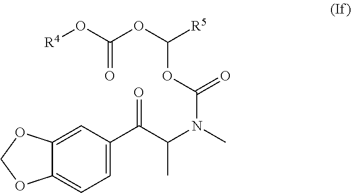

or a pharmaceutically acceptable salt thereof, wherein R⁴ is alkyl, alkenyl, heteroalkyl, cycloalkyl, haloalkyl, heterocycloalkyl, aryl, or heteroaryl, each of which is substituted or unsubstituted; and R⁵ is alkyl that is substituted or unsubstituted, or hydrogen. In one embodiment, compounds of Formula (If) may be referred to as N-acyloxymethylene carbonate prodrug compounds.

In some embodiments of compounds of Formula (If), R⁴ is heteroalkyl. In some embodiments of Formula (If), R⁴ is heterocycloalkyl. In some embodiments of Formula (If), R⁵ is hydrogen and R⁴ is heteroalkyl. In some embodiments of a compound of Formula (If), R⁵ is hydrogen and R⁴ is heterocycloalkyl. In some embodiments of Formula (If), R⁵ is hydrogen or C₁₋₆ alkyl and R⁴ is heteroalkyl. In some embodiments of Formula (If), R⁴ is heterocycloalkyl.

In some embodiments of Formulas (I) and (If), R⁴ is alkyl. In some embodiments of a compound of Formula (If), R⁴ is CH₂CF₃. In some embodiments of a compound of Formula (If), R⁴ is unsubstituted alkyl. In some embodiments of Formula (If), R⁴ is methyl, ethyl, n-propyl, isopropyl, n-butyl, tert-butyl, 3-methyl-1-butyl, n-pentyl, n-hexyl, n-heptyl, n-octyl, or n-nonyl. In some embodiments a compound of Formula (If) is one wherein R⁴ is cycloalkyl. In some embodiments of compound of Formula (If), R⁴ is unsubstituted cycloalkyl. In some embodiments of compounds of Formula (If), R⁴ is cyclopropyl, cyclobutyl, cyclopentyl, cyclohexyl, or cycloheptyl. In some embodiments of a compound of Formula (If), the compound is one wherein R⁴ is aryl. In some embodiments of Formula (If), wherein R⁴ is substituted or unsubstituted phenyl. In other embodiments of Formula (If), R⁴ is heteroaryl and in certain such some embodiments of Formula (If), R⁴ is 2-pyridyl, 3-pyridyl, 4-pyridyl, 2-pyrimidyl, 3-pyrimidyl, or 6-pyrimidyl.

In some embodiments of Formulas (I) and (If), R⁴ is R^{A} substituted with —OR³, —N(R¹⁸)R¹⁹, or —C(O)OR¹³, such as wherein R⁴ is alkyl, substituted with —OR¹³, —N(R¹⁸)R¹⁹, or —C(O)OR¹³. In some embodiments of Formula (If), R⁴ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (If) R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(O)CH₃. In one embodiment of Formula (If) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (If), R⁴ is heteroalkyl. In some embodiments of Formulas (I) and (If), R⁴ is CH₂CH₂OMe or CH₂CH₂SO₂Me. In some embodiments of Formulas (I) and (If), R⁴ is —(CH₂)ₙCO₂H, wherein n is 1, 2, 3, 4, 5, or 6. In some embodiments of Formulas (I) and (If), R⁴ is —(CH₂)ₙCO₂R¹³, wherein n is 1, 2, 3, 4, 5, or 6. In some embodiments of Formulas (I) and (If), R⁴ is —(CH₂)ₙCO₂R¹³, wherein R¹³ is alkyl. In some embodiments of Formulas (I) and (If), R⁴ is —(CH₂)ₙCO₂R¹³, wherein R¹³ is unsubstituted alkyl. In some embodiments of Formulas (I) and (If), R⁴ is —(CH₂)ₛCO₂R¹³, wherein R¹³ is methyl, ethyl, n-propyl, isopropyl, n-butyl, tert-butyl, or —CH(Et)₂.

In some embodiments of Formulas (I) and (If), R⁴ is —(CH₂)ₙOR¹³, wherein n is 1, 2, 3, 4, 5, 6 or 7. In some embodiments of Formulas (I) and (If), R⁴ is —(CH₂)ₙOR¹³, wherein R¹³ is alkyl. In some embodiments of Formulas (I) and (If), R⁴ is —(CH₂)ₙOR¹³, wherein R¹³ is unsubstituted alkyl. In some embodiments of Formulas (I) and (If), R⁴ is —(CH₂)ₛOR¹³, wherein R¹³ is methyl, ethyl, n-propyl, isopropyl, n-butyl, tert-butyl, or —CH(Et)₂.

In some embodiments of Formulas (I) and (If), R⁴ is —(CH₂)ₙN(R¹⁸)R¹⁹, wherein n is 1, 2, 3, 4, 5, 6 or 7. In some embodiments of Formulas (I) and (If), R⁴ is

—(CH₂)ₙ—N(R^{1′})R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments is a compound of Formula (If), wherein R⁵ is unsubstituted alkyl. In some embodiments is a compound of Formula (If), wherein R⁵ is methyl, ethyl, n-propyl, isopropyl, tert-butyl, or hydrogen. In some embodiments is a compound of Formula (If) or a pharmaceutically acceptable salt thereof, wherein R⁵ is methyl or hydrogen. In some embodiments is a compound of Formula (If), wherein R⁵ is methyl. In some embodiments is a compound of Formula (If), wherein R⁵ is hydrogen.

In some embodiments is a compound of Formula (If), wherein the compound is:

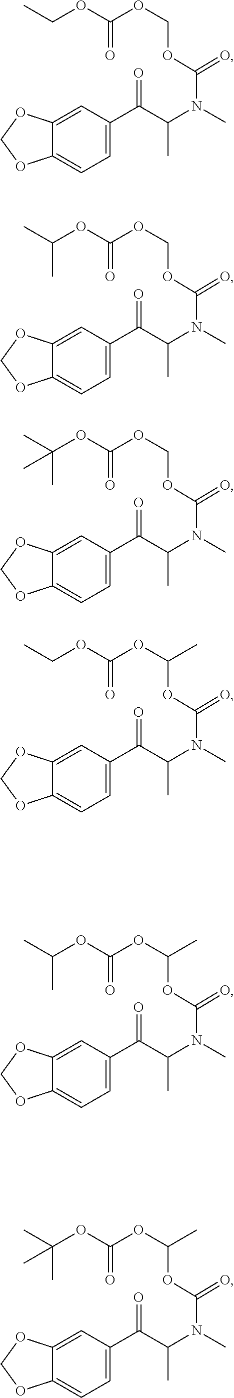

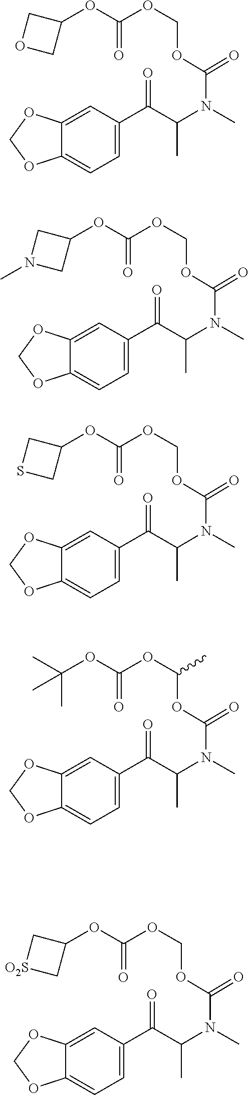

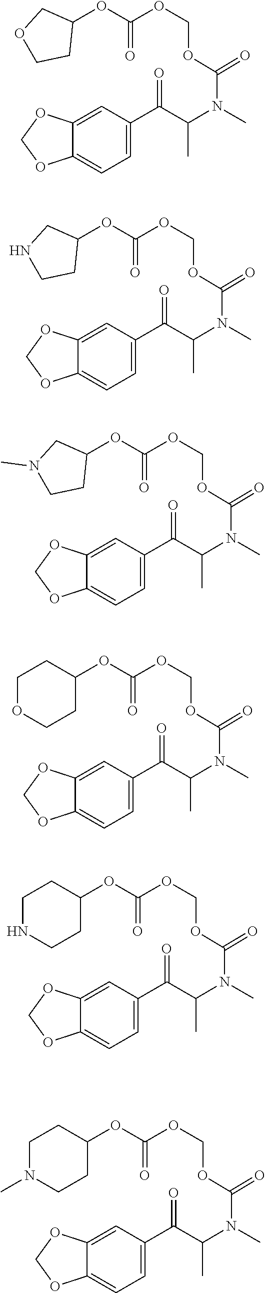

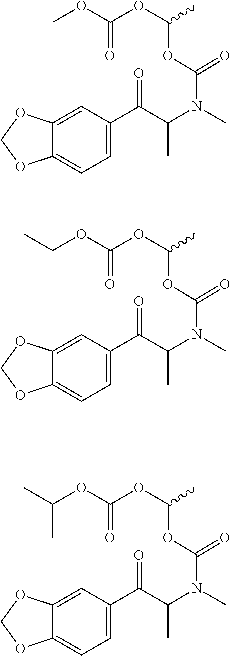

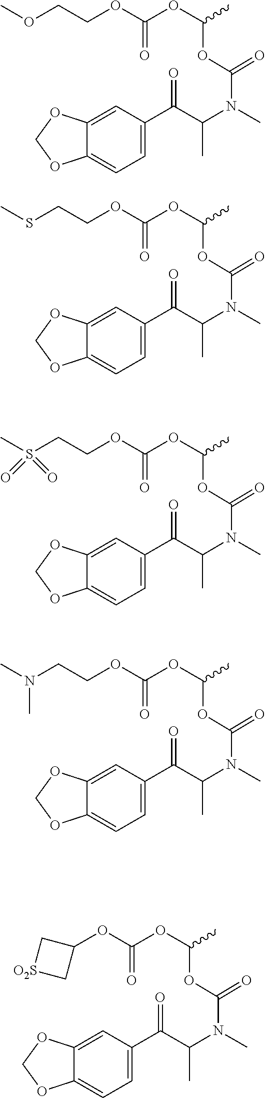

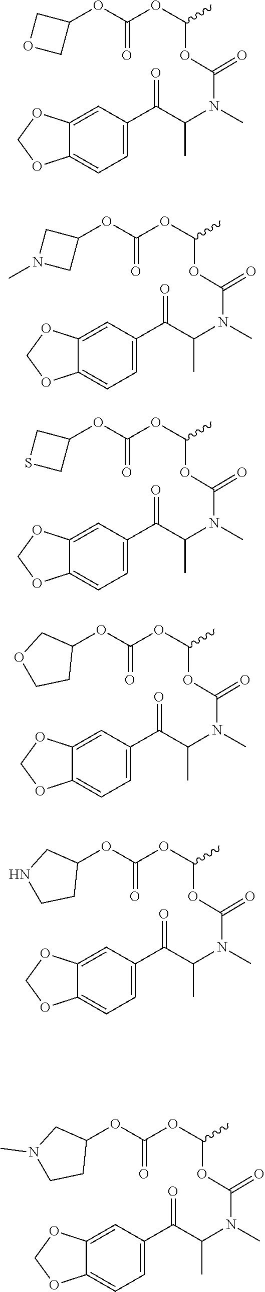

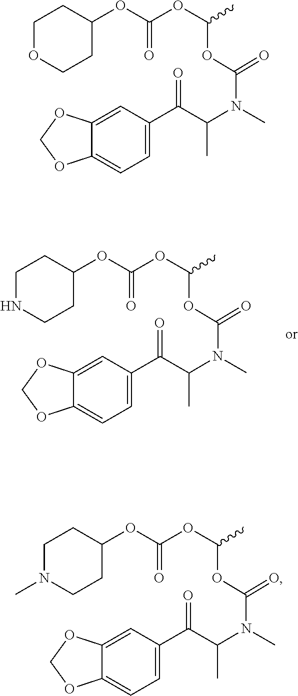

or a pharmaceutically acceptable salt thereof.

In some embodiments is a compound of Formula (If), wherein R⁴ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, —CH₂CH₂OMe, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (If), wherein R⁵ is hydrogen. In some embodiments is a compound of Formula (If), wherein R⁴ is methyl, ethyl, n-propyl, isopropyl, or —CH(Et)₂.

In some embodiments is a compound of Formula (If) having the structure of Formula (If-1), or a pharmaceutically acceptable salt thereof:

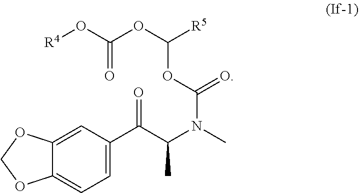

In some embodiments is a compound of Formula (If) having the structure of Formula (If-2), or a pharmaceutically acceptable salt thereof:

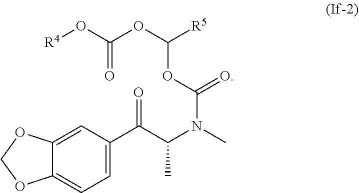

In another aspect, described herein is a compound of Formula (II):

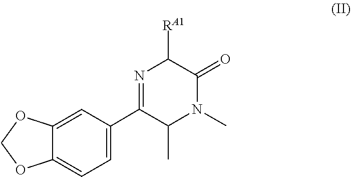

or a pharmaceutically acceptable salt thereof, wherein:
R^{A1} is an amino acid side chain.

In some embodiments, the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂. In some embodiments, the amino acid side chain is —CH₂CH₂CH₂CH₂—NH₂, —CH₂(CH₃)₂, —CH₂phenyl, or —CH₃.

In another aspect, described herein is a compound of Formula (III):

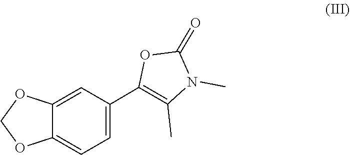 [SMILES: Cc1c(-c2ccc3c(c2)OCO3)oc(=O)n1C]

or a pharmaceutically acceptable salt thereof.

### Certain Terminology

Compounds herein can include all stereoisomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocycloalkyl groups, heteroaryl groups, cycloalkyl groups, acyl groups, acyloxy groups, carbamate groups, amide groups, ureido groups, epoxy groups, and ester groups.

Non-limiting examples of alkyl groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Alkyl groups can include branched and unbranched alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cycloalkyl groups include cyclopropyl, cyclobutyl, cyclopentyl, cyclohexyl, cycloheptyl, and cyclooctyl groups. Cycloalkyl groups also include fused-, bridged-, and spiro-bicycles and higher fused-, bridged-, and spiro-systems. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl groups include straight, branched, and cyclic alkenyl groups. The olefin or olefins of an alkenyl group can be, for example, E, Z, cis, trans, terminal, or exo-methylene. An alkenyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-i-en-1-yl, isopropenyl, but-i-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl groups include straight, branched, and cyclic alkynyl groups. The triple bond of an alkynyl group can be internal or terminal. An alkynyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl groups include ethynyl, prop-2-yn-1-yl, prop-i-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

“Alkyl” refers to an optionally substituted straight-chain, or optionally substituted branched-chain saturated hydrocarbon having from one to about ten carbon atoms, or from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon having one or more carbon-carbon double-bonds and having from two to about ten carbon atoms, more preferably two to about six carbon atoms, wherein an sp²-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)=CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkoxy” refers to a radical of the formula —ORₐ where Rₐ is an alkyl radical as defined. Non-limiting examples of alkoxy groups include methoxy, ethoxy, propoxy, isopropoxy, and isobutoxy. In some embodiments, alkoxy is C₁-C₆ alkoxy. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl. In some embodiments, haloalkyl is C₁-C₆ haloalkyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycle” refers to heteroaryl and heterocycloalkyl ring systems. A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. In some embodiments, heterocycloalkyl is aziridinyl, azetidinyl, morpholinyl, piperidinyl, piperazinyl, oxetanyl, tetrahydrofuryl, tetrahydropyranyl, or thiomorpholinyl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). In some embodiments, heteroaryl is imidazolyl, indazolyl, indolyl, pyrrolyl, pyrazolyl, pyridinyl, pyrazinyl, pyrimidinyl, pyridazinyl, triazolyl, or tetrazolyl. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Certain compounds according to Formula (I) disclosed herein are isotopically enriched, meaning that they have an isotope present in greater than its natural abundance at one or more position. The term “isotopic enrichment factor” as used herein means the ratio between the isotopic abundance and the natural abundance of a specified isotope. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds. In one embodiment, deuterated compounds disclosed herein have an isotopic enrichment factor for each designated atom of at least 3500 (52.5%), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In some embodiments, the present disclosure provides a deuterated analogue of any compound disclosed herein. A deuterated analogue can include a compound herein where one or more ¹H atoms is replaced with a deuterium atom. With reference to Formula (I), isotopically enriched compounds of the present disclosure according to Formula I include, without limitation, those having deuterium on the methylene dioxole moiety, for example wherein Formula (I) has the structure:

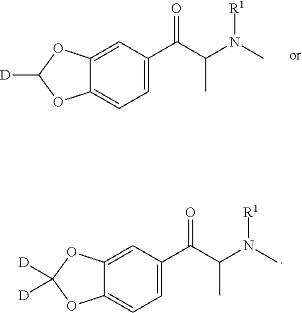

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

The present disclosure provides for pharmaceutically-acceptable salts of any compound described herein as well as the use of such salts. As is understood by those of skill in the art, any compound with an ionizable group, such as an acidic hydrogen, or a basic nitrogen, can be provided in the form of a salt, and pharmaceutically acceptable salt forms of such compounds are specifically contemplated herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine.

In some embodiments, an ammonium salt is a triethyl amine salt, trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, xinafoic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, a xinafoate salt, or a maleate salt.

### Pharmaceutical Compositions

According to another embodiment, the present disclosure provides a composition comprising a compound of the present disclosure and a pharmaceutically acceptable carrier, adjuvant, or vehicle. The amount of compound in the composition is an amount effective to treat the relevant disease, disorder, or condition in a patient in need thereof (an “effective amount”). In some embodiments, a composition of the present disclosure is formulated for oral administration to a patient.

The term “pharmaceutically acceptable carrier, adjuvant, or vehicle” refers to a non-toxic carrier, adjuvant, or vehicle that does not destroy the pharmacological activity of the agent with which it is formulated. Pharmaceutically acceptable carriers, adjuvants or vehicles that may be used in the disclosed compositions include, but are not limited to, ion exchangers, alumina, stearates such as aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally, enterally, intracistemally, intraperitoneally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the composition is administered orally, intraperitoneally, or intravenously. In some embodiments, the composition is a transmucosal formulation. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

To aid in delivery of the composition, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, may also be added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, pharmaceutically acceptable compositions may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, the pharmaceutically acceptable composition is formulated for oral administration. Such formulations may be administered with or without food. In some embodiments, the pharmaceutically acceptable composition is administered without food. In other embodiments, the pharmaceutically acceptable composition is administered with food.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. And isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinyl pyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Therapeutic agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Selected compounds of the disclosure are provided in TABLE 1.

TABLE 1

| Cpd | Structure |
|---|---|
| 1 | 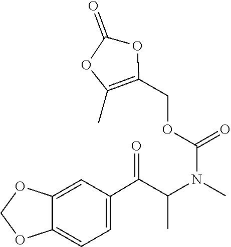 [SMILES: Cc1oc(=O)oc1COC(=O)N(C)C(C)C(=O)c1ccc2c(c1)OCO2] |
| 2 | 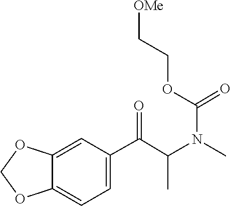 [SMILES: COCCOC(=O)N(C)C(C)C(=O)c1ccc2c(c1)OCO2] |
| 3 | 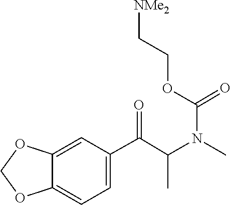 [SMILES: CC(C(=O)c1ccc2c(c1)OCO2)N(C)C(=O)OCCN(C)C] |
| 4 | 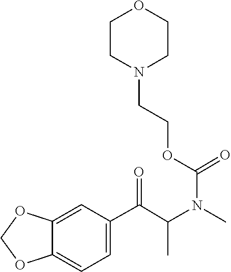 [SMILES: CC(C(=O)c1ccc2c(c1)OCO2)N(C)C(=O)OCCN1CCOCC1] |
| 5 | 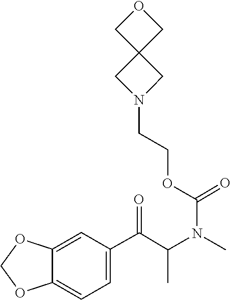 [SMILES: CC(C(=O)c1ccc2c(c1)OCO2)N(C)C(=O)OCCN1CC2(COC2)C1] |
| 6 | 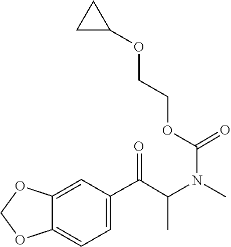 [SMILES: CC(C(=O)c1ccc2c(c1)OCO2)N(C)C(=O)OCCOC1CC1] |
| 7 | 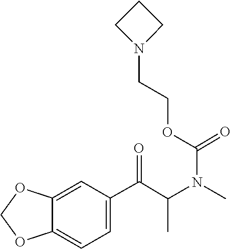 [SMILES: CC(C(=O)c1ccc2c(c1)OCO2)N(C)C(=O)OCCN1CCC1] |
| 8 | 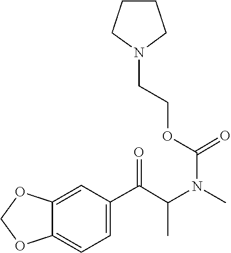 [SMILES: CC(C(=O)c1ccc2c(c1)OCO2)N(C)C(=O)OCCN1CCCC1] |
| 9 | 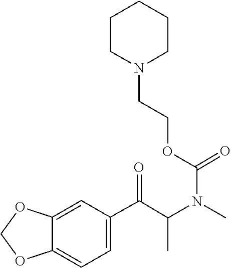 [SMILES: CC(C(=O)c1ccc2c(c1)OCO2)N(C)C(=O)OCCN1CCCCC1] |
| 10 | 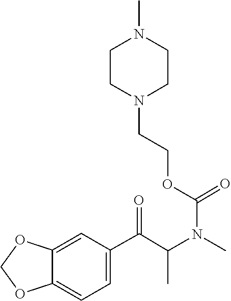 [SMILES: CC(C(=O)c1ccc2c(c1)OCO2)N(C)C(=O)OCCN1CCN(C)CC1] |
| 11 | 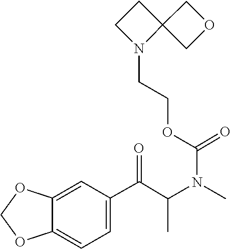 [SMILES: CC(C(=O)c1ccc2c(c1)OCO2)N(C)C(=O)OCCN1CCC12COC2] |
| 12 | 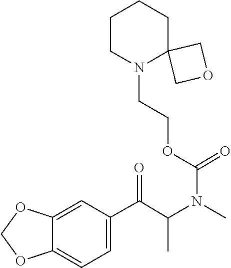 [SMILES: CC(C(=O)c1ccc2c(c1)OCO2)N(C)C(=O)OCCN1CCCCC12COC2] |
| 13 | 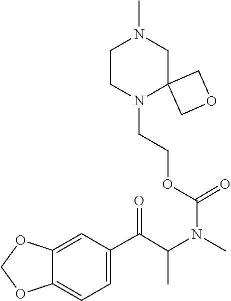 [SMILES: CC(C(=O)c1ccc2c(c1)OCO2)N(C)C(=O)OCCN1CCN(C)CC12COC2] |
| 14 | 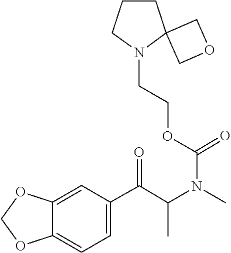 [SMILES: CC(C(=O)c1ccc2c(c1)OCO2)N(C)C(=O)OCCN1CCCC12COC2] |
| 15 | 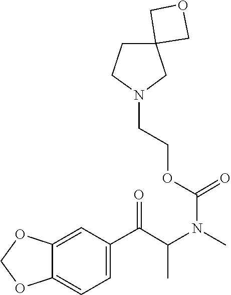 [SMILES: CC(C(=O)c1ccc2c(c1)OCO2)N(C)C(=O)OCCN1CCC2(COC2)C1] |
| 16 | 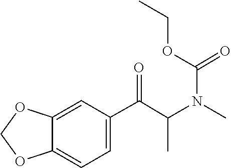 [SMILES: CCOC(=O)N(C)C(C)C(=O)c1ccc2c(c1)OCO2] |
| 17 | 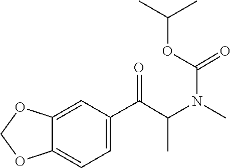 [SMILES: CC(C)OC(=O)N(C)C(C)C(=O)c1ccc2c(c1)OCO2] |
| 18 | 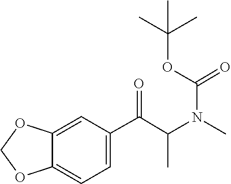 [SMILES: CC(C(=O)c1ccc2c(c1)OCO2)N(C)C(=O)OC(C)(C)C] |
| 19 | 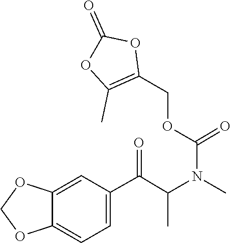 [SMILES: Cc1oc(=O)oc1COC(=O)N(C)C(C)C(=O)c1ccc2c(c1)OCO2] |
| 20 | 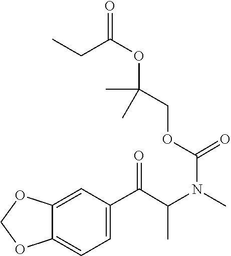 [SMILES: CCC(=O)OC(C)(C)COC(=O)N(C)C(C)C(=O)c1ccc2c(c1)OCO2] |
| 21 | 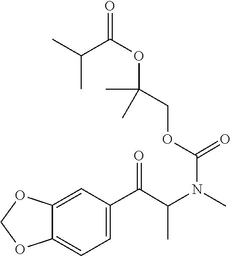 [SMILES: CC(C)C(=O)OC(C)(C)COC(=O)N(C)C(C)C(=O)c1ccc2c(c1)OCO2] |
| 22 | 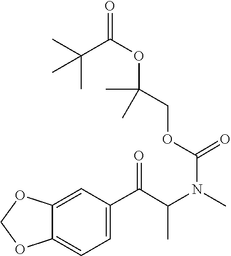 [SMILES: CC(C(=O)c1ccc2c(c1)OCO2)N(C)C(=O)OCC(C)(C)OC(=O)C(C)(C)C] |
| 23 | 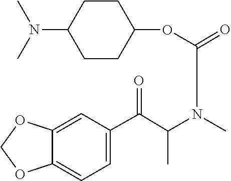 [SMILES: CC(C(=O)c1ccc2c(c1)OCO2)N(C)C(=O)OC1CCC(N(C)C)CC1] |
| 24 | 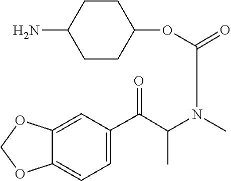 [SMILES: CC(C(=O)c1ccc2c(c1)OCO2)N(C)C(=O)OC1CCC(N)CC1] |
| 25 | 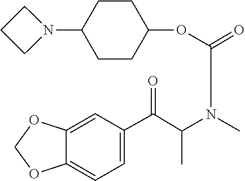 [SMILES: CC(C(=O)c1ccc2c(c1)OCO2)N(C)C(=O)OC1CCC(N2CCC2)CC1] |
| 26 | 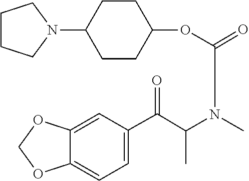 [SMILES: CC(C(=O)c1ccc2c(c1)OCO2)N(C)C(=O)OC1CCC(N2CCCC2)CC1] |
| 27 | 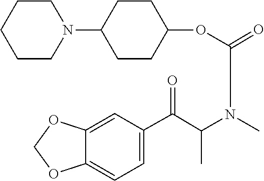 [SMILES: CC(C(=O)c1ccc2c(c1)OCO2)N(C)C(=O)OC1CCC(N2CCCCC2)CC1] |
| 28 | 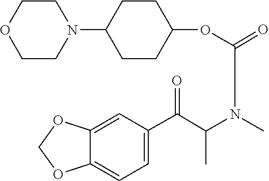 [SMILES: CC(C(=O)c1ccc2c(c1)OCO2)N(C)C(=O)OC1CCC(N2CCOCC2)CC1] |
| 29 | 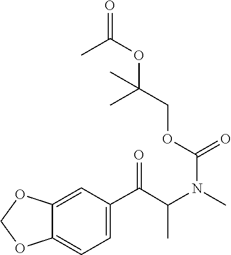 [SMILES: CC(=O)OC(C)(C)COC(=O)N(C)C(C)C(=O)c1ccc2c(c1)OCO2] |
| 30 | 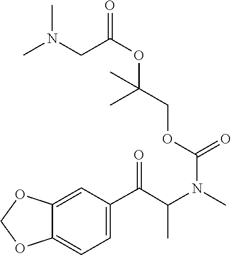 [SMILES: CC(C(=O)c1ccc2c(c1)OCO2)N(C)C(=O)OCC(C)(C)OC(=O)CN(C)C] |
| 31 | 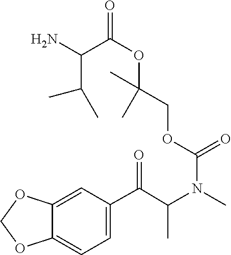 [SMILES: CC(C)C(N)C(=O)OC(C)(C)COC(=O)N(C)C(C)C(=O)c1ccc2c(c1)OCO2] |
| 32 | 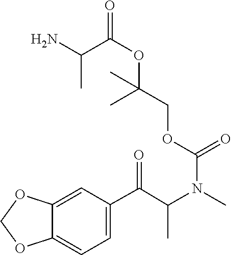 [SMILES: CC(N)C(=O)OC(C)(C)COC(=O)N(C)C(C)C(=O)c1ccc2c(c1)OCO2] |
| 33 | 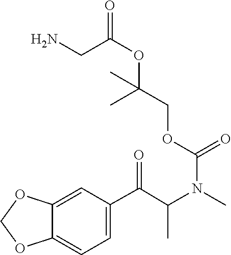 [SMILES: CC(C(=O)c1ccc2c(c1)OCO2)N(C)C(=O)OCC(C)(C)OC(=O)CN] |
| 34 | 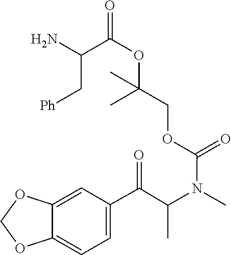 [SMILES: CC(C(=O)c1ccc2c(c1)OCO2)N(C)C(=O)OCC(C)(C)OC(=O)C(N)Cc1ccccc1] |
| 35 | 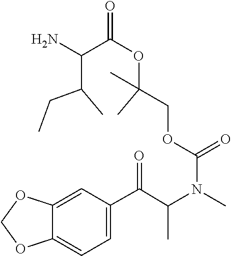 [SMILES: CCC(C)C(N)C(=O)OC(C)(C)COC(=O)N(C)C(C)C(=O)c1ccc2c(c1)OCO2] |
| 36 | 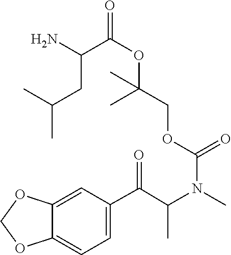 [SMILES: CC(C)CC(N)C(=O)OC(C)(C)COC(=O)N(C)C(C)C(=O)c1ccc2c(c1)OCO2] |
| 37 | 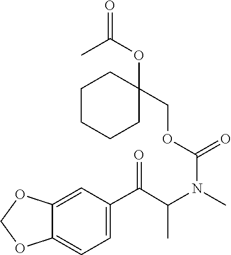 [SMILES: CC(=O)OC1(COC(=O)N(C)C(C)C(=O)c2ccc3c(c2)OCO3)CCCCC1] |
| 38 | 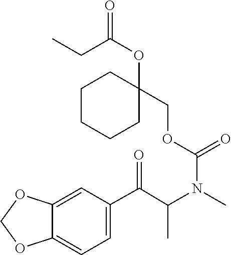 [SMILES: CCC(=O)OC1(COC(=O)N(C)C(C)C(=O)c2ccc3c(c2)OCO3)CCCCC1] |
| 39 | 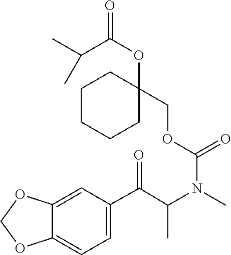 [SMILES: CC(C)C(=O)OC1(COC(=O)N(C)C(C)C(=O)c2ccc3c(c2)OCO3)CCCCC1] |
| 40 | 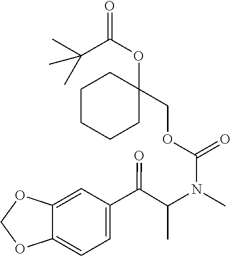 [SMILES: CC(C(=O)c1ccc2c(c1)OCO2)N(C)C(=O)OCC1(OC(=O)C(C)(C)C)CCCCC1] |
| 41 | 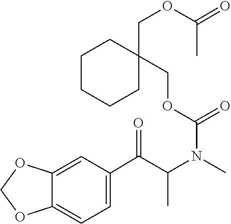 [SMILES: CC(=O)OCC1(COC(=O)N(C)C(C)C(=O)c2ccc3c(c2)OCO3)CCCCC1] |
| 42 | 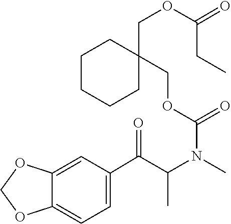 [SMILES: CCC(=O)OCC1(COC(=O)N(C)C(C)C(=O)c2ccc3c(c2)OCO3)CCCCC1] |
| 43 | 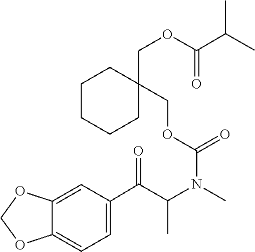 [SMILES: CC(C)C(=O)OCC1(COC(=O)N(C)C(C)C(=O)c2ccc3c(c2)OCO3)CCCCC1] |
| 44 | 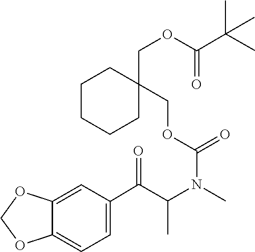 [SMILES: CC(C(=O)c1ccc2c(c1)OCO2)N(C)C(=O)OCC1(COC(=O)C(C)(C)C)CCCCC1] |
| 45 | 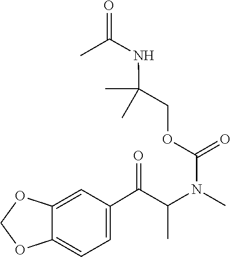 [SMILES: CC(=O)NC(C)(C)COC(=O)N(C)C(C)C(=O)c1ccc2c(c1)OCO2] |
| 46 | 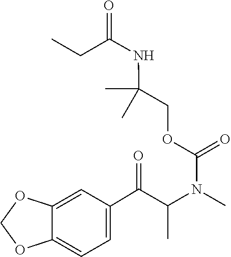 [SMILES: CCC(=O)NC(C)(C)COC(=O)N(C)C(C)C(=O)c1ccc2c(c1)OCO2] |
| 47 | 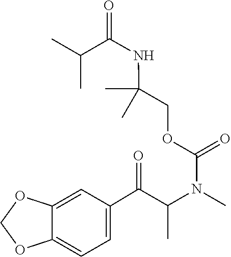 [SMILES: CC(C)C(=O)NC(C)(C)COC(=O)N(C)C(C)C(=O)c1ccc2c(c1)OCO2] |
| 48 | 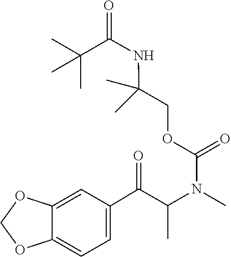 [SMILES: CC(C(=O)c1ccc2c(c1)OCO2)N(C)C(=O)OCC(C)(C)NC(=O)C(C)(C)C] |
| 49 | 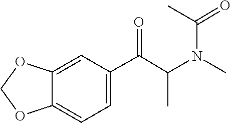 [SMILES: CC(=O)N(C)C(C)C(=O)c1ccc2c(c1)OCO2] |
| 50 | 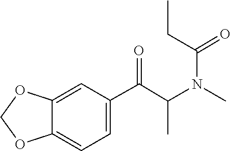 [SMILES: CCC(=O)N(C)C(C)C(=O)c1ccc2c(c1)OCO2] |
| 51 | 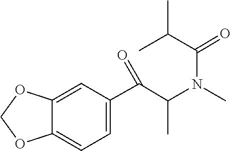 [SMILES: CC(C)C(=O)N(C)C(C)C(=O)c1ccc2c(c1)OCO2] |
| 52 | 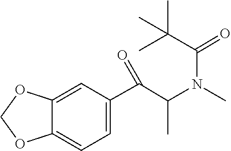 [SMILES: CC(C(=O)c1ccc2c(c1)OCO2)N(C)C(=O)C(C)(C)C] |
| 53 | 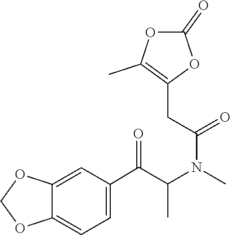 [SMILES: Cc1oc(=O)oc1CC(=O)N(C)C(C)C(=O)c1ccc2c(c1)OCO2] |
| 54 | 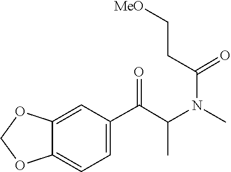 [SMILES: COCCC(=O)N(C)C(C)C(=O)c1ccc2c(c1)OCO2] |
| 55 | 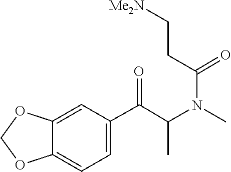 [SMILES: CC(C(=O)c1ccc2c(c1)OCO2)N(C)C(=O)CCN(C)C] |
| 56 | 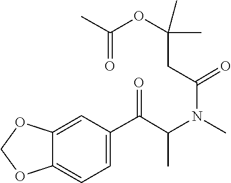 [SMILES: CC(=O)OC(C)(C)CC(=O)N(C)C(C)C(=O)c1ccc2c(c1)OCO2] |
| 57 | 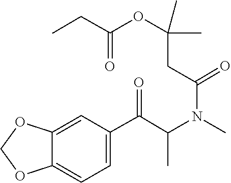 [SMILES: CCC(=O)OC(C)(C)CC(=O)N(C)C(C)C(=O)c1ccc2c(c1)OCO2] |
| 58 | 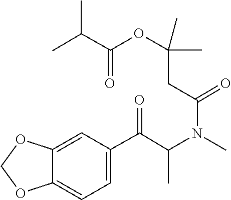 [SMILES: CC(C)C(=O)OC(C)(C)CC(=O)N(C)C(C)C(=O)c1ccc2c(c1)OCO2] |
| 59 | 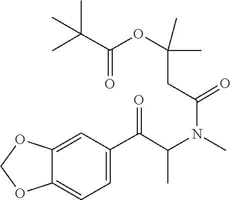 [SMILES: CC(C(=O)c1ccc2c(c1)OCO2)N(C)C(=O)CC(C)(C)OC(=O)C(C)(C)C] |
| 60 | 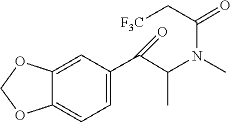 [SMILES: CC(C(=O)c1ccc2c(c1)OCO2)N(C)C(=O)CC(F)(F)F] |
| 61 | 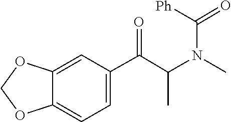 [SMILES: CC(C(=O)c1ccc2c(c1)OCO2)N(C)C(=O)c1ccccc1] |
| 62 | 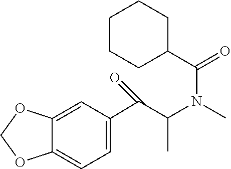 [SMILES: CC(C(=O)c1ccc2c(c1)OCO2)N(C)C(=O)C1CCCCC1] |
| 63 | 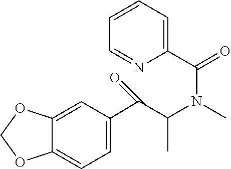 [SMILES: CC(C(=O)c1ccc2c(c1)OCO2)N(C)C(=O)c1ccccn1] |
| 64 | 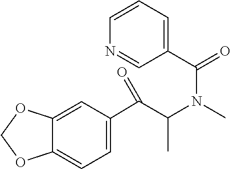 [SMILES: CC(C(=O)c1ccc2c(c1)OCO2)N(C)C(=O)c1cccnc1] |
| 65 | 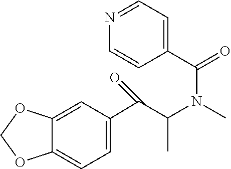 [SMILES: CC(C(=O)c1ccc2c(c1)OCO2)N(C)C(=O)c1ccncc1] |
| 66 | 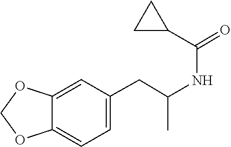 [SMILES: CC(Cc1ccc2c(c1)OCO2)NC(=O)C1CC1] |
| 67 | 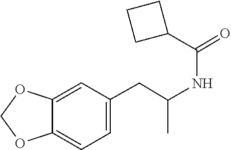 [SMILES: CC(Cc1ccc2c(c1)OCO2)NC(=O)C1CCC1] |
| 68 | 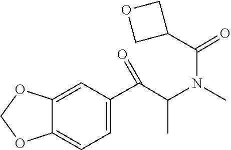 [SMILES: CC(C(=O)c1ccc2c(c1)OCO2)N(C)C(=O)C1COC1] |
| 69 | 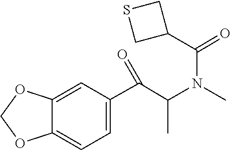 [SMILES: CC(C(=O)c1ccc2c(c1)OCO2)N(C)C(=O)C1CSC1] |
| 70 | 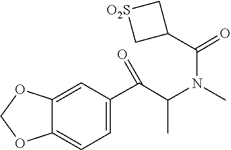 [SMILES: CC(C(=O)c1ccc2c(c1)OCO2)N(C)C(=O)C1CS(=O)(=O)C1] |
| 71 | 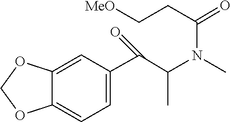 [SMILES: COCCC(=O)N(C)C(C)C(=O)c1ccc2c(c1)OCO2] |
| 72 | 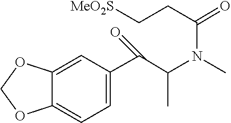 [SMILES: CC(C(=O)c1ccc2c(c1)OCO2)N(C)C(=O)CCS(C)(=O)=O] |
| 73 | 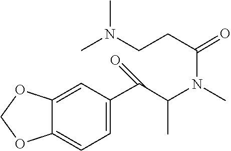 [SMILES: CC(C(=O)c1ccc2c(c1)OCO2)N(C)C(=O)CCN(C)C] |
| 74 | 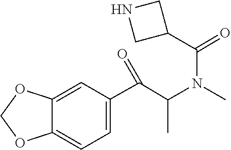 [SMILES: CC(C(=O)c1ccc2c(c1)OCO2)N(C)C(=O)C1CNC1] |
| 75 | 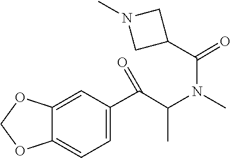 [SMILES: CC(C(=O)c1ccc2c(c1)OCO2)N(C)C(=O)C1CN(C)C1] |
| 76 | 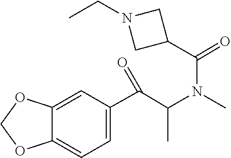 [SMILES: CCN1CC(C(=O)N(C)C(C)C(=O)c2ccc3c(c2)OCO3)C1] |
| 77 | 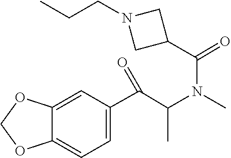 [SMILES: CCCN1CC(C(=O)N(C)C(C)C(=O)c2ccc3c(c2)OCO3)C1] |
| 78 | 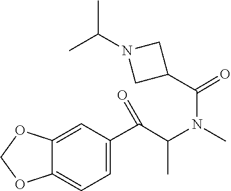 [SMILES: CC(C)N1CC(C(=O)N(C)C(C)C(=O)c2ccc3c(c2)OCO3)C1] |
| 79 | 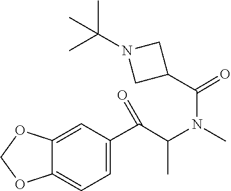 [SMILES: CC(C(=O)c1ccc2c(c1)OCO2)N(C)C(=O)C1CN(C(C)(C)C)C1] |
| 80 | 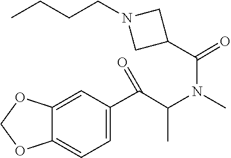 [SMILES: CCCCN1CC(C(=O)N(C)C(C)C(=O)c2ccc3c(c2)OCO3)C1] |
| 81 | 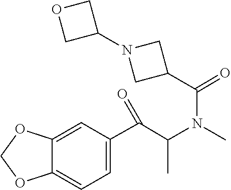 [SMILES: CC(C(=O)c1ccc2c(c1)OCO2)N(C)C(=O)C1CN(C2COC2)C1] |
| 82 | 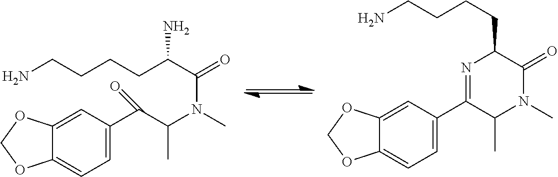 [SMILES: CC(C(=O)c1ccc2c(c1)OCO2)N(C)C(=O)[C@@H](N)CCCCN ; CC1C(c2ccc3c(c2)OCO3)=N[C@@H](CCCCN)C(=O)N1C] |
| 83 | 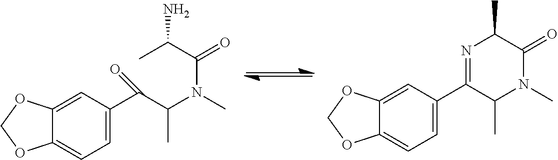 [SMILES: CC(C(=O)c1ccc2c(c1)OCO2)N(C)C(=O)[C@H](C)N ; CC1C(c2ccc3c(c2)OCO3)=N[C@@H](C)C(=O)N1C] |
| 84 | 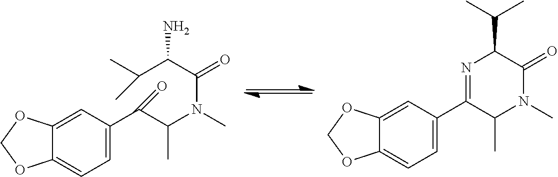 [SMILES: CC(C)[C@H](N)C(=O)N(C)C(C)C(=O)c1ccc2c(c1)OCO2 ; CC(C)[C@@H]1N=C(c2ccc3c(c2)OCO3)C(C)N(C)C1=O] |
| 85 | 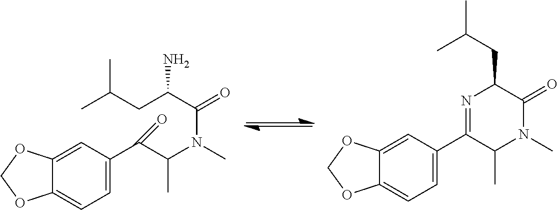 [SMILES: CC(C)C[C@H](N)C(=O)N(C)C(C)C(=O)c1ccc2c(c1)OCO2 ; CC(C)C[C@@H]1N=C(c2ccc3c(c2)OCO3)C(C)N(C)C1=O] |
| 86 | 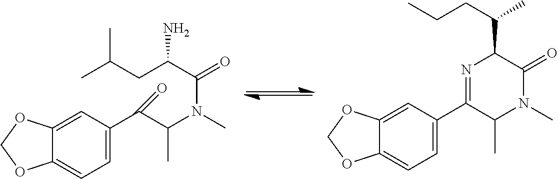 [SMILES: CC(C)C[C@H](N)C(=O)N(C)C(C)C(=O)c1ccc2c(c1)OCO2 ; CCC[C@H](C)[C@@H]1N=C(c2ccc3c(c2)OCO3)C(C)N(C)C1=O] |
| 87 | 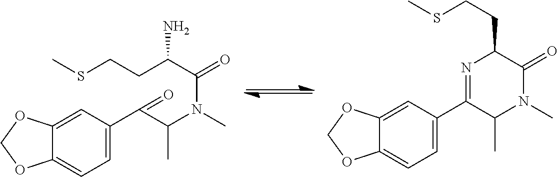 [SMILES: CSCC[C@H](N)C(=O)N(C)C(C)C(=O)c1ccc2c(c1)OCO2 ; CSCC[C@@H]1N=C(c2ccc3c(c2)OCO3)C(C)N(C)C1=O] |
| 88 | 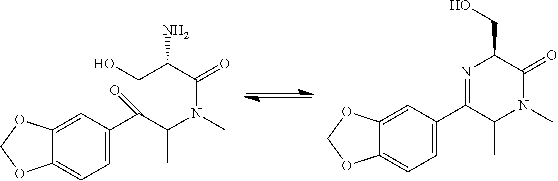 [SMILES: CC(C(=O)c1ccc2c(c1)OCO2)N(C)C(=O)[C@@H](N)CO ; CC1C(c2ccc3c(c2)OCO3)=N[C@@H](CO)C(=O)N1C] |
| 89 | 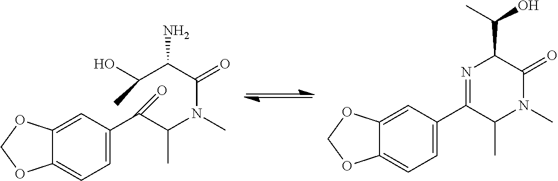 [SMILES: CC(C(=O)c1ccc2c(c1)OCO2)N(C)C(=O)[C@@H](N)[C@@H](C)O ; CC1C(c2ccc3c(c2)OCO3)=N[C@@H]([C@@H](C)O)C(=O)N1C] |
| 90 | 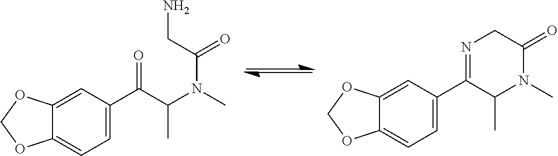 [SMILES: CC(C(=O)c1ccc2c(c1)OCO2)N(C)C(=O)CN ; CC1C(c2ccc3c(c2)OCO3)=NCC(=O)N1C] |
| 91 | 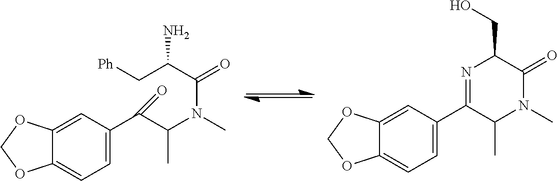 [SMILES: CC(C(=O)c1ccc2c(c1)OCO2)N(C)C(=O)[C@@H](N)Cc1ccccc1 ; CC1C(c2ccc3c(c2)OCO3)=N[C@@H](CO)C(=O)N1C] |
| 92 | 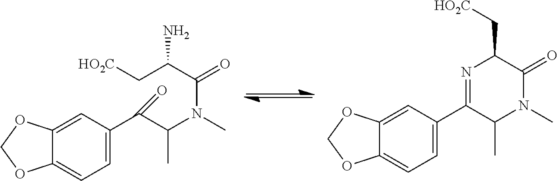 [SMILES: CC(C(=O)c1ccc2c(c1)OCO2)N(C)C(=O)[C@@H](N)CC(=O)O ; CC1C(c2ccc3c(c2)OCO3)=N[C@@H](CC(=O)O)C(=O)N1C] |
| 93 | 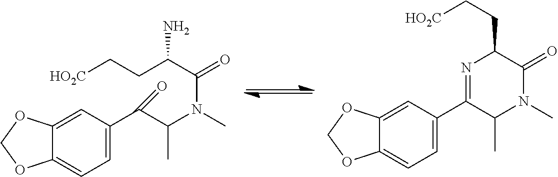 [SMILES: CC(C(=O)c1ccc2c(c1)OCO2)N(C)C(=O)[C@@H](N)CCC(=O)O ; CC1C(c2ccc3c(c2)OCO3)=N[C@@H](CCC(=O)O)C(=O)N1C] |
| 94 | 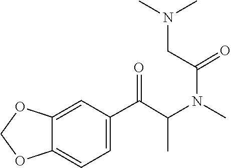 [SMILES: CC(C(=O)c1ccc2c(c1)OCO2)N(C)C(=O)CN(C)C] |
| 95 | 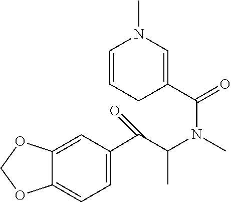 [SMILES: CC(C(=O)c1ccc2c(c1)OCO2)N(C)C(=O)C1=CN(C)C=CC1] |
| 96 | 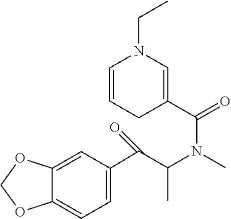 [SMILES: CCN1C=CCC(C(=O)N(C)C(C)C(=O)c2ccc3c(c2)OCO3)=C1] |
| 97 | 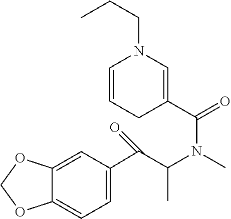 [SMILES: CCCN1C=CCC(C(=O)N(C)C(C)C(=O)c2ccc3c(c2)OCO3)=C1] |
| 98 | 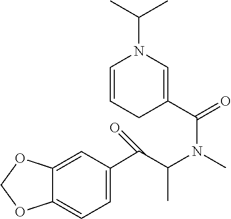 [SMILES: CC(C)N1C=CCC(C(=O)N(C)C(C)C(=O)c2ccc3c(c2)OCO3)=C1] |
| 99 | 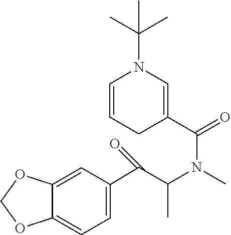 [SMILES: CC(C(=O)c1ccc2c(c1)OCO2)N(C)C(=O)C1=CN(C(C)(C)C)C=CC1] |
| 100 | 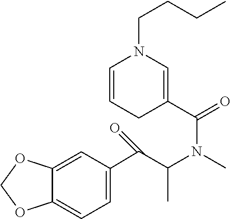 [SMILES: CCCCN1C=CCC(C(=O)N(C)C(C)C(=O)c2ccc3c(c2)OCO3)=C1] |
| 101 | 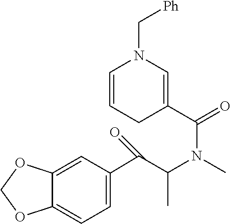 [SMILES: CC(C(=O)c1ccc2c(c1)OCO2)N(C)C(=O)C1=CN(Cc2ccccc2)C=CC1] |
| 102 | 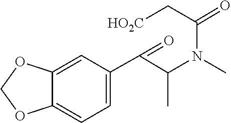 [SMILES: CC(C(=O)c1ccc2c(c1)OCO2)N(C)C(=O)CC(=O)O] |
| 103 | 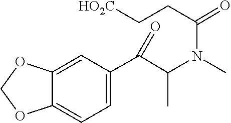 [SMILES: CC(C(=O)c1ccc2c(c1)OCO2)N(C)C(=O)CCC(=O)O] |
| 104 | 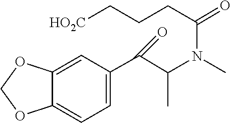 [SMILES: CC(C(=O)c1ccc2c(c1)OCO2)N(C)C(=O)CCCC(=O)O] |
| 105 | 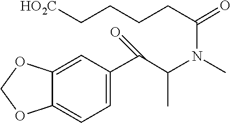 [SMILES: CC(C(=O)c1ccc2c(c1)OCO2)N(C)C(=O)CCCCC(=O)O] |
| 106 | 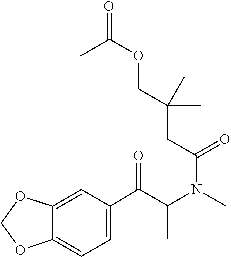 [SMILES: CC(=O)OCC(C)(C)CC(=O)N(C)C(C)C(=O)c1ccc2c(c1)OCO2] |
| 107 | 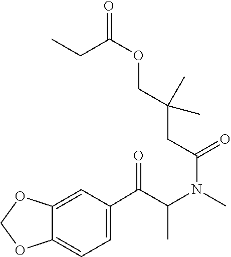 [SMILES: CCC(=O)OCC(C)(C)CC(=O)N(C)C(C)C(=O)c1ccc2c(c1)OCO2] |
| 108 | 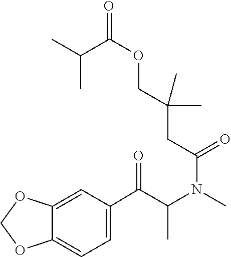 [SMILES: CC(C)C(=O)OCC(C)(C)CC(=O)N(C)C(C)C(=O)c1ccc2c(c1)OCO2] |
| 109 | 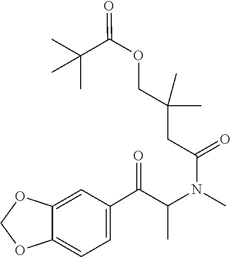 [SMILES: CC(C(=O)c1ccc2c(c1)OCO2)N(C)C(=O)CC(C)(C)COC(=O)C(C)(C)C] |
| 110 | 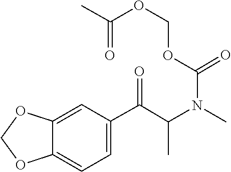 [SMILES: CC(=O)OCOC(=O)N(C)C(C)C(=O)c1ccc2c(c1)OCO2] |
| 111 | 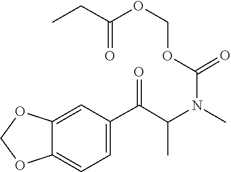 [SMILES: CCC(=O)OCOC(=O)N(C)C(C)C(=O)c1ccc2c(c1)OCO2] |
| 112 | 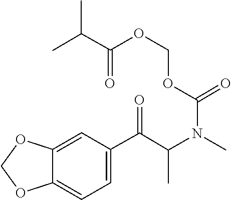 [SMILES: CC(C)C(=O)OCOC(=O)N(C)C(C)C(=O)c1ccc2c(c1)OCO2] |
| 113 | 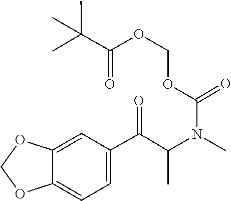 [SMILES: CC(C(=O)c1ccc2c(c1)OCO2)N(C)C(=O)OCOC(=O)C(C)(C)C] |
| 114 | 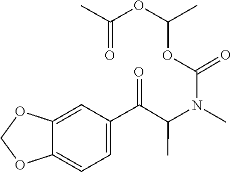 [SMILES: CC(=O)OC(C)OC(=O)N(C)C(C)C(=O)c1ccc2c(c1)OCO2] |
| 115 | 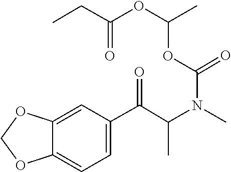 [SMILES: CCC(=O)OC(C)OC(=O)N(C)C(C)C(=O)c1ccc2c(c1)OCO2] |
| 116 | 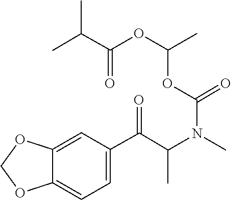 [SMILES: CC(OC(=O)C(C)C)OC(=O)N(C)C(C)C(=O)c1ccc2c(c1)OCO2] |
| 117 | 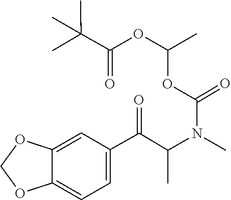 [SMILES: CC(OC(=O)N(C)C(C)C(=O)c1ccc2c(c1)OCO2)OC(=O)C(C)(C)C] |
| 118 | 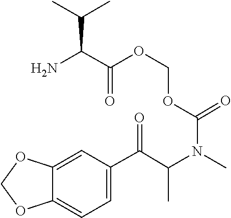 [SMILES: CC(C)[C@H](N)C(=O)OCOC(=O)N(C)C(C)C(=O)c1ccc2c(c1)OCO2] |
| 119 | 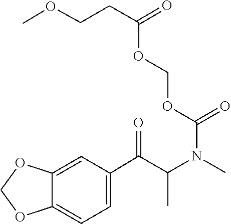 [SMILES: COCCC(=O)OCOC(=O)N(C)C(C)C(=O)c1ccc2c(c1)OCO2] |
| 120 | 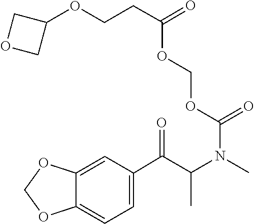 [SMILES: CC(C(=O)c1ccc2c(c1)OCO2)N(C)C(=O)OCOC(=O)CCOC1COC1] |
| 121 | 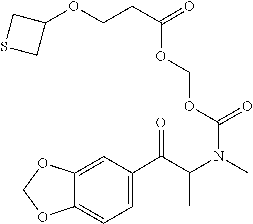 [SMILES: CC(C(=O)c1ccc2c(c1)OCO2)N(C)C(=O)OCOC(=O)CCOC1CSC1] |
| 122 | 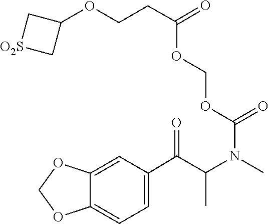 [SMILES: CC(C(=O)c1ccc2c(c1)OCO2)N(C)C(=O)OCOC(=O)CCOC1CS(=O)(=O)C1] |
| 123 | 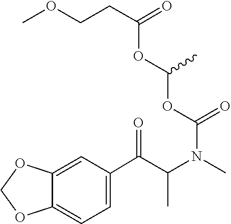 [SMILES: COCCC(=O)OC(C)OC(=O)N(C)C(C)C(=O)c1ccc2c(c1)OCO2] |
| 124 | 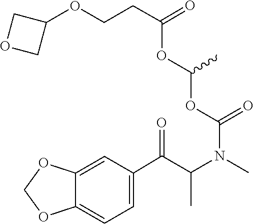 [SMILES: CC(OC(=O)CCOC1COC1)OC(=O)N(C)C(C)C(=O)c1ccc2c(c1)OCO2] |
| 125 | 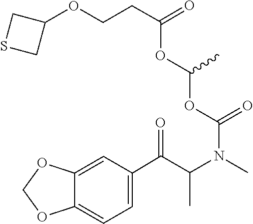 [SMILES: CC(OC(=O)CCOC1CSC1)OC(=O)N(C)C(C)C(=O)c1ccc2c(c1)OCO2] |
| 126 | 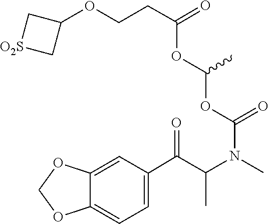 [SMILES: CC(OC(=O)CCOC1CS(=O)(=O)C1)OC(=O)N(C)C(C)C(=O)c1ccc2c(c1)OCO2] |
| 127 | 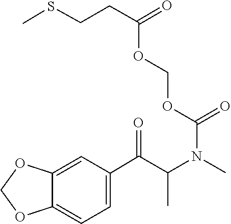 [SMILES: CSCCC(=O)OCOC(=O)N(C)C(C)C(=O)c1ccc2c(c1)OCO2] |
| 128 | 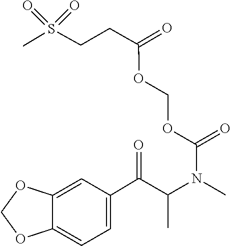 [SMILES: CC(C(=O)c1ccc2c(c1)OCO2)N(C)C(=O)OCOC(=O)CCS(C)(=O)=O] |
| 129 | 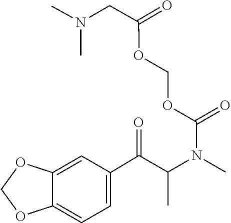 [SMILES: CC(C(=O)c1ccc2c(c1)OCO2)N(C)C(=O)OCOC(=O)CN(C)C] |
| 130 | 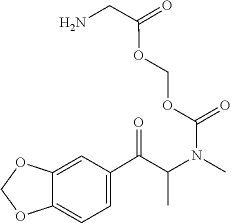 [SMILES: CC(C(=O)c1ccc2c(c1)OCO2)N(C)C(=O)OCOC(=O)CN] |
| 131 | 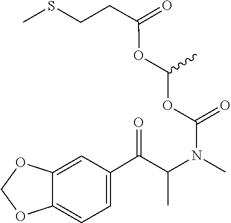 [SMILES: CSCCC(=O)OC(C)OC(=O)N(C)C(C)C(=O)c1ccc2c(c1)OCO2] |
| 132 | 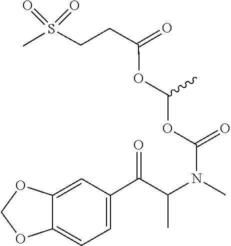 [SMILES: CC(OC(=O)CCS(C)(=O)=O)OC(=O)N(C)C(C)C(=O)c1ccc2c(c1)OCO2] |
| 133 | 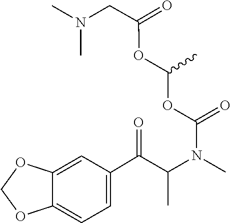 [SMILES: CC(OC(=O)CN(C)C)OC(=O)N(C)C(C)C(=O)c1ccc2c(c1)OCO2] |
| 134 | 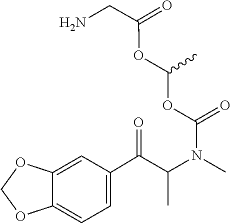 [SMILES: CC(OC(=O)CN)OC(=O)N(C)C(C)C(=O)c1ccc2c(c1)OCO2] |
| 135 | 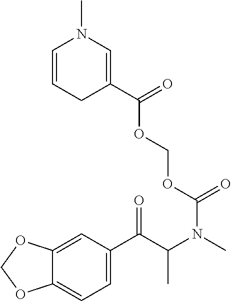 [SMILES: CC(C(=O)c1ccc2c(c1)OCO2)N(C)C(=O)OCOC(=O)C1=CN(C)C=CC1] |
| 136 | 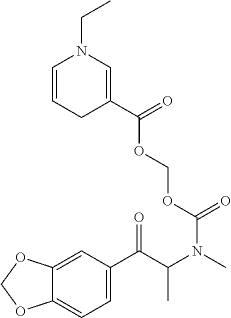 [SMILES: CCN1C=CCC(C(=O)OCOC(=O)N(C)C(C)C(=O)c2ccc3c(c2)OCO3)=C1] |
| 137 | 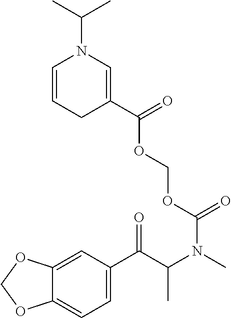 [SMILES: CC(C)N1C=CCC(C(=O)OCOC(=O)N(C)C(C)C(=O)c2ccc3c(c2)OCO3)=C1] |
| 138 | 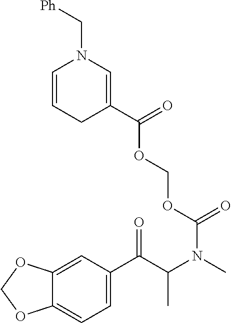 [SMILES: CC(C(=O)c1ccc2c(c1)OCO2)N(C)C(=O)OCOC(=O)C1=CN(Cc2ccccc2)C=CC1] |
| 139 | 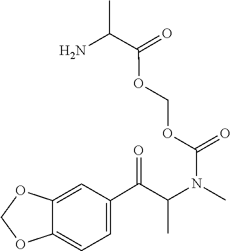 [SMILES: CC(N)C(=O)OCOC(=O)N(C)C(C)C(=O)c1ccc2c(c1)OCO2] |
| 140 | 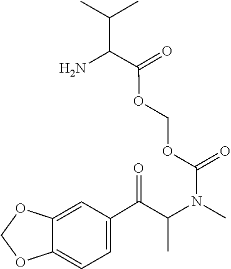 [SMILES: CC(C)C(N)C(=O)OCOC(=O)N(C)C(C)C(=O)c1ccc2c(c1)OCO2] |
| 141 | 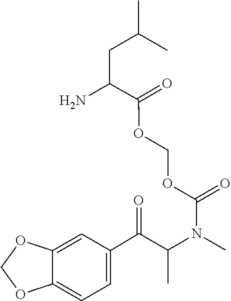 [SMILES: CC(C)CC(N)C(=O)OCOC(=O)N(C)C(C)C(=O)c1ccc2c(c1)OCO2] |
| 142 | 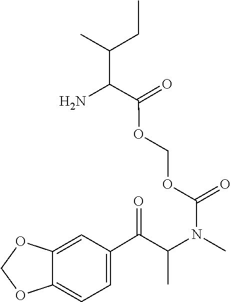 [SMILES: CCC(C)C(N)C(=O)OCOC(=O)N(C)C(C)C(=O)c1ccc2c(c1)OCO2] |
| 143 | 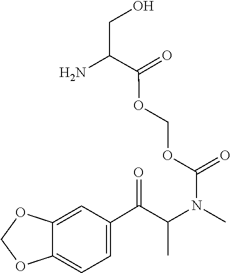 [SMILES: CC(C(=O)c1ccc2c(c1)OCO2)N(C)C(=O)OCOC(=O)C(N)CO] |
| 144 | 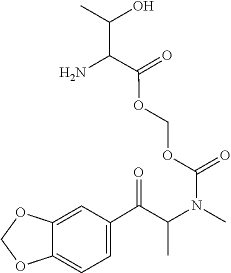 [SMILES: CC(O)C(N)C(=O)OCOC(=O)N(C)C(C)C(=O)c1ccc2c(c1)OCO2] |
| 145 | 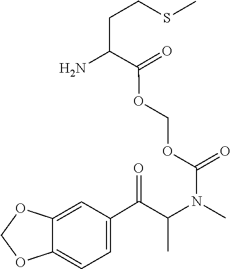 [SMILES: CSCCC(N)C(=O)OCOC(=O)N(C)C(C)C(=O)c1ccc2c(c1)OCO2] |
| 146 | 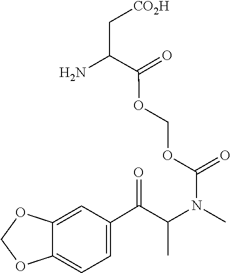 [SMILES: CC(C(=O)c1ccc2c(c1)OCO2)N(C)C(=O)OCOC(=O)C(N)CC(=O)O] |
| 147 | 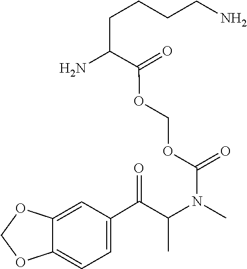 [SMILES: CC(C(=O)c1ccc2c(c1)OCO2)N(C)C(=O)OCOC(=O)C(N)CCCCN] |
| 148 | 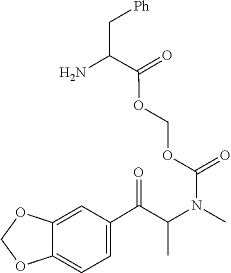 [SMILES: CC(C(=O)c1ccc2c(c1)OCO2)N(C)C(=O)OCOC(=O)C(N)Cc1ccccc1] |
| 149 | 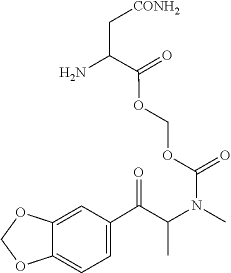 [SMILES: CC(C(=O)c1ccc2c(c1)OCO2)N(C)C(=O)OCOC(=O)C(N)CC(N)=O] |
| 150 | 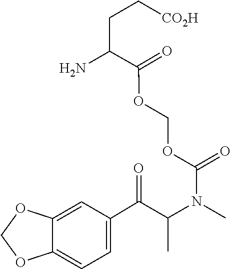 [SMILES: CC(C(=O)c1ccc2c(c1)OCO2)N(C)C(=O)OCOC(=O)C(N)CCC(=O)O] |
| 151 | 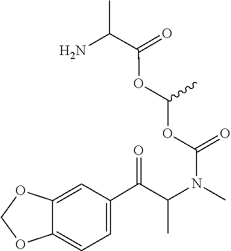 [SMILES: CC(OC(=O)C(C)N)OC(=O)N(C)C(C)C(=O)c1ccc2c(c1)OCO2] |
| 152 | 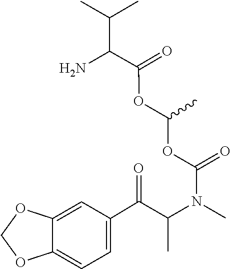 [SMILES: CC(OC(=O)C(N)C(C)C)OC(=O)N(C)C(C)C(=O)c1ccc2c(c1)OCO2] |
| 153 | 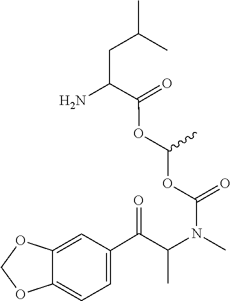 [SMILES: CC(C)CC(N)C(=O)OC(C)OC(=O)N(C)C(C)C(=O)c1ccc2c(c1)OCO2] |
| 154 | 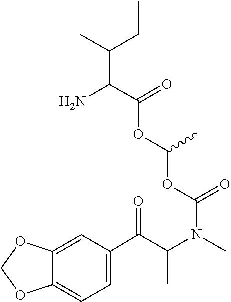 [SMILES: CCC(C)C(N)C(=O)OC(C)OC(=O)N(C)C(C)C(=O)c1ccc2c(c1)OCO2] |
| 155 | 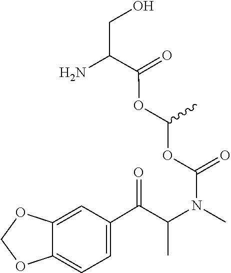 [SMILES: CC(OC(=O)C(N)CO)OC(=O)N(C)C(C)C(=O)c1ccc2c(c1)OCO2] |
| 156 | 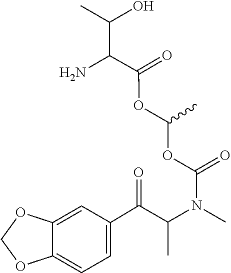 [SMILES: CC(OC(=O)C(N)C(C)O)OC(=O)N(C)C(C)C(=O)c1ccc2c(c1)OCO2] |
| 157 | 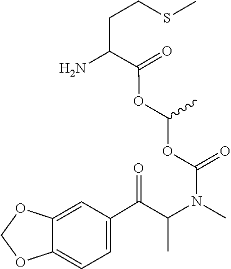 [SMILES: CSCCC(N)C(=O)OC(C)OC(=O)N(C)C(C)C(=O)c1ccc2c(c1)OCO2] |
| 158 | 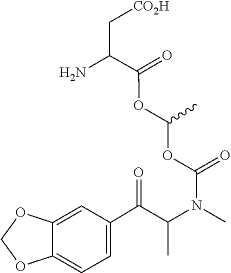 [SMILES: CC(OC(=O)C(N)CC(=O)O)OC(=O)N(C)C(C)C(=O)c1ccc2c(c1)OCO2] |
| 159 | 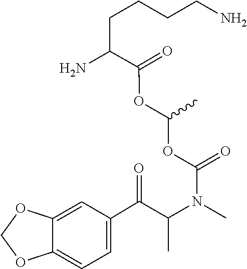 [SMILES: CC(OC(=O)C(N)CCCCN)OC(=O)N(C)C(C)C(=O)c1ccc2c(c1)OCO2] |
| 160 | 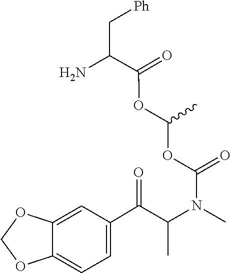 [SMILES: CC(OC(=O)C(N)Cc1ccccc1)OC(=O)N(C)C(C)C(=O)c1ccc2c(c1)OCO2] |
| 161 | 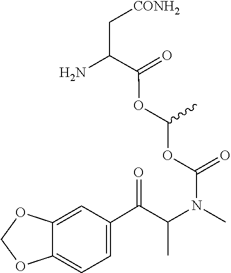 [SMILES: CC(OC(=O)C(N)CC(N)=O)OC(=O)N(C)C(C)C(=O)c1ccc2c(c1)OCO2] |
| 162 | 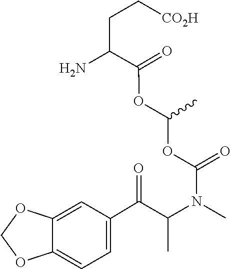 [SMILES: CC(OC(=O)C(N)CCC(=O)O)OC(=O)N(C)C(C)C(=O)c1ccc2c(c1)OCO2] |
| 163 | 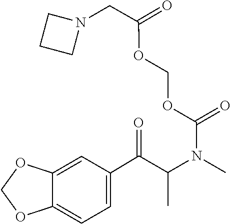 [SMILES: CC(C(=O)c1ccc2c(c1)OCO2)N(C)C(=O)OCOC(=O)CN1CCC1] |
| 164 | 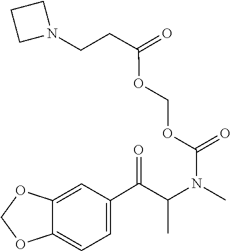 [SMILES: CC(C(=O)c1ccc2c(c1)OCO2)N(C)C(=O)OCOC(=O)CCN1CCC1] |
| 165 | 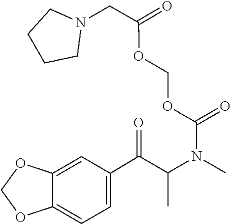 [SMILES: CC(C(=O)c1ccc2c(c1)OCO2)N(C)C(=O)OCOC(=O)CN1CCCC1] |
| 166 | 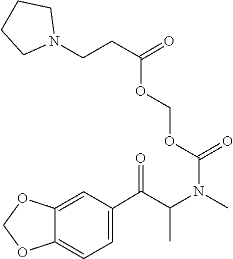 [SMILES: CC(C(=O)c1ccc2c(c1)OCO2)N(C)C(=O)OCOC(=O)CCN1CCCC1] |
| 167 | 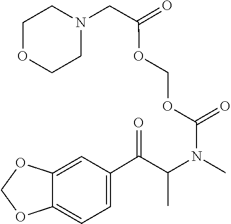 [SMILES: CC(C(=O)c1ccc2c(c1)OCO2)N(C)C(=O)OCOC(=O)CN1CCOCC1] |
| 168 | 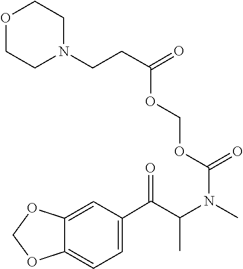 [SMILES: CC(C(=O)c1ccc2c(c1)OCO2)N(C)C(=O)OCOC(=O)CCN1CCOCC1] |
| 169 | 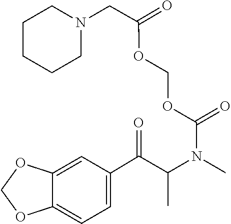 [SMILES: CC(C(=O)c1ccc2c(c1)OCO2)N(C)C(=O)OCOC(=O)CN1CCCCC1] |
| 170 | 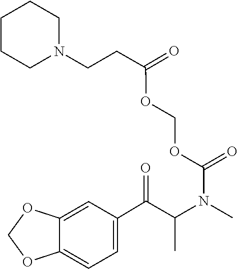 [SMILES: CC(C(=O)c1ccc2c(c1)OCO2)N(C)C(=O)OCOC(=O)CCN1CCCCC1] |
| 171 | 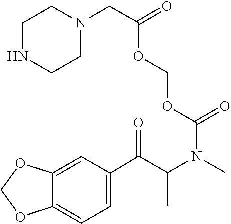 [SMILES: CC(C(=O)c1ccc2c(c1)OCO2)N(C)C(=O)OCOC(=O)CN1CCNCC1] |
| 172 | 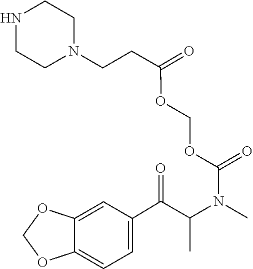 [SMILES: CC(C(=O)c1ccc2c(c1)OCO2)N(C)C(=O)OCOC(=O)CCN1CCNCC1] |
| 173 | 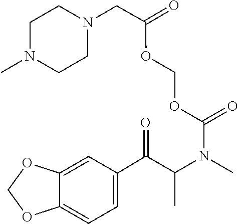 |
| 174 | 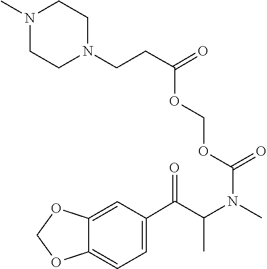 |
| 175 | 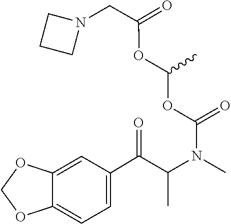 [SMILES: CC(OC(=O)CN1CCC1)OC(=O)N(C)C(C)C(=O)c1ccc2c(c1)OCO2] |
| 176 | 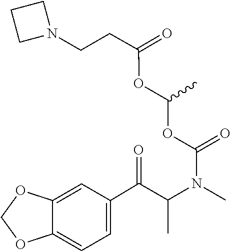 [SMILES: CC(OC(=O)CCN1CCC1)OC(=O)N(C)C(C)C(=O)c1ccc2c(c1)OCO2] |
| 177 | 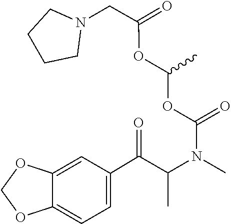 [SMILES: CC(OC(=O)CN1CCCC1)OC(=O)N(C)C(C)C(=O)c1ccc2c(c1)OCO2] |
| 178 | 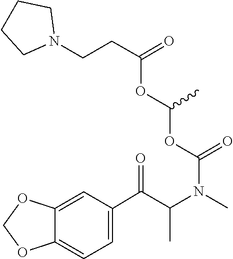 [SMILES: CC(OC(=O)CCN1CCCC1)OC(=O)N(C)C(C)C(=O)c1ccc2c(c1)OCO2] |
| 179 | 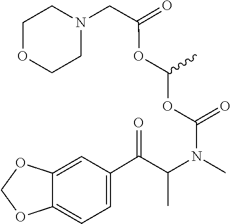 [SMILES: CC(OC(=O)CN1CCOCC1)OC(=O)N(C)C(C)C(=O)c1ccc2c(c1)OCO2] |
| 180 | 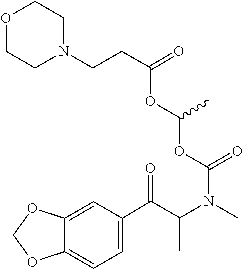 [SMILES: CC(OC(=O)CCN1CCOCC1)OC(=O)N(C)C(C)C(=O)c1ccc2c(c1)OCO2] |
| 181 | 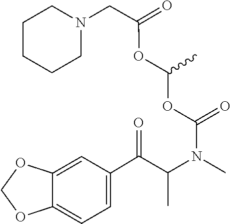 [SMILES: CC(OC(=O)CN1CCCCC1)OC(=O)N(C)C(C)C(=O)c1ccc2c(c1)OCO2] |
| 182 | 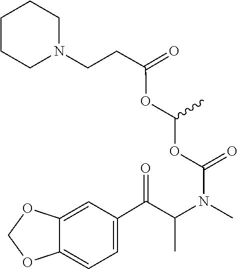 [SMILES: CC(OC(=O)CCN1CCCCC1)OC(=O)N(C)C(C)C(=O)c1ccc2c(c1)OCO2] |
| 183 | 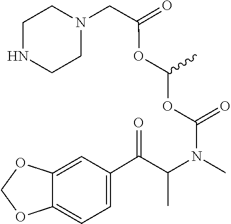 [SMILES: CC(OC(=O)CN1CCNCC1)OC(=O)N(C)C(C)C(=O)c1ccc2c(c1)OCO2] |
| 184 | 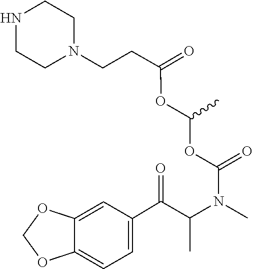 [SMILES: CC(OC(=O)CCN1CCNCC1)OC(=O)N(C)C(C)C(=O)c1ccc2c(c1)OCO2] |
| 185 | 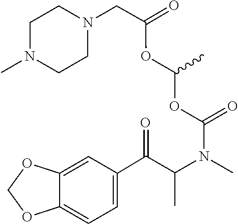 |
| 186 | 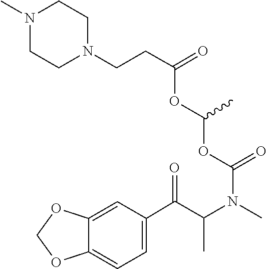 |
| 187 | 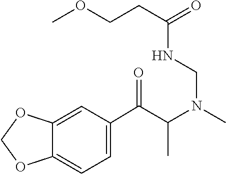 [SMILES: COCCC(=O)NCN(C)C(C)C(=O)c1ccc2c(c1)OCO2] |
| 188 | 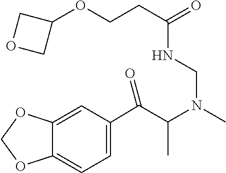 [SMILES: CC(C(=O)c1ccc2c(c1)OCO2)N(C)CNC(=O)CCOC1COC1] |
| 189 | 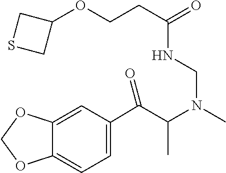 [SMILES: CC(C(=O)c1ccc2c(c1)OCO2)N(C)CNC(=O)CCOC1CSC1] |
| 190 | 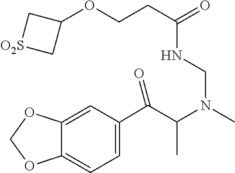 [SMILES: CC(C(=O)c1ccc2c(c1)OCO2)N(C)CNC(=O)CCOC1CS(=O)(=O)C1] |
| 191 | 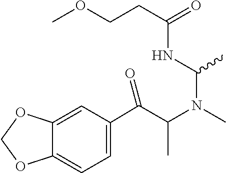 [SMILES: COCCC(=O)NC(C)N(C)C(C)C(=O)c1ccc2c(c1)OCO2] |
| 192 | 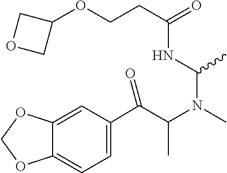 [SMILES: CC(NC(=O)CCOC1COC1)N(C)C(C)C(=O)c1ccc2c(c1)OCO2] |
| 193 | 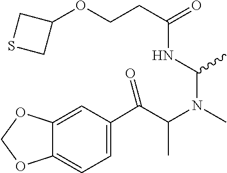 [SMILES: CC(NC(=O)CCOC1CSC1)N(C)C(C)C(=O)c1ccc2c(c1)OCO2] |
| 194 | 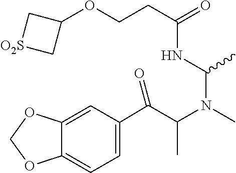 [SMILES: CC(NC(=O)CCOC1CS(=O)(=O)C1)N(C)C(C)C(=O)c1ccc2c(c1)OCO2] |
| 195 | 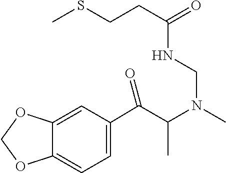 [SMILES: CSCCC(=O)NCN(C)C(C)C(=O)c1ccc2c(c1)OCO2] |
| 196 | 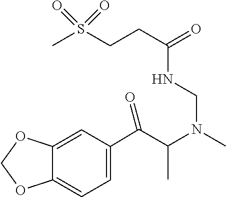 [SMILES: CC(C(=O)c1ccc2c(c1)OCO2)N(C)CNC(=O)CCS(C)(=O)=O] |
| 197 | 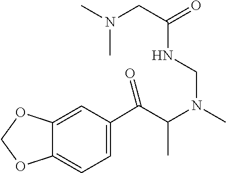 [SMILES: CC(C(=O)c1ccc2c(c1)OCO2)N(C)CNC(=O)CN(C)C] |
| 198 | 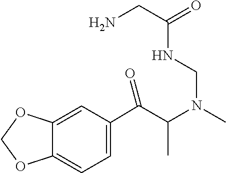 [SMILES: CC(C(=O)c1ccc2c(c1)OCO2)N(C)CNC(=O)CN] |
| 199 | 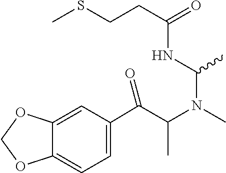 [SMILES: CSCCC(=O)NC(C)N(C)C(C)C(=O)c1ccc2c(c1)OCO2] |
| 200 | 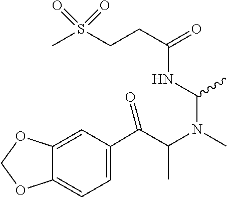 [SMILES: CC(NC(=O)CCS(C)(=O)=O)N(C)C(C)C(=O)c1ccc2c(c1)OCO2] |
| 201 | 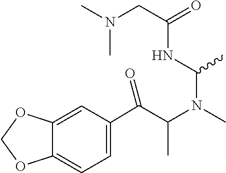 [SMILES: CC(NC(=O)CN(C)C)N(C)C(C)C(=O)c1ccc2c(c1)OCO2] |
| 202 | 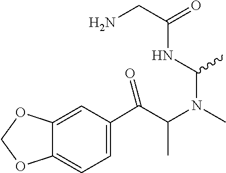 [SMILES: CC(NC(=O)CN)N(C)C(C)C(=O)c1ccc2c(c1)OCO2] |
| 203 | 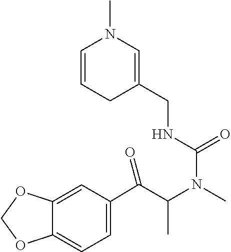 [SMILES: CC(C(=O)c1ccc2c(c1)OCO2)N(C)C(=O)NCC1=CN(C)C=CC1] |
| 204 | 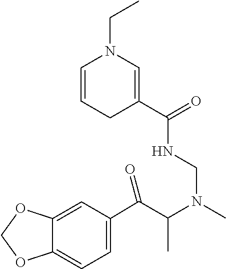 [SMILES: CCN1C=CCC(C(=O)NCN(C)C(C)C(=O)c2ccc3c(c2)OCO3)=C1] |
| 205 | 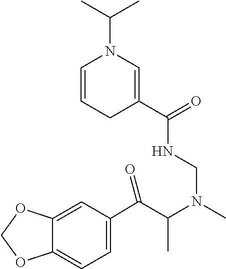 [SMILES: CC(C)N1C=CCC(C(=O)NCN(C)C(C)C(=O)c2ccc3c(c2)OCO3)=C1] |
| 206 | 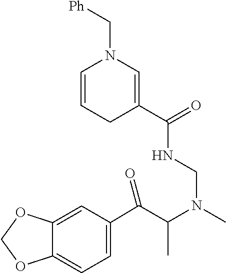 [SMILES: CC(C(=O)c1ccc2c(c1)OCO2)N(C)CNC(=O)C1=CN(Cc2ccccc2)C=CC1] |
| 207 | 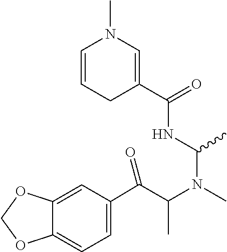 [SMILES: CC(NC(=O)C1=CN(C)C=CC1)N(C)C(C)C(=O)c1ccc2c(c1)OCO2] |
| 208 | 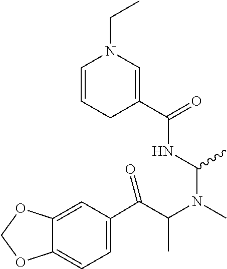 [SMILES: CCN1C=CCC(C(=O)NC(C)N(C)C(C)C(=O)c2ccc3c(c2)OCO3)=C1] |
| 209 | 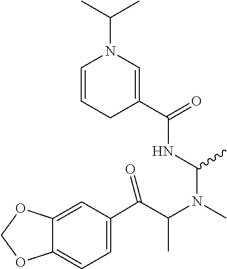 [SMILES: CC(C)N1C=CCC(C(=O)NC(C)N(C)C(C)C(=O)c2ccc3c(c2)OCO3)=C1] |
| 210 | 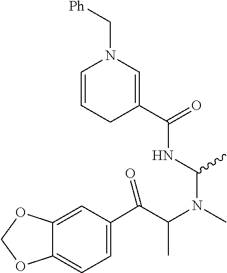 [SMILES: CC(NC(=O)C1=CN(Cc2ccccc2)C=CC1)N(C)C(C)C(=O)c1ccc2c(c1)OCO2] |
| 211 | 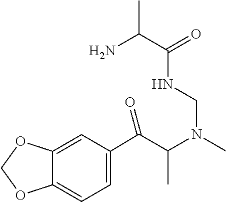 [SMILES: CC(N)C(=O)NCN(C)C(C)C(=O)c1ccc2c(c1)OCO2] |
| 212 | 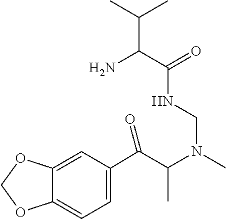 [SMILES: CC(C)C(N)C(=O)NCN(C)C(C)C(=O)c1ccc2c(c1)OCO2] |
| 213 | 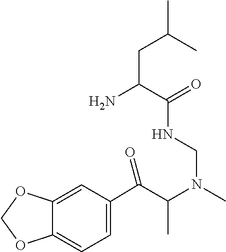 [SMILES: CC(C)CC(N)C(=O)NCN(C)C(C)C(=O)c1ccc2c(c1)OCO2] |
| 214 | 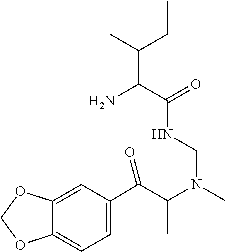 [SMILES: CCC(C)C(N)C(=O)NCN(C)C(C)C(=O)c1ccc2c(c1)OCO2] |
| 215 | 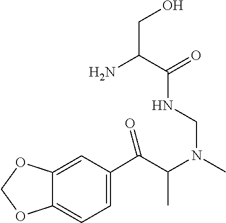 [SMILES: CC(C(=O)c1ccc2c(c1)OCO2)N(C)CNC(=O)C(N)CO] |
| 216 | 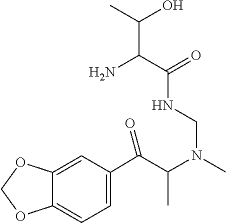 [SMILES: CC(O)C(N)C(=O)NCN(C)C(C)C(=O)c1ccc2c(c1)OCO2] |
| 217 | 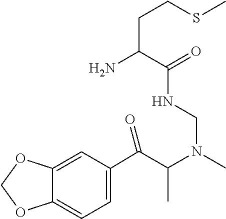 [SMILES: CSCCC(N)C(=O)NCN(C)C(C)C(=O)c1ccc2c(c1)OCO2] |
| 218 | 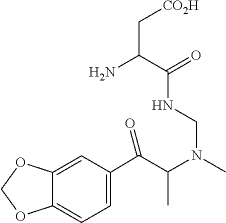 [SMILES: CC(C(=O)c1ccc2c(c1)OCO2)N(C)CNC(=O)C(N)CC(=O)O] |
| 219 | 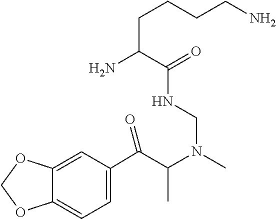 [SMILES: CC(C(=O)c1ccc2c(c1)OCO2)N(C)CNC(=O)C(N)CCCCN] |
| 220 | 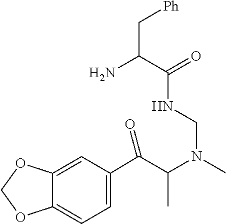 [SMILES: CC(C(=O)c1ccc2c(c1)OCO2)N(C)CNC(=O)C(N)Cc1ccccc1] |
| 221 | 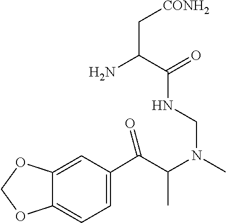 [SMILES: CC(C(=O)c1ccc2c(c1)OCO2)N(C)CNC(=O)C(N)CC(N)=O] |
| 222 | 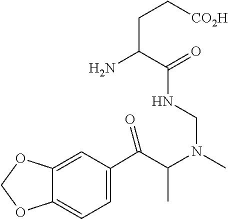 [SMILES: CC(C(=O)c1ccc2c(c1)OCO2)N(C)CNC(=O)C(N)CCC(=O)O] |
| 223 | 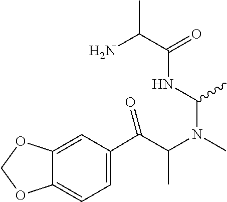 [SMILES: CC(N)C(=O)NC(C)N(C)C(C)C(=O)c1ccc2c(c1)OCO2] |
| 224 | 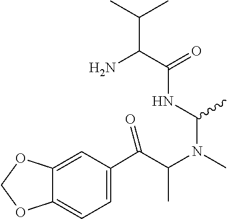 [SMILES: CC(C)C(N)C(=O)NC(C)N(C)C(C)C(=O)c1ccc2c(c1)OCO2] |
| 225 | 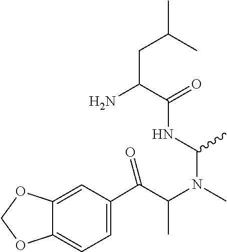 [SMILES: CC(C)CC(N)C(=O)NC(C)N(C)C(C)C(=O)c1ccc2c(c1)OCO2] |
| 226 | 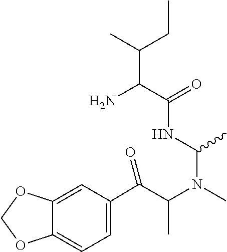 [SMILES: CCC(C)C(N)C(=O)NC(C)N(C)C(C)C(=O)c1ccc2c(c1)OCO2] |
| 227 | 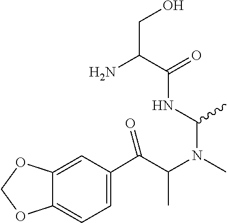 [SMILES: CC(NC(=O)C(N)CO)N(C)C(C)C(=O)c1ccc2c(c1)OCO2] |
| 228 | 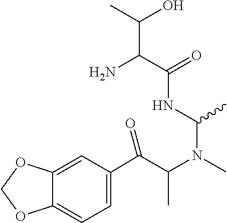 [SMILES: CC(O)C(N)C(=O)NC(C)N(C)C(C)C(=O)c1ccc2c(c1)OCO2] |
| 229 | 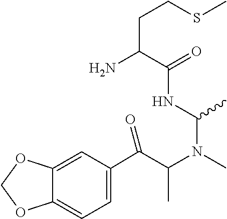 [SMILES: CSCCC(N)C(=O)NC(C)N(C)C(C)C(=O)c1ccc2c(c1)OCO2] |
| 230 | 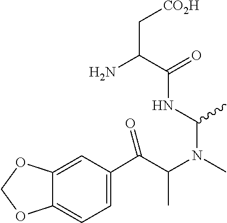 [SMILES: CC(NC(=O)C(N)CC(=O)O)N(C)C(C)C(=O)c1ccc2c(c1)OCO2] |
| 231 | 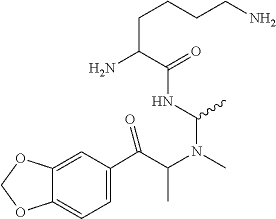 [SMILES: CC(NC(=O)C(N)CCCCN)N(C)C(C)C(=O)c1ccc2c(c1)OCO2] |
| 232 | 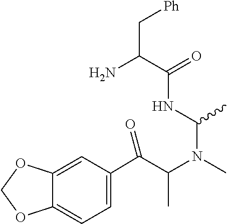 [SMILES: CC(NC(=O)C(N)Cc1ccccc1)N(C)C(C)C(=O)c1ccc2c(c1)OCO2] |
| 233 | 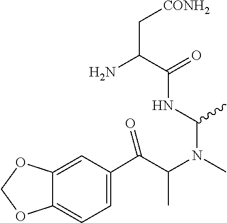 [SMILES: CC(NC(=O)C(N)CC(N)=O)N(C)C(C)C(=O)c1ccc2c(c1)OCO2] |
| 234 | 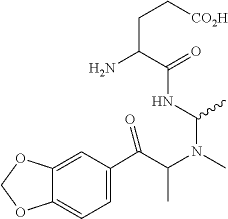 [SMILES: CC(NC(=O)C(N)CCC(=O)O)N(C)C(C)C(=O)c1ccc2c(c1)OCO2] |
| 235 | 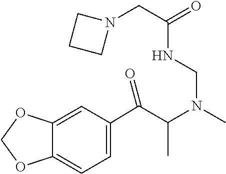 [SMILES: CC(C(=O)c1ccc2c(c1)OCO2)N(C)CNC(=O)CN1CCC1] |
| 236 | 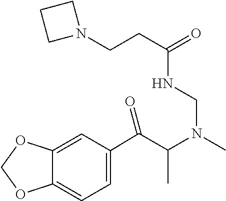 [SMILES: CC(C(=O)c1ccc2c(c1)OCO2)N(C)CNC(=O)CCN1CCC1] |
| 237 | 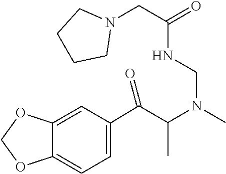 [SMILES: CC(C(=O)c1ccc2c(c1)OCO2)N(C)CNC(=O)CN1CCCC1] |
| 238 | 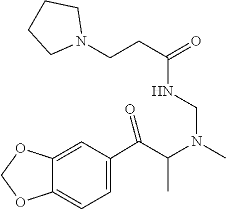 [SMILES: CC(C(=O)c1ccc2c(c1)OCO2)N(C)CNC(=O)CCN1CCCC1] |
| 239 | 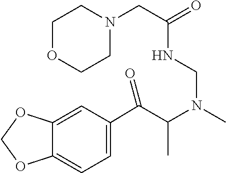 [SMILES: CC(C(=O)c1ccc2c(c1)OCO2)N(C)CNC(=O)CN1CCOCC1] |
| 240 | 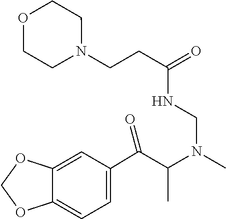 [SMILES: CC(C(=O)c1ccc2c(c1)OCO2)N(C)CNC(=O)CCN1CCOCC1] |
| 241 | 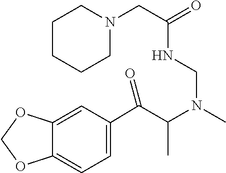 [SMILES: CC(C(=O)c1ccc2c(c1)OCO2)N(C)CNC(=O)CN1CCCCC1] |
| 242 | 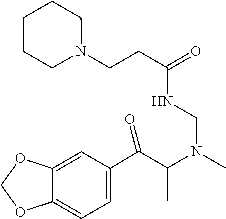 [SMILES: CC(C(=O)c1ccc2c(c1)OCO2)N(C)CNC(=O)CCN1CCCCC1] |
| 243 | 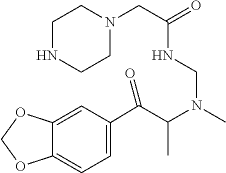 [SMILES: CC(C(=O)c1ccc2c(c1)OCO2)N(C)CNC(=O)CN1CCNCC1] |
| 244 | 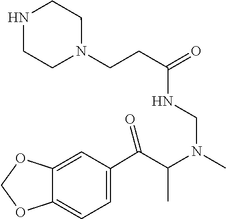 [SMILES: CC(C(=O)c1ccc2c(c1)OCO2)N(C)CNC(=O)CCN1CCNCC1] |
| 245 | 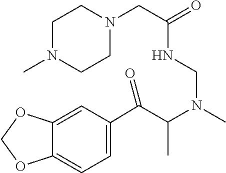 [SMILES: CC(C(=O)c1ccc2c(c1)OCO2)N(C)CNC(=O)CN1CCN(C)CC1] |
| 246 | 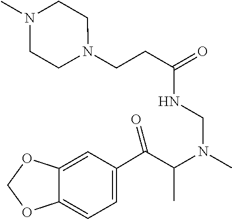 |
| 247 | 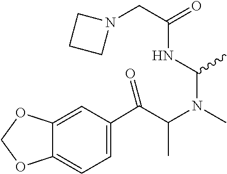 [SMILES: CC(NC(=O)CN1CCC1)N(C)C(C)C(=O)c1ccc2c(c1)OCO2] |
| 248 | 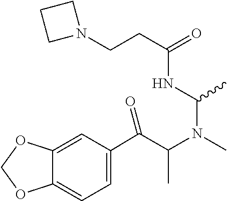 [SMILES: CC(NC(=O)CCN1CCC1)N(C)C(C)C(=O)c1ccc2c(c1)OCO2] |
| 249 | 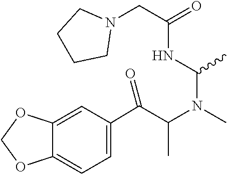 [SMILES: CC(NC(=O)CN1CCCC1)N(C)C(C)C(=O)c1ccc2c(c1)OCO2] |
| 250 | 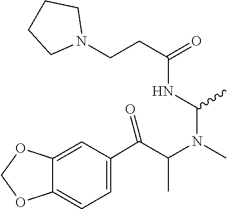 [SMILES: CC(NC(=O)CCN1CCCC1)N(C)C(C)C(=O)c1ccc2c(c1)OCO2] |
| 251 | 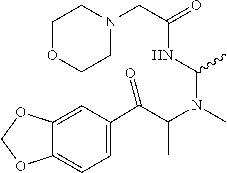 [SMILES: CC(NC(=O)CN1CCOCC1)N(C)C(C)C(=O)c1ccc2c(c1)OCO2] |
| 252 | 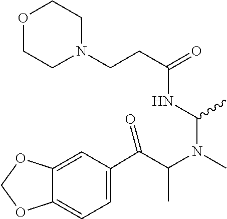 [SMILES: CC(NC(=O)CCN1CCOCC1)N(C)C(C)C(=O)c1ccc2c(c1)OCO2] |
| 253 | 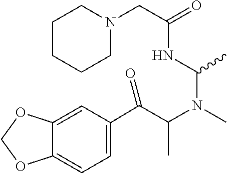 [SMILES: CC(NC(=O)CN1CCCCC1)N(C)C(C)C(=O)c1ccc2c(c1)OCO2] |
| 254 | 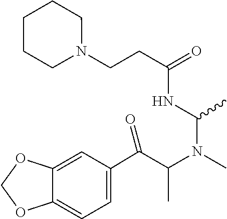 [SMILES: CC(NC(=O)CCN1CCCCC1)N(C)C(C)C(=O)c1ccc2c(c1)OCO2] |
| 255 | 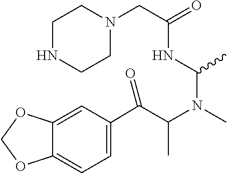 [SMILES: CC(NC(=O)CN1CCNCC1)N(C)C(C)C(=O)c1ccc2c(c1)OCO2] |
| 256 | 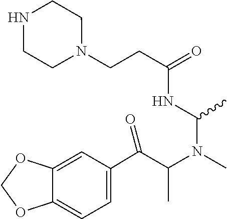 [SMILES: CC(NC(=O)CCN1CCNCC1)N(C)C(C)C(=O)c1ccc2c(c1)OCO2] |
| 257 | 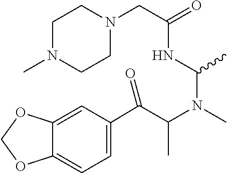 [SMILES: CC(NC(=O)CN1CCN(C)CC1)N(C)C(C)C(=O)c1ccc2c(c1)OCO2] |
| 258 | 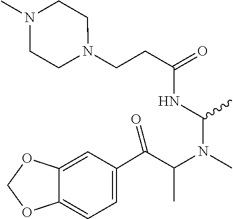 [SMILES: CC(NC(=O)CCN1CCN(C)CC1)N(C)C(C)C(=O)c1ccc2c(c1)OCO2] |
| 259 | 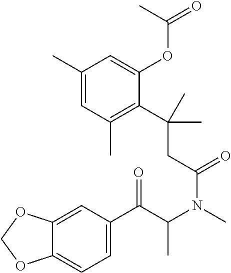 [SMILES: CC(=O)Oc1cc(C)cc(C)c1C(C)(C)CC(=O)N(C)C(C)C(=O)c1ccc2c(c1)OCO2] |
| 260 | 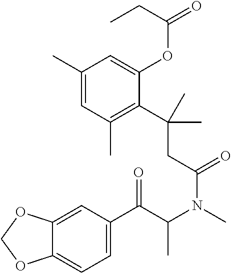 [SMILES: CCC(=O)Oc1cc(C)cc(C)c1C(C)(C)CC(=O)N(C)C(C)C(=O)c1ccc2c(c1)OCO2] |
| 261 | 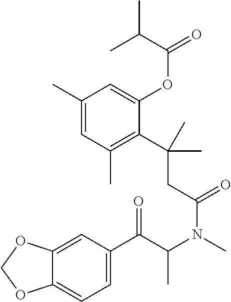 [SMILES: Cc1cc(C)c(C(C)(C)CC(=O)N(C)C(C)C(=O)c2ccc3c(c2)OCO3)c(OC(=O)C(C)C)c1] |
| 262 | 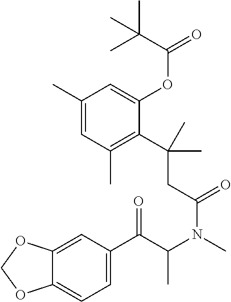 [SMILES: Cc1cc(C)c(C(C)(C)CC(=O)N(C)C(C)C(=O)c2ccc3c(c2)OCO3)c(OC(=O)C(C)(C)C)c1] |
| 263 | 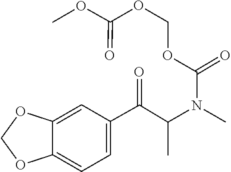 [SMILES: COC(=O)OCOC(=O)N(C)C(C)C(=O)c1ccc2c(c1)OCO2] |
| 264 | 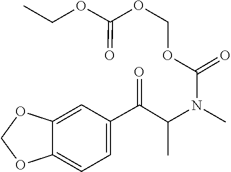 [SMILES: CCOC(=O)OCOC(=O)N(C)C(C)C(=O)c1ccc2c(c1)OCO2] |
| 266 | 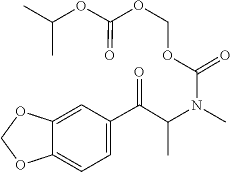 [SMILES: CC(C)OC(=O)OCOC(=O)N(C)C(C)C(=O)c1ccc2c(c1)OCO2] |
| 267 | 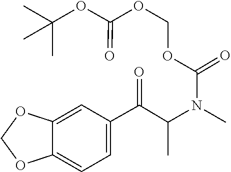 [SMILES: CC(C(=O)c1ccc2c(c1)OCO2)N(C)C(=O)OCOC(=O)OC(C)(C)C] |
| 268 | 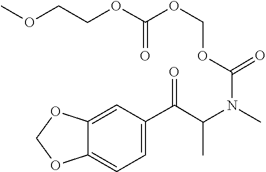 |
| 269 | 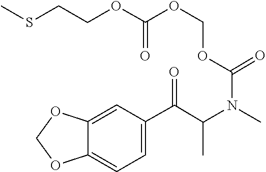 |
| 270 | 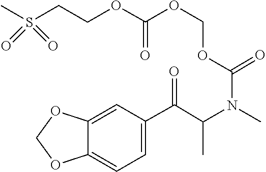 |
| 271 | 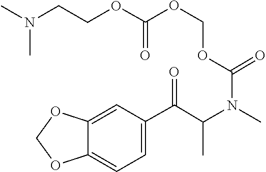 |
| 272 | 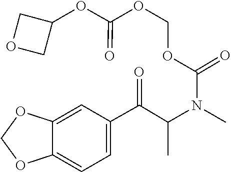 [SMILES: CC(C(=O)c1ccc2c(c1)OCO2)N(C)C(=O)OCOC(=O)OC1COC1] |
| 273 | 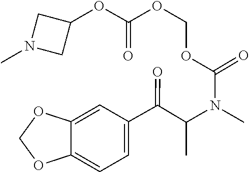 |
| 274 | 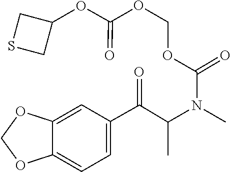 [SMILES: CC(C(=O)c1ccc2c(c1)OCO2)N(C)C(=O)OCOC(=O)OC1CSC1] |
| 275 | 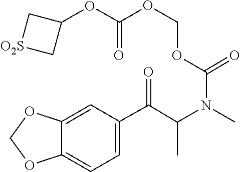 [SMILES: CC(C(=O)c1ccc2c(c1)OCO2)N(C)C(=O)OCOC(=O)OC1CS(=O)(=O)C1] |
| 276 | 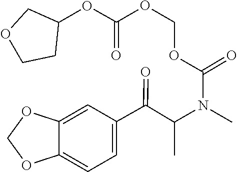 [SMILES: CC(C(=O)c1ccc2c(c1)OCO2)N(C)C(=O)OCOC(=O)OC1CCOC1] |
| 277 | 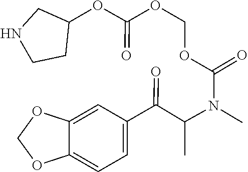 [SMILES: CC(C(=O)c1ccc2c(c1)OCO2)N(C)C(=O)OCOC(=O)OC1CCNC1] |
| 278 | 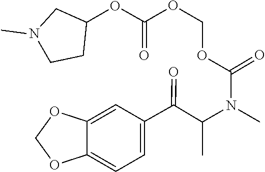 |
| 279 | 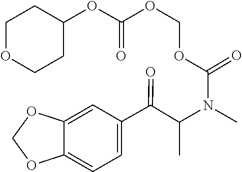 [SMILES: CC(C(=O)c1ccc2c(c1)OCO2)N(C)C(=O)OCOC(=O)OC1CCOCC1] |
| 280 | 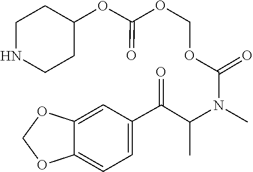 [SMILES: CC(C(=O)c1ccc2c(c1)OCO2)N(C)C(=O)OCOC(=O)OC1CCNCC1] |
| 281 | 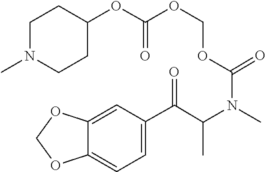 |
| 282 | 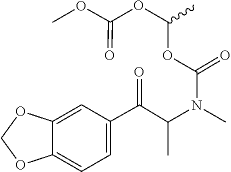 [SMILES: COC(=O)OC(C)OC(=O)N(C)C(C)C(=O)c1ccc2c(c1)OCO2] |
| 283 | 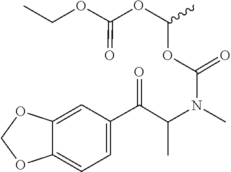 [SMILES: CCOC(=O)OC(C)OC(=O)N(C)C(C)C(=O)c1ccc2c(c1)OCO2] |
| 284 | 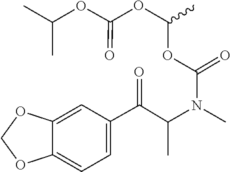 [SMILES: CC(C)OC(=O)OC(C)OC(=O)N(C)C(C)C(=O)c1ccc2c(c1)OCO2] |
| 285 | 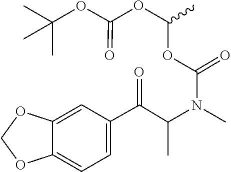 [SMILES: CC(OC(=O)OC(C)(C)C)OC(=O)N(C)C(C)C(=O)c1ccc2c(c1)OCO2] |
| 286 | 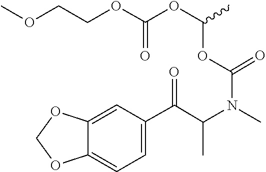 |
| 287 | 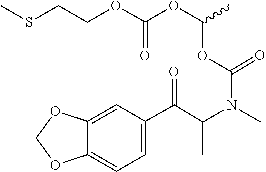 |
| 288 | 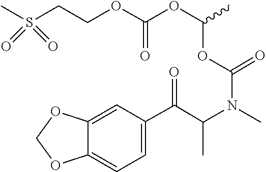 |
| 289 | 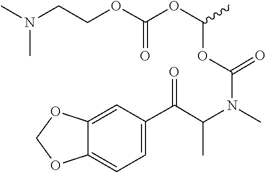 |
| 290 | 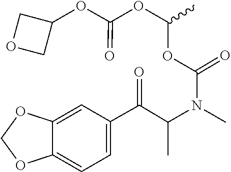 [SMILES: CC(OC(=O)OC1COC1)OC(=O)N(C)C(C)C(=O)c1ccc2c(c1)OCO2] |
| 291 | 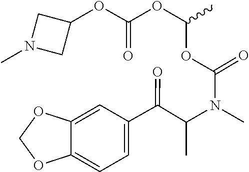 |
| 292 | 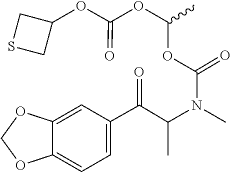 [SMILES: CC(OC(=O)OC1CSC1)OC(=O)N(C)C(C)C(=O)c1ccc2c(c1)OCO2] |
| 293 | 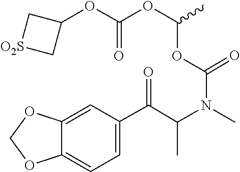 [SMILES: CC(OC(=O)OC1CS(=O)(=O)C1)OC(=O)N(C)C(C)C(=O)c1ccc2c(c1)OCO2] |
| 294 | 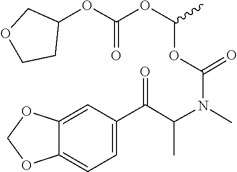 [SMILES: CC(OC(=O)OC1CCOC1)OC(=O)N(C)C(C)C(=O)c1ccc2c(c1)OCO2] |
| 295 | 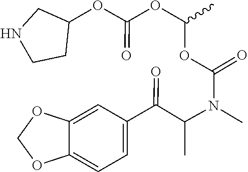 [SMILES: CC(OC(=O)OC1CCNC1)OC(=O)N(C)C(C)C(=O)c1ccc2c(c1)OCO2] |
| 296 | 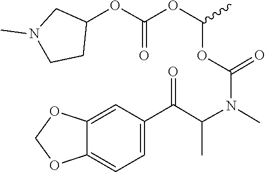 [SMILES: CC(OC(=O)OC1CCN(C)C1)OC(=O)N(C)C(C)C(=O)c1ccc2c(c1)OCO2] |
| 297 | 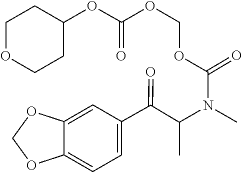 [SMILES: CC(C(=O)c1ccc2c(c1)OCO2)N(C)C(=O)OCOC(=O)OC1CCOCC1] |
| 298 | 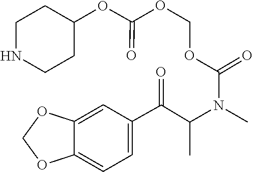 [SMILES: CC(C(=O)c1ccc2c(c1)OCO2)N(C)C(=O)OCOC(=O)OC1CCNCC1] |
| 299 | 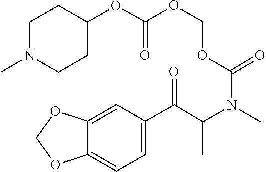 |
| 300 | 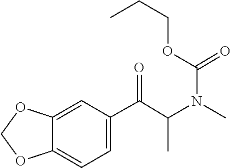 [SMILES: CCCOC(=O)N(C)C(C)C(=O)c1ccc2c(c1)OCO2] |
| 301 | 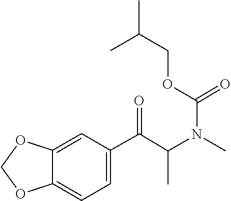 [SMILES: CC(C)COC(=O)N(C)C(C)C(=O)c1ccc2c(c1)OCO2] |
| 302 | 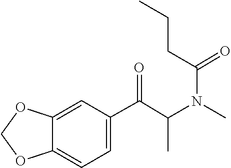 [SMILES: CCCC(=O)N(C)C(C)C(=O)c1ccc2c(c1)OCO2] |
| 303 | 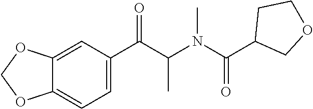 [SMILES: CC(C(=O)c1ccc2c(c1)OCO2)N(C)C(=O)C1CCOC1] |
| 304 | 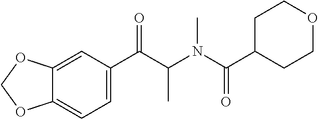 [SMILES: CC(C(=O)c1ccc2c(c1)OCO2)N(C)C(=O)C1CCOCC1] |
| 305 | 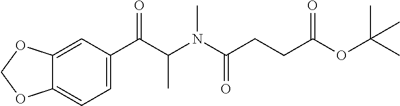 [SMILES: CC(C(=O)c1ccc2c(c1)OCO2)N(C)C(=O)CCC(=O)OC(C)(C)C] |
| 306 | 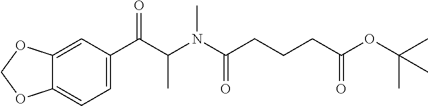 [SMILES: CC(C(=O)c1ccc2c(c1)OCO2)N(C)C(=O)CCCC(=O)OC(C)(C)C] |
| 307 | 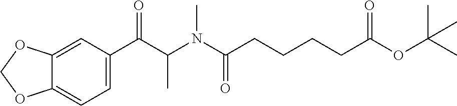 [SMILES: CC(C(=O)c1ccc2c(c1)OCO2)N(C)C(=O)CCCCC(=O)OC(C)(C)C] |
| 308 | 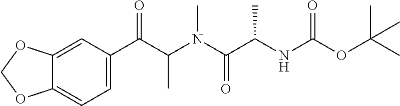 [SMILES: CC(C(=O)c1ccc2c(c1)OCO2)N(C)C(=O)[C@H](C)NC(=O)OC(C)(C)C] |
| 309 | 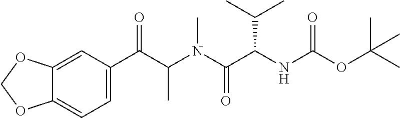 [SMILES: CC(C)[C@H](NC(=O)OC(C)(C)C)C(=O)N(C)C(C)C(=O)c1ccc2c(c1)OCO2] |
| 310 | 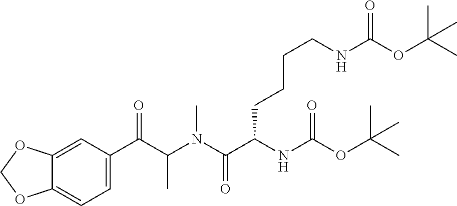 [SMILES: CC(C(=O)c1ccc2c(c1)OCO2)N(C)C(=O)[C@H](CCCCNC(=O)OC(C)(C)C)NC(=O)OC(C)(C)C] |
| 311 | 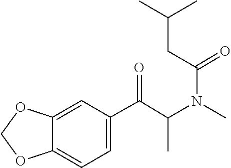 [SMILES: CC(C)CC(=O)N(C)C(C)C(=O)c1ccc2c(c1)OCO2] |
| 312 | 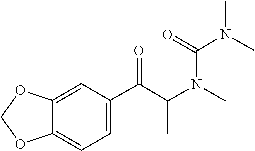 [SMILES: CC(C(=O)c1ccc2c(c1)OCO2)N(C)C(=O)N(C)C] |
| 313 | 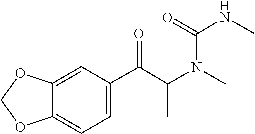 [SMILES: CNC(=O)N(C)C(C)C(=O)c1ccc2c(c1)OCO2] |
| 314 | 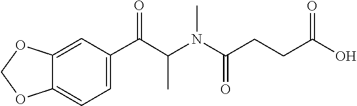 [SMILES: CC(C(=O)c1ccc2c(c1)OCO2)N(C)C(=O)CCC(=O)O] |
| 315 | 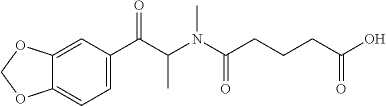 [SMILES: CC(C(=O)c1ccc2c(c1)OCO2)N(C)C(=O)CCCC(=O)O] |
| 316 | 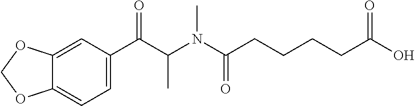 [SMILES: CC(C(=O)c1ccc2c(c1)OCO2)N(C)C(=O)CCCCC(=O)O] |
| 317 | 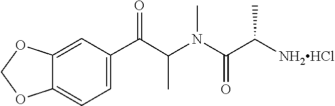 |
| 318 | 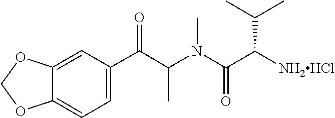 |
| 319 | 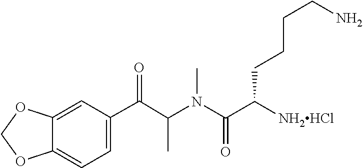 |
| 320 | 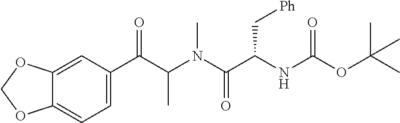 [SMILES: CC(C(=O)c1ccc2c(c1)OCO2)N(C)C(=O)[C@H](Cc1ccccc1)NC(=O)OC(C)(C)C] |
| 321 | 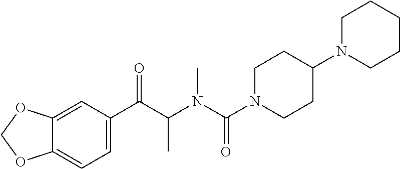 [SMILES: CC(C(=O)c1ccc2c(c1)OCO2)N(C)C(=O)N1CCC(N2CCCCC2)CC1] |
| 322 | 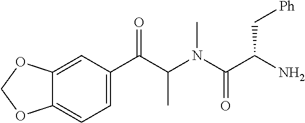 [SMILES: CC(C(=O)c1ccc2c(c1)OCO2)N(C)C(=O)[C@@H](N)Cc1ccccc1] |
| 323 | 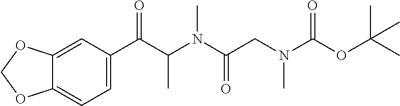 [SMILES: CC(C(=O)c1ccc2c(c1)OCO2)N(C)C(=O)CN(C)C(=O)OC(C)(C)C] |
| 324 | 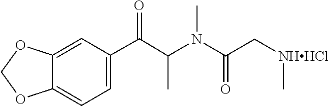 |
| 325 | 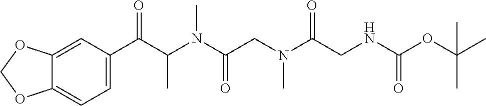 [SMILES: CC(C(=O)c1ccc2c(c1)OCO2)N(C)C(=O)CN(C)C(=O)CNC(=O)OC(C)(C)C] |
| 326 | 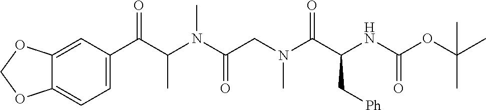 [SMILES: CC(C(=O)c1ccc2c(c1)OCO2)N(C)C(=O)CN(C)C(=O)[C@H](Cc1ccccc1)NC(=O)OC(C)(C)C] |
| 327 | 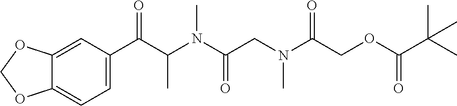 [SMILES: CC(C(=O)c1ccc2c(c1)OCO2)N(C)C(=O)CN(C)C(=O)COC(=O)C(C)(C)C] |
| 328 | 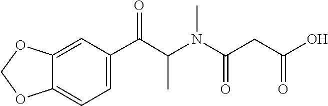 [SMILES: CC(C(=O)c1ccc2c(c1)OCO2)N(C)C(=O)CC(=O)O] |
| 329 | 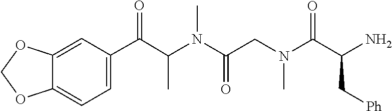 [SMILES: CC(C(=O)c1ccc2c(c1)OCO2)N(C)C(=O)CN(C)C(=O)[C@@H](N)Cc1ccccc1] |
| 330 | 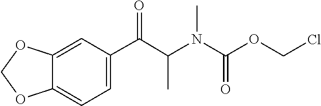 [SMILES: CC(C(=O)c1ccc2c(c1)OCO2)N(C)C(=O)OCCl] |
| 331 | 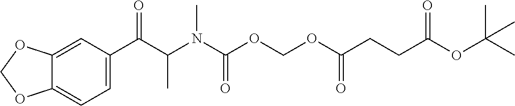 [SMILES: CC(C(=O)c1ccc2c(c1)OCO2)N(C)C(=O)OCOC(=O)CCC(=O)OC(C)(C)C] |
| 332 | 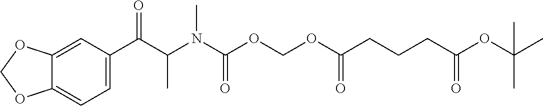 [SMILES: CC(C(=O)c1ccc2c(c1)OCO2)N(C)C(=O)OCOC(=O)CCCC(=O)OC(C)(C)C] |
| 333 | 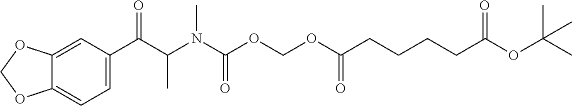 [SMILES: CC(C(=O)c1ccc2c(c1)OCO2)N(C)C(=O)OCOC(=O)CCCCC(=O)OC(C)(C)C] |
| 334 | 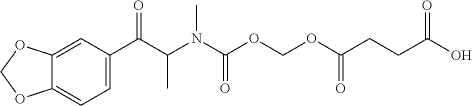 [SMILES: CC(C(=O)c1ccc2c(c1)OCO2)N(C)C(=O)OCOC(=O)CCC(=O)O] |
| 335 | 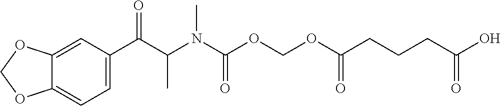 [SMILES: CC(C(=O)c1ccc2c(c1)OCO2)N(C)C(=O)OCOC(=O)CCCC(=O)O] |
| 336 | 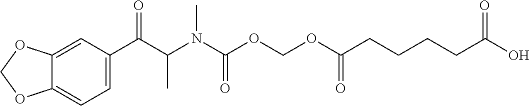 [SMILES: CC(C(=O)c1ccc2c(c1)OCO2)N(C)C(=O)OCOC(=O)CCCCC(=O)O] |
| 337 | 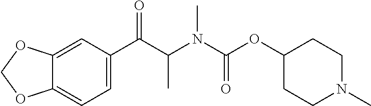 [SMILES: CC(C(=O)c1ccc2c(c1)OCO2)N(C)C(=O)OC1CCN(C)CC1] |
| 338 | 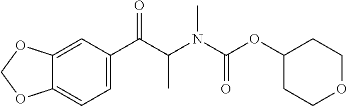 [SMILES: CC(C(=O)c1ccc2c(c1)OCO2)N(C)C(=O)OC1CCOCC1] |
| 339 | 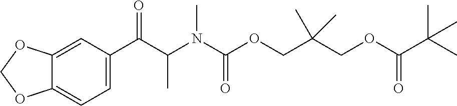 [SMILES: CC(C(=O)c1ccc2c(c1)OCO2)N(C)C(=O)OCC(C)(C)COC(=O)C(C)(C)C] |
| 340 | 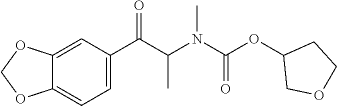 [SMILES: CC(C(=O)c1ccc2c(c1)OCO2)N(C)C(=O)OC1CCOC1] |
| 341 | 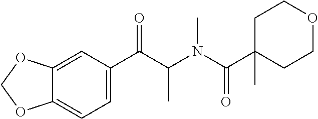 [SMILES: CC(C(=O)c1ccc2c(c1)OCO2)N(C)C(=O)C1(C)CCOCC1] |
| 342 | 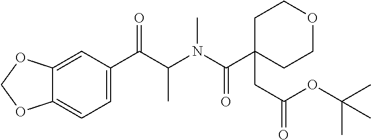 [SMILES: CC(C(=O)c1ccc2c(c1)OCO2)N(C)C(=O)C1(CC(=O)OC(C)(C)C)CCOCC1] |
| 343 | 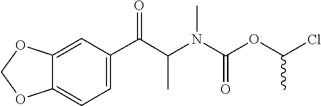 [SMILES: CC(Cl)OC(=O)N(C)C(C)C(=O)c1ccc2c(c1)OCO2] |
| 344 | 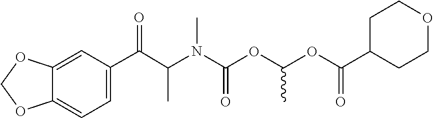 [SMILES: CC(OC(=O)C1CCOCC1)OC(=O)N(C)C(C)C(=O)c1ccc2c(c1)OCO2] |
| 345 | 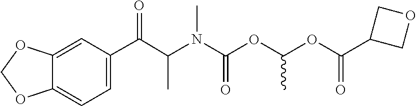 [SMILES: CC(OC(=O)C1COC1)OC(=O)N(C)C(C)C(=O)c1ccc2c(c1)OCO2] |
| 346 | 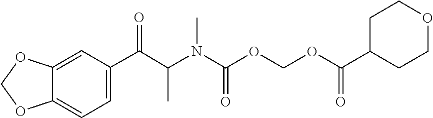 [SMILES: CC(C(=O)c1ccc2c(c1)OCO2)N(C)C(=O)OCOC(=O)C1CCOCC1] |
| 347 | 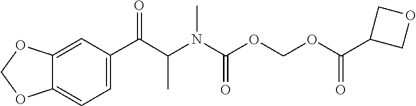 [SMILES: CC(C(=O)c1ccc2c(c1)OCO2)N(C)C(=O)OCOC(=O)C1COC1] |
| 348 | 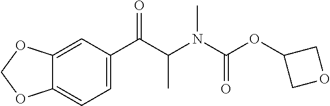 [SMILES: CC(C(=O)c1ccc2c(c1)OCO2)N(C)C(=O)OC1COC1] |
| 349 | 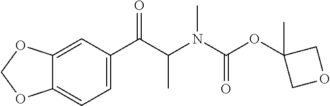 [SMILES: CC(C(=O)c1ccc2c(c1)OCO2)N(C)C(=O)OC1(C)COC1] |
| 350 | 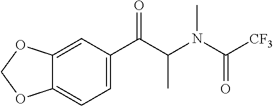 [SMILES: CC(C(=O)c1ccc2c(c1)OCO2)N(C)C(=O)C(F)(F)F] |
| 351 | 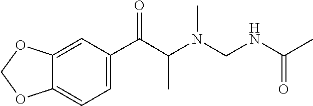 [SMILES: CC(=O)NCN(C)C(C)C(=O)c1ccc2c(c1)OCO2] |
| 352 | 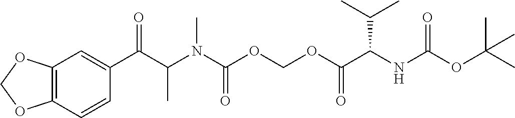 [SMILES: CC(C)[C@H](NC(=O)OC(C)(C)C)C(=O)OCOC(=O)N(C)C(C)C(=O)c1ccc2c(c1)OCO2] |
| 353 | 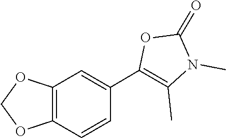 [SMILES: Cc1c(-c2ccc3c(c2)OCO3)oc(=O)n1C] |

In another aspect, the present disclosure provides a pharmaceutically acceptable composition comprising a compound according to any of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III), and a pharmaceutically acceptable excipient, carrier, adjuvant, or vehicle.

Pharmaceutical compositions of the present disclosure can comprise racemic, scalemic, or diasteromerically enriched mixtures of any compound described herein.

In some embodiments, the present disclosure provides a pharmaceutical composition comprising a mixture of diastereomers of a compound of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III), wherein at least about 60%, at least about 70%, at least about 80%, at least about 90%, at least about 95%, at least about 98%, at least about 99%, at least about 99.5%, or at least about 99.9% of molecules in the mixture comprise a (S)-3,4-methylenedioxy-N-methylcathinone moiety.

In some embodiments, the present disclosure provides a pharmaceutical composition comprising a mixture of diastereomers of a compound of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III), wherein at least about 60%, at least about 70%, at least about 80%, at least about 90%, at least about 95%, at least about 98%, at least about 99%, at least about 99.5%, or at least about 99.9% of molecules in the mixture comprise a (R)-3,4-methylenedioxy-N-methylcathinone moiety.

In some embodiments, the present disclosure provides a pharmaceutical composition comprising a mixture of diastereomers of a compound of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III), wherein about 50% of molecules in the mixture comprise a (R)-3,4-methylenedioxy-N-methylcathinone moiety.

In some embodiments, the present disclosure provides a pharmaceutical composition comprising a mixture of diastereomers of a compound of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III), wherein from about 48% to about 52% of molecules in the mixture comprise a (R)-3,4-methylenedioxy-N-methylcathinone moiety.

In some embodiments, the present disclosure provides a pharmaceutical composition comprising a mixture of diastereomers of a compound of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III), wherein from about 55% to about 99.99%, from about 60% to about 99.99%, from about 70% to about 99.99%, from about 80% to about 99.99%, from about 90% to about 99.99%, from about 95% to about 99.99%, from about 98% to about 99.99%, from about 99% to about 99.99%, from about 99.5% to about 99.99%, or from about 99.9% to about 99.99% of molecules in the mixture comprise a (R)-3,4-methylenedioxy-N-methylcathinone moiety.

In some embodiments, the present disclosure provides a pharmaceutical composition comprising a mixture of diastereomers of a compound of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III), wherein from about 55% to about 99.99%, from about 60% to about 99.99%, from about 70% to about 99.99%, from about 80% to about 99.99%, from about 90% to about 99.99%, from about 95% to about 99.99%, from about 98% to about 99.99%, from about 99% to about 99.99%, from about 99.5% to about 99.99%, or from about 99.9% to about 99.99% of molecules in the mixture comprise a (S)-3,4-methylenedioxy-N-methylcathinone.

### Methods of Treatment

In yet another aspect, the present disclosure provides a method of treating or preventing a disease, disorder, or condition in which an increased level of a phenethylamine psychedelic such as methylone is beneficial, comprising administering to a subject in need thereof an effective amount of a compound of Table 1, Formula (I), (Ia), (Ia-1), (Ia-2), (Ia1), (Ia1-1), (Ia1-2), (Ia2), (Ia2-1), (Ia2-2), (Ia3), (Ia3-1), (Ia3-2), (Ib), (Ib-1), (Ib-2), (Ib1), (Ib2), (Ib2-1), (Ib2-2), (Ib3), (Ib3-1), (Ib3-2), (Ic), (Ic-1), (Ic-2), (Ic1), (Ic1-1), (Ic1-2), (Ic2), (Ic2-1), (Ic2-2), (Ic3), (Ic3-1), (Ic3-2), (Ic4), (Ic4-1), (Ic4-2), (Ic5), (Ic5-1), (Ic5-2), (Id), (Id-1), (Id-2), (Id1), (Id1-1), (Id1-2), (Id2), (Id2-1), (Id2-2), (Id3), (Id3-1), (Id3-2), (Id4), (Id4-1), (Id4-1), (Id5), (Id5-1), (Id5-2), (Ie), (Ie-1), (Ie-2), (If), (If-2), (If-2), (II), and/or (III). In some embodiments, the condition comprises post-traumatic stress disorder, major depression, schizophrenia, Alzheimer's disease, frontotemporal dementia, Parkinson's disease, Parkinson's dementia, dementia, Lewy body dementia, multiple system atrophy, or substance abuse. In some embodiments, the condition comprises musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. The compounds of the present invention can also be used to treat any brain disease.

In some embodiments, a compound disclosed herein has activity as a 5-HT_{2A} modulator. In some embodiments a compound disclosed herein elicits a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity. 5-HT_{2A} antagonists abrogate the neuritogenesis and spinogenesis effects of hallucinogenic compounds with 5-HT_{2A} agonist activity, for example, DMT, LSD, and DOI. In some embodiments, a compound disclosed herein is a 5-HT_{2A} modulator and promotes neural plasticity (e.g., cortical structural plasticity). In some embodiments, a compound disclosed herein is a selective 5-HT_{2A} modulator and promotes neural plasticity (e.g., cortical structural plasticity). Promotion of neural plasticity can include, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT₂A agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. In some embodiments, a compound of the present disclosure functions as a 5-HT_{2A} agonist alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present disclosure to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl)piperazin-1-yl)propyl)-5-methyl-5-phenylimidazolidine-2,4-dione, 3-(3-(4-(3-Fluorophenyl)piperazin-1-yl)propyl)-5-methyl-5-phenylimidazolidine-2,4-dione, 3-(3-(4-(4-Fluorophenyl)piperazin-1-yl)propyl)-5-methyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-Fluorobenzoyl)piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl)piperazin-1-yl)propyl)-8-phenyl-1,3-diazaspiro[4.5]decane-2,4-dione, 3-(3-(4-(3-Fluorophenyl)piperazin-1-yl)propyl)-8-phenyl-1,3-diazaspiro[4.5]decane-2,4-dione, 3-(3-(4-(4-Fluorophenyl)piperazin-1-yl)propyl)-8-phenyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to administration of a compound disclosed herein, such as about three or about one hours prior to administration of the compound. In some embodiments, the serotonin receptor modulator is administered at most about one hour prior to the compound. In some embodiments, the second therapeutic agent is a serotonin receptor modulator. In some embodiments, the serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, a compound of the present disclosure is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT₂A receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT₂A modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

In some embodiments, a compound herein is given to patients in a low dose that is lower than would produce noticeable psychedelic effects but high enough to provide a therapeutic benefit. This dose range is predicted to be between 200 g (micrograms) and 2 mg.

In some embodiments, a compound described herein is used to treat a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present disclosure is used for increasing neuronal plasticity. In some embodiments, a compound described herein is used for treating a brain disorder. In some embodiments, a compound described herein is used for increasing translation, transcription, or secretion of neurotrophic factors.

A compound disclosed herein can also be useful for increasing neuronal plasticity in a subject. As used herein, “neuronal plasticity” can refer to the ability of the brain to change structure and/or function throughout a subject's life. New neurons can be produced and integrated into the central nervous system throughout the subject's life. Increasing neuronal plasticity can include, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound the present disclosure can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides a method for increasing neuronal plasticity, comprising contacting a neuronal cell with a compound of the present disclosure. In some embodiments, increasing neuronal plasticity improves a brain disorder described herein.

In some embodiments, a compound disclosed herein is used to increase neuronal plasticity and has, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). Brain disorders can include, for example, migraines, addiction (e.g., substance use disorder), depression, and anxiety.

In some embodiments, the experiment or assay to determine increased neuronal plasticity derived from the administration of any compound of the present disclosure is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present disclosure is a mouse head-twitch response (HTR) assay.

In some embodiments, the condition is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. In some embodiments, the present disclosure provides a method of treating a brain disorder, including administering to a subject in need thereof, a therapeutically effective amount of a compound of the present disclosure. In some embodiments, the present disclosure provides a method of treating a brain disorder with combination therapy, including administering to a subject in need thereof, a therapeutically effective amount of a compound of the present disclosure and at least one additional therapeutic agent.

In some embodiments, a compound of the present disclosure is used to treat brain disorders. In some embodiments, the compound has, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present disclosure provides a method of treating a brain disorder, comprising administering to a subject in need thereof a therapeutically effective amount of a compound disclosed herein. In some embodiments, the brain disorder is a neurodegenerative disorder, Alzheimer's disease, Parkinson's disease, a psychological disorder, depression, addiction, anxiety, post-traumatic stress disorder, treatment resistant depression, suicidal ideation, major depressive disorder, bipolar disorder, schizophrenia, stroke, traumatic brain injury, or a substance use disorder.

In some embodiments, the brain disorder is a neurodegenerative disorder, Alzheimer's disease or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent. Non-limiting examples of additional therapeutics suitable for administration with a compound of the present disclosure can include lithium, olanzapine (Zyprexa), quetiapine (Seroquel), risperidone (Risperdal), aripiprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In some embodiments, a compound of the present disclosure is used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

As used herein, the term “neurotrophic factor” can refer to a family of soluble peptides or proteins which support the survival, growth, and differentiation of developing and mature neurons. Increasing at least one of translation, transcription, or secretion of neurotrophic factors can be useful for, for example, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors increases neuronal plasticity. In some embodiments, increasing at least one of translation, transcription, or secretion of neurotrophic factors promotes neuronal growth, promotes neuritogenesis, promotes synaptogenesis, promotes dendritogenesis, increases dendritic arbor complexity, and/or increases dendritic spine density.

In some embodiments, a 5-HT_{2A} modulators (e.g., 5-HT_{2A} agonists) is used to increase at least one of translation, transcription, or secretion of neurotrophic factors. In some embodiments, a compound of the present disclosure is used to increase translation, transcription, or secretion of neurotrophic factors. In some embodiments, increasing translation, transcription or secretion of neurotrophic factors is sufficient for the treatment of migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, or addiction (e.g., substance use disorder).

An experiment or assay can be used to detect increased translation of neurotrophic factors, which can include, for example, ELISA, western blot, an immunofluorescence assay, a proteomic experiment, and mass spectrometry. In some embodiments, the experiment or assay used to detect increased transcription of neurotrophic factors is a gene expression assay, PCR, or microarray. In some embodiments, the experiment or assay used to detect increased secretion of neurotrophic factors is ELISA, western blot, an immunofluorescence assay, a proteomic experiment, or a mass spectrometry assay.

In some embodiments, the present disclosure provides a method for increasing translation, transcription, or secretion of neurotrophic factors, wherein the method comprises contacting a neuronal cell with a compound disclosed herein.

### Combination Therapy

In particular embodiments of treating the disorders described above, combination therapy is used as described herein. In such therapy a compound disclosed herein, including those described in Table 1, is administered in combination with a serotonin receptor modulator. In certain embodiments the serotonin receptor modulator is selected from the group consisting of altanserin, blonanserin, eplivanserin, glemanserin, volinanserin, ketanserin, ritanserin, pimavanserin, nelotanserin, pruvanserin, and flibanserin. In one embodiment, the serotonin receptor modulator is selected from the group consisting of serotonin receptor modulator is selected from the group consisting of eplivanserin, volinanserin, ketanserin, ritanserin, pimavanserin, nelotanserin, pruvanserin, flibanserin, olanzapine, quetiapine, risperidone, and buspirone.

In some embodiments, the serotonin receptor modulator for use with the compounds disclosed herein, including those described in Table 1, is eplivanserin, wherein the eplivanserin is administered in about 1 mg to about 40 mg, or about 5 mg to about 10 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is volinanserin, wherein the volinanserin is administered in about 1 mg to about 60 mg, or about 5 mg to about 20 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is ketanserin, wherein the ketanserin is administered in about 10 mg to about 80 mg, about 30 mg to about 50 mg, or about 40 mg and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is ritanserin, wherein the ritanserin is administered in about 1 mg to about 40 mg, or about 2.5 mg to about 10 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is pimavanserin, wherein the pimavanserin is administered in about 1 mg to about 60 mg, or about 17 mg to about 34 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is nelotanserin, wherein the nelotanserin is administered in about 1 mg to about 80 mg, or about 40 mg to about 80 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is pruvanserin, wherein the pruvanserin is administered in about 1 mg to about 40 mg, or about 3 mg to about 10 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is flibanserin, wherein the flibanserin is administered in about 10 mg to about 200 mg, or about 80 mg to about 120 mg, or about 100 mg and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is olanzapine, wherein the olanzapine is administered in about 2.5 mg to about 30 mg, or about 5 mg or about 10 mg, or about 20 mg or about 25 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is quetiapine, wherein the quetiapine is administered in about 25 mg to about 800 mg, or about 50 mg to about 100 mg, or about 150 mg or about 200 mg or about 250 mg or about 300 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is an extended-release of quetiapine, wherein the extended-release of quetiapine is administered in about 50 mg to about 300 mg, or about 50 mg or about 100 mg or about 200 mg, or about 300 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is risperidone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is an extended-release of risperidone including (RISPERDAL CONSTA), wherein the extended-release of risperidone is administered in about 12.5 mg, or about 25 mg, or about 37.5 mg, or about 50 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In some embodiments, the serotonin receptor modulator for use with the compounds disclosed herein, including those described in Table 1, is buspirone, wherein the buspirone is administered in about 1 mg to about 100 mg, or about 1 mg or about 2 mg, or about 3 mg, or about 4 mg, or about 5 mg, or about 6 mg, or about 7 mg, or about 7.5 mg, or about 10 mg, or about 15 mg, or about 22.5 mg, or about 30 mg, or about 37.5 mg, or about 45 mg, or about 52.5 mg, or about 60 mg, or about 1 mg to about 10 mg, or about 5 mg to about 10 mg, or about 10 mg to about 15 mg, or about 15 mg to about 30 mg, or about 30 mg to about 60 mg, or about 60 mg to about 80 mg, or about 80 mg to about 100 mg, and the compounds disclosed herein, including those described in Table 1, are administered between about 10 mg to about 500 mg, or about 100 mg to about 250 mg, or about 120 mg, or about 150 mg, or about 180 mg, or about 250 mg.

In certain embodiments, such as those described above, a compound disclosed herein, including those described in Table 1, is co-administered with a serotonin receptor modulator in the same or in separate compositions. In one embodiment, the compound disclosed herein, including those described in Table 1, is administered in a modified release formulation such that the subject is effectively pretreated with serotonin receptor modulator prior to release of an effective amount of the prodrug of methylone disclosed herein, including those described in Table 1. Thus, in some embodiments, the serotonin receptor modulator is administered or released from a composition provided herein prior to the administration and/or release of the psychedelic. This allows pretreatment to attenuate activation of the serotonin receptor by the psychedelic.

In some embodiments, the serotonin receptor modulator is administered or released from the composition provided herein to pretreat a subject by at least about at about 5 minutes, 10 minutes, 20 minutes, 30 minutes, 40 minutes, 50 minutes, 1 hour, 1.25 hours, 1.5 hours, 2 hours, or 3 hours prior to the release of the psychedelic. In some embodiments, the serotonin receptor modulator attenuates the activation of the serotonin receptor when the serotonin receptor modulator is used to pretreat at most about 3 hours, 4 hours, 5 hours, 6 hours, 7 hours, 8 hours, 9 hours, or more than 9 hours prior to the release of the psychedelic. In some embodiments, the serotonin receptor modulator attenuates the activation of the serotonin receptor when the serotonin receptor modulator is used to pretreat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour prior to the release of the psychedelic.

In a preferred embodiment, the serotonin receptor modulator is administered at about 1 hour to about 3 hours prior to the administration of the psychedelic.

In some embodiments, the serotonin receptor modulator is eplivanserin and the psychedelic is a prodrug of methylone disclosed herein, including those described in Table 1, wherein the eplivanserin is administered to pretreat at least 15 minutes prior to the administration of the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat between at least 30 minutes prior and 360 minutes prior to the release or administration of the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat between at least 60 minutes prior and 360 minutes prior to the release or administration of the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat between at least 90 minutes and 240 minutes prior to the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat at least 120 minutes prior to the prodrug of methylone disclosed herein, including those described in Table 1.

In some embodiments, the serotonin receptor modulator is eplivanserin and the psychedelic is a prodrug of methylone disclosed herein, including those described in Table 1, wherein the eplivanserin is administered to pretreat at least 150 minutes prior to the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat between about 15 minutes and about 150 minutes prior to the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat at least 180 minutes prior to the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat at least 210 minutes prior to the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat at least 240 minutes prior to the prodrug of methylone disclosed herein, including those described in Table 1.

In some embodiments, the serotonin receptor modulator is eplivanserin and the psychedelic is a prodrug of methylone disclosed herein, including those described in Table 1, wherein the eplivanserin is administered to pretreat at least 270 minutes prior to methylone. In some embodiments, the serotonin receptor modulator is eplivanserin and the psychedelic is a prodrug of methylone disclosed herein, including those described in Table 1, wherein the eplivanserin is administered to pretreat at least 300 minutes prior to the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat at least 330 minutes prior to the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to pretreat at least 360 minutes prior to the prodrug of methylone disclosed herein, including those described in Table 1.

In some preferred embodiments, the serotonin receptor modulator is eplivanserin and the psychedelic is a prodrug of methylone disclosed herein, including those described in Table 1, wherein eplivanserin is administered to pretreat between about 60 minutes and about 180 minutes prior to the administration of the prodrug of methylone disclosed herein, including those described in Table 1.

In some embodiments, the serotonin receptor modulator is volinanserin and the psychedelic is a prodrug of methylone disclosed herein, including those described in Table 1, wherein the volinanserin is administered to pretreat a subject between at least 15 minutes and 360 minutes prior to the administration or release of the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat between at least 30 minutes and 360 minutes prior to the administration or release of the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat between at least 60 minutes and 240 minutes prior to the administration or release of the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 90 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 120 minutes prior to the prodrug of methylone disclosed herein, including those described in Table 1.

In some embodiments, the serotonin receptor modulator is volinanserin and the psychedelic is a prodrug of methylone disclosed herein, including those described in Table 1, wherein the volinanserin is administered to pretreat at least 150 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat between about 15 minutes and about 150 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 180 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 210 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 240 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 270 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 300 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 330 minutes prior to the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to pretreat at least 360 minutes prior to the prodrug of methylone disclosed herein, including those described in Table 1. In some preferred embodiments, the serotonin receptor modulator is volinanserin and the psychedelic is a prodrug of methylone disclosed herein, including those described in Table 1, wherein volinanserin is administered to pretreat between about 60 minutes and about 180 minutes prior to the administration of the prodrug of methylone disclosed herein, including those described in Table 1.

In some embodiments, the serotonin receptor modulator is ketanserin and the psychedelic is a prodrug of methylone disclosed herein, including those described in Table 1, wherein the ketanserin is administered to pretreat at least 15 minutes prior to the administration of the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat between at least 30 minutes and 360 minutes prior to the administration or release of the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat between at least 60 minutes and 240 minutes prior to the administration or release of the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 90 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 120 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 150 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat between about 15 minutes and about 150 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 180 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 210 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 240 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 270 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 300 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 330 minutes prior to the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to pretreat at least 360 minutes prior to the prodrug of methylone disclosed herein, including those described in Table 1. In some preferred embodiments, the serotonin receptor modulator is ketanserin and the psychedelic is a prodrug of methylone disclosed herein, including those described in Table 1, wherein ketanserin is administered to pretreat between about 60 minutes and about 180 minutes prior to the administration of the prodrug of methylone disclosed herein, including those described in Table 1.

In some embodiments, the serotonin receptor modulator is ritanserin and the psychedelic is a prodrug of methylone disclosed herein, including those described in Table 1, wherein the ritanserin is administered to pretreat at least 15 minutes prior to the administration of the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 30 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat between at least 60 minutes and 240 minutes prior to the administration or release of the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 90 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 120 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 150 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat between about 15 minutes and about 150 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 180 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 210 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 240 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 270 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 300 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 330 minutes prior to the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to pretreat at least 360 minutes prior to the prodrug of methylone disclosed herein, including those described in Table 1. In some preferred embodiments, the serotonin receptor modulator is ritanserin and the psychedelic is a prodrug of methylone disclosed herein, including those described in Table 1, wherein ritanserin is administered to pretreat between about 60 minutes and about 180 minutes prior to the administration of the prodrug of methylone disclosed herein, including those described in Table 1.

In some embodiments, the serotonin receptor modulator is pimavanserin and the psychedelic is a prodrug of methylone disclosed herein, including those described in Table 1, wherein the pimavanserin is administered to pretreat at least 15 minutes prior to the administration of the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 30 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat between at least 60 minutes and 240 minutes prior to the administration or release of the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 90 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 120 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 150 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat between about 15 minutes and about 150 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 180 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 210 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 240 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 270 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 300 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 330 minutes prior to the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to pretreat at least 360 minutes prior to the prodrug of methylone disclosed herein, including those described in Table 1. In some preferred embodiments, the serotonin receptor modulator is pimavanserin and the psychedelic is a prodrug of methylone disclosed herein, including those described in Table 1, wherein pimavanserin is administered to pretreat between about 60 minutes and about 180 minutes prior to the administration of the prodrug of methylone disclosed herein, including those described in Table 1.

In some embodiments, the serotonin receptor modulator is nelotanserin and the psychedelic is a prodrug of methylone disclosed herein, including those described in Table 1, wherein the nelotanserin is administered to pretreat at least 15 minutes prior to the administration of the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 30 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat between at least 60 minutes and 240 minutes prior to the administration or release of the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 90 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 120 minutes prior to the prodrug of methylone disclosed herein, including those described in Table 1.

In some embodiments, the serotonin receptor modulator is nelotanserin and the psychedelic is a prodrug of methylone disclosed herein, including those described in Table 1, wherein the nelotanserin is administered to pretreat at least 150 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat between about 15 minutes and about 150 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 180 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 210 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 240 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 270 minutes prior to the prodrug of methylone disclosed herein, including those described in Table 1.

In some embodiments, the serotonin receptor modulator is nelotanserin and the psychedelic is a prodrug of methylone disclosed herein, including those described in Table 1, wherein the nelotanserin is administered to pretreat at least 300 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 330 minutes prior to the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to pretreat at least 360 minutes prior to the prodrug of methylone disclosed herein, including those described in Table 1. In some preferred embodiments, the serotonin receptor modulator is nelotanserin and the psychedelic is a prodrug of methylone disclosed herein, including those described in Table 1, wherein nelotanserin is administered to pretreat between about 60 minutes and about 180 minutes prior to the administration of the prodrug of methylone disclosed herein, including those described in Table 1.

In some embodiments, the serotonin receptor modulator is pruvanserin and the psychedelic is a prodrug of methylone disclosed herein, including those described in Table 1, wherein the pruvanserin is administered to pretreat at least 15 minutes prior to the administration of the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 30 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat between at least 60 minutes and 240 minutes prior to the administration or release of the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 90 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 120 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 150 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat between about 15 minutes and about 150 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 180 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 210 minutes prior to the prodrug of methylone disclosed herein, including those described in Table 1.

In some embodiments, the serotonin receptor modulator is pruvanserin and the psychedelic is a prodrug of methylone disclosed herein, including those described in Table 1, wherein the pruvanserin is administered to pretreat at least 240 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 270 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 300 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 330 minutes prior to the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to pretreat at least 360 minutes prior to the prodrug of methylone disclosed herein, including those described in Table 1. In some preferred embodiments, the serotonin receptor modulator is pruvanserin and the psychedelic is a prodrug of methylone disclosed herein, including those described in Table 1, wherein pruvanserin is administered to pretreat between about 60 minutes and about 180 minutes prior to the administration of the prodrug of methylone disclosed herein, including those described in Table 1.

In some embodiments, the serotonin receptor modulator is flibanserin and the psychedelic is a prodrug of methylone disclosed herein, including those described in Table 1, wherein the flibanserin is administered to pretreat at least 15 minutes prior to the administration of the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 30 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat between at least 60 minutes and 240 minutes prior to the administration or release of the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 90 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 120 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 150 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat between about 15 minutes and about 150 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 180 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 210 minutes prior to the prodrug of methylone disclosed herein, including those described in Table 1.

In some embodiments, the serotonin receptor modulator is flibanserin and the psychedelic is a prodrug of methylone disclosed herein, including those described in Table 1, wherein the flibanserin is administered to pretreat at least 240 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 270 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 300 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 330 minutes prior to the prodrug of methylone disclosed herein, including those described in Table 1. In some embodiments, the serotonin receptor modulator is flibanserin and the psychedelic is a prodrug of methylone disclosed herein, including those described in Table 1, wherein the flibanserin is administered to pretreat at least 360 minutes prior to the prodrug of methylone disclosed herein, including those described in Table 1. In some preferred embodiments, the serotonin receptor modulator is flibanserin and the psychedelic is a prodrug of methylone disclosed herein, including those described in Table 1, wherein flibanserin is administered to pretreat between about 60 minutes and about 180 minutes prior to the administration of the prodrug of methylone disclosed herein, including those described in Table 1.

In some embodiments, the serotonin receptor modulator is olanzapine and the psychedelic is a prodrug of methylone disclosed herein, including those described in Table 1, wherein the olanzapine is administered to pretreat at least 15 minutes prior to the administration of the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 30 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat between at least 60 minutes and 240 minutes prior to the administration or release of the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 90 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 120 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 150 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat between about 15 minutes and about 150 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 180 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 210 minutes prior to the prodrug of methylone disclosed herein, including those described in Table 1.

In some embodiments, the serotonin receptor modulator is olanzapine and the psychedelic is a prodrug of methylone disclosed herein, including those described in Table 1, wherein the olanzapine is administered to pretreat at least 240 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 270 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 300 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 330 minutes prior to the prodrug of methylone disclosed herein, including those described in Table 1. In some embodiments, the serotonin receptor modulator is olanzapine and the psychedelic is a prodrug of methylone disclosed herein, including those described in Table 1, wherein the olanzapine is administered to pretreat at least 360 minutes prior to the prodrug of methylone disclosed herein, including those described in Table 1. In some preferred embodiments, the serotonin receptor modulator is olanzapine and the psychedelic is a prodrug of methylone disclosed herein, including those described in Table 1, wherein olanzapine is administered to pretreat between about 60 minutes and about 180 minutes prior to the administration of the prodrug of methylone disclosed herein, including those described in Table 1.

In some embodiments, the serotonin receptor modulator is risperidone and the psychedelic is a prodrug of methylone disclosed herein, including those described in Table 1, wherein the risperidone is administered to pretreat at least 15 minutes prior to the administration of the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 30 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat between at least 60 minutes and 240 minutes prior to the administration or release of the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 90 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 120 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 150 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat between about 15 minutes and about 150 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 180 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 210 minutes prior to the prodrug of methylone disclosed herein, including those described in Table 1.

In some embodiments, the serotonin receptor modulator is risperidone and the psychedelic is a prodrug of methylone disclosed herein, including those described in Table 1, wherein the risperidone is administered to pretreat at least 240 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 270 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 300 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 330 minutes prior to the prodrug of methylone disclosed herein, including those described in Table 1. In some embodiments, the serotonin receptor modulator is risperidone and the psychedelic is a prodrug of methylone disclosed herein, including those described in Table 1, wherein the risperidone is administered to pretreat at least 360 minutes prior to the prodrug of methylone disclosed herein, including those described in Table 1. In some preferred embodiments, the serotonin receptor modulator is risperidone and the psychedelic is a prodrug of methylone disclosed herein, including those described in Table 1, wherein risperidone is administered to pretreat between about 60 minutes and about 180 minutes prior to the administration of the prodrug of methylone disclosed herein, including those described in Table 1.

In some embodiments, the serotonin receptor modulator is quetiapine and the psychedelic is a prodrug of methylone disclosed herein, including those described in Table 1, wherein the quetiapine is administered to pretreat at least 15 minutes prior to the administration of the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 30 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat between at least 60 minutes and 240 minutes prior to the administration or release of the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 90 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 120 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 150 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat between about 15 minutes and about 150 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 180 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 210 minutes prior to the prodrug of methylone disclosed herein, including those described in Table 1.

In some embodiments, the serotonin receptor modulator is quetiapine and the psychedelic is a prodrug of methylone disclosed herein, including those described in Table 1, wherein the quetiapine is administered to pretreat at least 240 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 270 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 300 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 330 minutes prior to the prodrug of methylone disclosed herein, including those described in Table 1. In some embodiments, the serotonin receptor modulator is quetiapine and the psychedelic is a prodrug of methylone disclosed herein, including those described in Table 1, wherein the quetiapine is administered to pretreat at least 360 minutes prior to the prodrug of methylone disclosed herein, including those described in Table 1. In some preferred embodiments, the serotonin receptor modulator is quetiapine and the psychedelic is a prodrug of methylone disclosed herein, including those described in Table 1, wherein quetiapine is administered to pretreat between about 60 minutes and about 180 minutes prior to the administration of the prodrug of methylone disclosed herein, including those described in Table 1.

In some embodiments, the serotonin receptor modulator is buspirone and the psychedelic is a prodrug of methylone disclosed herein, including those described in Table 1, wherein the buspirone is administered to pretreat at least 15 minutes prior to the administration of the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 30 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat between at least 60 minutes and 240 minutes prior to the administration or release of the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 90 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 120 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 150 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat between about 15 minutes and about 150 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 180 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 210 minutes prior to the prodrug of methylone disclosed herein, including those described in Table 1.

In some embodiments, the serotonin receptor modulator is buspirone and the psychedelic is a prodrug of methylone disclosed herein, including those described in Table 1, wherein the buspirone is administered to pretreat at least 240 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 270 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 300 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 330 minutes prior to the prodrug of methylone disclosed herein, including those described in Table 1. In some embodiments, the serotonin receptor modulator is buspirone and the psychedelic is a prodrug of methylone disclosed herein, including those described in Table 1, wherein the buspirone is administered to pretreat at least 360 minutes prior to the prodrug of methylone disclosed herein, including those described in Table 1. In some preferred embodiments, the serotonin receptor modulator is buspirone and the psychedelic is a prodrug of methylone disclosed herein, including those described in Table 1, wherein buspirone is administered to pretreat between about 60 minutes and about 180 minutes prior to the administration of the prodrug of methylone disclosed herein, including those described in Table 1.

In certain embodiments, such as those described above, a compound disclosed herein, including those described in Table 1, is co-administered with a serotonin receptor modulator in the same or in separate compositions. In one embodiment, the serotonin receptor modulator is administered after the compound disclosed herein, including those described in Table 1. In one embodiment, the compound disclosed herein, including those described in Table 1, is administered in a modified release formulation such that the subject is effectively post-treated with serotonin receptor modulator post to release of an effective amount of the prodrug of methylone disclosed herein, including those described in Table 1. In some embodiments, the serotonin receptor modulator is part of a single fixed dose formulation that releases the prodrug of methylone disclosed herein, including those described in Table 1, first followed by serotonin receptor modulator on two different release profiles. In another embodiment, the compound disclosed herein, including those described in Table 1, is administered first as a single dosage and, after a length of time, serotonin receptor modulator is administered as a second dosage separate from the first dosage. Thus, in some embodiments, the serotonin receptor modulator is administered or released from a composition provided herein after the administration and/or release of the psychedelic. This allows post-treatment to attenuate activation of the serotonin receptor by the psychedelic.

In some embodiments, the serotonin receptor modulator is administered or released from the composition provided herein to post-treat a subject by at least about at about 5 minutes, 10 minutes, 20 minutes, 30 minutes, 40 minutes, 50 minutes, 1 hour, 1.25 hours, 1.5 hours, 2 hours, or 3 hours after the release of the psychedelic. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours after the release of the psychedelic. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour after the release of the psychedelic.

In a preferred embodiment, the serotonin receptor modulator is administered at about 1 hour to about 3 hours after the administration of the psychedelic.

In some embodiments, the serotonin receptor modulator is eplivanserin and the psychedelic is a prodrug of methylone disclosed herein, including those described in Table 1, wherein the eplivanserin is administered to post-treat at least 15 minutes after the administration of the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat between at least 30 minutes after and 360 minutes after the release or administration of the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat between at least 60 minutes after and 360 minutes after the release or administration of the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat between at least 90 minutes and 240 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat at least 120 minutes after the prodrug of methylone disclosed herein, including those described in Table 1.

In some embodiments, the serotonin receptor modulator is eplivanserin and the psychedelic is a prodrug of methylone disclosed herein, including those described in Table 1, wherein the eplivanserin is administered to post-treat at least 150 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat between about 15 minutes and about 150 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat at least 180 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat at least 210 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat at least 240 minutes after the prodrug of methylone disclosed herein, including those described in Table 1.

In some embodiments, the serotonin receptor modulator is eplivanserin and the psychedelic is a prodrug of methylone disclosed herein, including those described in Table 1, wherein the eplivanserin is administered to post-treat at least 270 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat at least 300 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat at least 330 minutes after the prodrug of methylone disclosed herein, including those described in Table 1. In some embodiments, the serotonin receptor modulator is eplivanserin and the psychedelic is a prodrug of methylone disclosed herein, including those described in Table 1, wherein the eplivanserin is administered to post-treat at least 360 minutes after the prodrug of methylone disclosed herein, including those described in Table 1.

In some preferred embodiments, the serotonin receptor modulator is eplivanserin and the psychedelic is a prodrug of methylone disclosed herein, including those described in Table 1, wherein eplivanserin is administered to post-treat between about 60 minutes and about 180 minutes after the administration of the prodrug of methylone disclosed herein, including those described in Table 1.

In some embodiments, the serotonin receptor modulator is volinanserin and the psychedelic is a prodrug of methylone disclosed herein, including those described in Table 1, wherein the volinanserin is administered to post-treat a subject between at least 15 minutes and 360 minutes after the administration or release of the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat between at least 30 minutes and 360 minutes after the administration or release of the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat between at least 60 minutes and 240 minutes after the administration or release of the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 90 minutes after methylone. In some embodiments, the serotonin receptor modulator is volinanserin and the psychedelic is a prodrug of methylone disclosed herein, including those described in Table 1, wherein the volinanserin is administered to post-treat at least 120 minutes after the prodrug of methylone disclosed herein, including those described in Table 1.

In some embodiments, the serotonin receptor modulator is volinanserin and the psychedelic is a prodrug of methylone disclosed herein, including those described in Table 1, wherein the volinanserin is administered to post-treat at least 150 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat between about 15 minutes and about 150 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 180 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 210 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 240 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 270 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 300 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 330 minutes after the prodrug of methylone disclosed herein, including those described in Table 1. In some embodiments, the serotonin receptor modulator is volinanserin and the psychedelic is a prodrug of methylone disclosed herein, including those described in Table 1, wherein the volinanserin is administered to post-treat at least 360 minutes after the prodrug of methylone disclosed herein, including those described in Table 1. In some preferred embodiments, the serotonin receptor modulator is volinanserin and the psychedelic is a prodrug of methylone disclosed herein, including those described in Table 1, wherein volinanserin is administered to post-treat between about 60 minutes and about 180 minutes after the administration of the prodrug of methylone disclosed herein, including those described in Table 1.

In some embodiments, the serotonin receptor modulator is ketanserin and the psychedelic is a prodrug of methylone disclosed herein, including those described in Table 1, wherein the ketanserin is administered to post-treat at least 15 minutes after the administration of the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat between at least 30 minutes and 360 minutes after the administration or release of the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat between at least 60 minutes and 240 minutes after the administration or release of the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 90 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 120 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 150 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat between about 15 minutes and about 150 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 180 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 210 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 240 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 270 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 300 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 330 minutes after the prodrug of methylone disclosed herein, including those described in Table 1. In some embodiments, the serotonin receptor modulator is ketanserin and the psychedelic is a prodrug of methylone disclosed herein, including those described in Table 1, wherein the ketanserin is administered to post-treat at least 360 minutes after the prodrug of methylone disclosed herein, including those described in Table 1. In some preferred embodiments, the serotonin receptor modulator is ketanserin and the psychedelic is a prodrug of methylone disclosed herein, including those described in Table 1, wherein ketanserin is administered to post-treat between about 60 minutes and about 180 minutes after the administration of the prodrug of methylone disclosed herein, including those described in Table 1.

In some embodiments, the serotonin receptor modulator is ritanserin and the psychedelic is a prodrug of methylone disclosed herein, including those described in Table 1, wherein the ritanserin is administered to post-treat at least 15 minutes after the administration of the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 30 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat between at least 60 minutes and 240 minutes after the administration or release of the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 90 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 120 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 150 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat between about 15 minutes and about 150 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 180 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 210 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 240 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 270 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 300 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 330 minutes after the prodrug of methylone disclosed herein, including those described in Table 1. In some embodiments, the serotonin receptor modulator is ritanserin and the psychedelic is a prodrug of methylone disclosed herein, including those described in Table 1, wherein the ritanserin is administered to post-treat at least 360 minutes after the prodrug of methylone disclosed herein, including those described in Table 1. In some preferred embodiments, the serotonin receptor modulator is ritanserin and the psychedelic is a prodrug of methylone disclosed herein, including those described in Table 1, wherein ritanserin is administered to post-treat between about 60 minutes and about 180 minutes after the administration of the prodrug of methylone disclosed herein, including those described in Table 1.

In some embodiments, the serotonin receptor modulator is pimavanserin and the psychedelic is a prodrug of methylone disclosed herein, including those described in Table 1, wherein the pimavanserin is administered to post-treat at least 15 minutes after the administration of the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 30 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat between at least 60 minutes and 240 minutes after the administration or release of the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 90 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 120 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 150 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat between about 15 minutes and about 150 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 180 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 210 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 240 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 270 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 300 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 330 minutes after the prodrug of methylone disclosed herein, including those described in Table 1. In some embodiments, the serotonin receptor modulator is pimavanserin and the psychedelic is a prodrug of methylone disclosed herein, including those described in Table 1, wherein the pimavanserin is administered to post-treat at least 360 minutes after the prodrug of methylone disclosed herein, including those described in Table 1. In some preferred embodiments, the serotonin receptor modulator is pimavanserin and the psychedelic is a prodrug of methylone disclosed herein, including those described in Table 1, wherein pimavanserin is administered to post-treat between about 60 minutes and about 180 minutes after the administration of the prodrug of methylone disclosed herein, including those described in Table 1.

In some embodiments, the serotonin receptor modulator is nelotanserin and the psychedelic is a prodrug of methylone disclosed herein, including those described in Table 1, wherein the nelotanserin is administered to post-treat at least 15 minutes after the administration of the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 30 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat between at least 60 minutes and 240 minutes after the administration or release of the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 90 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 120 minutes after the prodrug of methylone disclosed herein, including those described in Table 1.

In some embodiments, the serotonin receptor modulator is nelotanserin and the psychedelic is a prodrug of methylone disclosed herein, including those described in Table 1, wherein the nelotanserin is administered to post-treat at least 150 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat between about 15 minutes and about 150 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 180 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 210 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 240 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 270 minutes after the prodrug of methylone disclosed herein, including those described in Table 1.

In some embodiments, the serotonin receptor modulator is nelotanserin and the psychedelic is a prodrug of methylone disclosed herein, including those described in Table 1, wherein the nelotanserin is administered to post-treat at least 300 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 330 minutes after the prodrug of methylone disclosed herein, including those described in Table 1. In some embodiments, the serotonin receptor modulator is nelotanserin and the psychedelic is a prodrug of methylone disclosed herein, including those described in Table 1, wherein the nelotanserin is administered to post-treat at least 360 minutes after the prodrug of methylone disclosed herein, including those described in Table 1. In some preferred embodiments, the serotonin receptor modulator is nelotanserin and the psychedelic is a prodrug of methylone disclosed herein, including those described in Table 1, wherein nelotanserin is administered to post-treat between about 60 minutes and about 180 minutes after the administration of the prodrug of methylone disclosed herein, including those described in Table 1.

In some embodiments, the serotonin receptor modulator is pruvanserin and the psychedelic is a prodrug of methylone disclosed herein, including those described in Table 1, wherein the pruvanserin is administered to post-treat at least 15 minutes after the administration of the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 30 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat between at least 60 minutes and 240 minutes after the administration or release of the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 90 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 120 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 150 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat between about 15 minutes and about 150 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 180 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 210 minutes after the prodrug of methylone disclosed herein, including those described in Table 1.

In some embodiments, the serotonin receptor modulator is pruvanserin and the psychedelic is a prodrug of methylone disclosed herein, including those described in Table 1, wherein the pruvanserin is administered to post-treat at least 240 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 270 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 300 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 330 minutes after the prodrug of methylone disclosed herein, including those described in Table 1. In some embodiments, the serotonin receptor modulator is pruvanserin and the psychedelic is a prodrug of methylone disclosed herein, including those described in Table 1, wherein the pruvanserin is administered to post-treat at least 360 minutes after the prodrug of methylone disclosed herein, including those described in Table 1. In some preferred embodiments, the serotonin receptor modulator is pruvanserin and the psychedelic is a prodrug of methylone disclosed herein, including those described in Table 1, wherein pruvanserin is administered to post-treat between about 60 minutes and about 180 minutes after the administration of the prodrug of methylone disclosed herein, including those described in Table 1.

In some embodiments, the serotonin receptor modulator is flibanserin, wherein the flibanserin is administered to post-treat at least 15 minutes post to the administration of the prodrug of methylone disclosed herein, including those described in Table 1. In some embodiments, the serotonin receptor modulator is flibanserin, wherein the flibanserin is administered to post-treat at least 30 minutes post to the prodrug of methylone disclosed herein, including those described in Table 1. In some embodiments, the serotonin receptor modulator is flibanserin, wherein the flibanserin is administered to post-treat between at least 60 minutes and 240 minutes post to the administration or release of the prodrug of methylone disclosed herein, including those described in Table 1. In some embodiments, the serotonin receptor modulator is flibanserin, wherein the flibanserin is administered to post-treat at least 90 minutes post to the prodrug of methylone disclosed herein, including those described in Table 1. In some embodiments, the serotonin receptor modulator is flibanserin, wherein the flibanserin is administered to post-treat at least 120 minutes post to the prodrug of methylone disclosed herein, including those described in Table 1. In some embodiments, the serotonin receptor modulator is flibanserin, wherein the flibanserin is administered to post-treat at least 150 minutes post to the prodrug of methylone disclosed herein, including those described in Table 1. In some embodiments, the serotonin receptor modulator is flibanserin, wherein the flibanserin is administered to post-treat between about 15 minutes and about 150 minutes post to the prodrug of methylone disclosed herein, including those described in Table 1. In some embodiments, the serotonin receptor modulator is flibanserin, wherein the flibanserin is administered to post-treat at least 180 minutes post to the prodrug of methylone disclosed herein, including those described in Table 1. In some embodiments, the serotonin receptor modulator is flibanserin, wherein the flibanserin is administered to post-treat at least 210 minutes post to the prodrug of methylone disclosed herein, including those described in Table 1.

In some embodiments, the serotonin receptor modulator is flibanserin, wherein the flibanserin is administered to post-treat at least 240 minutes post to the prodrug of methylone disclosed herein, including those described in Table 1. In some embodiments, the serotonin receptor modulator is flibanserin, wherein the flibanserin is administered to post-treat at least 270 minutes post to the prodrug of methylone disclosed herein, including those described in Table 1. In some embodiments, the serotonin receptor modulator is flibanserin, wherein the flibanserin is administered to post-treat at least 300 minutes post to the prodrug of methylone disclosed herein, including those described in Table 1. In some embodiments, the serotonin receptor modulator is flibanserin, wherein the flibanserin is administered to post-treat at least 330 minutes post to the prodrug of methylone disclosed herein, including those described in Table 1. In some embodiments, the serotonin receptor modulator is flibanserin, wherein the flibanserin is administered to post-treat at least 360 minutes post to the prodrug of methylone disclosed herein, including those described in Table 1. In some preferred embodiments, the serotonin receptor modulator is flibanserin, wherein flibanserin is administered to post-treat between about 60 minutes and about 180 minutes post to the administration of the prodrug of methylone disclosed herein, including those described in Table 1.

In some embodiments, the serotonin receptor modulator is olanzapine, wherein the olanzapine is administered to post-treat at least 15 minutes post to the administration of the prodrug of methylone disclosed herein, including those described in Table 1. In some embodiments, the serotonin receptor modulator is olanzapine, wherein the olanzapine is administered to post-treat at least 30 minutes post to the prodrug of methylone disclosed herein, including those described in Table 1. In some embodiments, the serotonin receptor modulator is olanzapine, wherein the olanzapine is administered to post-treat between at least 60 minutes and 240 minutes post to the administration or release of the prodrug of methylone disclosed herein, including those described in Table 1. In some embodiments, the serotonin receptor modulator is olanzapine, wherein the olanzapine is administered to post-treat at least 90 minutes post to the prodrug of methylone disclosed herein, including those described in Table 1. In some embodiments, the serotonin receptor modulator is olanzapine, wherein the olanzapine is administered to post-treat at least 120 minutes post to the prodrug of methylone disclosed herein, including those described in Table 1. In some embodiments, the serotonin receptor modulator is olanzapine, wherein the olanzapine is administered to post-treat at least 150 minutes post to the prodrug of methylone disclosed herein, including those described in Table 1. In some embodiments, the serotonin receptor modulator is olanzapine, wherein the olanzapine is administered to post-treat between about 15 minutes and about 150 minutes post to the prodrug of methylone disclosed herein, including those described in Table 1. In some embodiments, the serotonin receptor modulator is olanzapine, wherein the olanzapine is administered to post-treat at least 180 minutes post to the prodrug of methylone disclosed herein, including those described in Table 1. In some embodiments, the serotonin receptor modulator is olanzapine, wherein the olanzapine is administered to post-treat at least 210 minutes post to the prodrug of methylone disclosed herein, including those described in Table 1.

In some embodiments, the serotonin receptor modulator is olanzapine, wherein the olanzapine is administered to post-treat at least 240 minutes post to the prodrug of methylone disclosed herein, including those described in Table 1. In some embodiments, the serotonin receptor modulator is olanzapine, wherein the olanzapine is administered to post-treat at least 270 minutes post to the prodrug of methylone disclosed herein, including those described in Table 1. In some embodiments, the serotonin receptor modulator is olanzapine, wherein the olanzapine is administered to post-treat at least 300 minutes post to the prodrug of methylone disclosed herein, including those described in Table 1. In some embodiments, the serotonin receptor modulator is olanzapine, wherein the olanzapine is administered to post-treat at least 330 minutes post to the prodrug of methylone disclosed herein, including those described in Table 1. In some embodiments, the serotonin receptor modulator is olanzapine, wherein the olanzapine is administered to post-treat at least 360 minutes post to the prodrug of methylone disclosed herein, including those described in Table 1. In some preferred embodiments, the serotonin receptor modulator is olanzapine, wherein olanzapine is administered to post-treat between about 60 minutes and about 180 minutes post to the administration of the prodrug of methylone disclosed herein, including those described in Table 1.

In some embodiments, the serotonin receptor modulator is quetiapine, wherein the quetiapine is administered to post-treat at least 15 minutes post to the administration of the prodrug of methylone disclosed herein, including those described in Table 1. In some embodiments, the serotonin receptor modulator is quetiapine, wherein the quetiapine is administered to post-treat at least 30 minutes post to the prodrug of methylone disclosed herein, including those described in Table 1. In some embodiments, the serotonin receptor modulator is quetiapine, wherein the quetiapine is administered to post-treat between at least 60 minutes and 240 minutes post to the administration or release of the prodrug of methylone disclosed herein, including those described in Table 1. In some embodiments, the serotonin receptor modulator is quetiapine, wherein the quetiapine is administered to post-treat at least 90 minutes post to the prodrug of methylone disclosed herein, including those described in Table 1. In some embodiments, the serotonin receptor modulator is quetiapine, wherein the quetiapine is administered to post-treat at least 120 minutes post to the prodrug of methylone disclosed herein, including those described in Table 1. In some embodiments, the serotonin receptor modulator is quetiapine, wherein the quetiapine is administered to post-treat at least 150 minutes post to the prodrug of methylone disclosed herein, including those described in Table 1. In some embodiments, the serotonin receptor modulator is quetiapine, wherein the quetiapine is administered to post-treat between about 15 minutes and about 150 minutes post to the prodrug of methylone disclosed herein, including those described in Table 1. In some embodiments, the serotonin receptor modulator is quetiapine, wherein the quetiapine is administered to post-treat at least 180 minutes post to the prodrug of methylone disclosed herein, including those described in Table 1. In some embodiments, the serotonin receptor modulator is quetiapine, wherein the quetiapine is administered to post-treat at least 210 minutes post to the prodrug of methylone disclosed herein, including those described in Table 1.

In some embodiments, the serotonin receptor modulator is quetiapine, wherein the quetiapine is administered to post-treat at least 240 minutes post to the prodrug of methylone disclosed herein, including those described in Table 1. In some embodiments, the serotonin receptor modulator is quetiapine, wherein the quetiapine is administered to post-treat at least 270 minutes post to the prodrug of methylone disclosed herein, including those described in Table 1. In some embodiments, the serotonin receptor modulator is quetiapine, wherein the quetiapine is administered to post-treat at least 300 minutes post to the prodrug of methylone disclosed herein, including those described in Table 1. In some embodiments, the serotonin receptor modulator is quetiapine, wherein the quetiapine is administered to post-treat at least 330 minutes post to the prodrug of methylone disclosed herein, including those described in Table 1. In some embodiments, the serotonin receptor modulator is quetiapine, wherein the quetiapine is administered to post-treat at least 360 minutes post to the prodrug of methylone disclosed herein, including those described in Table 1. In some preferred embodiments, the serotonin receptor modulator is quetiapine, wherein quetiapine is administered to post-treat between about 60 minutes and about 180 minutes post to the administration of the prodrug of methylone disclosed herein, including those described in Table 1.

In some embodiments, the serotonin receptor modulator is risperidone, wherein the risperidone is administered to post-treat at least 15 minutes post to the administration of the prodrug of methylone disclosed herein, including those described in Table 1. In some embodiments, the serotonin receptor modulator is risperidone, wherein the risperidone is administered to post-treat at least 30 minutes post to the prodrug of methylone disclosed herein, including those described in Table 1. In some embodiments, the serotonin receptor modulator is risperidone, wherein the risperidone is administered to post-treat between at least 60 minutes and 240 minutes post to the administration or release of the prodrug of methylone disclosed herein, including those described in Table 1. In some embodiments, the serotonin receptor modulator is risperidone, wherein the risperidone is administered to post-treat at least 90 minutes post to the prodrug of methylone disclosed herein, including those described in Table 1. In some embodiments, the serotonin receptor modulator is risperidone, wherein the risperidone is administered to post-treat at least 120 minutes post to the prodrug of methylone disclosed herein, including those described in Table 1. In some embodiments, the serotonin receptor modulator is risperidone, wherein the risperidone is administered to post-treat at least 150 minutes post to the prodrug of methylone disclosed herein, including those described in Table 1. In some embodiments, the serotonin receptor modulator is risperidone, wherein the risperidone is administered to post-treat between about 15 minutes and about 150 minutes post to the prodrug of methylone disclosed herein, including those described in Table 1. In some embodiments, the serotonin receptor modulator is risperidone, wherein the risperidone is administered to post-treat at least 180 minutes post to the prodrug of methylone disclosed herein, including those described in Table 1. In some embodiments, the serotonin receptor modulator is risperidone, wherein the risperidone is administered to post-treat at least 210 minutes post to the prodrug of methylone disclosed herein, including those described in Table 1.

In some embodiments, the serotonin receptor modulator is risperidone, wherein the risperidone is administered to post-treat at least 240 minutes post to the prodrug of methylone disclosed herein, including those described in Table 1. In some embodiments, the serotonin receptor modulator is risperidone, wherein the risperidone is administered to post-treat at least 270 minutes post to the prodrug of methylone disclosed herein, including those described in Table 1. In some embodiments, the serotonin receptor modulator is risperidone, wherein the risperidone is administered to post-treat at least 300 minutes post to the prodrug of methylone disclosed herein, including those described in Table 1. In some embodiments, the serotonin receptor modulator is risperidone, wherein the risperidone is administered to post-treat at least 330 minutes post to the prodrug of methylone disclosed herein, including those described in Table 1. In some embodiments, the serotonin receptor modulator is risperidone, wherein the risperidone is administered to post-treat at least 360 minutes post to the prodrug of methylone disclosed herein, including those described in Table 1. In some preferred embodiments, the serotonin receptor modulator is risperidone, wherein risperidone is administered to post-treat between about 60 minutes and about 180 minutes post to the administration of the prodrug of methylone disclosed herein, including those described in Table 1.

In some embodiments, the serotonin receptor modulator is buspirone, wherein the buspirone is administered to post-treat at least 15 minutes post to the administration of the prodrug of methylone disclosed herein, including those described in Table 1. In some embodiments, the serotonin receptor modulator is buspirone, wherein the buspirone is administered to post-treat at least 30 minutes post to the prodrug of methylone disclosed herein, including those described in Table 1. In some embodiments, the serotonin receptor modulator is buspirone, wherein the buspirone is administered to post-treat between at least 60 minutes and 240 minutes post to the administration or release of the prodrug of methylone disclosed herein, including those described in Table 1. In some embodiments, the serotonin receptor modulator is buspirone, wherein the buspirone is administered to post-treat at least 90 minutes post to the prodrug of methylone disclosed herein, including those described in Table 1. In some embodiments, the serotonin receptor modulator is buspirone, wherein the buspirone is administered to post-treat at least 120 minutes post to the prodrug of methylone disclosed herein, including those described in Table 1. In some embodiments, the serotonin receptor modulator is buspirone, wherein the buspirone is administered to post-treat at least 150 minutes post to the prodrug of methylone disclosed herein, including those described in Table 1. In some embodiments, the serotonin receptor modulator is buspirone, wherein the buspirone is administered to post-treat between about 15 minutes and about 150 minutes post to the prodrug of methylone disclosed herein, including those described in Table 1. In some embodiments, the serotonin receptor modulator is buspirone, wherein the buspirone is administered to post-treat at least 180 minutes post to the prodrug of methylone disclosed herein, including those described in Table 1. In some embodiments, the serotonin receptor modulator is buspirone, wherein the buspirone is administered to post-treat at least 210 minutes post to the prodrug of methylone disclosed herein, including those described in Table 1.

In some embodiments, the serotonin receptor modulator is buspirone, wherein the buspirone is administered to post-treat at least 240 minutes post to the prodrug of methylone disclosed herein, including those described in Table 1. In some embodiments, the serotonin receptor modulator is buspirone, wherein the buspirone is administered to post-treat at least 270 minutes post to the prodrug of methylone disclosed herein, including those described in Table 1. In some embodiments, the serotonin receptor modulator is buspirone, wherein the buspirone is administered to post-treat at least 300 minutes post to the prodrug of methylone disclosed herein, including those described in Table 1. In some embodiments, the serotonin receptor modulator is buspirone, wherein the buspirone is administered to post-treat at least 330 minutes post to the prodrug of methylone disclosed herein, including those described in Table 1. In some embodiments, the serotonin receptor modulator is buspirone, wherein the buspirone is administered to post-treat at least 360 minutes post to the prodrug of methylone disclosed herein, including those described in Table 1. In some preferred embodiments, the serotonin receptor modulator is buspirone, wherein buspirone is administered to post-treat between about 60 minutes and about 180 minutes post to the administration of the prodrug of methylone disclosed herein, including those described in Table 1.

### EXAMPLES

### I. Chemical Synthesis

### General Conditions:

Mass spectra were run on LC-MS systems using electrospray ionization. These were run using a Waters Acquity Classic UPLC with PDA and SQ mass detection or a Waters Acquity H-Class UPLC with PDA and QDA mass detection. [M+H]⁺ refers to mono-isotopic molecular weights.

NMR spectra were run on either a Bruker Ultrashield 400 MHz or 500 MHz NMR spectrometer. Spectra were recorded at 298 K, unless otherwise stated, and were referenced using the solvent peak.

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., MS and NMIR.

Abbreviations used are those conventional in the art. If not defined, the terms have their generally accepted meanings.

### Abbreviations

app apparent
Boc tert-butyl carbamate
Boc-Sar-OH Boc-sarcosine
br broad
CDCl₃ d₃-chloroform
d doublet
dd doublet of doublets
DCM dichloromethane
DIPEA diisopropylethylamine
DMA dimethylacetamide
DMAP 4-dimethylaminopyridine
DMF N,N-dimethylformamide
DMSO dimethyl sulfoxide
EtOAc ethyl acetate
EDC HCl N-(3-dimethylaminopropyl)-N′-ethylcarbodiimide hydrochloride
HATU 1-[Bis(dimethylamino)methylene]-1H-1,2,3-triazolo [4,5-b]pyridinium 3-oxid hexafluorophosphate
HCl hydrochloric acid
h hextet; sextet
hept heptet
HPLC high pressure liquid chromatography
LC-MS liquid chromatography and mass spectrometry
MeOH MeOH
MeCN acetonitrile
MgSO₄ magnesium sulfate
MS mass spectrometry
m multiplet
min(s) minute(s)
mL milliliter(s)
L microliter(s)
m/z mass to charge ratio
N₂ nitrogen
NaHCO₃ sodium hydrogen carbonate
Na₂SO₄ sodium sulfate
NMP N-methyl-2-pyrrolidone
NMR nuclear magnetic resonance
p pentet
q quartet
Rt retention time
s singlet
sar sarcosine
t triplet
tert tertiary
THF tetrahydrofuran

The various starting materials, intermediates, and compounds of the preferred embodiments may be isolated and purified, where appropriate, using conventional techniques such as precipitation, filtration, crystallization, evaporation, distillation, and chromatography. Salts may be prepared from compounds by known salt-forming procedures. Unless otherwise stated, all starting materials are obtained from commercial suppliers and used without further purification. More specific compounds required for the syntheses are listed below:

5-(tert-Butoxy)-5-oxopentanoic acid (cas 63128-51-8) purchased from Sigma Aldrich (catalogue number SY3H3D678586).

6-(tert-Butoxy)-6-oxohexanoic acid (cas 52221-07-5) purchased from BLDpharm (catalogue number BD00759729).

3-(2-Acetoxy-4,6-dimethylphenyl)-3-methylbutyric acid (cas 134098-68-3) purchased from Sigma Aldrich (catalogue number 756377).

2-Methoxyethyl chloroformate (cas 628-12-6) purchased from Enamine (catalogue number EN300-222696).

### HPLC Methods

If not indicated otherwise, the analytical HPLC conditions are as follows:

### Instrument: LC-MS-1:

### Method 2A

Column: Acquity UPLC BEH C18 2.1×50 mm 1.7 μm
Column Temp: 50° C.
Flow rate: 0.8 mL/min.
Eluents: A: H₂O, 0.1% formic acid, B: MeCN
Gradient: 0.0-1.8 min 2-98% B, 1.8-2.1 min 98% B, 2.1-2.5 98% A.

### Method 2B

Column: Acquity UPLC BEH C18 2.1×50 mm 1.7 μm
Column Temp: 50° C.
Flow rate: 0.8 mL/min.
Eluents: A: H₂O, 0.1% ammonia B: MeCN
Gradient: 0.0-1.8 min 2-98% B, 1.8-2.1 min 98% B, 2.1-2.5 98% A.

### Instrument: LC-MS-2:

### Method 2A

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% formic acid
Gradient: 0.0-1.7 mins 0-95% B, 5% C; 1.7-2.1 mins 95% B, 5% C 2.1-2.5 mins 95% A, 5% C.

### Method 2B

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% ammonia (aq.)
Gradient: 0.0-1.7 mins 0-95% B, 5% D; 1.7-2.1 mins 95% B, 5% D 2.1-2.5 mins 95% A, 5% D.

### Example 1: Ethyl N-[2-(1,3-benzodioxol-5-yl)-1-methyl-2-oxo-ethyl]-N-methyl-carbamate (Compound 1A)

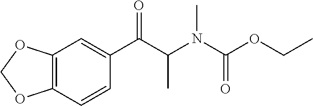 [SMILES: CCOC(=O)N(C)C(C)C(=O)c1ccc2c(c1)OCO2]

Ethyl chloroformate (81 mg, 0.74 mmol, 71 μL) was added dropwise over 2 min to a stirred solution of 1-(1,3-benzodioxol-5-yl)-2-(methylamino)propan-1-one hydrochloride (151 mg, 0.62 mmol) and DIPEA (187 mg, 1.45 mmol, 252 μL) in DCM (5 mL) at 0° C. under N₂. The mixture was stirred at 0° C. for 1 h and then warmed to rt overnight. DCM (25 mL) and 1M aqueous HCl (25 mL) were added to the mixture. The separated aqueous phase was extracted with DCM (25 mL) and the combined organic fractions were then washed with saturated aqueous NaHCO₃ (25 mL), dried over Na₂SO₄ and concentrated in vacuo. The residue was purified by column chromatography on silica gel, eluting with 40% EtOAc in iso-hexane, to leave ethyl N-[2-(1,3-benzodioxol-5-yl)-1-methyl-2-oxo-ethyl]-N-methyl-carbamate (146 mg, 84%) as an oil. Spectroscopic data of the title compound was obtained as a mixture of two rotational isomers. LC-MS (LCMS2: Method 2A): Rt 1.49 mins; m/z 280.0=[M+H]⁺; ¹H NMR (400 MHz, CDCl₃) δ 7.67 (dd, J=8.2, 1.8 Hz, 0.7H), 7.55 (d, J=8.2 Hz, 0.3H), 7.48 (d, J=1.8 Hz, 0.7H), 7.40 (s, 0.3H), 6.84 (d, J=8.2 Hz, 1H), 6.03 (s, 2H), 5.68 (q, J=6.9 Hz, 0.7H), 5.38 (q, J=7.0 Hz, 0.3H), 4.28-4.13 (m, 2H), 2.77 (s, 0.9H), 2.69 (s, 2.1H), 1.39-1.32 (m, 3H), 1.31-1.23 (m, 3H).

The compounds of the following table were prepared analogously to Example 1 from 1-(1,3-benzodioxol-5-yl)-2-(methylamino)propan-1-one hydrochloride and the appropriate chloroformate or acid chloride.

TABLE Ex1

| Cmpd | | |
|---|---|---|
| No. | Structure and Name | Characterization Data |
| 1B | 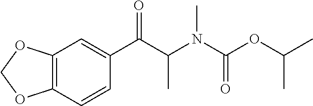 [SMILES: CC(C)OC(=O)N(C)C(C)C(=O)c1ccc2c(c1)OCO2] | LC-MS (LCMS2: Method 2A): Rt 1.58 mins; m/z 294.1 = [M + H]⁺ ¹H NMR (400 MHz, CDCl₃) δ 7.67 (d, J = 8.2 Hz, 0.65H), 7.56 (d, J = 8.2 Hz, 0.35H), 7.47 (s, 0.65H), 7.41 (s, 0.35H), 6.83 (d, J = 8.2 Hz, 1H), 6.06-6.00 (m, 2H), 5.67 (q, J = 6.8 Hz, 0.65H), 5.34 (q, J = 6.7 Hz, 0.35H), |
| | iso-Propyl N-[2-(1,3-benzodioxol-5- | 5.04-4.88 (m, 1H), 2.75 (s, 1.05H), 2.67 (s, |
| | yl)-1-methyl-2-oxo-ethyl]-N-methyl- | 1.95H), 1.37-1.33 (m, 3H), 1.30-1.21 (m, |
| | carbamate | 6H). |
| 1C | 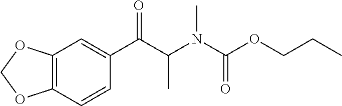 [SMILES: CCCOC(=O)N(C)C(C)C(=O)c1ccc2c(c1)OCO2] | LC-MS (LCMS2: Method 2B): Rt 1.57 mins; MS m/z 294.1 = [M + H]⁺ ¹H NMR (400 MHz, CDCl₃) δ 7.67 (dd, J = 8.2, 1.8 Hz, 0.7H), 7.55 (d, J = 8.2 Hz, 0.3H), 7.47 (d, J = 1.8 Hz, 0.7H), 7.40 (s, 0.3H), 6.84 (d, J = 8.2 Hz, 1H), 6.07-6.00 (m, 2H), 5.67 (q, J = 6.9 Hz, 0.7H), 5.37 (q, J = 6.9 |
| | Propyl N-[2-(1,3-benzodioxol-5-yl)- | Hz, 0.3H), 4.18-4.02 (m, 2H), 2.77 (s, |
| | 1-methyl-2-oxo-ethyl]-N-methyl- | 0.9H), 2.70 (s, 2.1H), 1.72-1.60 (m, 2H), |
| | carbamate | 1.39-1.33 (m, 3H), 0.98-0.90 (m, 3H). |
| 1D | 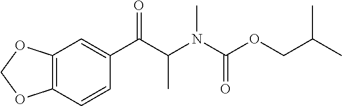 [SMILES: CC(C)COC(=O)N(C)C(C)C(=O)c1ccc2c(c1)OCO2] | LC-MS (LCMS2: Method 2A): Rt 1.66 mins; m/z 308.1 = [M + H]⁺ ¹H NMR (400 MHz, CDCl₃) δ 7.67 (dd, J = 8.2, 1.7 Hz, 0.7H), 7.55 (d, J = 8.2 Hz, 0.3H), 7.47 (d, J = 1.7 Hz, 0.7H), 7.40 (s, 0.3H), 6.84 (d, J = 8.2 Hz, 1H), 6.09-5.99 (m, 2H), 5.67 (q, J = 6.9 Hz, 0.7H), 5.38 (q, J = 6.9 |
| | iso-Butyl N-[2-(1,3-benzodioxol-5- | Hz, 0.3H), 4.00-3.84 (m, 2H), 2.78 (s, |
| | yl)-1-methyl-2-oxo-ethyl]-N-methyl- | 0.9H), 2.71 (s, 2.1H), 2.01-1.87 (m, 1H), |
| | carbamate | 1.41-1.32 (m, 3H), 0.98-0.89 (m, 6H). |
| 1E | 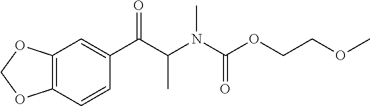 [SMILES: COCCOC(=O)N(C)C(C)C(=O)c1ccc2c(c1)OCO2] | LC-MS (LCMS2: Method 2A): Rt 1.26 mins; m/z 310.0 = [M + H]⁺ LC-MS (LCMS2: Method 2B): Rt 1.32 mins; m/z 310.0 = [M + H]⁺ ¹H NMR (400 MHz, CDCl₃) δ 7.66 (dd, J = 8.2, 1.8 Hz, 0.65H), 7.61 (d, J = 8.2 Hz, 0.35H), 7.47 (d, J = 1.8 Hz, 0.65H), 7.43 (s, |
| | 2-Methoxyethyl N-[2-(1,3- | 0.35H), 6.84 (d, J = 8.2 Hz, 1H), 6.07-6.01 |
| | benzodioxol-5-yl)-1-methyl-2-oxo- | (m, 2H), 5.66 (q, J = 6.9 Hz, 0.65H), 5.49 (q, |
| | ethyl]-N-methyl-carbamate | J = 6.8 Hz, 0.35H), 4.42-4.20 (m, 2H), 3.64- |
| | | 3.57 (m, 2H), 3.39 (s, 1.05H), 3.37 (s, |
| | | 1.95H), 2.75 (s, 1.05H), 2.73 (s, 1.95H), |
| | | 1.39-1.33 (m, 3H). |
| 1F | 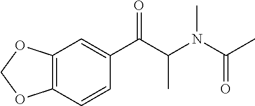 [SMILES: CC(=O)N(C)C(C)C(=O)c1ccc2c(c1)OCO2] | LC-MS (LCMS2: Method 2A): Rt 1.14 mins; m/z 249.9 = [M + H]⁺ ¹H NMR (400 MHz, CDCl₃) δ 7.65 (dd, J = 8.2, 1.8 Hz, 1H), 7.46 (d, J = 1.8 Hz, 1H), 6.84 (d, J = 8.2 Hz, 1H), 6.10 (q, J = 6.9 Hz, 1H), 6.06 (s, 0.1H), 6.03 (s, 1.9H), 2.79 (s, 2.85H), 2.76 (s, 0.15H), 2.25 (s, 0.15H), 2.09 |
| | N-[2-(1,3-Benzodioxol-5-yl)-1- | (s, 2.85H), 1.47 (d, J = 6.9 Hz, 0.15H), 1.32 |
| | methyl-2-oxo-ethyl]-N-methyl- | (d, J = 6.9 Hz, 2.85H). |
| | acetamide | |
| 1G | 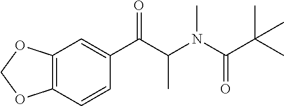 [SMILES: CC(C(=O)c1ccc2c(c1)OCO2)N(C)C(=O)C(C)(C)C] | LC-MS (LCMS2: Method 2B): Rt 1.49 mins; m/z 292.1 = [M + H]⁺ No rotational isomers observed: ¹H NMR (400 MHz, CDCl₃) δ 7.53 (dd, J = 8.2, 1.7 Hz, 1H), 7.39 (d, J = 1.7 Hz, 1H), 6.81 (d, J = 8.2 Hz, 1H), 6.04-6.01 (m, 2H), 5.97 (q, J = 6.8 Hz, 1H), 2.90 (s, 3H), 1.32 (d, J = 6.8 |
| | N-[2-(1,3-Benzodioxol-5-yl)-1- | Hz, 3H), 1.24 (s, 9H). |
| | methyl-2-oxo-ethyl]-N-2,2- | |
| | trimethyl-propanamide | |

### Example 2: tert-Butyl N-[2-(1,3-benzodioxol-5-yl)-1-methyl-2-oxo-ethyl]-N-methyl-carbamate (Compound 2A)

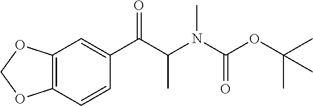 [SMILES: CC(C(=O)c1ccc2c(c1)OCO2)N(C)C(=O)OC(C)(C)C]

Di-tert-butyl dicarbonate (162 mg, 0.74 mmol) was added in several portion over 10 min to a stirred solution of 1-(1,3-benzodioxol-5-yl)-2-(methylamino)propan-1-one hydrochloride (150 mg, 0.62 mmol) and DIPEA (159 mg, 1.23 mmol, 215 μL) in DCM (5 mL) at 0° C. under N₂. The mixture was stirred at 0° C. for 10 min, warmed to rt and then stirred at this temperature overnight. Water (20 mL) and DCM (25 mL) were added to the mixture. The separated aqueous phase was extracted with DCM (2×25 mL) and the combined organic fractions were then washed with saturated aqueous NaHCO₃ (25 mL) and brine (25 mL), dried over Na₂SO₄ and concentrated in vacuo. The residue was purified by column chromatography on silica gel, eluting with 20% EtOAc in iso-hexane, to leave tert-butyl N-[2-(1,3-benzodioxol-5-yl)-1-methyl-2-oxo-ethyl]-N-methyl-carbamate (142 mg, 75%) as an oil. Spectroscopic data of the title compound was obtained as a mixture of two rotational isomers. LC-MS (LCMS2: Method 2A): Rt 1.66 mins; m/z 252.1=[M-^{t}Bu+H]⁺; ¹H NMR (400 MHz, CDCl₃) δ 7.68 (d, J=8.2 Hz, 0.65H), 7.56 (d, J=8.2 Hz, 0.35H), 7.48 (s, 0.65H), 7.41 (s, 0.35H), 6.83 (d, J=8.2 Hz, 1H), 6.07-5.99 (m, 2H), 5.63 (q, J=6.8 Hz, 0.65H), 5.18 (q, J=7.0 Hz, 0.35H), 2.75 (s, 1.05H), 2.62 (s, 1.95H), 1.46 (s, 9H), 1.38-1.30 (m, 3H).

The compounds of the following table were prepared analogously to Example 2 from 1-(1,3-benzodioxol-5-yl)-2-(methylamino)propan-1-one hydrochloride and the appropriate anhydride.

TABLE Ex2

| Cmpd No. | Structure and Name | Characterization Data |
|---|---|---|
| 2B | 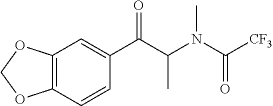 [SMILES: CC(C(=O)c1ccc2c(c1)OCO2)N(C)C(=O)C(F)(F)F] | LC-MS (LCMS2: Method 2A): Rt 1.53 mins; m/z 304.0 = [M + H]⁺ ¹H NMR (400 MHz, CDCl₃) δ 7.55 (dd, J = 8.2, 1.8 Hz, 0.9H), 7.47 (dd, J = 8.2, 1.8 Hz, 0.1H), 7.41 (d, J = 1.8 Hz, 0.9H), 7.35 (d, J = 1.8 Hz, 0.1H), 6.90-6.83 (m, 1H), 6.07 (s, 0.2H), 6.06 (s, 1.8H), 5.94 (q, J = 7.0 Hz, 0.9H), 5.44 (q, J = 7.0 Hz, 0.1H), 3.03 (s, 0.3H), 2.98 (q, |
| | N-[2-(1,3-Benzodioxol- | J = 1.6 Hz, 2.7H), 1.57 (d, J = 7.0 Hz, 0.3H), 1.43 (d, J = |
| | 5-yl)-1-methyl-2-oxo- | 7.0 Hz, 2.7H). |
| | ethyl]-2,2,2-trifluoro-N- | |
| | methyl-acetamide | |

### Example 3: [2-[3-[[2-(1,3-Benzodioxol-5-yl)-1-methyl-2-oxo-ethyl]-methyl-amino]-1,1-dimethyl-3-oxo-propyl]-3,5-dimethyl-phenyl]acetate (Compound 3A)

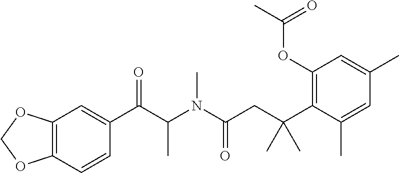 [SMILES: CC(=O)Oc1cc(C)cc(C)c1C(C)(C)CC(=O)N(C)C(C)C(=O)c1ccc2c(c1)OCO2]

HATU (358 mg, 0.94 mmol) was added in one portion, followed by DIPEA (487 mg, 3.77 mmol, 656 μL) which was added dropwise over 2 min to a stirred solution of 1-(1,3-benzodioxol-5-yl)-2-(methylamino)propan-1-one hydrochloride (153 mg, 0.63 mmol) and 3-(2-acetoxy-4,6-dimethylphenyl)-3-methylbutyric acid (249 mg, 0.94 mmol) in DMF (5 mL) at rt under N₂. The mixture was stirred at rt overnight. Water (50 mL) and EtOAc (40 mL) were added to the mixture. The separated organic phase was washed with brine (2×25 mL), dried over Na₂SO₄ and concentrated in vacuo. The residue was purified by column chromatography on silica gel, eluting with a gradient of 40-50% EtOAc in hexane, to leave [2-[3-[[2-(1,3-benzodioxol-5-yl)-1-methyl-2-oxo-ethyl]-methyl-amino]-1,1-dimethyl-3-oxo-propyl]-3,5-dimethyl-phenyl]acetate (264 mg, 93%) as an oil. LC-MS (LCMS2: Method 2B): Rt 1.83 mins; m z 454.2=[M+H]+; ¹H NMR (400 MHz, CDCl₃) δ 7.56 (dd, J=8.2, 1.7 Hz, 1H), 7.42 (d, J=1.7 Hz, 1H), 6.82 (d, J=2.2 Hz, 1H), 6.75 (d, J=8.2 Hz, 1H), 6.56 (d, J=2.2 Hz, 1H), 6.07 (q, J=6.8 Hz, 1H), 6.03 (d, J=1.3 Hz, 1H), 6.02 (d, J=1.3 Hz, 1H), 2.87 (d, J=16.0 Hz, 1H), 2.77 (d, J=16.0 Hz, 1H), 2.70 (s, 3H), 2.53 (s, 3H), 2.23 (s, 3H), 2.21 (s, 3H), 1.57 (s, 3H), 1.55 (s, 3H), 1.24 (d, J=6.8 Hz, 3H).

The compounds of the following table were prepared analogously to Example 3 from 1-(1,3-benzodioxol-5-yl)-2-(methylamino)propan-1-one hydrochloride and the appropriate carboxylic acid.

TABLE Ex3

| Cmpd | | |
|---|---|---|
| No. | Structure and Name | Characterization Data |
| 3B | 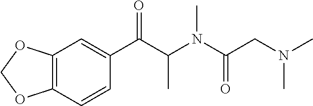 [SMILES: CC(C(=O)c1ccc2c(c1)OCO2)N(C)C(=O)CN(C)C] | LC-MS (LCMS2: Method 2B): Rt 1.24 mins; m/z 293.1 = [M + H]⁺ Mixture of two rotational isomers: ¹H NMR (400 MHz, CDCl₃) δ 7.68- 7.61 (m, 1H), 7.56 (d, J = 1.8 Hz, 0.15H), 7.45 (d, J = 1.8 Hz, 0.85H), 6.85 (d, J = 8.2 Hz, 0.15H), 6.82 (d, |
| | N-[2-(1,3-Benzodioxol-5-yl)-1-methyl-2- | J = 8.2 Hz, 0.85H), 6.10-6.01 (m, |
| | oxo-ethyl]-2-(dimethylamino)-N-methyl- | 2.85H), 5.68 (q, J = 7.1 Hz, 0.15H), |
| | acetamide | 3.36 (d, J = 12.9 Hz, 0.15H), 3.09 (s, |
| | | 1.70H), 2.98 (d, J = 12.9 Hz, 0.15H), |
| | | 2.88 (s, 0.45H), 2.84 (s, 2.55H), 2.27 |
| | | (s, 5.10H), 2.15 (s, 0.90H), 1.47 (d, |
| | | J = 7.1 Hz, 0.45H), 1.32 (d, J = 7.1 Hz, |
| | | 2.55H). |
| 3C | 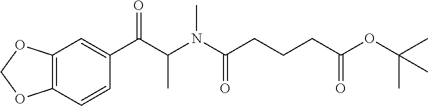 [SMILES: CC(C(=O)c1ccc2c(c1)OCO2)N(C)C(=O)CCCC(=O)OC(C)(C)C] | LC-MS (LCMS2: Method 2A): Rt 1.62 mins; m/z 378.2 = [M + H]⁺ ¹H NMR (400 MHz, CDCl₃) δ 7.62 (dd, J = 8.2, 1.8 Hz, 1H), 7.44 (d, J = 1.8 Hz, 1H), 6.83 (d, J = 8.2 Hz, 1H), 6.09 (q, J = 6.8 Hz, 1H), 6.03 (s, 2H), 2.78 (s, 3H), 2.37-2.31 (m, 2H), |
| | tert-Butyl 4-{[2-(2H-1,3-benzodioxol-5-yl)- | 2.31-2.25 (m, 2H), 1.91 (p, J = 7.3 |
| | 1-methyl-2-oxo-ethyl]-N- | Hz, 2H), 1.43 (s, 9H), 1.32 (d, J = 6.8 |
| | methylcarbamoyl}butyrate | Hz, 3H). |
| 3D | 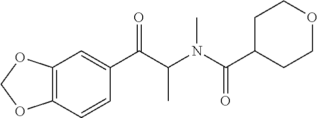 [SMILES: CC(C(=O)c1ccc2c(c1)OCO2)N(C)C(=O)C1CCOCC1] | LC-MS (LCMS2: Method 2A): Rt 1.24 mins; m/z 320.2 = [M + H]⁺ ¹H NMR (400 MHz, CDCl₃) δ 7.58 (dd, J = 8.2, 1.7 Hz, 1H), 7.41 (d, J = 1.7 Hz, 1H), 6.82 (d, J = 8.2 Hz, 1H), 6.07 (q, J = 6.9 Hz, 1H), 6.03 (s, 2H), 4.04 (ddd, J = 11.5, 4.4, 2.4 Hz, 1H), 3.98 (ddd, J = 11.5, 4.4, 2.4 Hz, 1H), |
| | N-[2-(1,3-Benzodioxol-5-yl)-1-methyl-2- | 3.47-3.35 (m, 2H), 2.83 (s, 3H), |
| | oxo-ethyl]-N-methyl-tetrahydropyran-4- | 2.70 (tt, J = 11.2, 3.8 Hz, 1H), 2.02- |
| | carboxamide | 1.91 (m, 1H), 1.86-1.74 (m, 1H), |
| | | 1.66-1.59 (m, 1H), 1.50-1.43 (m, |
| | | 1H), 1.33 (d, J = 6.9 Hz, 3H). |
| 3E | 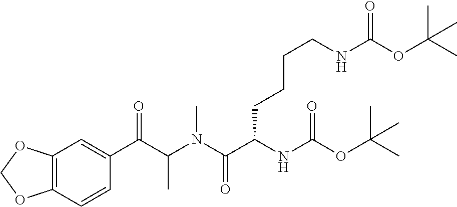 [SMILES: CC(C(=O)c1ccc2c(c1)OCO2)N(C)C(=O)[C@H](CCCCNC(=O)OC(C)(C)C)NC(=O)OC(C)(C)C] | LC-MS (LCMS2: Method 2B): Rt 1.71 mins; m/z 536.3 = [M + H]⁺ Mixture of diastereoisomers and rotational isomers: ¹H NMR (400 MHz, CDCl₃) δ 7.59-7.53 (m, 1H), 7.42 (d, J = 1.8 Hz, 0.2H), 7.39 (d, J = 1.8 Hz, 0.8H), 6.85-6.78 (m, 1H), 6.06-5.97 (m, 2.8H), 5.92 (q, J = 6.9 Hz, 0.2H), 5.42-5.29 (m, 1H), 4.68- 4.48 (m, 2H), 3.12-2.98 (m, 2H), 2.88 (s, 0.6H), 2.85 (s, 2.4H), 1.49- 1.30 (m, 27H). |
| | tert-Butyl N-[(5S)-6-[[2-(1,3-benzodioxol- | |
| | 5-yl)-1-methyl-2-oxo-ethyl]-methyl-amino]- | |
| | 5-(tert-butoxycarbonylamino)-6-oxo- | |
| | hexyl]carbamate | |
| 3F | 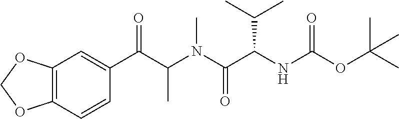 [SMILES: CC(C)[C@H](NC(=O)OC(C)(C)C)C(=O)N(C)C(C)C(=O)c1ccc2c(c1)OCO2] | LC-MS (LCMS2: Method 2B): Rt 1.62 and 1.64 mins; m/z 407.3 = [M + H]⁺ Mixture of diastereoisomers and rotational isomers: ¹H NMR (400 MHz, CDCl₃) δ 7.62-7.55 (m, 1H), 7.46-7.39 (m, 1H), 6.84-6.76 (m, 1H), 6.08-6.01 (m, 2.5H), 5.96 (q, |
| | tert-Butyl N-[(1S)-1-[[2-(1,3-benzodioxol- | J = 6.8 Hz, 0.5H), 5.26 (d, J = 9.4 Hz, |
| | 5-yl)-1-methyl-2-oxo-ethyl]-methyl- | 0.5H), 5.20 (d, J = 9.4 Hz, 0.5H), 4.43 |
| | carbamoyl]-2-methyl-propyl]carbamate | (dd, J = 9.4, 5.1 Hz, 0.5H), 4.38 (dd, |
| | | J = 9.4, 6.3 Hz, 0.5H), 2.90 (s, 1.5H), |
| | | 2.87 (s, 1.5H), 2.00-1.91 (m, 0.5H), |
| | | 1.85-1.76 (m, 0.5H), 1.44 (s, 4.5H), |
| | | 1.39 (s, 4.5H), 1.35-1.32 (m, 3H), |
| | | 0.95 (d, J = 6.8 Hz, 1.5H), 0.91-0.83 |
| | | (m, 4.5H). |
| 3G | 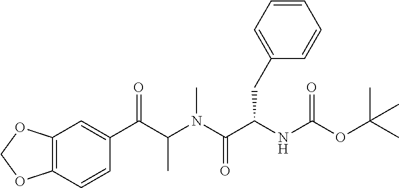 [SMILES: CC(C(=O)c1ccc2c(c1)OCO2)N(C)C(=O)[C@H](Cc1ccccc1)NC(=O)OC(C)(C)C] | LC-MS (LCMS2: Method 2A): Rt 1.73 mins; m/z 455.3 = [M + H]⁺ Mixture of diastereoisomers and rotational isomers: ¹H NMR (400 MHz, CDCl₃) δ 7.63 (dd, J = 8.2, 1.7 Hz, 0.5H), 7.57 (dd, J = 8.2, 1.7 Hz, 0.5H), 7.46 (d, J = 1.7 Hz, 0.5H), 7.39 (d, J = 1.7 Hz, 0.5H), 7.31-7.27 (m, 1.5H), 7.23-7.18 (m, 1H), 7.18- 7.07 (m, 1.5H), 7.08-7.01 (m, 1H), 6.85 (d, J = 8.2 Hz, 0.5H), 6.79 (d, J = 8.2 Hz, 0.5H), 6.10-5.99 (m, 2.5H), |
| | tert-Butyl N-[(1S)-2-[[2-(1,3-benzodioxol- | 5.95 (q, J = 6.9 Hz, 0.5H), 5.39-5.24 |
| | 5-yl)-1-methyl-2-oxo-ethyl]-methyl-amino]- | (m, 1H), 4.88-4.65 (m, 1H), 2.98 (d, |
| | 1-benzyl-2-oxo-ethyl]carbamate | J = 7.4 Hz, 1H), 2.85 (dd, J = 13.6, |
| | | 6.9 Hz, 0.5H), 2.78 (dd, J = 13.6, 7.0 |
| | | Hz, 0.5H), 2.68 (s, 1.5H), 2.45 (s, |
| | | 1.5H), 1.39 (s, 4.5H), 1.38 (s, 4.5H), |
| | | 1.32 (d, J = 6.9 Hz, 1.5H), 1.13 (d, J = |
| | | 6.9 Hz, 1.5H). |
| 3H | 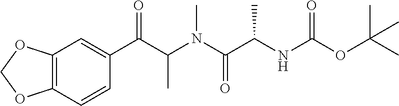 [SMILES: CC(C(=O)c1ccc2c(c1)OCO2)N(C)C(=O)[C@H](C)NC(=O)OC(C)(C)C] | LC-MS (LCMS2: Method 2A): Rt 1.58 and 1.60 mins; MS m/z 379.3 = [M + H]⁺ LC-MS (LCMS2: Method 2B): Rt 1.50 mins; m/z 379.3 = [M + H]⁺ Mixture of diastereoisomers and rotational isomers: ¹H NMR (400 |
| | tert-Butyl N-[(1S)-2-[[2-(1,3-benzodioxol- | MHz, CDCl₃) δ 7.59 (dd, J = 8.2, 1.7 |
| | 5-yl)-1-methyl-2-oxo-ethyl]-methyl-amino]- | Hz, 0.5H), 7.53 (dd, J = 8.2, 1.7 Hz, |
| | 1-methyl-2-oxo-ethyl]carbamate | 0.5H), 7.43 (d, J = 1.7 Hz, 0.5H), 7.38 |
| | | (d, J = 1.7 Hz, 0.5H), 6.85-6.78 (m, |
| | | 1H), 6.05-5.99 (m, 2.5H), 5.95 (q, |
| | | J = 6.8 Hz, 0.5H), 5.46 (d, J = 8.1 Hz, |
| | | 0.5H), 5.41 (d, J = 8.1 Hz, 0.5H), 4.62- |
| | | 4.50 (m, 1H), 2.87 (s, 1.5H), 2.81 (s, |
| | | 1.5H), 1.44 (s, 4.5H), 1.42 (s, 4.5H), |
| | | 1.37-1.32 (m, 3H), 1.29 (d, J = 6.8 |
| | | Hz, 1.5H), 1.15 (d, J = 6.8 Hz, 1.5H). |

### Example 4: (2S)-2-(3-Aminobutyl)-6-(1,3-benzodioxol-5-yl)-4,5-dimethyl-2,5-dihydropyrazin-3-one dihydrochloride (Compound 4A)

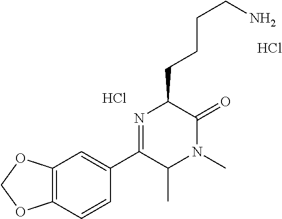 [SMILES: CC1C(c2ccc3c(c2)OCO3)=N[C@@H](CCCCN)C(=O)N1C ; Cl ; Cl]

A solution of HCl in dioxane (6.07 mmol, 1.52 mL, 4 M) was added dropwise over 5 min to a stirred solution of tert-butyl N-[(5S)-6-[[2-(1,3-benzodioxol-5-yl)-1-methyl-2-oxo-ethyl]-methyl-amino]-5-(tert-butoxycarbonylamino)-6-oxo-hexyl]carbamate (130 mg, 0.24 mmol) in 1,4-dioxane (1 mL) at 0° C. under N₂. The mixture was stirred at 0° C. for 1 hour and then warmed to rt overnight. The mixture was stirred at 40° C. for 4 h. The mixture was concentrated in vacuo and the residue azeotroped with chloroform (3×15 mL) and then DCM (3×15 mL). The compound was further dried under vacuum at 50° C. to leave (2S)-2-(3-aminobutyl)-6-(1,3-benzodioxol-5-yl)-4,5-dimethyl-2,5-dihydropyrazin-3-one dihydrochloride (88 mg, 93%) as a solid. Spectroscopic data of the title compound was obtained as a mixture of diastereoisomers and rotational isomers. LC-MS (LCMS2: Method 2B): Rt 1.22 and 1.30 mins; m z 318.2=[M+H]+; ¹H NMR (400 MHz, D₂O) δ 7.66-7.46 (m, 1H), 7.46-7.31 (m, 1H), 7.20-7.00 (m, 1H), 6.27-6.03 (m, 2H), 5.82 (q, J=6.9 Hz, 0.2H), 5.50-5.38 (m, 0.8H), 4.63-4.39 (m, 1H), 3.26 (s, 0.2H), 3.15 (s, 2.1H), 3.09-2.80 (m, 2.7H), 2.38-2.04 (m, 1.6H), 1.83-1.52 (m, 5.4H), 1.47-1.16 (m, 2H). NH₂ and both HCl salts not observed.

The compounds of the following table were prepared analogously to Example 4 from the appropriate Boc-protected amine.

TABLE Ex4

| Cmpd | | |
|---|---|---|
| No. | Structure and Name | Characterization Data |
| 4B | 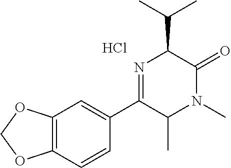 [SMILES: CC(C)[C@@H]1N=C(c2ccc3c(c2)OCO3)C(C)N(C)C1=O ; Cl] | LC-MS (LCMS2: Method 2B): Rt 1.33 and 1.48 mins; m/z 289.1 = [M + H]⁺ ¹H NMR (400 MHz, D₂O) δ 7.60-7.46 (m, 1H), 7.43-7.31 (m, 1H), 7.18-7.08 (m, 0.65H), 7.02- 6.96 (m, 0.35H), 6.24-6.04 (m, 2H), 5.82 (q, J = 6.9 Hz, 0.4H), 5.51-5.44 (m, 0.25H), 5.42-5.35 (m, 0.25H), 5.01 (q, J = 6.9 Hz, 0.1H), 4.60-4.57 (m, 0.25H), 4.46-4.39 (m, 0.4H), 4.32 (d, J = 4.7 Hz, 0.25H), 4.28 (d, J = 4.5 Hz, 0.1H), 3.27 (s, 0.3H), 3.15 (s, 1.2H), 3.12 (s, 0.75H), 2.99 (s, 0.75H), 2.76- 2.67 (m, 0.25H), 2.50-2.39 (m, 0.4H), 2.18-2.07 |
| | (2S)-6-(1,3-Benzodioxol-5- | (m, 0.35H), 1.62-1.40 (m, 3H), 1.22-1.09 (m, |
| | yl)-2-iso-propyl-4,5- | 3.7H), 1.01-0.88 (m, 2H), 0.55 (d, J = 6.9 Hz, |
| | dimethyl-2,5- | 0.3H). HCl not observed. |
| | dihydropyrazin-3-one | |
| | hydrochloride | |
| 4C | 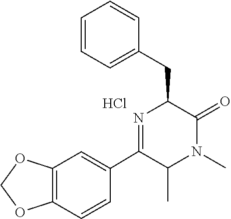 [SMILES: CC1C(c2ccc3c(c2)OCO3)=N[C@@H](Cc2ccccc2)C(=O)N1C ; Cl] | LC-MS (LCMS2: Method 2A): Rt 1.31 and 1.43 mins; m/z 337.0 = [M + H]⁺ LC-MS (LCMS2: Method 2B): Rt 1.37 and 1.47 mins; m/z 337.0 = [M + H]⁺ ¹H NMR (400 MHz, MeOD-d4) δ 7.69-7.52 (m, 1H), 7.44-7.34 (m, 2.3H), 7.31-7.09 (m, 3.8H), 6.99-6.89 (m, 0.9H), 6.24-6.00 (m, 2.6H), 5.78 (q, J = 7.1 Hz, 0.4H), 4.66-4.32 (m, 1H), 3.16-2.88 (m, 2.5H), 2.73 (s, 1.2H), 2.65 (s, 1.2H), 2.37 (s, 0.1H), 1.63-1.33 (m, 1.6H), 1.28 (d, J = 7.1 Hz, 1.3H), 1.05 (d, J = 7.1 Hz, 0.1H). HCl not observed. |
| | (2S)-6-(1,3-Benzodioxol-5- | |
| | yl)-2-benzyl-4,5-dimethyl- | |
| | 2,5-dihydropyrazin-3-one | |
| | hydrochloride | |
| 4D | 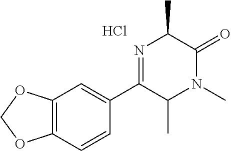 [SMILES: CC1C(c2ccc3c(c2)OCO3)=N[C@@H](C)C(=O)N1C ; Cl] | LC-MS (LCMS2: Method 2B): Rt 1.18 and 1.21 mins; m/z 261.1 = [M + H]⁺ ¹H NMR (400 MHz, D₂O) δ 7.67-7.59 (m, 0.9H), 7.49-7.38 (m, 0.9H), 7.14 (d, J = 8.2 Hz, 0.9H), 7.01-6.89 (m, 0.3H), 6.40-6.03 (m, 2.2H), 5.47- 5.36 (m, 0.8H), 4.77-4.62 (m, 1H), 3.64 (s, 0.3H), 3.15 (s, 1.5H), 3.14 (s, 1.2H), 2.42 (s, 0.3H), 2.36 (s, 0.3H), 1.76-1.68 (m, 2.7H), 1.63 (d, J = 7.2 Hz, 1.5H), 1.54 (d, J = 7.2 Hz, 1.2H). HCl not observed. |
| | (2S)-6-(1,3-Benzodioxol-5- | |
| | yl)-2,4,5-trimethyl-2,5- | |
| | dihydropyrazin-3-one | |
| | hydrochloride | |

### Example 5: Chloromethyl N-[2-(1,3-benzodioxol-5-yl)-1-methyl-2-oxo-ethyl]-N-methyl-carbamate (Compound 5A)

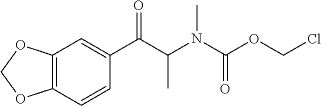 [SMILES: CC(C(=O)c1ccc2c(c1)OCO2)N(C)C(=O)OCCl]

Triethylamine (1.87 g, 18.5 mmol, 2.58 mL) was added dropwise over 5 min to a stirred suspension of 1-(1,3-benzodioxol-5-yl)-2-(methylamino)propan-1-one hydrochloride (1.50 g, 6.17 mmol) in DCM (60 mL) at 0° C. under N₂. The mixture was stirred at 0° C. for 30 min and then chloromethyl chloroformate (954 mg, 7.40 mmol, 658 μL) was added dropwise over 10 min. The mixture was stirred at 0° C. for 90 min. DCM (40 mL), chloroform (50 mL) and water (100 mL) were added to the mixture. The separated aqueous phase was extracted with chloroform (100 mL) and the combined organic fractions were then dried over Na₂SO₄ and concentrated in vacuo. The residue was purified by column chromatography on silica gel, eluting with a gradient of 0-40% EtOAc in petroleum ether, to leave chloromethyl N-[2-(1,3-benzodioxol-5-yl)-1-methyl-2-oxo-ethyl]-N-methyl-carbamate (1.49 g, 80%) as a gum. Spectroscopic data of the title compound was obtained as a mixture of two rotational isomers. LC-MS (LCMS2: Method 2A): Rt 1.57 mins; m/z 300.0=[M+H]*; LC-MS (LCMS2: Method 2B): Rt 1.50 mins; m/z 300.0=[M+H]⁺; ¹H NMR (400 MHz, CDCl₃) δ 7.63 (dd, J=8.2, 1.8 Hz, 0.7H), 7.55 (dd, J=8.2, 1.8 Hz, 0.3H), 7.45 (d, J=1.8 Hz, 0.7H), 7.39 (d, J=1.8 Hz, 0.3H), 6.85 (d, J=8.2 Hz, 1H), 6.07-6.03 (m, 2H), 5.91 (d, J=5.9 Hz, 0.3H), 5.82-5.77 (m, 1.7H), 5.67 (q, J=6.9 Hz, 0.7H), 5.46 (q, J=6.9 Hz, 0.3H), 2.80 (s, 0.9H), 2.77 (s, 2.1H), 1.42-1.37 (m, 3H).

The compounds of the following table were prepared analogously to Example 5 from 1-(1,3-benzodioxol-5-yl)-2-(methylamino)propan-1-one hydrochloride and the appropriate chloroformate.

TABLE Ex5

| Cmpd | | |
|---|---|---|
| No. | Structure and Name | Characterization Data |
| 5B | 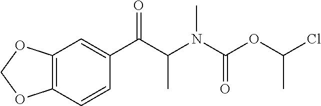 [SMILES: CC(Cl)OC(=O)N(C)C(C)C(=O)c1ccc2c(c1)OCO2] | LC-MS (LCMS2: Method 2A): Rt 1.57 mins; m/z 314.0 = [M + H]⁺ LC-MS (LCMS2: Method 2B): Rt 1.57 mins; m/z 314.0 = [M + H]⁺ Mixture of diastereoisomers and rotational isomers: ¹H NMR (400 MHz, CDCl₃) δ 7.68- 7.52 (m, 1H), 7.46-7.36 (m, 1H), 6.89-6.80 |
| | 1-Chloroethyl N-[2-(1,3- | (m, 1H), 6.67 (q, J = 5.8 Hz, 0.15H), 6.64-6.54 |
| | benzodioxol-5-yl)-1-methyl-2- | (m, 0.85H), 6.07-6.02 (m, 2H), 5.72-5.62 (m, |
| | oxo-ethyl]-N-methyl-carbamate | 0.7H), 5.47-5.38 (m, 0.3H), 2.87 (s, 0.45H), |
| | | 2.81 (s, 0.9H), 2.74 (s, 0.45H), 2.71 (s, 1.2H), |
| | | 1.87 (d, J = 5.8 Hz, 0.45H), 1.83-1.77 (m, |
| | | 2.55H), 1.43-1.35 (m, 3H). |

### Example 6: [[2-(1,3-Benzodioxol-5-yl)-1-methyl-2-oxo-ethyl]-methyl-carbamoyl]oxymethyl 2,2-dimethylpropanoate (Compound 6A)

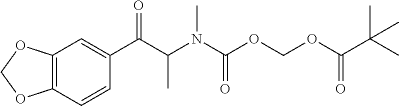 [SMILES: CC(C(=O)c1ccc2c(c1)OCO2)N(C)C(=O)OCOC(=O)C(C)(C)C]

A solution of 1M tetrabutylammonium hydroxide in MeOH (0.97 mmol, 966 μL) was added dropwise over 5 min to a stirred solution of pivalic acid (99 mg, 0.97 mmol) in MeOH (3 mL) at rt under N₂. The mixture was stirred at rt for 1 h and then concentrated in vacuo. THF (2 mL) was added to the residue. A solution of chloromethyl N-[2-(1,3-benzodioxol-5-yl)-1-methyl-2-oxo-ethyl]-N-methyl-carbamate (193 mg, 0.64 mmol) in THF (2 mL) was added dropwise over 5 min to the stirred mixture at rt under N₂, and the resulting mixture was then stirred at rt for 2 h. EtOAc (25 mL) was added to the mixture and the organic phase was then washed with brine (25 mL), water (2×25 mL) and brine (25 mL). The organic phase was dried over Na₂SO₄ and concentrated in vacuo. The residue was purified by chromatography on silica, eluting with a gradient of 0-5% EtOAc in DCM, to leave [[2-(1,3-benzodioxol-5-yl)-1-methyl-2-oxo-ethyl]-methyl-carbamoyl]oxymethyl 2,2-dimethylpropanoate (190 mg, 80%) as a gum. Spectroscopic data of the title compound was obtained as a mixture of two rotational isomers. LC-MS (LCMS2: Method 2B): Rt 1.66 mins; m/z 366.2=[M+H]⁺; ¹H NMR (400 MHz, CDCl₃) δ 7.64 (dd, J=8.2, 1.7 Hz, 0.7H), 7.53 (dd, J=8.2, 1.7 Hz, 0.3H), 7.45 (d, J=1.7 Hz, 0.7H), 7.38 (d, J=1.7 Hz, 0.3H), 6.87-6.79 (m, 1H), 6.06-6.01 (m, 2H), 5.85 (d, J=5.5 Hz, 0.3H), 5.82-5.77 (m, 1.7H), 5.65 (q, J=6.9 Hz, 0.7H), 5.44 (q, J=6.9 Hz, 0.3H), 2.80 (s, 0.9H), 2.71 (s, 2.1H), 1.36 (d, J=6.9 Hz, 3H), 1.20 (s, 2.7H), 1.18 (s, 6.3H).

The compounds of the following table were prepared analogously to Example 6 from the appropriate chloride and the appropriate carboxylic acid.

TABLE Ex6

| Cmpd | | |
|---|---|---|
| No. | Structure and Name | Characterization Data |
| 6B | 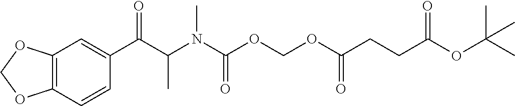 [SMILES: CC(C(=O)c1ccc2c(c1)OCO2)N(C)C(=O)OCOC(=O)CCC(=O)OC(C)(C)C] | LC-MS (LCMS2: Method 2A): Rt 1.67 mins; m/z 455.2 = [M + NH₄]⁺ ¹H NMR (400 MHz, CDCl₃) δ 7.64 (dd, J = 8.2, 1.8 Hz, 0.7H), 7.54 (dd, J = 8.2, 1.8 Hz, 0.3H), 7.45 (d, J = 1.8 Hz, 0.7H), 7.36 (d, J = 1.8 Hz, 0.3H), 6.89-6.83 (m, 1H), 6.07- |
| | {[2-(2H-1,3-Benzodioxol-5-yl)-1-methyl-2- | 6.02 (m, 2H), 5.85 (d, J = 5.6 Hz, |
| | oxo-ethyl]-N- | 0.3H), 5.84-5.77 (m, 1.7H), 5.66 |
| | methylaminocarbonyloxy}methyl tert-butyl | (q, J = 6.9 Hz, 0.7H), 5.47 (q, J = 6.9 |
| | succinate | Hz, 0.3H), 2.79 (s, 0.9H), 2.74 (s, |
| | | 2.1H), 2.67-2.59 (m, 2H), 2.57- |
| | | 2.52 (m, 2H), 1.44 (s, 2.7H), 1.43 (s, |
| | | 6.3H), 1.38 (d, J = 6.9 Hz, 3H). |
| 6C | 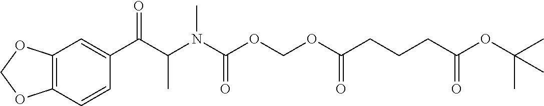 [SMILES: CC(C(=O)c1ccc2c(c1)OCO2)N(C)C(=O)OCOC(=O)CCCC(=O)OC(C)(C)C] | LC-MS (LCMS2: Method 2A): Rt 1.73 mins; m/z 469.2 = [M + NH₄]⁺ ¹H NMR (400 MHz, CDCl₃) δ 7.64 (dd, J = 8.2, 1.7 Hz, 0.7H), 7.53 (dd, J = 8.2, 1.7 Hz, 0.3H), 7.45 (d, J = 1.7 Hz, 0.7H), 7.36 (d, J = 1.7 Hz, 0.3H), 6.85 (d, J = 8.2 Hz, 1H), 6.07- |
| | {[2-(2H-1,3-Benzodioxol-5-yl)-1-methyl-2- | 6.03 (m, 2H), 5.85-5.77 (m, 2H), |
| | oxo-ethyl]-N- | 5.66 (q, J = 6.9 Hz, 0.7H), 5.46 (q, |
| | methylaminocarbonyloxy}methyl tert-butyl | J = 6.9 Hz, 0.3H), 2.80 (s, 0.9H), 2.74 |
| | glutarate | (s, 2.1H), 2.45-2.38 (m, 2H), 2.29- |
| | | 2.24 (m, 2H), 1.91 (p, J = 7.4 Hz, |
| | | 2H), 1.44 (s, 9H), 1.40-1.35 (m, |
| | | 3H). |
| 6D | 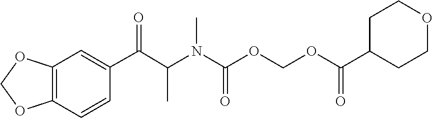 [SMILES: CC(C(=O)c1ccc2c(c1)OCO2)N(C)C(=O)OCOC(=O)C1CCOCC1] | LC-MS (LCMS2: Method 2B): Rt 1.43 mins; m/z 394.2 = [M + H]⁺ ¹H NMR (400 MHz, CDCl₃) δ 7.64 (dd, J = 8.2, 1.8 Hz, 0.7H), 7.53 (dd, J = 8.2, 1.8 Hz, 0.3H), 7.45 (d, J = 1.8 Hz, 0.7H), 7.36 (d, J = 1.8 Hz, 0.3H), 6.84 (d, J = 8.2 Hz, 1H), 6.07- 6.02 (m, 2H), 5.85 (d, J = 5.7 Hz, |
| | [[2-(1,3-Benzodioxol-5-yl)-1-methyl-2-oxo- | 0.3H), 5.83-5.78 (m, 1.7H), 5.65 |
| | ethyl]-methyl-carbamoyl]oxymethyl | (q, J = 6.9 Hz, 0.7H), 5.43 (q, J = 6.9 |
| | tetrahydropyran-4-carboxylate | Hz, 0.3H), 3.98-3.90 (m, 2H), 3.45- |
| | | 3.37 (m, 2H), 2.82 (s, 0.9H), 2.74 |
| | | (s, 2.1H), 2.58 (tt, J = 10.7, 4.4 Hz, |
| | | 1H), 1.86-1.70 (m, 4H), 1.38 (d, |
| | | J = 6.9 Hz, 3H). |
| 6E | 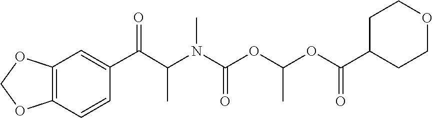 [SMILES: CC(OC(=O)C1CCOCC1)OC(=O)N(C)C(C)C(=O)c1ccc2c(c1)OCO2] | LC-MS (LCMS2: Method 2A): Rt 1.54 mins; m/z 430.2 = [M + Na]⁺ LC-MS (LCMS2: Method 2B): Rt 1.50 mins; m/z 430.1 = [M + Na]⁺ Mixture of diastereoisomers and rotational isomers: ¹H NMR (400 MHz, CDCl₃) δ 7.68-7.51 (m, 1H), 7.48-7.35 (m, 1H), 6.90-6.82 (m, |
| | 1-[[2-(1,3-Benzodioxol-5-yl)-1-methyl-2- | 2H), 6.07-6.02 (m, 2H), 5.68-5.61 |
| | oxo-ethyl]-methyl-carbamoyl]oxyethyl | (m, 0.7H), 5.32 (q, J = 6.8 Hz, 0.3H), |
| | tetrahydropyran-4-carboxylate | 3.99-3.88 (m, 2H), 3.47-3.36 (m, |
| | | 2H), 2.81 (s, 0.45H), 2.76 (s, 0.45H), |
| | | 2.74 (s, 1.05H), 2.71 (s, 1.05H), 2.59- |
| | | 2.47 (m, 1H), 1.86-1.71 (m, 4H), |
| | | 1.52-1.47 (m, 3H), 1.40-1.34 (m, |
| | | 3H). |
| 6F | 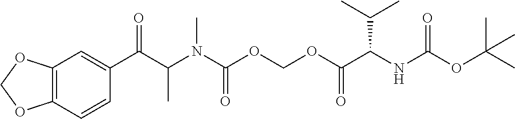 [SMILES: CC(C)[C@H](NC(=O)OC(C)(C)C)C(=O)OCOC(=O)N(C)C(C)C(=O)c1ccc2c(c1)OCO2] | LC-MS (LCMS2: Method 2A): Rt 1.74 mins; m/z 503.2 = [M + Na]⁺ LC-MS (LCMS2: Method 2B): Rt 1.71 mins; m/z 498.3 = [M + NH₄]⁺ Mixture of diastereoisomers and rotational isomers: ¹H NMR (400 MHz, CDCl₃) δ 7.67-7.51 (m, 1H), 7.47-7.36 (m, 1H), 6.89-6.83 (m, |
| | [[2-(1,3-Benzodioxol-5-yl)-1-methyl-2-oxo- | 1H), 6.09-6.02 (m, 2H), 5.95-5.78 |
| | ethyl]-methyl-carbamoyl]oxymethyl (2S)-2- | (m, 2H), 5.70-5.62 (m, 0.7H), |
| | (tert-butoxycarbonylamino)-3-methyl- | 5.49-5.42 (m, 0.3H), 5.08-4.89 (m, |
| | butanoate | 1H), 4.35-4.20 (m, 1H), 2.83 (s, |
| | | 0.45H), 2.78 (s, 0.45H), 2.73 (s, |
| | | 1.2H), 2.72 (s, 0.9H), 2.21-2.02 (m, |
| | | 1H), 1.50-1.40 (m, 9H), 1.40-1.33 |
| | | (m, 3H), 0.98-0.84 (m, 5.1H), 0.76 |
| | | (d, J = 6.9 Hz, 0.9H). |

### Example 7: {[2-(2H-1,3-Benzodioxol-5-yl)-1-methyl-2-oxo-ethyl]-N-methylaminocarbonyloxy}methyl tert-butyl adipate and 5-(1,3-benzodioxol-5-yl)-3,4-dimethyl-oxazol-2-one (Compounds 7A and 7B)

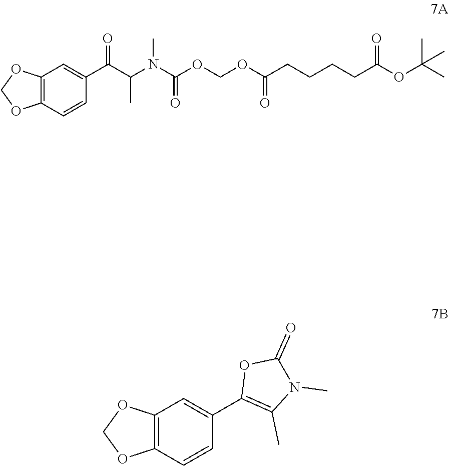 [SMILES: CC(C(=O)c1ccc2c(c1)OCO2)N(C)C(=O)OCOC(=O)CCCCC(=O)OC(C)(C)C ; Cc1c(-c2ccc3c(c2)OCO3)oc(=O)n1C]

A solution of 1M tetrabutyl ammonium hydroxide in MeOH (1.01 mmol, 1.01 mL) was added dropwise over 5 min to a stirred solution of 6-(tert-butoxy)-6-oxohexanoic acid (203 mg, 1.01 mmol) in MeOH (3 mL) at rt under N₂. The mixture was stirred at rt for 1 h and then concentrated in vacuo. THF (2 mL) was added to the residue. A solution of chloromethyl N-[2-(1,3-benzodioxol-5-yl)-1-methyl-2-oxo-ethyl]-N-methyl-carbamate (201 mg, 0.67 mmol) in THF (2 mL) was added dropwise over 5 min to the stirred mixture at rt under N₂, and the resulting mixture was then stirred at rt overnight. EtOAc (25 mL) was added to the mixture and the organic phase was then washed with brine (25 mL), water (2×25 mL) and brine (25 mL). The organic phase was dried over Na₂SO₄ and concentrated in vacuo. The residue was purified by column chromatography on silica gel, eluting with a gradient of 45-50% EtOAc in hexane, to leave {[2-(2H-1,3-benzodioxol-5-yl)-1-methyl-2-oxo-ethyl]-N-methylaminocarbonyloxy}methyl tert-butyl adipate (7A, 200 mg, 64%) as an oil and 5-(1,3-benzodioxol-5-yl)-3,4-dimethyl-oxazol-2-one (7B, 20 mg, 13%) as a solid.

7A: {[2-(2H-1,3-Benzodioxol-5-yl)-1-methyl-2-oxo-ethyl]-N-methylaminocarbonyloxy}methyl tert-butyl adipate: Spectroscopic data of the title compound was obtained as a mixture of two rotational isomers. LC-MS (LCMS2: Method 2A): Rt 1.79 mins; m/z 483.3=[M+NH₄]⁺; ¹H NMR (400 MHz, CDCl₃) δ 7.65 (dd, J=8.2, 1.8 Hz, 0.7H), 7.54 (dd, J=8.2, 1.8 Hz, 0.3H), 7.45 (d, J=1.8 Hz, 0.7H), 7.36 (d, J=1.8 Hz, 0.3H), 6.88-6.83 (m, 1H), 6.08-6.02 (m, 2H), 5.85-5.76 (m, 2H), 5.67 (q, J=6.9 Hz, 0.7H), 5.46 (q, J=6.9 Hz, 0.3H), 2.80 (s, 0.9H), 2.75 (s, 2.1H), 2.41-2.34 (m, 2H), 2.26-2.18 (m, 2H), 1.70-1.58 (m, 4H), 1.44 (s, 9H), 1.42-1.33 (m, 3H).

7B: 5-(1,3-Benzodioxol-5-yl)-3,4-dimethyl-oxazol-2-one: LC-MS (LCMS2: Method 2A): Rt 1.28 mins; m/z 234.0=[M+H]⁺; ¹H NMR (400 MHz, CDCl₃) δ 6.97-6.91 (m, 2H), 6.84 (d, J=8.5 Hz, 1H), 5.99 (s, 2H), 3.23 (s, 3H), 2.24 (s, 3H).

### Example 8: 5-({[2-(2H-1,3-Benzodioxol-5-yl)-1-methyl-2-oxo-ethyl]-N-methylaminocarbonyloxy}methoxycarbonyl)valeric acid (Compound 8A)

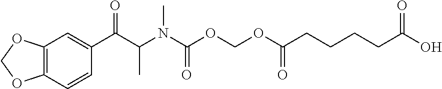 [SMILES: CC(C(=O)c1ccc2c(c1)OCO2)N(C)C(=O)OCOC(=O)CCCCC(=O)O]

A solution {[2-(2H-1,3-benzodioxol-5-yl)-1-methyl-2-oxoethyl]-N-methylaminocarbonyloxy}methyl tert-butyl adipate (156 mg, 0.34 mmol) in formic acid (2.78 g, 60.5 mmol, 2.28 mL) was stirred at rt under N₂ for 4 h. The mixture was concentrated in vacuo and the residue azeotroped with chloroform (3×5 mL) and then DCM (3×5 mL). The compound was further dried under vacuum at 50° C. to leave 5-({[2-(2H-1,3-benzodioxol-5-yl)-1-methyl-2-oxo-ethyl]-N-methylaminocarbonyloxy}methoxycarbonyl)valeric acid (129 mg, 94%) as an oil. Spectroscopic data of the title compound was obtained as a mixture of two rotational isomers. LC-MS (LCMS2: Method 2A): Rt 1.35 mins; m/z 410.1=[M+H]⁺; ¹H NMR (400 MHz, CDCl₃) δ 7.64 (dd, J=8.2, 1.8 Hz, 0.7H), 7.53 (dd, J=8.2, 1.8 Hz, 0.3H), 7.45 (d, J=1.8 Hz, 0.7H), 7.35 (d, J=1.8 Hz, 0.3H), 6.87-6.82 (m, 1H), 6.07-6.02 (m, 2H), 5.84 (d, J=5.6 Hz, 0.3H), 5.82-5.76 (m, 1.7H), 5.66 (q, J=7.0 Hz, 0.7H), 5.45 (q, J=7.0 Hz, 0.3H), 2.81 (s, 0.9H), 2.75 (s, 2.1H), 2.42-2.34 (m, 4H), 1.72-1.63 (m, 4H), 1.40-1.35 (in, 3H). CO₂H not observed.

The compounds of the following table were prepared analogously to Example 8 from the appropriate tert-butyl ester or tert-butyl carbamate.

TABLE Ex8

| Cmpd | | |
|---|---|---|
| No. | Structure and Name | Characterization Data |
| 8B | 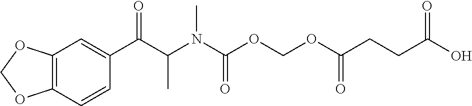 [SMILES: CC(C(=O)c1ccc2c(c1)OCO2)N(C)C(=O)OCOC(=O)CCC(=O)O] | LC-MS (LCMS2: Method 2A): Rt 1.31 mins; m/z 382.1 = [M + H]⁺ ¹H NMR (400 MHz, CDCl₃) δ 7.64 (dd, J = 8.2, 1.8 Hz, 0.7H), 7.54 (dd, J = 8.2, 1.8 Hz, 0.3H), 7.44 (d, J = 1.8 Hz, 0.7H), 7.35 (d, J = 1.8 Hz, 0.3H), 6.86 (d, J = 8.2 Hz, 1H), 6.06-6.02 |
| | 3-({[2-(2H-1,3-Benzodioxol-5-yl)-1- | (m, 2H), 5.87 (d, J = 5.7 Hz, 0.3H), |
| | methyl-2-oxo-ethyl]-N- | 5.83-5.78 (m, 1.7H), 5.65 (q, J = 7.0 |
| | methylaminocarbonyloxy}- | Hz, 0.7H), 5.45 (q, J = 7.0 Hz, 0.3H), |
| | methoxycarbonyl)propionic acid | 2.80 (s, 0.9H), 2.76 (s, 2.1H), 2.72- |
| | | 2.64 (m, 4H), 1.41-1.36 (m, 3H). |
| | | CO₂H not observed. |
| 8C | 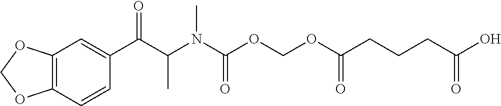 [SMILES: CC(C(=O)c1ccc2c(c1)OCO2)N(C)C(=O)OCOC(=O)CCCC(=O)O] | LC-MS (LCMS2: Method 2A): Rt 1.31 mins; m/z 396.1 = [M + H]⁺ ¹H NMR (400 MHz, CDCl₃) δ 7.63 (dd, J = 8.2, 1.8 Hz, 0.7H), 7.53 (dd, J = 8.2, 1.8 Hz, 0.3H), 7.44 (d, J = 1.8 Hz, 0.7H), 7.35 (d, J = 1.8 Hz, 0.3H), 6.85 (d, J = 8.2 Hz, 1H), 6.10-6.01 |
| | 4-({[2-(2H-1,3-Benzodioxol-5-yl)-1- | (m, 2H), 5.86-5.77 (m, 2H), 5.66 (q, |
| | methyl-2-oxo-ethyl]-N- | J = 6.9 Hz, 0.7H), 5.45 (q, J = 6.9 Hz, |
| | methylaminocarbonyloxy}- | 0.3H), 2.81 (s, 0.9H), 2.76 (s, 2.1H), |
| | methoxycarbonyl)butyric acid | 2.52-2.39 (m, 4H), 2.01-1.91 (m, |
| | | 2H), 1.38 (d, J = 6.9 Hz, 3H). CO₂H |
| | | not observed. |
| 8D | 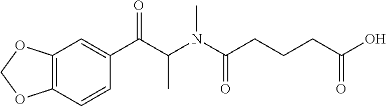 [SMILES: CC(C(=O)c1ccc2c(c1)OCO2)N(C)C(=O)CCCC(=O)O] | LC-MS (LCMS2: Method 2A): Rt 1.12 mins; m/z 322.0 = [M + H]⁺ LC-MS (LCMS2: Method 2B): Rt 0.84 mins; m/z 322.0 = [M + H]⁺ ¹H NMR (400 MHz, CDCl₃) δ 7.62 (dd, J = 8.2, 1.7 Hz, 1H), 7.43 (d, J = 1.7 Hz, 1H), 6.83 (d, J = 8.2 Hz, 1H), |
| | | 6.08 (q, J = 6.9 Hz, 1H), 6.03 (s, 2H), |
| | | 2.81 (s, 3H), 2.47-2.34 (m, 4H), 2.02- |
| | | 1.93 (m, 2H), 1.33 (d, J = 6.9 Hz, |
| | | 3H). CO₂H not observed. |
| | 4-{[2-(2H-1,3-Benzodioxol-5-yl)-1- | |
| | methyl-2-oxo-ethyl]-N- | |
| | methylcarbamoyl}butyric acid | |
| 8E | 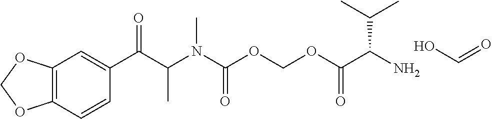 | LC-MS (LCMS2: Method 2A): Rt 1.17 mins; m/z 381.2 = [M + H]⁺ LC-MS (LCMS2: Method 2B): Rt 1.37 mins; m/z 381.2 = [M + H]⁺ Mixture of diastereoisomers and rotational isomers: ¹H NMR (400 MHz, CDCl₃) δ 8.08 (br. s, 1H), 7.66- 7.53 (m, 1H), 7.46-7.35 (m, 1H), 6.94- |
| | [[2-(1,3-Benzodioxol-5-yl)-1-methyl-2- | 6.84 (m, 1H), 6.11-6.04 (m, 2H), |
| | oxo-ethyl]-methyl-carbamoyl]oxymethyl | 5.95-5.77 (m, 2H), 5.70-5.62 (m, |
| | (2S)-2-amino-3-methyl-butanoate formate | 0.7H), 5.49-5.41 (m, 0.3H), 4.52 (br. |
| | | s, 3H), 3.54-3.38 (m, 1H), 2.85- |
| | | 2.68 (m, 3H), 2.19-2.03 (m, 1H), 1.60- |
| | | 1.36 (m, 3H), 1.09-0.70 (m, 6H). |

### Example 9: N-[2-(1,3-Benzodioxol-5-yl)-1-methyl-2-oxo-ethyl]-N-methyl-4-(1-piperidyl)piperidine-1-carboxamide (Compound 9A)

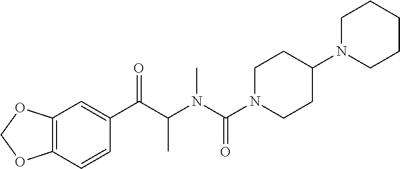 [SMILES: CC(C(=O)c1ccc2c(c1)OCO2)N(C)C(=O)N1CCC(N2CCCCC2)CC1]

1-Chlorocarbonyl-4-piperidinopiperidine hydrochloride (245 mg, 0.92 mmol) was added in one portion to a stirred solution of 1-(1,3-benzodioxol-5-yl)-2-(methylamino)propan-1-one hydrochloride (149 mg, 0.61 mmol) and DIPEA (711 mg, 5.50 mmol, 959 μL) in DCM (5 mL) at rt under N₂. The mixture was stirred at rt overnight. The mixture was directly purified by column chromatography on silica gel, eluting with a gradient of 0-10% MeOH in DCM with ammonia, to leave an oil. The crude compound was re-purified by column chromatography on silica gel, eluting with a gradient of 0-10% MeOH in DCM with ammonia, to leave N-[2-(1,3-benzodioxol-5-yl)-1-methyl-2-oxo-ethyl]-N-methyl-4-(1-piperidyl)piperidine-1-carboxamide (166 mg, 68% yield) as a gum. LC-MS (LCMS2: Method 2A): Rt 1.09 mins; m/z 402.3=[M+H]+; LC-MS (LCMS2: Method 2B): Rt 1.52 mins; m/z 402.3=[M+H]+; ¹H NMR (400 MHz, CDCl₃) δ 7.56 (dd, J=8.2, 1.8 Hz, 1H), 7.42 (d, J=1.8 Hz, 1H), 6.82 (d, J=8.2 Hz, 1H), 6.05-6.00 (m, 2H), 5.39 (q, J=6.9 Hz, 1H), 3.63-3.54 (m, 2H), 2.76-2.59 (m, 5H), 2.55-2.43 (m, 4H), 2.41-2.30 (m, 1H), 1.83-1.72 (m, 2H), 1.63-1.54 (m, 4H), 1.52-1.40 (m, 4H), 1.39 (d, J=6.9 Hz, 3H).

### II. Biological Evaluation

### Example A: Pharmacokinetics of Selected Compounds Following a Single Intravenous or Oral Administration in Rats

A pharmacokinetic (PK) study was performed in three male Sprague-Dawley (SD) rats following intravenous (IV) and oral (PO) administration of methylone at 1 mg/kg (IV) and 10 mg/kg (PO) respectively, or test compounds (prodrugs of methylone) at 10 mg/kg (PO). Parent compound (methylone) was measured in plasma.

### In Vivo Methods

### Regulatory

All animal experiments were performed under UK Home Office Licenses and with local ethical committee clearance. All experiments were performed by technicians that have completed parts A and B of the Home Office Personal License course and hold a current personal license. All experiments were performed in dedicated Biohazard 2 facilities with full AAALAC accreditation.

TABLE 2
Protocol: Serial tail vein bleed PK study of Methylone Prodrugs in SD rats

| Protocol | | Serial PK study at 1 dose level |
|---|---|---|
| Test Compound(s) | | Rac-methylone, Methylone Prodrugs |
| Dosing Route | | PO (all compounds) & IV (rac-methylone only) |
| Overnight food withdrawal | | Yes |
| Animals | Type | rat |
| | Strain | Sprague Dawley rats |
| | Sex | male |
| | Weight (g) | 160-300 g |
| | N per cpd | 3 |
| | Preparation | None |
| | Cage | PK cages |

| | |
|---|---|
| Dose | 10 mg/kg PO & 1 mg/kg IV |
| Dosing Soln. Conc. | 2 mg/mL (PO) or 0.5 mg/mL (IV) |
| Dosing Volume | 5 mL/kg (PO) or 2 mL/kg (IV) |
| Formulation checks required? | No |
| Vehicle | DMSO/PEG400/ H2O (10:20:70) |
| | Rac-Methylone (PO): Water; Rac-Methylone (IV): Saline |
| Sampling time points (h) | PO: 0.5, 1, 2, 4, 7 & 24 h |
| | IV: 0.25, 0.5, 0.75, 1, 2, 4, 7 & 24 h |
| Blood sampling method | Serial via tail vein |
| Alternative method if required | n/a |
| Sample format required | >230 μL blood + 5 μL EDTA (93 mg/mL) to give 2 × 50 |
| | μL plasma |
| Sample processing | Centrifugation for plasma ASAP at 4° C. Place 110 μL |
| | plasma into Eppendorf tube on ice containing 11 μL 10% |
| | phosphoric acid. Gently mix before taking 2 × 50 μL |
| | aliquots into duplicate 96 well plates on dry ice. |
| Anticoagulant | EDTA (93 mg/mL): 5 μL per tube |
| Centrifugation | 10,000 rpm × 3 min at 4° C. |
| Additional samples | n/a |
| Perfusion/rinsing required | n/a |
| Euthanasia method | n/a |
| Plasma sample tubes | 96 well plates |
| Pre-freezer storage | Blood: ice (<30 min), |
| | Acidified Plasma: dry ice |
| Freezer storage | −80° C. |
| Dose formulation samples | 100 μL from vortex dose solution in Eppendorf |
| Number of samples per | PO: 18 × acidified plasma (50 μL in duplicate), 1 dose |
| cpd at 1 dose level | soln |
| | IV: 24 × acidified plasma (50 μL in duplicate), 1 dose |
| | soln |

### Analysis

Samples were sent for method optimization and measurement of parent compound (methylone) via unique calibration lines and following acceptance QC's. Dose formulation concentrations were also measured, and PK parameters were determined (Cmax (ng/mL), Tmax (hr), Cl (ml/min/kg), Vdss (L/kg), t½(hr), AUC0-t (ng/mL*hr), AUC0-inf (ng/mL*hr), MIRT (hr), Bioavailability (0% F) where warranted) using WinNon Lin software. Data (including bioanalytical results and assay performance) were reported in a tabulated format.

### Additional Formulation Details for PK Study

Phosphoric acid. Diluted 85% phosphoric acid 8.5-fold to give a 10% solution.

Formulation for PO Administration: For PO dosing, the prodrug was formulated in 10% DMSO/20% PEG400/70% water to a concentration of 2 mg free metabolite material/mL. This provided a dose of 10 mg free metabolite/kg when the prodrug was administered PO in 5 mL/kg dosing volumes.

Formulation for IV administration: For IV dosing, rac-methylone was formulated as solution in saline to a concentration of 0.5 mg free metabolite material/mL. This provided a dose of 1 mg free metabolite/kg when administered IV in 2 mL/kg dosing volumes.

### Example A-1: Measurement of Concentration of Methylone after IV or Oral Administration of Methylone Prodrugs In Vivo

The pharmacokinetic properties of the synthesized methylone prodrugs after oral administration in a rat model were assessed. The concentration of methylone was measured in each rat at various sampling timepoints after IV or oral administration of rac-methylone or the synthesized methylone prodrugs to rats.

Dose formulations were made at equivalent concentrations of active compound (methylone) adjusted for molecular weight of the compounds. The synthesized methylone prodrugs or analogs were dosed at 10 mg/kg oral (PO) nominal dose. Nominal doses were used in PK parameter determinations. The parent compound (methylone) was dosed at 1 mg/kg intravenous (IV) and 10 mg/kg (PO).

Results for representative prodrugs are found in the following examples:

### Example A-1-1: Methylone Parent Compound (IV & PO)

| | |
|---|---|
| Species | Rat |
| Dose Route: | IV & PO |
| Nominal Dose Concentration: | 1 mg/kg (IV), 10 mg/kg (PO) |

Chemical name: 3,4-Methylenedioxy-N-methylcathinone (Methylone)
Structural class: parent
Mechanistic class: n/a—parent compound

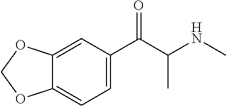

TABLE 3
Methylone (IV & PO) Pharmacokinetic Parameters

| | | | T½ | Cmax | Tmax | Tlast | AUC0-last | AUC0-inf |
|---|---|---|---|---|---|---|---|---|
| Analyte | Dose | Animal ID | (h) | (ng/mL) | (h) | (h) | (h*ng/mL) | (h*ng/mL) |
| Methylone | IV | R31 | 0.41 | 192 | 0.250 | 2.0 | 163 | 168 |
| | | R32 | 0.38 | 165 | 0.250 | 2.0 | 134 | 136 |
| | | R33 | 0.40 | 159 | 0.250 | 2.0 | 128 | 131 |
| | | Mean | 0.395 | 172 | 0.250 | 2.0 | 142 | 145 |
| | Oral | R34 | 2.25 | 130 | 1.00 | 7.00 | 636 | 729 |
| | | R35 | NR | 119 | 2.00 | 7.00 | 520 | NR |
| | | R36 | 2.66 | 260 | 0.500 | 7.00 | 888 | 1050 |
| | | Mean | 2.46 | 170 | 1.17 | 7.00 | 681 | 890 |

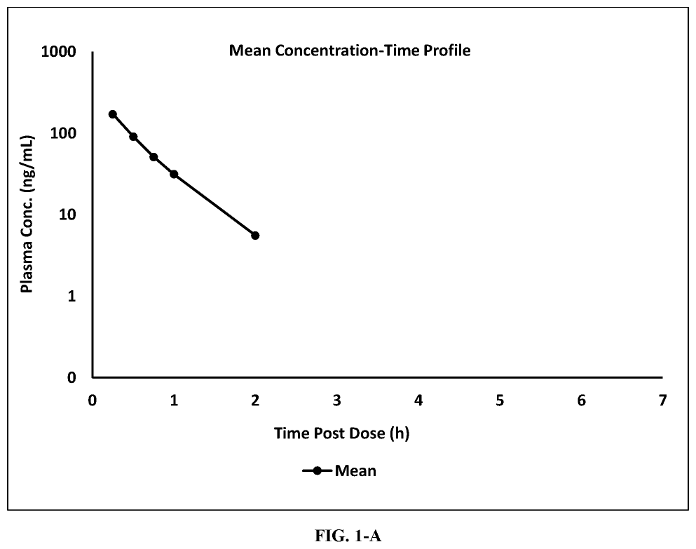

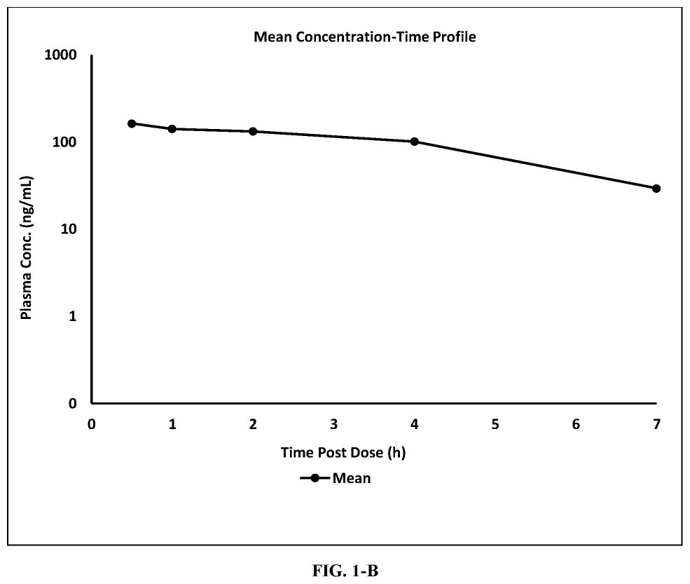

FIG. 1-A shows mean concentration-time profiles of methylone following IV dosing of methylone (1 mg/kg) to male Sprague Dawley (SD) rats.

FIG. 1-B shows mean concentration-time profiles of methylone following oral dosing of methylone (10 mg/Kg) to male Sprague Dawley (SD) rats.

### Example A-1-2: Methylone Ethyl Carbamate Prodrug

| Species: | Rat |
|---|---|
| Dose Route: | PO |
| Nominal Dose Concentration (mg/kg): | 10 |

Chemical name: Ethyl N-[2-(1,3-benzodioxol-5-yl)-1-methyl-2-oxo-ethyl]-N-methyl-carbamate (Compound 1A)
Structural class: carbamate
Mechanistic class: presumed esterase

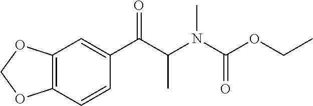 [SMILES: CCOC(=O)N(C)C(C)C(=O)c1ccc2c(c1)OCO2]

TABLE 4
Mean Concentration-Time Profile of Metabolite Methylone Following
Oral Dosing of Methylone ethyl carbamate Prodrug

Bioanalytical Data:
Plasma Concentrations (ng/mL)
Following Oral Dosing
Animal

| Time (h) | R37 | R38 | R39 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-3: Methylone Acetamide Prodrug

| Species: | Rat |
|---|---|
| Dose Route: | PO |
| Nominal Dose Concentration (mg/kg): | 10 |

Chemical name: N-[2-(1,3-Benzodioxol-5-yl)-1-methyl-2-oxo-ethyl]-N-methyl-acetamide (Compound 1F)
Structural class: amide
Mechanistic class: presumed amidase

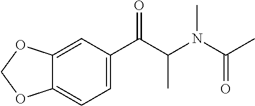 [SMILES: CC(=O)N(C)C(C)C(=O)c1ccc2c(c1)OCO2]

TABLE 5
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R40 | 0.818 | 0.500 | 324 | 4.00 | 542 | 564 |
| | | R41 | 1.26 | 0.500 | 244 | 4.00 | 305 | 346 |
| | | R42 | NR | NR | NR | NR | NR | NR |
| | | Mean | 1.04 | 0.500 | 284 | 4.00 | 424 | 455 |

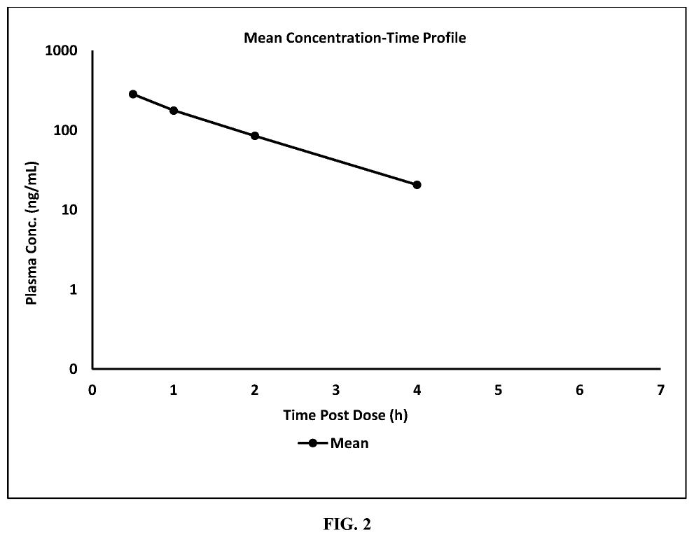

FIG. 2 shows mean concentration-time profiles of methylone following oral dosing of methylone acetamide prodrug (10 mg/kg) to male SD rats.

### Example A-1-4: Methylone t-Butyl Carbamate Prodrug

| Species: | Rat |
|---|---|
| Dose Route: | PO |
| Nominal Dose Concentration (mg/kg): | 10 |

Chemical name: tert-Butyl N-[2-(1,3-benzodioxol-5-yl)-1-methyl-2-oxo-ethyl]-N-methyl-carbamate (Compound 2A)
Structural class: carbamate
Mechanistic class: presumed esterase

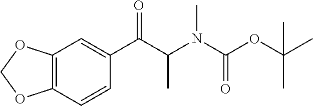 [SMILES: CC(C(=O)c1ccc2c(c1)OCO2)N(C)C(=O)OC(C)(C)C]

TABLE 6
Mean Concentration-Time Profile of Metabolite Methylone Following
Oral Dosing of Methylone t-butyl carbamate Prodrug

Bioanalytical Data:
Plasma Concentrations (ng/mL)
Following Oral Dosing
Animal

| Time (h) | R43 | R44 | R45 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-5: Methylone i-Propyl Carbamate Prodrug

| Species: | Rat |
|---|---|
| Dose Route: | PO |
| Nominal Dose Concentration (mg/kg): | 10 |

Chemical name: iso-Propyl N-[2-(1,3-benzodioxol-5-yl)-1-methyl-2-oxo-ethyl]-N-methyl-carbamate (Compound 1B)
Structural class: carbamate
Mechanistic class: presumed esterase

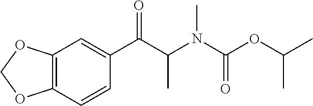 [SMILES: CC(C)OC(=O)N(C)C(C)C(=O)c1ccc2c(c1)OCO2]

TABLE 7
Mean Concentration-Time Profile of Metabolite Methylone Following
Oral Dosing of Methylone i-propyl carbamate Prodrug

Bioanalytical Data:
Plasma Concentrations (ng/mL)
Following Oral Dosing
Animal

| Time (h) | R46 | R47 | R48 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-6: Methylone Trifluoroacetamide Prodrug

| Species: | Rat |
|---|---|
| Dose Route: | PO |
| Nominal Dose Concentration (mg/kg): | 10 |

Chemical name: N-[2-(1,3-Benzodioxol-5-yl)-1-methyl-2-oxo-ethyl]-2,2,2-trifluoro-N-methyl-acetamide (Compound 2B)
Structural class: amide
Mechanistic class: presumed amidase

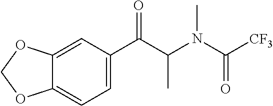 [SMILES: CC(C(=O)c1ccc2c(c1)OCO2)N(C)C(=O)C(F)(F)F]

TABLE 8
Mean Concentration-Time Profile of Metabolite Methylone Following
Oral Dosing of Methylone trifluoroacetamide Prodrug

Bioanalytical Data:
Plasma Concentrations (ng/mL)
Following Oral Dosing
Animal

| Time (h) | R1 | R2 | R3 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-7: Methylone Dimethylglycine Prodrug

| Species: | Rat |
|---|---|
| Dose Route: | PO |
| Nominal Dose Concentration (mg/kg): | 10 |

Chemical name: N-[2-(1,3-Benzodioxol-5-yl)-1-methyl-2-oxo-ethyl]-2-(dimethylamino)-N-methyl-acetamide (Compound 3B)
Structural class: amide
Mechanistic class: presumed amidase

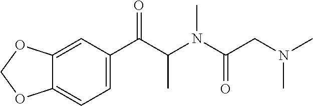 [SMILES: CC(C(=O)c1ccc2c(c1)OCO2)N(C)C(=O)CN(C)C]

TABLE 9
Mean Concentration-Time Profile of Metabolite Methylone Following
Oral Dosing of Methylone dimethylglycine Prodrug

Bioanalytical Data:
Plasma Concentrations (ng/mL)
Following Oral Dosing
Animal

| Time (h) | R4 | R5 | R6 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-8: Methylone Methyl Glycol Prodrug

| Species: | Rat |
|---|---|
| Dose Route: | PO |
| Nominal Dose Concentration (mg/kg): | 10 |

Chemical name: 2-Methoxyethyl N-[2-(1,3-benzodioxol-5-yl)-1-methyl-2-oxo-ethyl]-N-methyl-carbamate (Compound PE)
Structural class: carbamate
Mechanistic class: presumed esterase

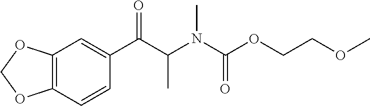 [SMILES: COCCOC(=O)N(C)C(C)C(=O)c1ccc2c(c1)OCO2]

TABLE 10
Mean Concentration-Time Profile of Metabolite Methylone
Following Oral Dosing of Methylone methyl glycol Prodrug

Bioanalytical Data:
Plasma Concentrations (ng/mL)
Following Oral Dosing
Animal

| Time (h) | R7 | R8 | R9 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-9: Methylone Hemi-Gluturate Prodrug

| Species: | Rat |
|---|---|
| Dose Route: | PO |
| Nominal Dose Concentration (mg/kg): | 10 |

Chemical name: 4-{[2-(2H-1,3-Benzodioxol-5-yl)-1-methyl-2-oxo-ethyl]-N-methylcarbamoyl}butyric acid (Compound 8D)
Structural class: amide
Mechanistic class: presumed amidase and/or intramolecular cyclization

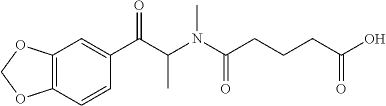 [SMILES: CC(C(=O)c1ccc2c(c1)OCO2)N(C)C(=O)CCCC(=O)O]

TABLE 11
Mean Concentration-Time Profile of Metabolite Methylone
Following Oral Dosing of Methylone hemi-gluturate Prodrug

Bioanalytical Data:
Plasma Concentrations (ng/mL)
Following Oral Dosing
Animal

| Time (h) | R10 | R11 | R12 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-10: Methylone t-Butyl Glutarate Prodrug

| Species: | Rat |
|---|---|
| Dose Route: | PO |
| Nominal Dose Concentration (mg/kg): | 10 |

Chemical name: tert-Butyl 4-{[2-(2H-1,3-benzodioxol-5-yl)-1-methyl-2-oxo-ethyl]-N-methylcarbamoyl}butyrate (Compound 3C)
Structural class: amide
Mechanistic class: presumed amidase and/or presumed esterase+intramolecular cyclization

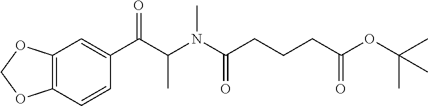 [SMILES: CC(C(=O)c1ccc2c(c1)OCO2)N(C)C(=O)CCCC(=O)OC(C)(C)C]

TABLE 12
Mean Concentration-Time Profile of Metabolite Methylone Following
Oral Dosing of Methylone t-butyl glutarate Prodrug

Bioanalytical Data:
Plasma Concentrations (ng/mL)
Following Oral Dosing
Animal

| Time (h) | R13 | R14 | R15 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-11: Methylone t-Butyl Amide Prodrug

| Species: | Rat |
|---|---|
| Dose Route: | PO |
| Nominal Dose Concentration (mg/kg): | 10 |

Chemical name: N-[2-(1,3-Benzodioxol-5-yl)-1-methyl-2-oxo-ethyl]-N-2,2-trimethyl-propanamide (Compound PG)
Structural class: amide
Mechanistic class: presumed amidase

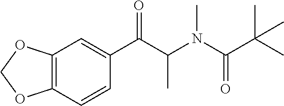 [SMILES: CC(C(=O)c1ccc2c(c1)OCO2)N(C)C(=O)C(C)(C)C]

TABLE 13
Mean Concentration-Time Profile of Metabolite Methylone
Following Oral Dosing of Methylone t-butyl amide Prodrug

Bioanalytical Data:
Plasma Concentrations (ng/mL)
Following Oral Dosing
Animal

| Time (h) | R19 | R20 | R21 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-12: Methylone Trimethyl Lock Prodrug

| Species: | Rat |
|---|---|
| Dose Route: | PO |
| Nominal Dose Concentration (mg/kg): | 10 |

Chemical name: [2-[3-[[2-(1,3-Benzodioxol-5-yl)-1-methyl-2-oxo-ethyl]-methyl-amino]-1,1-dimethyl-3-oxo-propyl]-3,5-dimethyl-phenyl]acetate (Compound 3A)
Structural class: amide
Mechanistic class: presumed esterase+intramolecular cyclization

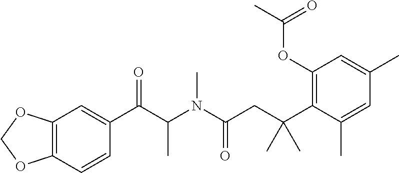 [SMILES: CC(=O)Oc1cc(C)cc(C)c1C(C)(C)CC(=O)N(C)C(C)C(=O)c1ccc2c(c1)OCO2]

TABLE 14
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R52 | NR | 4 | 111 | 7 | 562 | NR |
| | | R53 | NR | 2 | 21.0 | 7 | 94.0 | NR |
| | | R54 | NR | 2 | 38.0 | 4 | 117 | NR |
| | | Mean | NR | 2.7 | 56.7 | 6 | 258 | NR |

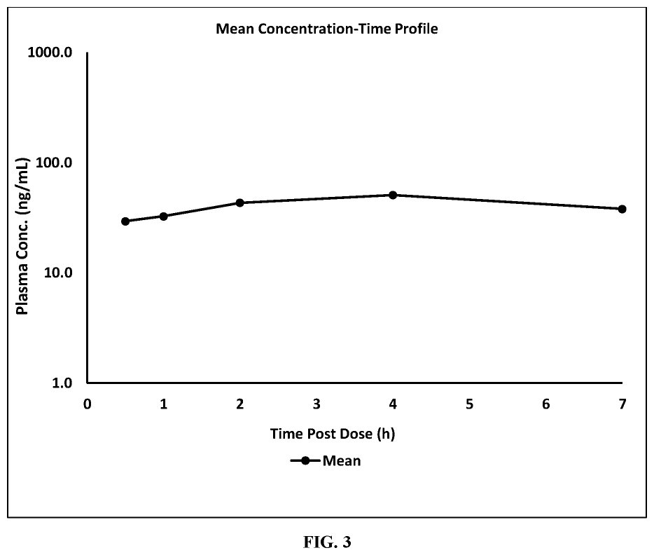

FIG. 3 shows mean concentration-time profiles of methylone following oral dosing of methylone trimethyl lock prodrug (10 mg/kg) to male SD rats.

### Example A-1-13: Methylone Isobutyl Carbamate Prodrug

| Species: | Rat |
|---|---|
| Dose Route: | PO |
| Nominal Dose Concentration (mg/kg): | 10 |

Chemical name: iso-Butyl N-[2-(1,3-benzodioxol-5-yl)-1-methyl-2-oxo-ethyl]-N-methyl-carbamate (Compound 1D)
Structural class: carbamate
Mechanistic class: presumed esterase

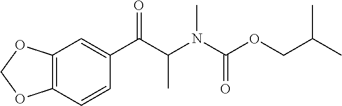 [SMILES: CC(C)COC(=O)N(C)C(C)C(=O)c1ccc2c(c1)OCO2]

TABLE 15
Mean Concentration-Time Profile of Metabolite Methylone Following
Oral Dosing of Methylone isobutyl carbamate Prodrug

Bioanalytical Data:
Plasma Concentrations (ng/mL)
Following Oral Dosing
Animal

| Time (h) | R25 | R26 | R27 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-14: Methylone Methyl Pivalate Prodrug

| Species: | Rat |
|---|---|
| Dose Route: | PO |
| Nominal Dose Concentration (mg/kg): | 10 |

Chemical name: [[2-(1,3-Benzodioxol-5-yl)-1-methyl-2-oxo-ethyl]-methyl-carbamoyl]oxymethyl 2,2-dimethylpropanoate (Compound 6A)
Structural class: acyloxymethyl
Mechanistic class: presume esterase+chemical breakdown

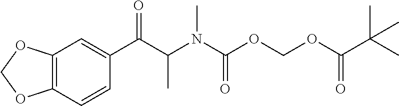 [SMILES: CC(C(=O)c1ccc2c(c1)OCO2)N(C)C(=O)OCOC(=O)C(C)(C)C]

TABLE 16
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R28 | NC | 7.00 | 118 | 7 | 550 | NC |
| | | R29 | 38.1 | 1.00 | 150 | 7 | 858 | 7060 |
| | | R30 | NC | 4.00 | 127 | 7 | 711 | NC |
| | | Mean | 38.1 | 4.00 | 132 | 7 | 706 | 7060 |

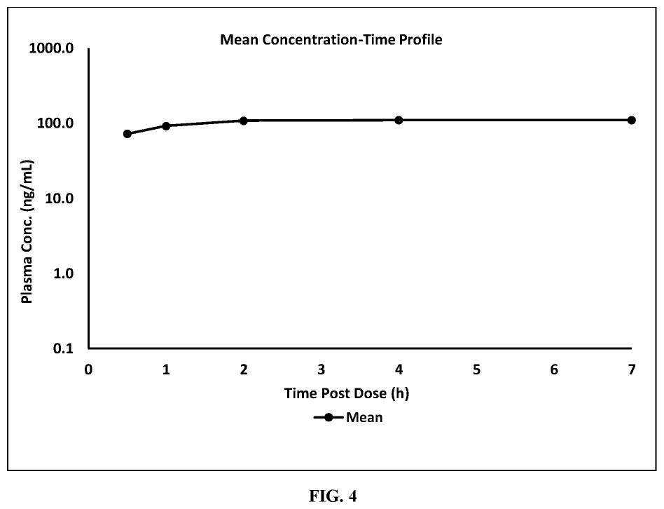

FIG. 4 shows mean concentration-time profiles of methylone following oral dosing of methylone methyl pivalate prodrug (10 mg/kg) to male SD rats.

### Example A-1-15: Methylone Boc-Lysine Prodrug

| Species: | Rat |
|---|---|
| Dose Route: | PO |
| Nominal Dose Concentration (mg/kg): | 10 |

Chemical name: tert-Butyl N-[(5S′)-6-[[2-(1,3-benzodioxol-5-yl)-1-methyl-2-oxo-ethyl]-methyl-amino]-5-(tert-butoxycarbonylamino)-6-oxo-hexyl]carbamate (Compound 3E)
Structural class: amide
Mechanistic class: presumed amidase

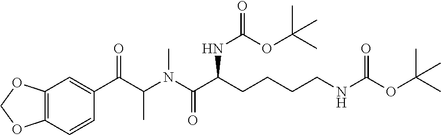 [SMILES: CC(C(=O)c1ccc2c(c1)OCO2)N(C)C(=O)[C@H](CCCCNC(=O)OC(C)(C)C)NC(=O)OC(C)(C)C]

TABLE 17
Mean Concentration-Time Profile of Metabolite Methylone
Following Oral Dosing of Methylone Boc-lysine Prodrug

Bioanalytical Data:
Plasma Concentrations (ng/mL)
Following Oral Dosing
Animal

| Time (h) | R31 | R32 | R33 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-16: Methylone Cyclic Lysine Dihydrochloride Prodrug

| Species: | Rat |
|---|---|
| Dose Route: | PO |
| Nominal Dose Concentration (mg/kg): | 10 |

Chemical name: (2S)-2-(3-Aminobutyl)-6-(1,3-benzodioxol-5-yl)-4,5-dimethyl-2,5-dihydropyrazin-3-one dihydrochloride (Compound 4A)
Structural class: cyclic amide
Mechanistic class: presumed amidase+chemical breakdown

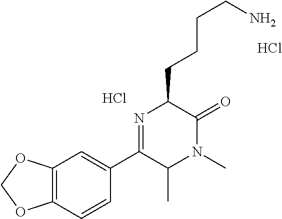 [SMILES: CC1C(c2ccc3c(c2)OCO3)=N[C@@H](CCCCN)C(=O)N1C ; Cl ; Cl]

TABLE 18
Mean Concentration-Time Profile of Metabolite Methylone Following
Oral Dosing of Methylone cyclic lysine dihydrochloride Prodrug

Bioanalytical Data:
Plasma Concentrations (ng/mL)
Following Oral Dosing
Animal

| Time (h) | R34 | R35 | R36 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-17: Methylone Dipiperidyl Urea Prodrug

| Species: | Rat |
|---|---|
| Dose Route: | PO |
| Nominal Dose Concentration (mg/kg): | 10 |

Chemical name: N-[2-(1,3-Benzodioxol-5-yl)-1-methyl-2-oxo-ethyl]-N-methyl-4-(1-piperidyl)piperidine-1-carboxamide (Compound 9A)
Structural class: urea
Mechanistic class: presumed amidase

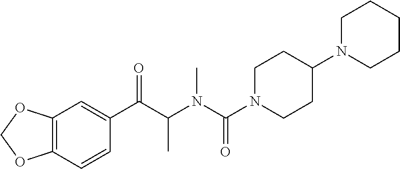 [SMILES: CC(C(=O)c1ccc2c(c1)OCO2)N(C)C(=O)N1CCC(N2CCCCC2)CC1]

TABLE 19
Mean Concentration-Time Profile of Metabolite Methylone Following
Oral Dosing of Methylone dipiperidyl urea Prodrug

Bioanalytical Data:
Plasma Concentrations (ng/mL)
Following Oral Dosing
Animal

| Time (h) | R37 | R38 | R39 | Mean |
|---|---|---|---|---|
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-18: Methylone THP Methyl Ester Prodrug

| Species: | Rat |
|---|---|
| Dose Route: | PO |
| Nominal Dose Concentration (mg/kg): | 10 |

Chemical name: [[2-(1,3-Benzodioxol-5-yl)-1-methyl-2-oxo-ethyl]-methyl-carbamoyl]oxymethyl tetrahydropyran-4-carboxylate (Compound 6D)
Structural class: acyloxymethyl carbamate
Mechanistic class: presumed esterase+chemical breakdown

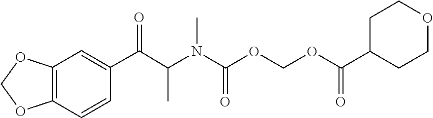 [SMILES: CC(C(=O)c1ccc2c(c1)OCO2)N(C)C(=O)OCOC(=O)C1CCOCC1]

TABLE 20
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R40 | 2.03 | 1.00 | 215 | 7.00 | 738 | 826 |
| | | R41 | 12.6 | 1.00 | 81.0 | 7.00 | 472 | 1550 |
| | | R42 | 1.59 | 1.00 | 357 | 7.00 | 1130 | 1200 |
| | | Mean | 5.41 | 1.00 | 218 | 7.00 | 780 | 1192 |

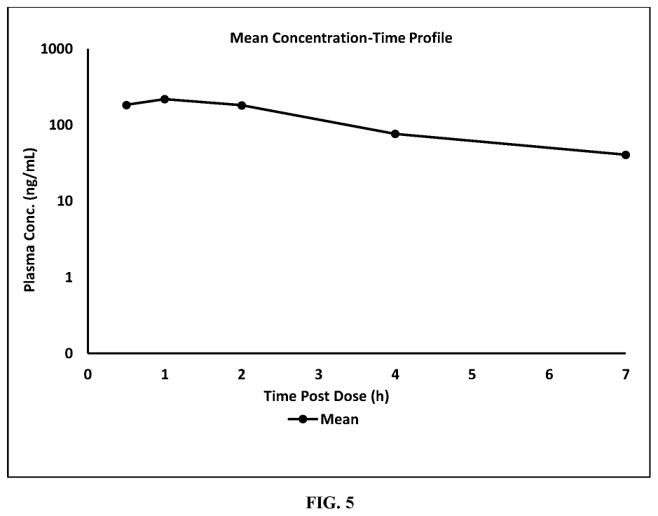

FIG. 5 shows mean concentration-time profiles of methylone following oral dosing of methylone THP methyl ester prodrug (10 mg/kg) to male SD rats.

### Example A-1-19: Methylone t-Butyl Methyl Glutarate Prodrug

| Species: | Rat |
|---|---|
| Dose Route: | PO |
| Nominal Dose Concentration (mg/kg): | 10 |

Chemical name: {[2-(2H-1,3-Benzodioxol-5-yl)-1-methyl-2-oxo-ethyl]-N-methylaminocarbonyloxy}methyl tert-butyl glutarate (Compound 6C)
Structural class: acyloxymethyl carbamate
Mechanistic class: presumed esterase+chemical breakdown and/or presumed esterase+intramolecular cyclization+chemical breakdown

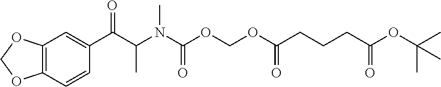 [SMILES: CC(C(=O)c1ccc2c(c1)OCO2)N(C)C(=O)OCOC(=O)CCCC(=O)OC(C)(C)C]

TABLE 21
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R43 | NC | 2.00 | 187 | 7.00 | 715 | NC |
| | | R44 | 1.06 | 1.00 | 227 | 7.00 | 755 | 767 |
| | | R45 | NC | 2.00 | 188 | 7.00 | 1050 | NC |
| | | Mean | 1.06 | 1.67 | 201 | 7.00 | 840 | 767 |

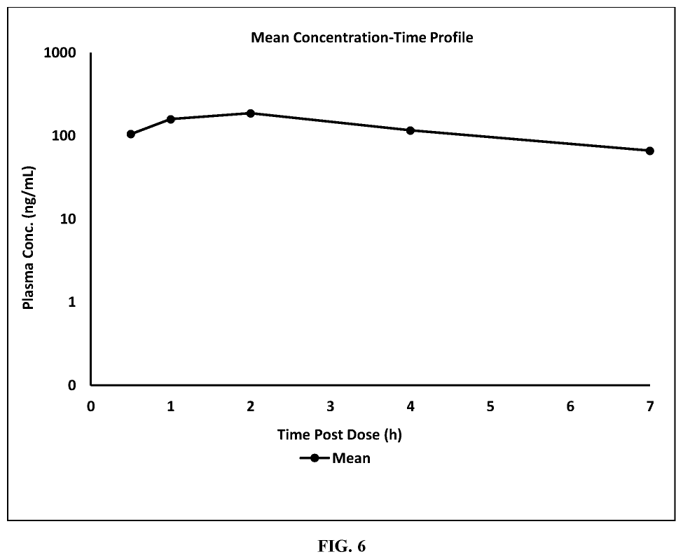

FIG. 6 shows mean concentration-time profiles of methylone following oral dosing of methylone t-butyl methyl glutarate prodrug (10 mg/kg) to male SD rats.

### Example A-1-20: Methylone Methyl Hemi-Glutarate Prodrug

| Species: | Rat |
|---|---|
| Dose Route: | PO |
| Nominal Dose Concentration (mg/kg): | 10 |

Chemical name: 4-({[2-(2H-1,3-Benzodioxol-5-yl)-1-methyl-2-oxo-ethyl]-N-methylaminocarbonyloxy}methoxycarbonyl)butyric acid (Compound 8C)
Structural class: acyloxymethyl carbamate
Mechanistic class: presumed esterase+chemical breakdown and/or presumed pH-controlled intramolecular cyclization+chemical breakdown

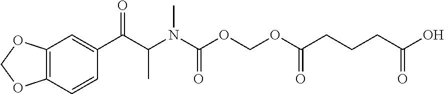 [SMILES: CC(C(=O)c1ccc2c(c1)OCO2)N(C)C(=O)OCOC(=O)CCCC(=O)O]

TABLE 22
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R46 | 3.70 | 0.500 | 244 | 7.00 | 954 | 1300 |
| | | R47 | 1.70 | 0.500 | 156 | 7.00 | 720 | 767 |
| | | R48 | 6.32 | 0.500 | 89 | 7.00 | 385 | 702 |
| | | Mean | 3.91 | 0.500 | 163 | 7.00 | 686 | 923 |

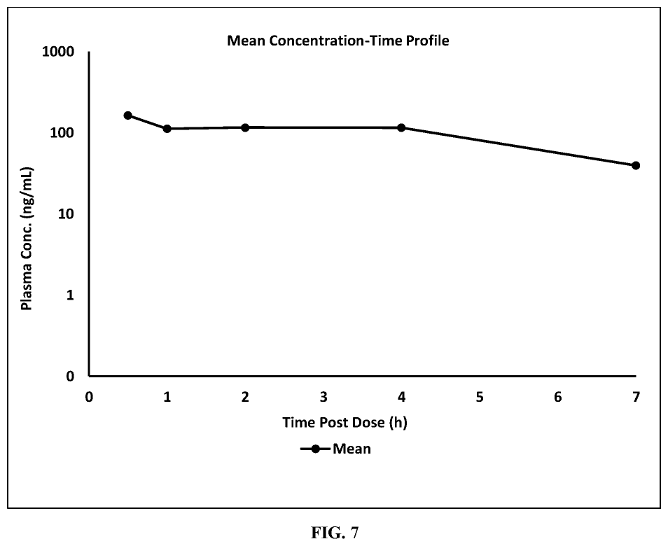

FIG. 7 shows mean concentration-time profiles of methylone following oral dosing of methylone methyl hemi-glutarate prodrug (10 mg/kg) to male SD rats.

### Example A-1-21: Methylone Oxazolone Prodrug

| Species: | Rat |
|---|---|
| Dose Route: | PO |
| Nominal Dose Concentration (mg/kg): | 10 |

Chemical name: 5-(1,3-benzodioxol-5-yl)-3,4-dimethyl-oxazol-2-one (Compound 7B)
Structural class: oxazolone—cyclic carbamate
Mechanistic class: presumed esterase

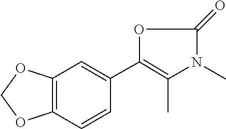 [SMILES: Cc1c(-c2ccc3c(c2)OCO3)oc(=O)n1C]

TABLE 23
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R1 | NR | 0.500 | 1.00 | 0.500 | 0.250 | NR |
| | | R2 | NR | NR | NR | NR | NR | NR |
| | | R3 | NR | NR | NR | NR | NR | NR |
| | | Mean | NR | 0.500 | 1.00 | 0.500 | 0.250 | NR |

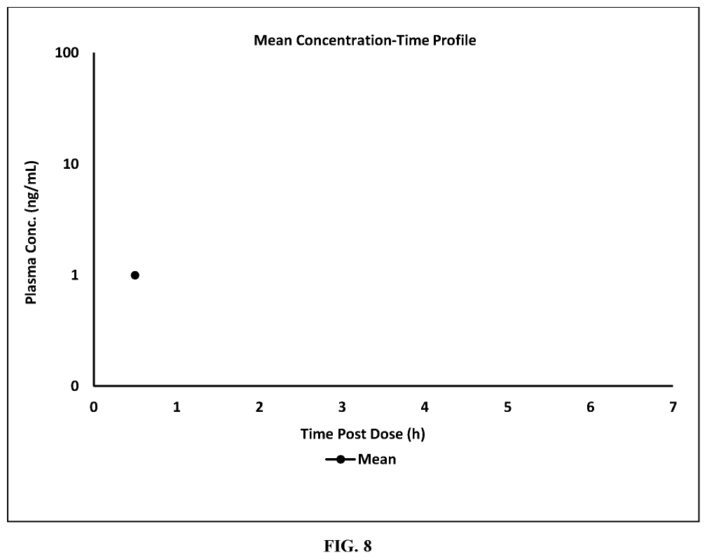

FIG. 8 shows mean concentration-time profiles of methylone following oral dosing of methylone oxazolone prodrug (10 mg/kg) to male SD rats.

### Example A-1-22: Methylone THP 1-Ethyl Ester Prodrug

| Species: | Rat |
|---|---|
| Dose Route: | PO |
| Nominal Dose Concentration (mg/kg): | 10 |

Chemical name: 1-[[2-(1,3-Benzodioxol-5-yl)-1-methyl-2-oxo-ethyl]-methyl-carbamoyl]oxyethyl tetrahydropyran-4-carboxylate (Compound 6E)
Structural class: acyloxymethyl carbamate
Mechanistic class: presumed esterase+chemical breakdown

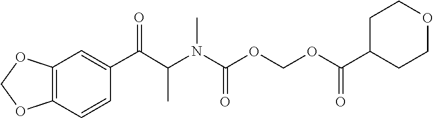 [SMILES: CC(C(=O)c1ccc2c(c1)OCO2)N(C)C(=O)OCOC(=O)C1CCOCC1]

TABLE 24
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R4 | 5.23 | 1.00 | 80.3 | 7.00 | 432 | 719 |
| | | R5 | 32.5 | 0.500 | 64.8 | 7.00 | 390 | 2840 |
| | | R6 | NR | 1.00 | 104 | 7.00 | 499 | NR |
| | | Mean | 18.9 | 0.833 | 83.0 | 7.00 | 440 | 1780 |

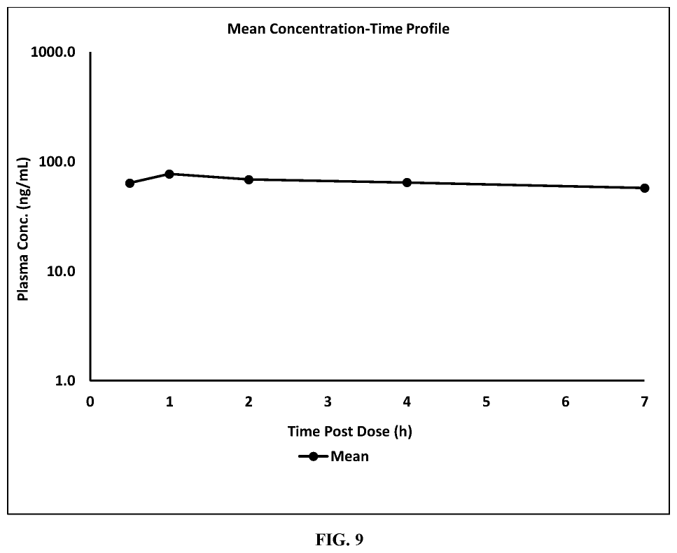

FIG. 9 shows mean concentration-time profiles of methylone following oral dosing of methylone THP 1-ethyl ester prodrug (10 mg/kg) to male SD rats.

### Example A-1-23: Methylone Methyl Hemi-Adipate Prodrug

| Species: | Rat |
|---|---|
| Dose Route: | PO |
| Nominal Dose Concentration (mg/kg): | 10 |

Chemical name: {[2-(2H-1,3-Benzodioxol-5-yl)-1-methyl-2-oxo-ethyl]-N-methylaminocarbonyloxy}methyl tert-butyl adipate (Compound 7A)
Structural class: acyloxymethyl carbamate
Mechanistic class: presumed esterase+chemical breakdown and/or presumed pH-controlled intramolecular cyclization+chemical breakdown

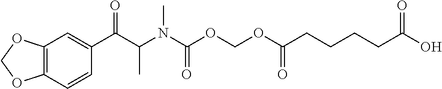 [SMILES: CC(C(=O)c1ccc2c(c1)OCO2)N(C)C(=O)OCOC(=O)CCCCC(=O)O]

TABLE 25
Methylone Pharmacokinetic Parameters

| | Dose | | T½ | Tmax | Cmax | Tlast | AUClast | AUCINF_obs |
|---|---|---|---|---|---|---|---|---|
| Analyte | Route | Animal ID | (hr) | (hr) | (ng/mL) | (hr) | (ng/ml*hr) | (ng/ml*hr) |
| Methylone | Oral | R7 | NR | 4.00 | 67.5 | 24.0 | 660 | NR |
| | | R8 | NR | 2.00 | 130 | 7.00 | 511 | NR |
| | | R9 | NR | 2.00 | 106 | 7.00 | 532 | NR |
| | | Mean | NR | 2.67 | 101 | 12.7 | 568 | NR |

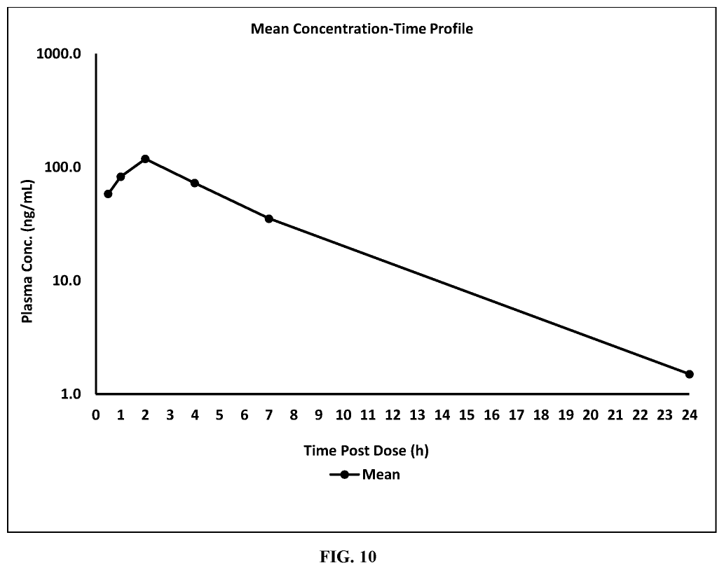

FIG. 10 shows mean concentration-time profiles of methylone following oral dosing of methylone methyl hemi-adipate prodrug (10 mg/kg) to male SD rats.

### Example B: Zero Maze Study

### Background

The rat zero-maze model is a refined alternative to the plus-maze, the most widely used animal model of anxiety, and consists of an elevated annular platform, divided equally into four quadrants. Two opposite quadrants are enclosed by Perspex walls on both the inner and the outer edges of the platform, while the remaining two opposite quadrants are open being enclosed only by a Perspex “lip”. Animals will show a preference for the closed areas, and avoidance of the open sections is assumed to stem from a rodent's natural aversion to open, exposed spaces. The primary index of anxiety, which reflect changes in open arm preference, is the percentage of time spent on the open areas. A reduction in the amount of activity on the open areas is considered to reflect an increase in anxiety. Other, ethologically-based behaviours are also assessed as indices of anxiety. One of the most important is stretched attend postures (SAP) from closed to open quadrants. Increase in SAPs is indicative of an anxiogenic effect and a decrease in SAPs is indicative of an anxiolytic effect. Shepherd, J K; et al., (1994) Behavioural and pharmacological validation of the elevated “zero-maze” as an animal model of anxiety. Psychopharmacol., 116:56-64.

### Animals

Male Sprague-Dawley 200-250 g (Envigo UK) rats were used. Animals were group-housed (5 per cage; cage size: 40×40×20 cm) in a temperature-controlled environment (22±2° C.), under a 12 h light-dark cycle (lights on: 08:00 hours) for one week prior to testing. Food and water were freely available. Number of animals per group=5. Animals were moved into the experimental room 16-24 hours before testing.

### Apparatus

The elevated 0-maze comprises a black Perspex annular platform (105 cm diameter, 10 cm width) elevated to 65 cm above ground level, divided equally into four quadrants. Two opposite quadrants are enclosed by clear red Perspex walls (27 cm high) on both the inner and outer edges of the platform, while the remaining two opposite quadrants are surrounded only by a Perspex “lip” (1 cm high) which serves as a tactile guide to animals on these open areas.

### Procedure

Subjects were weighed and tail marked before being injected. After a specified pre-treatment time, subjects were placed in a closed quadrant and a 5-min test period were recorded on videotape for subsequent analysis. The maze were cleaned with 5% methanol/water solution and dried thoroughly between test sessions. Behavioural measures comprise percentage time spent on the open areas (% TO) and frequency of stretched attend postures (SAP) from closed to open quadrants. Since the control groups were all treated identically with the same vehicle these were combined to increase power. The Chlordiazepoxide groups were also treated identically with the same dose so these were also combined to increase power. Animals are scored as being in the open area when all four paws were in an open quadrant and in the closed area only when all four paws have passed over the open-closed divide. All testing were carried out between 9.00 and 16.00 hours.

### Drug Versus Vehicle Treatments

For each study, a 1-way ANOVA was conducted across vehicle, CDP, and drug treatment groups. Each group was compared to the vehicle group and a p-value for treatment determined by Fishers Least Significant Difference (LSD) test. This analysis was performed in GraphPad Prism (Version 9).

### Formulations:

Intraperitoneal (IP): Racemic methylone HCl was formulated in Vehicle 1 (Saline) for injection to concentrations of 1, 3 and 6 mg/mL to provide doses of 5, 15 and 30 mg/kg when administered ip in 5 mL/kg dosing volumes.

Intraperitoneal (IP): S-methylone HCl was formulated in Vehicle 1 (Saline) for injection to concentrations of 0.5, 1, 1.5, 3 and 6 mg/mL to provide doses of 2.5, 5, 7.5, 15 and 30 mg/kg when administered ip in 5 mL/kg dosing volumes.

Intraperitoneal (IP): R-methylone HCl was formulated in Vehicle 1 (Saline) for injection to concentrations of 1, 3 and 6 mg/mL to provide doses of 5, 15 and 30 mg/kg when administered ip in 5 mL/kg dosing volumes.

Chlordiazepoxide was formulated in Vehicle 1 (saline) to a concentration of 1.2 mg/mL to provide a dose of 6 mg/kg when administered ip in 5 mL/kg dosing volumes.

### Effect of Administration of Methylone HCl and Chlordiazepoxide on Behavior in a Rat 0-Maze Study

Protocol: 25 male Sprague-Dawley rats in treatment groups of 5, were intraperitoneally dosed with either Vehicle 1 (saline) or methylone HCl at 1 of 3 dose levels (5, 15 & 30 mg/kg) or chlordiazepoxide (6 mg/kg) in 5 mL/kg injection volumes. Following injection, each rat was placed in a separate cage with opaque walls in a room adjacent to the study room. Thirty min later at T=0, rats were individually placed in a closed arm of the zero-maze and behaviour assessed by a “blind” observer using remote video monitoring over the subsequent 5 min. The animal was then removed and the maze carefully wiped with 5% methanol/water solution before the next test was begun.

Terminal sampling. At the end of the study, terminal blood was collected from 2 of the vehicle treated rats (>5 mL) and from all methylone HCl treated rats (500 uL) by CP under CO₂. Blood samples were placed into Eppendorf tubes on ice containing 22 μL/mL blood (vehicle treated) or 11 μL EDTA (93 mg/mL) (Methylone HCl treated rats) and gently mixed. After no longer than 30 min, blood samples were centrifuged at 10,000 rpm×3 min and plasma samples (>2 mL (vehicle) or 2×100 μL (Methylone HCl treated) placed into Eppendorf tubes (Vehicle) or 96 well plates on dry ice. Immediately after blood sampling, brains were excised, rinsed in saline, blotted, weighed and snap-frozen in liquid N₂. All samples were stored at −20

$$Total number of plasma samples = 15 + 2 vehicle controls .$$

$$Total number of brain samples = 15 + 2 vehicle controls .$$

Synopsis of testing schedule methylone HCl and chlordiazepoxide
in the rat elevated zero maze model of anxiety.

| | Rat Strain | 0.5 h Pretest in | T = −0.5 | T = 0 | T = 0.25 |
|---|---|---|---|---|---|
| n | & sex | Veh 1 5 mL/kg ip | to T = 0 | Zero-maze | Terminal sampling |
| 5 | Male SD | Vehicle 1 (Saline) | After dosing, | Test | Plasma & brain |
| | | | place rats | | from 2 rats |
| 5 | Male SD | Methylone HCl | in separate | Test | Plasma & brain |
| | | 5 mg/kg | opaque- | | |
| 5 | Male SD | Methylone HCl | walled | Test | Plasma & brain |
| | | 15 mg/kg | cages in | | |
| 5 | Male SD | Methylone HCl | adjacent | Test | Plasma & brain |
| | | 30 mg/kg | room | | |
| 5 | Male SD | CDP 6 mg/kg | | Test | n/a |

### Effect of Administration of S-Methylone HC and Chlordiazepoxide on Behavior in a Rat 0-Maze Study

Protocol: 35 male Sprague-Dawley rats in treatment groups of 5, were intraperitoneally dosed with either Vehicle 1 (saline) or S-methylone HCl at 1 of 5 dose levels (2.5, 5, 7.5, 15 & 30 mg/kg) or chlordiazepoxide (6 mg/kg) in 5 mL/kg injection volumes. Thirty min later at T=0, rats were individually placed in a closed arm of the zero-maze and behaviour assessed by a “blind” observer using remote video monitoring over the subsequent 5 min. The animal was then removed and the maze carefully wiped with 5% methanol/water solution before the next test was begun.

Terminal sampling. At the end of the study, terminal blood was collected from 2 of the vehicle treated rats (>5 mL) and from all S-methylone HCl treated rats (500 uL) by CP under CO₂. Blood samples were placed into Eppendorf tubes on ice containing 22 μL/mL blood (vehicle treated) or 11 μL EDTA (93 mg/mL) (S-methylone HCl treated rats) and gently mixed. After no longer than 30 min, blood samples were centrifuged at 10,000 rpm×3 min and plasma samples (>2 mL (vehicle) or 2×100 μL (S-methylone HCl treated) placed into Eppendorf tubes (Vehicle) or 96 well plates on dry ice. Immediately after blood sampling, brains were excised, rinsed in saline, blotted, weighed and snap-frozen in liquid N₂. All samples were stored at −20

$$Total number of plasma samples = 25 + 2 vehicle controls .$$

$$Total number of brain samples = 25 + 2 vehicle controls .$$

Synopsis of testing schedule S-methylone HCl and chlordiazepoxide
in the rat elevated zero maze model of anxiety.

| | Rat Strain | 0.5 Pretest in | T = 0 | T = 0.25 |
|---|---|---|---|---|
| n | & sex | Veh 1 5 mL/kg ip | Zero-maze | Terminal sampling |
| 5 | Male SD | Vehicle 1 (Saline) | Test | Plasma & brain |
| | | | | from 2 rats |
| 5 | Male SD | S-methylone HCl | Test | Plasma & brain |
| | | 2.5 mg/kg | | |
| 5 | Male SD | S-methylone HCl | Test | Plasma & brain |
| | | 5 mg/kg | | |
| 5 | Male SD | S-methylone HCl | Test | Plasma & brain |
| | | 7.5 mg/kg | | |
| 5 | Male SD | S-methylone HCl | Test | Plasma & brain |
| | | 15 mg/kg | | |
| 5 | Male SD | S-methylone HCl | Test | Plasma & brain |
| | | 30 mg/kg | | |
| 5 | Male SD | CDP 6 mg/kg | Test | n/a |

### Effect of Administration of R-Methylone HC and Chlordiazepoxide on Behavior in a Rat 0-Maze Study

Protocol: 25 male Sprague-Dawley rats in treatment groups of 5, were intraperitoneally dosed with either Vehicle 1 (saline) or R-methylone HCl at 1 of 3 dose levels (5, 15 & 30 mg/kg) or chlordiazepoxide (6 mg/kg) in 5 mL/kg injection volumes. Thirty min later at T=0, rats were individually placed in a closed arm of the zero-maze and behaviour assessed by a “blind” observer using remote video monitoring over the subsequent 5 min. The animal was then removed and the maze carefully wiped with 5% methanol/water solution before the next test is begun.

Terminal sampling. At the end of the study, terminal blood samples were collected from 2 of the vehicle treated rats (>5 mL) and from all R-methylone HCl treated rats (500 uL) by CP under CO₂. Blood samples were placed into Eppendorf tubes on ice containing 22 μL/mL blood (vehicle treated) or 11 μL EDTA (93 mg/mL) (R-methylone HCl treated rats) and gently mixed. After no longer than 30 min, blood samples were centrifuged at 10,000 rpm×3 min and plasma samples (>2 mL (vehicle) or 2×100 μL (R-methylone HCl treated) placed into Eppendorf tubes (Vehicle) or 96 well plates on dry ice. Immediately after blood sampling, brains were excised, rinsed in saline, blotted, weighed and snap-frozen in liquid N₂. All samples were stored at −20° C. until dispatched on dry ice for analysis.

$$Total number of plasma samples = 15 + 2 vehicle controls .$$

$$Total number of brain samples = 15 + 2 vehicle controls .$$

Synopsis of testing schedule R-methylone HCl and chlordiazepoxide
in the rat elevated zero maze model of anxiety.

| | Rat Strain | 0.5 Pretest in | T = 0 | T = 0.25 |
|---|---|---|---|---|
| n | & sex | Veh 1 5 mL/kg ip | Zero-maze | Terminal sampling |
| 5 | Male SD | Vehicle 1 (Saline) | Test | Plasma & brain |
| | | | | from 2 rats |
| 5 | Male SD | R-methylone HCl | Test | Plasma & brain |
| | | 5 mg/kg | | |
| 5 | Male SD | R-methylone HCl | Test | Plasma & brain |
| | | 15 mg/kg | | |
| 5 | Male SD | R-methylone HCl | Test | Plasma & brain |
| | | 30 mg/kg | | |
| 5 | Male SD | CDP 6 mg/kg | Test | n/a |

### Discussion

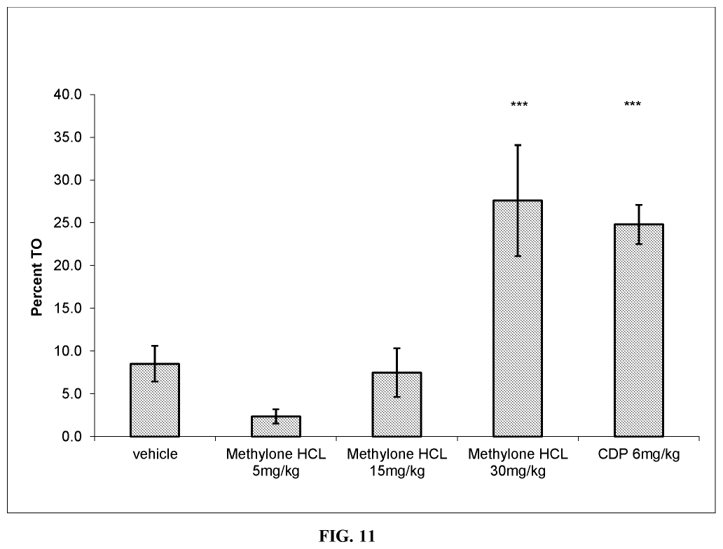

The results show that specific doses of racemic methylone, S-methylone, and R-methylone all increase time in the open arms and decreased the frequency of SAPs as effectively as the benzodiazepine chlordiazepoxide (FIGS. 11-16). This shows that at a sufficient dose, racemic methylone, S-methylone, and R-methylone are all effective anxiolytics and supports their development in these indications. This is the first in vivo data showing methylone is effective in these indications. However, there were some unexpected findings that show they are not equivalent in regard to side effects that further inform dose selection for their therapeutic use in humans.

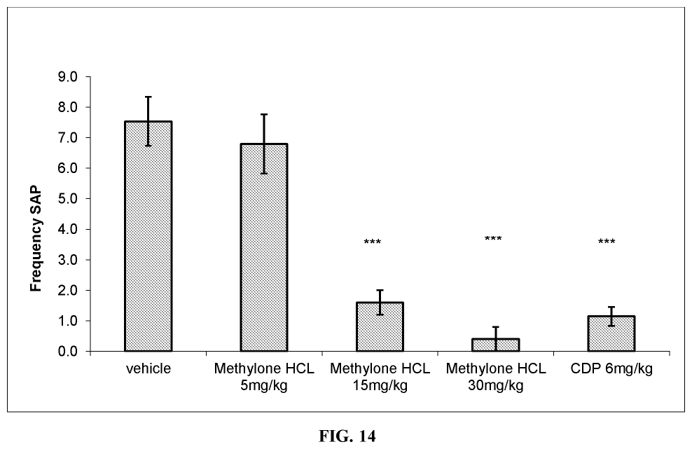

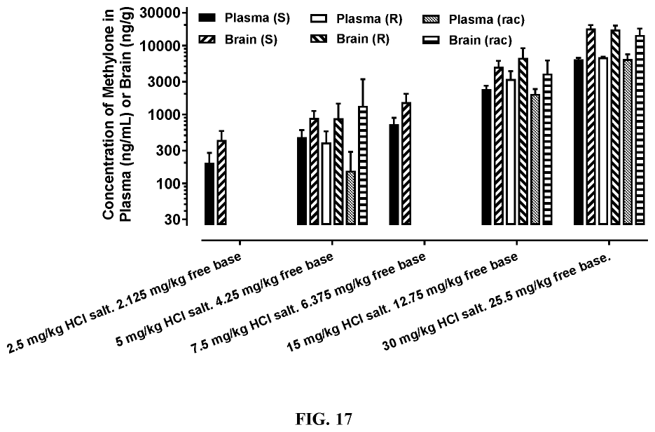

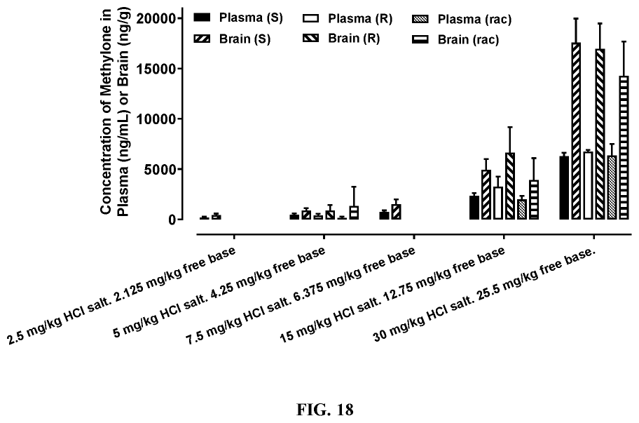

First, for racemic methylone, the lowest dose tested (5 mg/kg) showed a decrease in time spent in the open arms vs placebo (FIG. 11). This indicates that this low dose of methylone had an anxiogenic effect. Similarly, the middle dose of 15 mg/kg showed no difference from placebo in time spent in the open arms but showed a dose dependent decrease in SAPs indicating a mixed anxiogenic anxiolytic effect at this dose range (FIG. 14). However, the decrease in SAPs was not as effective as chlordiazepoxide at this dose. In contrast, the 30 mg/kg dose showed both a significant decrease in time spent in the open arms as well as a decrease in SAPs. This indicates that for racemic methylone, low doses can paradoxically increase anxiety and that this anxiogenic effect shifts to an anxiolytic effect at doses that are sufficiently high to induce a therapeutic effect. This data is supported by the plasma and brain samples verifying the dose of methylone indeed increased in both brain and plasma at the higher doses (FIG. 17 and FIG. 18).

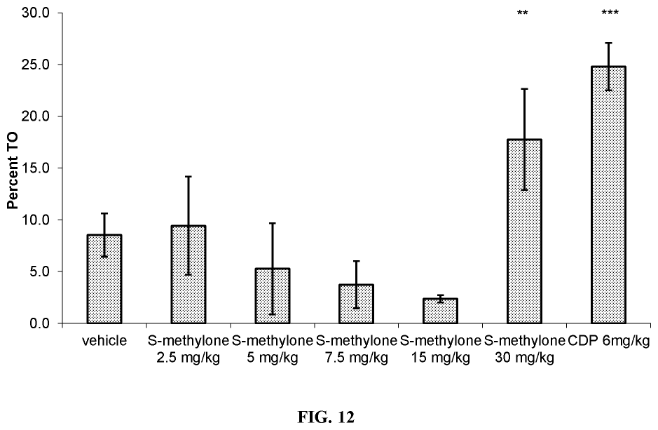

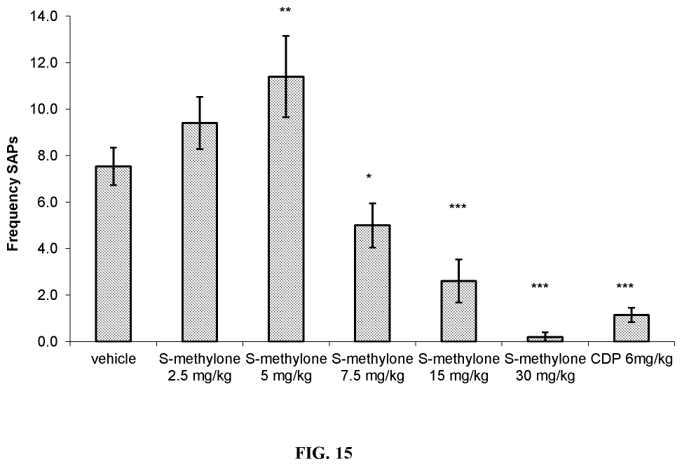

Since we hypothesized that S-methylone would be the more potent enantiomer, the dose range was expanded to even lower doses to investigate the dose range that may induce anxiogenic effects or mixed effects. There was an even stronger dose dependent anxiogenic effect observed with S-methylone than racemic methylone. For S-methylone, the time spent in the open arms decreased as the dose increased with the 5 mg/kg, 7.5 mg/kg and 15 mg/kg indicating an increasing anxiogenic effect. This was reversed at 30 mg/kg at which point the time in the open arms increased (FIG. 12) and was as effective as chlordiazepoxide at increasing time spent in the open arms. This indicated that this dose level was the most effective anxiolytic dose of S-methylone. Similarly, a dose dependent increase in SAPs at the 2.5 mg/kg and 5 mg/kg doses which then switch to a dose dependent decrease in SAPS at 7.5 mg/kg, 15 mg/kg and 30 mg/kg was observed (FIG. 15). Compared to SAPs, the time spent in the open arms is the primary measure of anxiety indicating that the mixed effects on SAPs did not decrease the overall effect on anxiety at 7.5 mg/kg and 15 mg/kg. The data show that the lowest doses of methylone induce a purely anxiogenic effect with middle doses inducing a mixed anxiogenic-anxiolytic effect and higher doses exerting a purely anxiolytic effect. The 30 mg/kg dose was an effective anxiolytic on all measures and was even more effective than chlordiazepoxide at reducing SAPs.

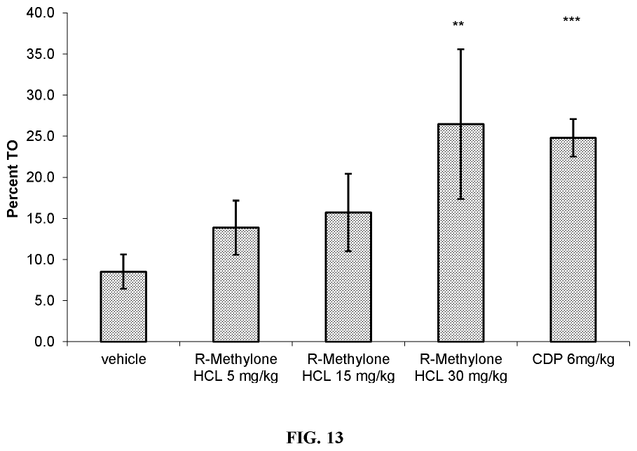

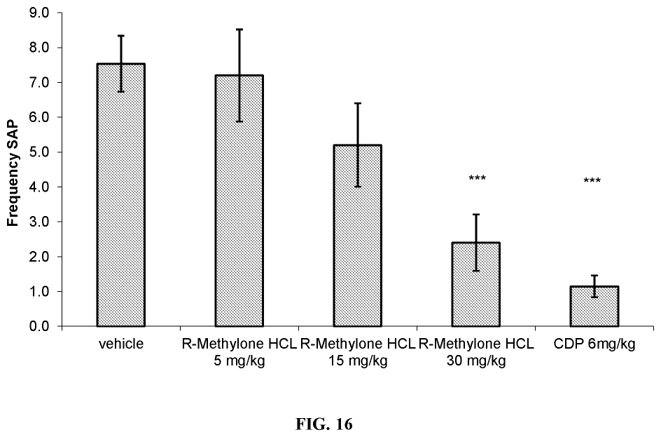

Surprisingly, in contrast to racemic methylone and S-methylone, R-methylone did not show any anxiogenic effect at any dose. In fact, R-methylone showed a dose dependent increase in time spent in the open arms that was as effective as chlordiazepoxide at 30 mg/kg and also showed a simultaneous dose dependent decrease in SAPs across this range with 30 mg/kg significantly decreasing SAPs to the same level as chlordiazepoxide (FIG. 13 and FIG. 16). This surprising result shows that R-methylone does not have the anxiogenic side effects seen with racemic methylone and S-methylone. Since racemic methylone is comprised of equal amounts of S-methylone and R-methylone, this indicates that the anxiogenic side effects seen with lower doses of racemic methylone are due to the anxiogenic effects of S-methylone.

The data shows that while racemic methylone, S-methylone and R-methylone all have anxiolytic effects as effective as chlordiazepoxide at the high dose level, racemic methylone and S-methylone show anxiogenic effects at lower doses, an effect not seen with R-methylone. There are several critical implications of this finding. The first is that patients treated with racemic methylone or S-methylone must receive a dose high enough to reach the anxiolytic threshold since lower doses may cause anxiety as a side effect and result in worsening of the disorder being treated. This could have especially severe implications for anxiety disorders or depressive disorders including post-traumatic stress disorder, generalized anxiety disorder, panic disorder, major depressive disorder, or treatment resistant depression. All of these indications are associated with an increased level of anxiety. In these cases, a drug-induced increase in anxiety due to improper dosing of racemic methylone or S-methylone could have severe side effects on patients and worsen their underlying disorder. The data presented herein show that patients treated with a racemic methylone or S-methylone must be carefully titrated to avoid the anxiogenic effects and to reach the anxiolytic effect level. The data show that in some embodiments a Risk Evaluation and Mitigation Strategy (REMS) program should be utilized so that patients treated with racemic methylone or S-methylone should undergo an initial dose titration to determine the effective range specific to that patient. This dose titrating protocol would decrease the side effects related to underdosing racemic methylone or S-methylone.

The data also inform Phase 2 and Phase 3 clinical trial design. Clinical trials for neurological and psychiatric disorders often include one or more low dose arms to show a dose dependent effect of the full dose on the disease of interest. However, this data shows that racemic and S-methylone should only be dosed at the full effective dose and a low dose arm should not be included as a comparator as this may lead to harmful side effects on the patients. This data shows that studies of racemic and S-methylone should only use inactive matched placebo or a different standard of care therapeutic as a control. In clinical trials methylone should only be dosed at its effective dose range to avoid harmful side effects to the patients. This would be especially critical in clinical studies of anxiety disorders or depression including post-traumatic stress disorder, generalized anxiety disorder, panic disorder, major depressive disorder, or treatment resistant depression where increased anxiety could worsen the underlying disorder and lead to potentially devastating effects on the patients.

The data show that there is an advantage of R-methylone which is anxiolytic without any anxiogenic effects. In some embodiments, a clinician treating a patient with R-methylone does not need to utilize a specific dose titration protocol to reduce anxiogenic effects. In some embodiments clinical studies of R-methylone have a greater safety margin and are able to use lower doses in different arms of the study to demonstrate a dose dependent effect on the disease of interest. In some embodiments, R-methylone allows greater flexibility in clinical trial design including the safe use of a low dose active comparator to reduce expectancy bias. In some embodiments, R-methylone would be preferred to racemic methylone or S-methylone to treat patients with anxiety or depressive disorders including post-traumatic stress disorder, generalized anxiety disorder, panic disorder, major depressive disorder, or treatment resistant depression. In some embodiments R-methylone is a safer alternative to racemic methylone or S-methylone for the treatment of neurological and psychiatric disorders. Prodrugs of methylone are expected to have the same effect post-cleavage of the pro-portion so these results with methylone would be expected to apply to dosing the prodrugs as well.

### Example C: Methylone Dose Titration Risk Evaluation and Mitigation Strategy (REMS) Protocol

### General Information on Methylone Treatment Session

The methylone doses presented in examples C and D are based on the effective dose of methylone. Prodrugs of methylone are expected to achieve efficacious levels of methylone post-cleavage of the pro-portion once the dose of the prodrug is adjusted to account for the extra weight of the pro-portion and the pharmacokinetic or pharmacodynamic advantages of the prodrug. Thus, the doses in these sections apply to the active amount of methylone released by the prodrug. Initial methylone dosing and subsequent dosing adjustments must be done under the supervision of a qualified healthcare professional in a clinic or inpatient setting. The patient must remain under supervision of the healthcare professional for at least 6 hours and up to approximately 24 hours after the final methylone dose adjustment. The patient will be assessed periodically during the session for anxiety and other effects of methylone. Dose adjustments within a methylone treatment session will be based on changes from baseline levels of anxiety. Postdose anxiety measurement timing and duration of observation after dosing are based on the following information reported by the World Health Organization (WHO 2014):

Duration of effects of methylone

| Effects | Time After Dose |
|---|---|
| Onset | 15 min-60 min |
| Coming Up | 30 min-45 min |
| Plateau | 60 min-90 min |
| Coming Down | 60 min-120 min |
| Duration | 2 hours-3.5 hours |
| Normal After Effects | 6 hours-24 hours |
| Total Duration | 3 hours-5 hours |

Methylone dosing is modified from information reported by the World Health Organization (WHO 2014):

Methylone dosages

| Drug Activity Level | Oral Methylone Dose |
|---|---|
| Threshold | 60 mg-100 mg |
| Light | 100 mg-150 mg |
| Common | 150 mg-200 mg |
| Strong | 200 mg-270 mg |
| Very Strong | ≥270 mg |

In some embodiments, the methylone dosing above is used with racemic methylone. In some embodiments, the methylone dosing above is used with (S)-methylone.

### Predose Assessment

The patient's baseline level of anxiety will be measured and recorded.

### Initial Methylone Dosing

The patient will receive an initial single oral dose of methylone in the range of approximately 100 mg-200 mg based on oral doses reported as producing moderate effects (Poyatos et al., 2021).

### Postdose Assessment

Change from baseline anxiety level will be measured at approximately 1 to 2 hours after dosing based on reported time to achieve peak effects (Poyatos et al., 2021).

### Methylone Dose Adjustment

Methylone effects have been maintained by taking a larger initial dose followed by smaller doses (30 mg to 100 mg p.o.) (WHO 2014). Re-dose of one-third to one-half the initial dose usually prolongs duration for approximately one hour (WHO 2014). Accordingly, the dose of methylone will be adjusted based on change from baseline in anxiety as follows:

Methylone dose adjustment

| Change from | |
|---|---|
| Baseline Anxiety | Methylone Dose Adjustment |
| Increased | Increase dose 30%-50% and reassess anxiety in approximately 1 hour |
| No Change | Increase dose 30%-50% and reassess anxiety in approximately 1 hour |
| Decreased | Maintain dose if therapeutic effect achieved or |
| | Increase to a maximum of 250 mg total dose to optimize therapeutic effect |

### Methylone Discontinuation

The patient will be observed for at least 6 hours after final methylone dose is administered.

The patient may be confined to the inpatient unit for prolonged observation up to approximately 24 hours after last methylone dose if indicated based on persistent effects.

Anxiety that appears after the final methylone titration dose is administered can be managed with an appropriate anxiolytic agent (Prosser and Nelson, 2012). If this is necessary, the patient must remain under observation and undergo periodic reassessment until the supervising healthcare professional determines the patient can be discharged from care.

Poyatos L, et al., A Comparison of Acute Pharmacological Effects of Methylone and MDMA Administration in Humans and Oral Fluid Concentrations as Biomarkers of Exposure. Biology (Basel). 2021 Aug. 17; 10(8):788. Prosser J M, Nelson L S. The toxicology of bath salts: a review of synthetic cathinones. J Med Toxicol. 2012 March; 8(1):33-42. World Health Organization (WHO). Methylone (bk-MDMA). Critical Review Report; WHO: Geneva, Switzerland, 2014.

### Example D: A Double-Blind, Randomized, Placebo-Controlled Clinical Trial of Methylone-Assisted Psychotherapy in PTSD

A multicenter, randomized, double-blind, placebo-controlled trial is conducted to assess the efficacy and safety of methylone-assisted psychotherapy versus psychotherapy with placebo control in participants diagnosed with at least moderate post-traumatic stress disorder (PTSD).

### Rationale

PTSD is a debilitating and often times chronic disorder associated with profound mental, physical, occupational, and functional impairment. PTSD can develop due to exposure to a traumatic event or persistent or recurring threats to an individual. Studies indicate that approximately 10% of individuals exposed to a traumatic event eventually go on to be diagnosed with PTSD (American Psychiatric Association. Diagnostic and statistical manual of mental disorders, 5^{th} edition, 2013). PTSD is a complex psychiatric disorder characterized by symptom heterogeneity including avoidance of trauma-related material, emotional blunting and distancing, hyper-vigilance, hyper-arousal, persistent negative alterations in mood, persistent alterations in cognition, disturbing thoughts, disruptions in sleep and/or dreams, and physical or mental distress. Symptoms can be severe and long lasting. Although this symptom heterogeneity may suggest a wide spectrum of separate disturbances, emotional dysregulation is considered to be a core component of this disorder. Particularly germane to the pathogenesis and progression of PTSD, emotional dysregulation in affected individuals is believed to give rise to observable and measurable features such as presence of hypervigilance and attentional biases, enhanced startle response, hyper-arousal, apathetic feeling or emotional numbness, irritability, enhanced memories associated with traumatic events, difficulty in discerning danger versus safety, a generalization of fear, and avoidance of reminders of trauma. Emotional dysregulation may be defined and also measured by elevated emotional reactivity based on abnormal detection or appraisal of emotional triggers involving bottom-up sensory detection and neuronal processing. Biochemical alterations found in individuals diagnosed with PTSD suggest abnormalities in the hypothalamic-pituitary-adrenal (HPA) axis. The HPA axis is known to regulate reactions to stress and controls significant aspects of the neuroendocrine system impacting many homeostatic systems in the body. In a typical flight-or-flight response in a healthy individual, catecholamine and cortisol levels detected in urine rise after exposure to a stressor. In PTSD, many individuals show a low secretion of cortisol and high secretion of catecholamine in response to a stressor indicating a change in catecholamine to cortisol ratio in the urine. More evidence that the HPA axis is impacted in PTSD is found in elevated levels of catecholamines and corticotropin-releasing factor in the brain of many affected individuals.

The initiation and/or maintenance of emotional dysregulation in PTSD may be due to abnormalities in top-down control of emotional responses indicating that cognitive influences and higher order representations may impinge on information and emotional processing. Certainly, some aspects of abnormalities in neuronal processing in PTSD occur either implicitly (e.g., unconsciously) or explicitly (e.g., consciously) indicating involvement of distinct cognitive processes. Exaggerated responses in the amygdala and insular cortex have been demonstrated in meta-analyses in PTSD pathology, as have decreases in activity in other brain regions including the anterior cingulate cortex and aspects the prefrontal cortex including the ventromedial prefrontal cortex. In addition to changes in patterns of neuronal activity in individuals with PTSD, several neuroanatomical changes in PTSD have also been demonstrated. A reduction of total brain volume, intracranial volume, and the volumes in regions such as the hippocampus (particularly localized to the CA3 and dentate gyrus regions), insular cortex, and anterior cingulate cortex have been indicated in occurring in some individuals with PTSD through meta-analyses of structural MRI studies. Animal studies have shown that severe chronic stress leads to atrophy of apical dendrites in the CA3 region of the hippocampus, reduced hippocampus neurogenesis, and elevated granule cell death in the dentate gyrus due to elevated levels of glucocorticoids (Gould E. and Tanapat. (1999). Stress and hippocampal neurogenesis. Biol. Psychiatry 46, 1472-1479.) Connections between brain areas such as the amygdala, hippocampus, prefrontal cortex, and hypothalamus can facilitate activation of the HPA axis to illustrate interactions between brain regions with structural changes and affected biochemical regulatory systems in PTSD.

Methylone is a synthetic analog of the psychedelic phenethylamine class of compounds known to act as a mixed reuptake inhibitor/releasing agent of serotonin, norepinephrine, and dopamine and administration of methylone can produce acute modulations of neurotransmission. Methylone administration also has indirect effects on neurohormone release. Methylone can function as a psychoplastogen promoting neuronal growth, modulating neuronal connectivity, and regulating neuronal plasticity through longer term neuronal changes. The combined neurobiological effects of methylone administration on individuals reduce fear of emotional injury or distress, enhance introspection and communication, and increase empathetic feelings and compassion. Additionally, methylone may serve to enhance fear extinction. These combined effects may yield acute and longer-term productive psychological states to enhance behavioral or cognitive-behavioral therapies. Methylone administration may enhance neuronal function at the biochemical and cellular levels to generate or restore favorable neural network pathways and connectivity to increase behavioral or cognitive-behavioral therapy productiveness.

### Study Design

This multicenter, randomized, double-blind, placebo-controlled trial is conducted at various sites in the United States with IRB approval from each study site. A flexible dose of methylone hydrochloride salt or placebo, followed by a supplemental half-dose unless contraindicated by patient's previous response or medical history, is also administered during the Treatment Period with psychotherapy in at least 3 blinded monthly Experimental Sessions. The Supplemental Dose extends the duration of drug effects on the participants during an Experimental Session. Methylone test groups are further subdivided into specific groups receiving only racemic methylone hydrochloride salt, S-methylone hydrochloride salt, or R-methylone hydrochloride salt. An optional Risk Evaluation and Mitigation Strategy (REMS) Protocol may be implemented for the racemic methylone, S-methylone, and placebo-groups. The Treatment Period lasts for approximately 12 weeks. During the Treatment Period, each Experimental Session is followed by three Intervening Sessions of non-drug psychotherapy. Each Experimental Session involves an overnight stay. The Primary Outcome measure, the change in Clinician Administered PTSD Scale for DSM-5 (CAPS-5), is determined by a blinded Independent Rater (IR) pool multiple times throughout the study. The study consists of separate periods for each participant. Initially, prospective participants undergo a Screening Period involving an initial eligibility assessment, a medical history intake, informed consent, and enrollment of eligible participants. Next, a Preparation Period is undertaken for enrolled participants involving medication tapering and clinical baseline assessments to confirm each participant meets enrollment criteria. As part of the Preparation Period, a detailed assessment of co-morbidities to PTSD is recorded. Participants may remain on prescribed courses of selective serotonin reuptake inhibitor (SSRI) or serotonin and norepinephrine reuptake inhibitor (SNRI) treatment. Dosages and/or frequency of administration of a prescribed SSRI or SNRI may be adjusted to fit within study parameters. Participants may be required to taper a prescribed course of medication in order to maintain eligibility within the study. The Treatment Period consists of three monthly Experimental Sessions and associated Intervening Sessions of integrative behavioral psychotherapy. The Treatment Period lasts approximately 12 weeks. Following the Treatment Period is a Follow-up Period and Study Conclusion. During the Follow-up Period and Study Conclusion, participants complete 4 weeks with no study visits, followed by a Study Conclusion visit.

Screening Period - from initial consent to beginning of enrollment (approx. 4 weeks)

| Study Visit | | Visit Timing | Description |
|---|---|---|---|
| Screening | Screening | Several visits | Informed consent obtained and assessment |
| | | taking place | measures of pre-study medications, complete |
| | | 5-30 days | personal and family medical history and all |
| | | after initial | assessed screening measures undertaken. These |
| | | phone call | measures may include any of: PTSD checklist for |
| | | screen | DSM-5 (PCL-5), Columbia-Suicide Severity |
| | | | Rating Scale (C-SSRS), Montgomery-Asberg |
| | | | Depression Rating Scale (MADRS), Hamilton |
| | | | Depression Rating Scale (HAM-D), Hamilton |
| | | | Anxiety Rating Scale (HAM-A), General Anxiety |
| | | | Disorder-7 (GAD-7), Beck Anxiety Inventory |
| | | | (BAI), Impact of Events Scale (IES), State-Trait |
| | | | Anxiety Inventory (STAI), Edinburgh Postnatal |
| | | | Depression Scale (EPDS), Clinical Global |
| | | | Impressions Scale (CGI-I), Epworth Sleepiness |
| | | | Scale (ESS), and Pittsburgh Sleep Quality Scale. |
| | | | Medical providers are contacted and medical |
| | | | records and laboratory results are obtained. All |
| | | | results and records are reviewed along with |
| | | | interview notes. If eligible, results of Life Events |
| | | | Checklist for DSM-5 (LEC-5) and Structured |
| | | | Clinical Interview for DSM-5 Personality |
| | | | Questionnaire (SCID-5-SPQ) are forwarded to IR. |
| | IR | 2-10 days | Initial eligibility after PCL-5 and initial eligibility |
| | Screening | after initial | are reviewed. Next, IR conducts a since last visit |
| | | eligibility | C-SSRS, SCID-5-PD, Dissociative Disorders |
| | | determined | Interview Schedule (DDIS), and/or International |
| | | during | Neuropsychiatric Interview (MINI). Results of IR |
| | | Screening | assessment confirmed over Preparatory Period. |
| | | Period | |
| Enrollment | Enrollment | 1-14 days | Prior to enrollment, all screening measures are |
| | | after IR | reviewed and any clarification needed with |
| | | Screening | participant is completed by telephone interview. If |
| | | | enrolled, and if it has been determined to taper an |
| | | | ongoing medication, begin a tapering treatment |
| | | | plan of at least 5 half-lives plus at least 5 days for |
| | | | stabilization. Begin collection of Adverse Events |
| | | | (AE). |

Preparation Period (between 1-12 weeks)

| Study Visit | | Visit Timing | Description |
|---|---|---|---|
| Preparatory | Preparatory | Undertaken | Schedule visit timing according to medication |
| Period | Session 1 | 0-14 days | tapering needs. Schedule calls in between |
| | | post- | visits for safety concerns, tapering questions, |
| | | enrollment | or other issues related to medical history. |
| | | | Confirm enrollment. |
| | Preparatory | Undertaken | Schedule upcoming visits if medication |
| | Session 2 | 2-21 days | tapering is not needed or is already completed. |
| | | following | If still tapering, schedule additional telephone |
| | | Preparation | call for continuing assessment of readiness to |
| | | Session 1 | enter study. |
| | Taper | 0-7 days | Schedule baseline CAPS-5. |
| | follow-up | following end | |
| | | of medication | |
| | | taper | |
| Baseline and | Baseline | Following | Complete CAPS-5, Sheehan Disability Score |
| Enrollment | Assessments | Preparatory | (SDS), and Dissociative Subtype of PTSD |
| Confirmation | | Session 2 | Scale (DSPS) by IR via in-person or |
| | | | telemedicine appointment. Scores forwarded |
| | | | to therapy monitoring team. Resumption of |
| | | | tapered medicine in symptom management |
| | | | requires. Withdrawal of participants not |
| | | | meeting eligibility criteria at this point. |
| | Preparatory | 1-7 days | Participants complete baseline self-report |
| | Session 3 | following | metrics and schedule Experimental Session 1. |
| | | baseline | |
| | | CAPS-5 | |

The Treatment Period schedule follows the Screening Period and the Preparatory Period

Treatment Period (lasts approximately 12 weeks)

| Study Visit | | Visit Timing | Description |
|---|---|---|---|
| Treatment 1 | Randomization | 0-10 days | Complete following verification participant |
| | | following | is still enrolled and Experimental Session 1 |
| | | Baseline | is scheduled. Double-blind randomization. |
| | | assessments | |
| | Experimental | 8 hours plus | Patient's weight is determined for dosage |
| | Session 1 | overnight | calculation. Baseline STAI assessment. |
| | | observation | Dose is 100-200 mg p.o. Placebo |
| | | | administered in placebo group at same time |
| | | | interval. |
| | | Optional. | REMS Protocol: Only considered for |
| | | Following | administration of racemic methylone and S- |
| | | anxiety | methylone treatment groups and associated |
| | | assessment | placebo controls. Underdosing of racemic |
| | | 0.75-2 hours | methylone or S-methylone may lead to |
| | | after first dose | exacerbation of anxiogenic features |
| | | administration | necessitating careful assessment of |
| | | | worsening of symptoms. Patients are |
| | | | assessed for anxiety about 0.75 hours after |
| | | | first dose. With no change or increase in |
| | | | anxiety, patients are given a dose of 30%- |
| | | | 50% of first dose and anxiety reassessed in |
| | | | about 1 hour. Dose titration up to a |
| | | | maximum cumulative dose of 250 mg. |
| | | | Placebo administered in placebo group at |
| | | | same time interval. If anxiety is decreased, |
| | | | no REMS protocol dose is given at this |
| | | | time. |
| | | 2-2.5 hours | Supplemental Dose: Supplemental half-dose |
| | | after first dose | of 50-100 mg administered 2 to 2.5 hours |
| | | administration | following initial dose administration unless |
| | | | contraindicated. If initial plus REMS |
| | | | protocol doses total a cumulative dose of |
| | | | 250 mg, no Supplemental Dose is given. If |
| | | | initial plus any REMS protocol dose is less |
| | | | than 250 mg, Supplemental Dose is |
| | | | administered 2 to 2.5 hours following initial |
| | | | dose administration. Placebo administered |
| | | | in placebo group at same time intervals. |
| | Intervening | Morning | Behavioral or cognitive-behavioral therapy |
| | Session 1A | following | session lasting 90-120 minutes. Assessment |
| | | Experimental | of potential anxiogenic, mixed anxiogenic- |
| | | Session 1 | anxiolytic, or anxiolytic treatment effects. |
| | | | CAPS-5 assessment. Instructions for |
| | | | participants to complete C-SSRS |
| | | | assessments on every two days following |
| | | | Experimental Session 1. |
| | Intervening | 3 to 14 days | Behavioral or cognitive-behavioral therapy |
| | Session 1B | following | session lasting 60-120 minutes. CAPS-5 |
| | | Experimental | assessment. |
| | | Session 1 | |
| | Intervening | 18-34 days | Behavioral or cognitive-behavioral therapy |
| | Session 1C | following | session lasting 60-120 minutes. |
| | | Experimental | Assessments include LEC-5, HAM-A, C- |
| | | Session 1 | SSRS and CAPS-5. |
| Treatment 2 | Experimental | 8 hours plus | Patient's weight is determined for dosage |
| | Session 2 | overnight | calculation. Baseline STAI assessment. |
| | | observation. | Dose is 100-200 mg p.o. Placebo |
| | | 19-35 days | administered in placebo group at same time |
| | | following | interval. |
| | | Experimental | |
| | | Session 1. | |
| | | Optional. | REMS Protocol: Only considered for |
| | | Following | administration of racemic methylone and S- |
| | | anxiety | methylone treatment groups and associated |
| | | assessment | placebo controls. Underdosing of racemic |
| | | 0.75-2 hours | methylone or S-methylone may lead to |
| | | after first dose | exacerbation of anxiogenic features |
| | | administration | necessitating careful assessment of |
| | | | worsening of symptoms. Patients are |
| | | | assessed for anxiety about 0.75 hours after |
| | | | first dose. With no change or increase in |
| | | | anxiety, patients are given a dose of 30%- |
| | | | 50% of first dose and anxiety reassessed in |
| | | | about 1 hour. Dose titration up to a |
| | | | maximum cumulative dose of 250 mg. |
| | | | Placebo administered in placebo group at |
| | | | same time interval. If anxiety is decreased, |
| | | | no REMS protocol dose is given at this |
| | | | time. |
| | | 2-2.5 hours | Supplemental Dose: Supplemental half-dose |
| | | after first dose | of 50-100 mg administered 2 to 2.5 hours |
| | | administration | following initial dose administration unless |
| | | | contraindicated. If initial plus REMS |
| | | | protocol doses total a cumulative dose of |
| | | | 250 mg, no Supplemental Dose is given. If |
| | | | initial plus any REMS protocol dose is less |
| | | | than 250 mg, Supplemental Dose is |
| | | | administered 2 to 2.5 hours following initial |
| | | | dose administration. Placebo administered |
| | | | in placebo group at same time intervals. |
| | Intervening | Morning | Behavioral or cognitive-behavioral therapy |
| | Session 2A | following | session lasting 90-120 minutes. |
| | | Experimental | Assessment of potential anxiogenic, mixed |
| | | Session 2 | anxiogenic-anxiolytic, or anxiolytic |
| | | | treatment effects. CAPS-5 assessment. |
| | | | Instructions for participants to complete C- |
| | | | SSRS assessments on every two days |
| | | | following Experimental Session 1. |
| | Intervening | 3 to 14 days | Behavioral or cognitive-behavioral therapy |
| | Session 2B | following | session lasting 60-120 minutes. CAPS-5 |
| | | Experimental | assessment. |
| | | Session 2 | |
| | Intervening | 18-34 days | Behavioral or cognitive-behavioral therapy |
| | Session 2C | following | session lasting 60-120 minutes. |
| | | Experimental | Assessments include LEC-5, HAM-A, and |
| | | Session 2 | C-SSRS and CAPS-5 . . . |
| Treatment 3 | Experimental | 8 hours plus | Patient's weight is determined for dosage |
| | Session 3 | overnight | calculation. Baseline STAI assessment. |
| | | observation. | Dose is 100-200 mg p.o. Placebo |
| | | 19-35 days | administered in placebo group at same time |
| | | following | interval. |
| | | Experimental | |
| | | Session 2. | |
| | | Optional. | REMS Protocol: Only considered for |
| | | Following | administration of racemic methylone and S- |
| | | anxiety | methylone treatment groups and associated |
| | | assessment | placebo controls. Underdosing of racemic |
| | | 0.75-2 hours | methylone or S-methylone may lead to |
| | | after first dose | exacerbation of anxiogenic features |
| | | administration | necessitating careful assessment of |
| | | | worsening of symptoms. Patients are |
| | | | assessed for anxiety about 0.75 hours after |
| | | | first dose. With no change or increase in |
| | | | anxiety, patients are given a dose of 30%- |
| | | | 50% of first dose and anxiety reassessed in |
| | | | about 1 hour. Dose titration up to a |
| | | | maximum cumulative dose of 250 mg. |
| | | | Placebo administered in placebo group at |
| | | | same time interval. If anxiety is decreased, |
| | | | no REMS protocol dose is given at this |
| | | | time. |
| | | 2-2.5 hours | Supplemental Dose: Supplemental half-dose |
| | | after first dose | of 50-100 mg administered 2 to 2.5 hours |
| | | administration | following initial dose administration unless |
| | | | contraindicated. If initial plus REMS |
| | | | protocol doses total a cumulative dose of |
| | | | 250 mg, no Supplemental Dose is given. If |
| | | | initial plus any REMS protocol dose is less |
| | | | than 250 mg, Supplemental Dose is |
| | | | administered 2 to 2.5 hours following initial |
| | | | dose administration. Placebo administered |
| | | | in placebo group at same time intervals. |
| | Intervening | Morning | Behavioral or cognitive-behavioral therapy |
| | Session 3A | following | session lasting 90-120 minutes. |
| | | Experimental | Assessment of potential anxiogenic, mixed |
| | | Session 3 | anxiogenic-anxiolytic, or anxiolytic |
| | | | treatment effects. CAPS-5 assessment. |
| | | | Instructions for participants to complete C- |
| | | | SSRS assessments on every two days |
| | | | following Experimental Session 1. |
| | Intervening | 3 to 14 days | Behavioral or cognitive-behavioral therapy |
| | Session 3B | following | session lasting 60-120 minutes. CAPS-5 |
| | | Experimental | assessment. |
| | | Session 3 | |
| | Intervening | 18-34 days | Behavioral or cognitive-behavioral therapy |
| | Session 3C | following | session lasting 60-120 minutes. |
| | | Experimental | Assessments include LEC-5, HAM-A, C- |
| | | Session 3 | SSRS and CAPS-5. |

The Follow-up Period schedule and Study Conclusion follow the Screening Period and the Treatment Period.

Follow-up Period and Study Conclusion

| Study Visit | Visit Timing | Description |
|---|---|---|
| Follow-up Period | Occurs 2-10 days | Occurs about 100-150 days following Baseline |
| | after Intervening | assessment. Complete self-reported |
| | Session 3C. | assessments and patient safety measures. |
| | | Create exit treatment plan for participant based |
| | | on results. Final CAPS-5 assessment. Final |
| | | SDS, HAM-D, and ESS assessments. |
| Study Conclusion | At time of | Inform participants who finished study |
| | unblinding of group. | protocol of unblinding of groups. If a |
| | | participant was in a placebo group, offer |
| | | opportunity to enroll in a open-label safety |
| | | extension study using either racemic |
| | | methylone, S-methylone, or R-methylone. |

### Dose Selection

This study compares the effects of three blinded Experimental Sessions of psychotherapy in combination with flexible doses of methylone or placebo administered as described below. Non-drug preparatory and intervening psychotherapy sessions are also included. Patient's weight is determined for dosage calculation. Initial dose is 100 mg unless this will result in a dosage of less than 1.5 mg/kg of patient weight. Initial dose thereby adjusted upward in 25 mg increments to deliver the lowest dose possible of at least 1.5 mg/kg of patient weight. Initial dose for Experimental Session 2 and 3 is cumulative dose calculated by adding the initial dose plus REMS protocol dose used the previous Experimental Session for each patient.

| | | | Optional | | |
|---|---|---|---|---|---|
| | | | (REMS) | | |
| | | | protocol: | | |
| Double- | | | Dose | | |
| blinded | | | Titration if | Supplemental | |
| treatment | Experimental | | underdosing | Dose (unless | Cumulative |
| group | Session | Initial Dose | occurs | contraindicated) | Dose |
| Racemic | 1 | 100-200 mg | 30-100 mg | 50-100 mg | 100-300 mg |
| methylone | | | | | |
| Racemic | 2 | 100-200 mg | 30-100 mg | 50-100 mg | 100-300 mg |
| methylone | | | | | |
| Racemic | 3 | 100-200 mg | 30-100 mg | 50-100 mg | 100-300 mg |
| methylone | | | | | |
| S-methylone | 1 | 100-200 mg | 30-100 mg | 50-100 mg | 100-300 mg |
| S-methylone | 2 | 100-200 mg | 30-100 mg | 50-100 mg | 100-300 mg |
| S-methylone | 3 | 100-200 mg | 30-100 mg | 50-100 mg | 100-300 mg |
| R-methylone | 1 | 100-200 mg | N/A | 50-100 mg | 100-300 mg |
| R-methylone | 2 | 100-200 mg | N/A | 50-100 mg | 100-300 mg |
| R-methylone | 3 | 100-200 mg | N/A | 50-100 mg | 100-300 mg |

### Randomization and Masking

Randomization occurs prior to the initiation of Experimental Session 1. Each participant is provided the next randomized number in a sequence by a blinded study monitor. Participants are then randomized, according to a computer-generated randomization schedule, 1:1:1:1 to racemic methylone, S-methylone, R-methylone, or placebo. The randomization schedule is prepared and implemented by an independent statistician. Participants, clinicians, and study teams are blinded to treatment allocation. Racemic methylone and S-methylone treatment groups may be subjected to anxiogenic effects due to underdosing of participants. As such, an optional dose titration schedule (REMS protocol) exists for racemic methylone and S-methylone treatment groups if a participant displays no change or a significant worsening of assessed anxiety symptomatology. Participants are assessed for general well-being and anxiety by a medical practitioner about 0.75 hours after the first dose is administered. Assessments performed may include general assessments of physical and mental well-being, a structured clinical interview for DSM-5 (SCID-5) module A1, and/or a STAI assessment and may continue throughout the period of overnight observation.

Subjects then undergo three Intervening Sessions with the first session the morning after the initial dose administration. R-methylone treatment group or placebo group participants qualifying with a significant worsening of assessed anxiety symptomatology would undergo a placebo dose titration administration. Subjects would then undergo three Intervening Sessions with the first session the morning after the placebo dose titration administration. The pharmacist at each site, who prepares the treatments according to the randomization schedule, and an unblinded monitor, who performs drug accountability during the study, are unblinded. No other study personnel are unblinded until after formal locking of the study database. In the event of a medical emergency, the pharmacist is to reveal actual treatment contents to the primary investigator, who is to alert the Sponsor of the emergency. If the participant or study center personnel are unblinded, the subject is to be removed from the study.

### Outcomes

The primary objective of this study is to evaluate the efficacy and safety of methylone treatment combined with psychotherapy to treat moderate to severe PTSD compared to identical psychotherapy combined with placebo treatment. Methylone treatment is further subdivided into three separate treatment groups (racemic methylone, S-methylone, and R-methylone) with each treatment subgroup only receiving administration of the single assigned drug. Treatment outcomes are determined based on a change in CAPS-5 Total Severity.

Several secondary objectives are designed for this study. One is an evaluation of clinician-rated functional impairment of methylone treatment combined with psychotherapy to treat moderate to severe PTSD compared to identical psychotherapy combined with placebo treatment. Methylone treatment is further subdivided into three separate treatment groups (racemic methylone, S-methylone, and R-methylone) with each treatment subgroup only receiving administration of the single assigned drug. Treatment outcomes are determined based on a change in SDS. Another secondary objective of this study is to evaluate clinician-rated depression of methylone treatment combined with psychotherapy to treat moderate to severe PTSD compared to identical psychotherapy combined with placebo treatment. Identical study parameters are in place as for the clinician-rated functional impairment assessment except that treatment outcomes are determined based on a change in HAM-D. An additional secondary objective of this study is to evaluate sleep assessments of methylone treatment combined with psychotherapy to treat moderate to severe PTSD compared to identical psychotherapy combined with placebo treatment. Identical study parameters are in place as for the clinician-rated functional impairment assessment except that treatment outcomes are determined based on a change in ESS. Co-morbidities present in participants with a strong positive response to methylone treatment are correlated. Co-morbidities present in participants with weak-to-no positive response to methylone treatment are correlated. Changes to presence or severity of co-morbidities from the Preparation Period to the Study Conclusion are recorded to determine if methylone treatment combined with psychotherapy in moderate to severe PTSD subjects affects co-morbid phenotypes not falling under the constellation of PTSD symptoms.

### Participant Populations

Participants are recruited through referrals by other treatment providers or through print or internet advertisements. The Sponsor monitors demographics of individuals assessed for enrollment to encourage diversity and an unbiased representation of the total PTSD population. Participants must be 18 years of age or older, have a confirmed diagnosis of at least moderate PTSD according to PCL-5 at the Screening Period. Medical history intake must indicate a presence of PTSD symptoms for at least 6 months prior to the Screening Period. Participants may be enrolled in the study while remaining on a treatment regimen involving SSRI or SNRI treatment prescribed for PTSD. In some cases, enrolled participants currently taking an SSRI, an SNRI, or another medication are tapered off these medications and stabilized prior to baseline assessments. Participants with a confirmed personality disorder diagnosis are excluded from this study. Participants must be in good general physical health without one or more severe chronic conditions that could affect the safety or tolerability of methylone treatment.

### Statistical Analysis

The change from baseline in CAPS-5, SDS, HAM-D, and ESS in participants is analyzed using a mixed effects model for repeated measures (MMRM) to obtain covariance parameter estimates. The model includes treatment center, treatment subtype, baseline assessments, assessment time point, and time point-by-treatment as explanatory variables. Treatment center is treated as a random effect; all other explanatory variables are treated as fixed effects. Model-based point estimates (e.g., least squares means, 95% confidence intervals, and p-values) are reported for each time point. With a sample size of 50 participants per treatment group, this study has 90% power to detect a significant treatment effect, using a two-sided test with an alpha value of 0.05. Additional participants may be enrolled with conditional power analysis conducted at a group-unblinded interim analysis time point for efficacy when 200 participants are enrolled and at least 60% of the blinded participants (N=120) have completed a final CAPS-5 assessment and reached Study Conclusion.

### Results

The results may indicate that the primary objective is achieved. At the point of Study Conclusion, racemic methylone-treated, S-methylone-treated, and R-methylone-treated participants may demonstrate a significant mean reduction in CAPS-5 assessment compared to the placebo group. The S-methylone-treated subgroup may achieve a significant mean reduction in CAPS-5 assessment with a lower total dosage of drug compared to the racemic methylone-treated subgroup. The R-methylone-treated subgroup may achieve a significant mean reduction in CAPS-5 assessment with a lower total dosage of drug compared to the racemic methylone-treated subgroup. The R-methylone-treated subgroup may achieve a significant mean reduction in CAPS-5 assessment with a higher total dosage of drug compared to the racemic methylone-treated subgroup. Significant improvements in CAPS-5 assessments may be observed for racemic methylone-treated, S-methylone-treated, and R-methylone-treated participants at time points of Intervening Session 1C, Intervening Session 2C, Intervening Session 3C and Study Conclusion, compared to placebo-treated controls. Significant improvements in CAPS-5 assessments may be observed for R-methylone-treated participants at time points of Intervening Session 1C, Intervening Session 2C, Intervening Session 3C, compared to placebo-treated controls without a significant increase in adverse anxiogenic incidents in R-methylone-treated participants.

The results may indicate that the secondary objectives of this study are also achieved. At the point of Study Conclusion, racemic methylone-treated, S-methylone-treated, and R-methylone-treated participants may demonstrate a significant improvement in clinician-rated functional impairment score as measured by SDS compared to placebo-treated controls. At the point of Study Conclusion, racemic methylone-treated, S-methylone-treated, and R-methylone-treated participants may demonstrate a significant improvement depression as measured by HAM-D compared to placebo-treated controls. At the point of Study Conclusion, racemic methylone-treated, S-methylone-treated, and R-methylone-treated participants may demonstrate a significant improvement in lessening daytime sleepiness as measured by ESS. At the point of Study Conclusion, R-methylone-treated participants may demonstrate a significant improvement in clinician-rated functional impairment score, in depression, and in lessening daytime sleepiness compared to placebo-treated controls without a significant increase in adverse anxiogenic incidents in R-methylone-treated participants.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A compound of Formula (I): 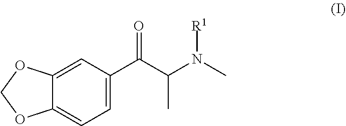
or a pharmaceutically acceptable salt thereof, wherein:
R¹ is —C(O)OR³, —C(O)R⁴, —CH(R⁵)OR⁶, —C(O)OCH(R⁵)OC(O)R⁴, —C(O)OCH(R⁵)OC(O)OR⁴, —C(O)OCH(R⁵)OC(O)NHR⁴, —CH(R⁵)NHC(O)R⁶, —CH(R⁵)C(O)R⁶, —S(O)₂R⁷, —S(O)₂OR⁷, —P(O)OR⁸[N(R⁹)R¹⁰], —C(O)N(R⁹)R¹⁰, —P(O)OR¹¹(OR¹²), —CH(R⁴)OP(O)OR⁸[N(R⁹)R¹⁰], or —CH(R⁴)OP(O)OR¹¹(OR¹²);
each of R³, R⁴, R⁶, R⁷, and R¹ is independently alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
R⁵ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
or R³ is 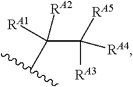
wherein each of R^{A1}, R^{A2}, R^{A3}, and R^{A4} is independently hydrogen or alkyl; and R^{A5} is heteroalkyl, heterocycloalkyl, heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶;
or R⁴ is 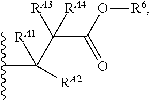
wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; and R⁶ is hydrogen, alkyl, cycloalkyl, aryl, or heteroaryl;
or R⁴ is 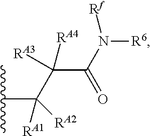
wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; R^{f} is hydrogen or alkyl; and R⁶ is hydrogen, alkyl, cycloalkyl, aryl, or heteroaryl;
each of R⁹ and R¹⁰ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
each of R¹¹ and R¹² is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R^{1′})R¹⁹], —C(O)N(R¹⁸)R¹⁹, —OC(O)N(R^{1′})R¹⁹, or —OP(O)OR²⁰(OR²¹), wherein alkyl, heteroalkyl, cycloalkyl, heterocycloalkyl, aryl, or heteroaryl is unsubstituted or substituted with alkyl, aryl, halogen, —OR¹³, —NR(R¹⁸)R¹⁹, —C(O)R¹⁴, —OC(O)R¹⁵, —OC(O)OR¹⁶, —NHC(O)OR¹⁶, or —OC(O)N(R¹⁸)R¹⁹;
each of R¹³, R¹⁴, R¹⁵, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰ and R²¹ is independently hydrogen, alkyl, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and
each R^{B} is independently halogen, amino, cyano, hydroxyl, alkyl, heteroalkyl, cycloalkyl, heterocycloalkyl, aryl, heteroaryl, arylalkyl, —C(O)CH₃, —C(O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

2. The compound of claim 1, wherein the compound is a compound of Formula (Ia), or a pharmaceutically acceptable salt thereof:

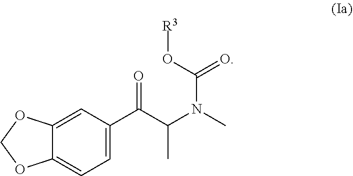

3. The compound of claim 1 or claim 2, wherein the compound is a compound of Formula (Ia-1), or a pharmaceutically acceptable salt thereof:

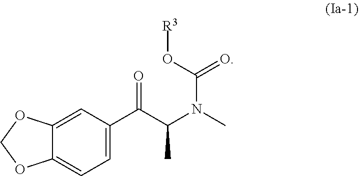

4. The compound of claim 1 or claim 2, wherein the compound is a compound of Formula (Ia-2), or a pharmaceutically acceptable salt thereof:

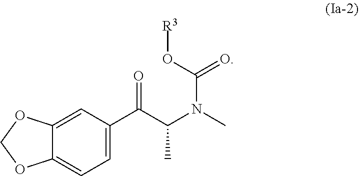

5. The compound of claim 1 or claim 2, wherein the compound is a compound of Formula (Ia1): 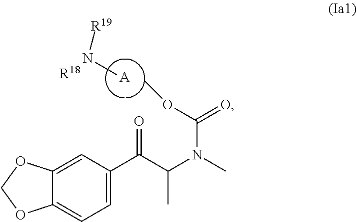
or a pharmaceutically acceptable salt thereof, wherein: 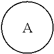
is cycloalkyl or heterocycloalkyl; and
each of R¹⁸ and R¹⁹ is independently hydrogen, alkyl, cycloalkyl, or heteroalkyl;
or R¹⁸ and R¹⁹ together with the atom to which they are attached form a heterocycloalkyl ring.

6. The compound of claim 5, wherein the compound is a compound of Formula (Ia2), or a pharmaceutically acceptable salt thereof:

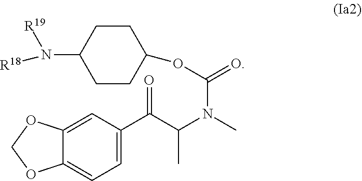

7. The compound of claim 1, wherein the compound is a compound of Formula (Ia3): 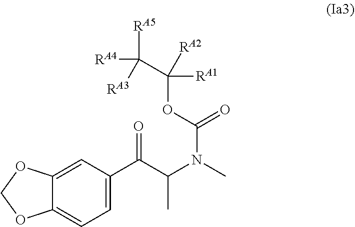
or a pharmaceutically acceptable salt thereof, wherein:
each of R^{A1}, R^{A2}, R^{A3}, and R^{A4} is independently hydrogen or alkyl, and
R^{A5} is heteroalkyl, heterocycloalkyl, heteroaryl, or —C(O)OR³, —N(R¹³)C(O)OR¹⁴, —N(R¹³)C(O)R¹⁴, —C(O)R¹⁴, —OC(O)R¹⁵, or —OC(O)OR¹⁶.

8. The compound of claim 1, wherein the compound is a compound of Formula (Ib), or a pharmaceutically acceptable salt thereof:

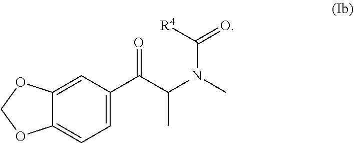

9. The compound of claim 1, wherein the compound is a compound of Formula (Ib2), or a pharmaceutically acceptable salt thereof:

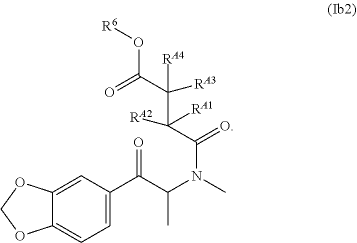

10. The compound of claim 8, wherein the compound is: 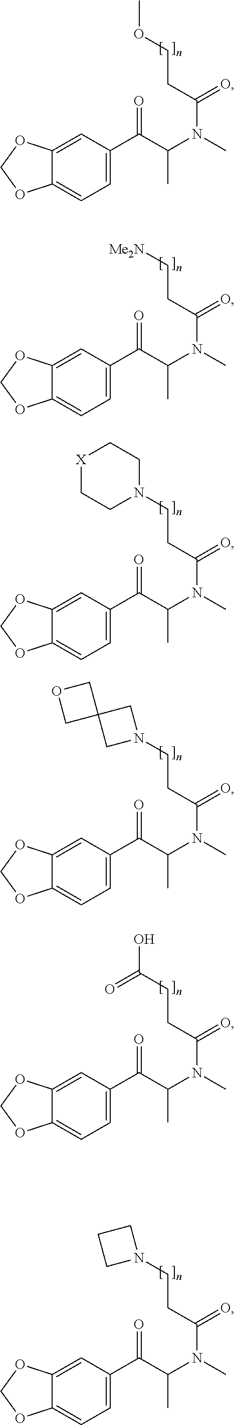 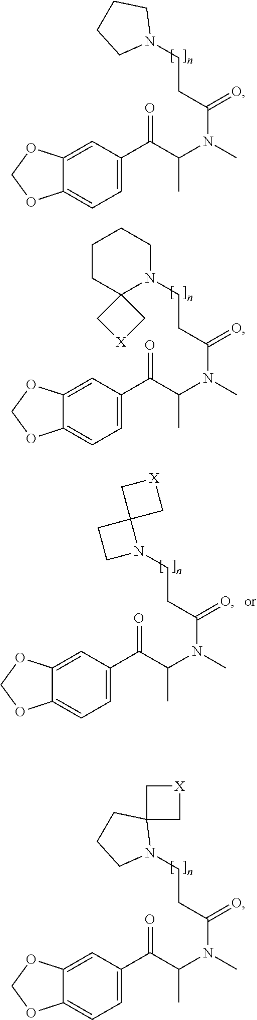
or a pharmaceutically acceptable salt thereof, wherein each n is independently 1, 2, 3, 4, 5, or 6; and each X is independently —O—, —S—, —S(O)—, —S(O)₂—, —NH—, or —NR^{B}, wherein R^{B} is selected from alkyl, heteroalkyl, —C(O)CH₃ and —C(O)Ph, each of which is substituted or unsubstituted.

11. The compound of claim 1, wherein the compound is a compound of Formula (Ib3):

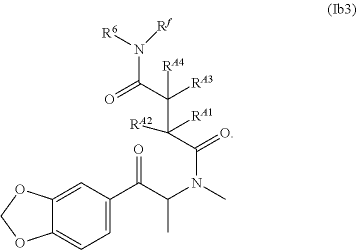

12. The compound of claim 1, or a pharmaceutically acceptable salt thereof, wherein R¹ is —CH(R⁴)OP(O)OR¹¹(OR¹²).

13. The compound of claim 1, wherein the compound is a compound of Formula (Ic): 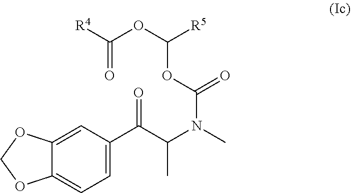
or a pharmaceutically acceptable salt thereof, wherein:
R⁴ is alkyl, alkenyl, haloalkyl, cycloalkyl, heterocycloalkyl, aryl, heteroaryl, or heteroalkyl.

14. The compound of claim 1, wherein the compound is a compound of Formula (Id): 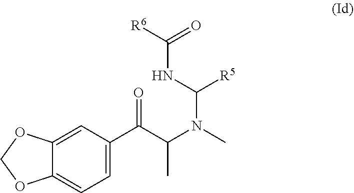
or a pharmaceutically acceptable salt thereof, wherein:
R⁶ is alkyl, alkenyl, heteroalkyl, cycloalkyl, heterocycloalkyl, aryl, or heteroaryl, each of which is substituted or unsubstituted.

15. The compound of claim 1, wherein the compound is a compound of Formula (Ie): 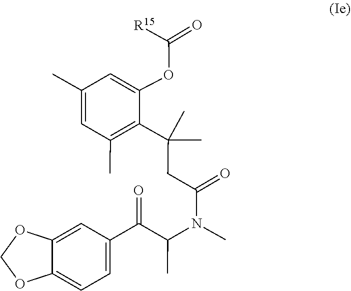
or a pharmaceutically acceptable salt thereof, wherein:
R¹⁵ is alkyl, heteroalkyl, cycloalkyl, heterocycloalkyl, aryl, or heteroaryl, each of which is substituted or unsubstituted.

16. The compound of claim 1, wherein the compound is a compound of Formula (If): 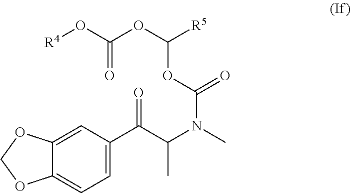
or a pharmaceutically acceptable salt thereof, wherein:
R⁵ is hydrogen or alkyl that is substituted or unsubstituted.

17. The compound of claim 1, or a pharmaceutically acceptable salt thereof, wherein:
each of R³, R⁴, R⁶, R⁷, and R⁸ is independently C₁-C₁₀ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A};
R⁵ is hydrogen, C₁-C₁₀ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A};
or R³ is 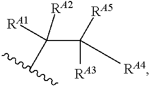
wherein each of R^{A1}, R^{A2}, R^{A3}, and R^{A4} is independently hydrogen or C₁-C₁₀ alkyl; and R^{A5} is C₃-C₆ heteroalkyl, 3- to 6-membered heterocycloalkyl, monocyclic heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶;
or R⁴ is 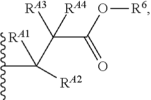
wherein each of R^{A1} and R^{A2} is independently hydrogen, C₁-C₁₀ alkyl, C₃-C₆ cycloalkyl, phenyl, or monocyclic heteroaryl, or R^{A1} and R^{A2} together with the atom to which they are attached form a C₃-C₆ cycloalkyl ring; each of R^{A3} and R^{A4} is independently hydrogen, C₁-C₁₀ alkyl, C₃-C₆ cycloalkyl, phenyl, or monocyclic heteroaryl, or R^{A3} and R^{A4} together with the atom to which they are attached form a C₃-C₆ cycloalkyl ring; and R⁶ is hydrogen, C₁-C₁₀ alkyl, C₃-C₆ cycloalkyl, phenyl, or monocyclic heteroaryl;
or R⁴ is 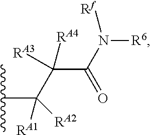
wherein each of R^{A1} and R^{A2} is independently hydrogen, C₁-C₁₀ alkyl, C₃-C₆ cycloalkyl, phenyl, or monocyclic heteroaryl, or R^{A1} and R^{A2} together with the atom to which they are attached form a C₃-C₆ cycloalkyl ring; each of R^{A3} and R^{A4} is independently hydrogen, C₁-C₁₀ alkyl, C₃-C₆ cycloalkyl, phenyl, or monocyclic heteroaryl, or R^{A3} and R^{A4} together with the atom to which they are attached form a C₃-C₆ cycloalkyl ring; R^{f} is hydrogen or C₁-C₁₀ alkyl; and R⁶ is hydrogen, C₁-C₁₀ alkyl, C₃-C₆ cycloalkyl, phenyl, or monocyclic heteroaryl;
each of R⁹ and R¹⁰ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R⁹ and R¹⁰ together with the atom to which they are attached form a 3- to 6-membered heterocycloalkyl ring or a heteroaryl ring that is unsubstituted or substituted with one to five R^{A};
each of R¹¹ and R¹² is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R¹¹ and R¹² together with the atoms to which they are attached form a 3- to 6-membered heterocycloalkyl ring that is unsubstituted or substituted with one to five R^{A};
each R^{A} is independently C₁-C₁₀ alkyl, C₃-C₆ heteroalkyl, C₃-C₈ cycloalkyl, 3- to 6-membered heterocycloalkyl, phenyl, monocyclic heteroaryl, an amino acid side chain, —OR¹³, —N(Rig)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR¹³, —NR(R¹⁸)R¹⁹, —C(O)R¹⁴, —OC(O)R¹⁵, —OC(O)OR¹⁶, —OC(O)N(R¹⁸)R¹⁹, or —OP(O)OR²⁰(OR²¹);
each of R¹³, R¹⁴, R¹⁵, R¹⁶, or R¹⁷ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, and heteroaryl is unsubstituted or substituted with one to five R^{B};
each of R¹⁸ and R¹⁹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}; or R¹⁸ and R¹⁹ together with the atom to which they are attached form a 3- to 6-membered heterocycloalkyl ring or heteroaryl ring, each of which is unsubstituted or substituted with one to five R^{B};
each of R²⁰ and R²¹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}, or R²⁰ and R²¹ together with the atoms to which they are attached form a 3- to 6-membered heterocycloalkyl ring that is unsubstituted or substituted with one to five R^{B}; and
each R^{B} is independently halogen, amino, cyano, hydroxyl, C₁-C₁₀ alkyl, C₃-C₆ heteroalkyl, C₃-C₈ cycloalkyl, 3- to 6-membered heterocycloalkyl, phenyl, monocyclic heteroaryl, benzyl, —C(O)CH₃, —C(O)Ph, or (monocyclic heteroaryl)-C₁-C₄ alkyl wherein cycloalkyl, heterocycloalkyl, phenyl, or heteroaryl is unsubstituted or substituted with one to five halogen, amino, cyano, hydroxyl, C₁-C₆ alkyl, C₁-C₆ acetyl, or benzoyl.

18. The compound of claim 1, or a pharmaceutically acceptable salt thereof, wherein:
each of R³, R⁴, R⁶, R⁷, and R¹ is independently C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A};
R⁵ is hydrogen, C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A};
each of R⁹ and R¹⁰ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R⁹ and R¹⁰ together with the atom to which they are attached form a 3- to 6-membered heterocycloalkyl ring or a heteroaryl ring that is unsubstituted or substituted with one to three R^{A};
each of R¹¹ and R¹² is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R¹¹ and R¹² together with the atoms to which they are attached form a 3- to 6-membered heterocycloalkyl ring that is unsubstituted or substituted with one to three R^{A};
each R^{A} is independently C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR¹³, —NR(R¹⁵)R¹⁹, —C(O)R¹⁴, —OC(O)R¹⁵, —OC(O)OR¹⁶, —OC(O)N(Rig)R¹⁹, or —OP(O)OR²⁰(OR²¹);
each of R¹³, R¹⁴, R¹⁵, R¹⁶, or R¹⁷ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, and heteroaryl is unsubstituted or substituted with one to three R^{B};
each of R¹⁸ and R¹⁹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}; or R¹⁸ and R¹⁹ together with the atom to which they are attached form a 3- to 6-membered heterocycloalkyl ring or heteroaryl ring, each of which is unsubstituted or substituted with one to three R^{B};
each of R²⁰ and R²¹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}, or R²⁰ and R²¹ together with the atoms to which they are attached form a 3- to 6-membered heterocycloalkyl ring that is unsubstituted or substituted with one to three R^{B}; and
each R^{B} is independently halogen, amino, cyano, hydroxyl, C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, benzyl, —C(O)CH₃, —C(O)Ph, or (5- or 6-membered monocyclic heteroaryl)-CH₂—, wherein cycloalkyl, heterocycloalkyl, phenyl, or heteroaryl is unsubstituted or substituted with one to three halogen, amino, cyano, hydroxyl, C₁-C₆ alkyl, C₁-C₆ acetyl, or benzoyl.

19. A compound of Formula (II): 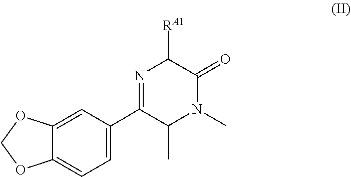
or a pharmaceutically acceptable salt thereof, wherein:
R^{A1} is an amino acid side chain.

20. The compound of claim 19, or a pharmaceutically acceptable salt thereof, wherein:
the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂.

21. A compound of Formula (III): 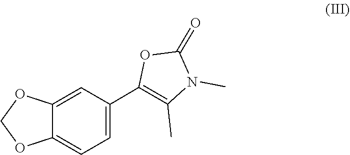 [SMILES: Cc1c(-c2ccc3c(c2)OCO3)oc(=O)n1C]
or a pharmaceutically acceptable salt thereof.

22. A compound selected from: 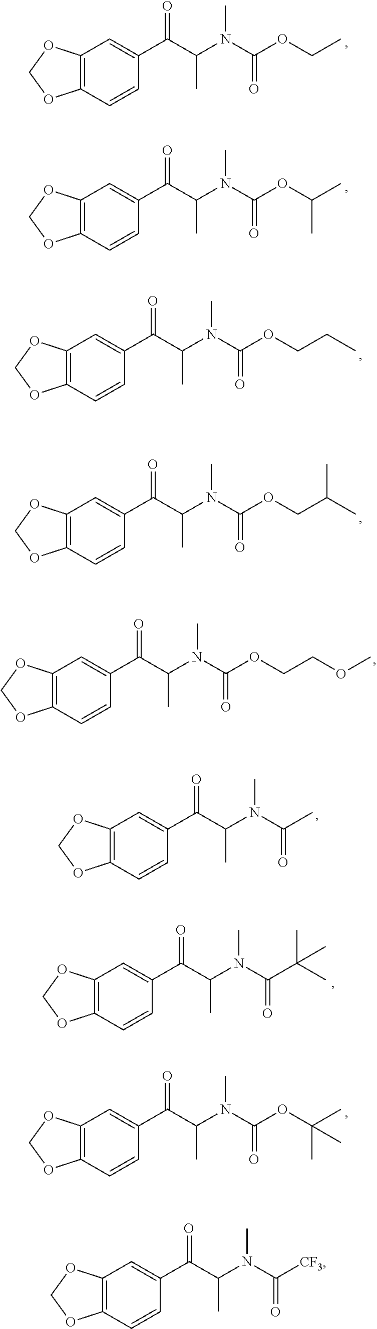 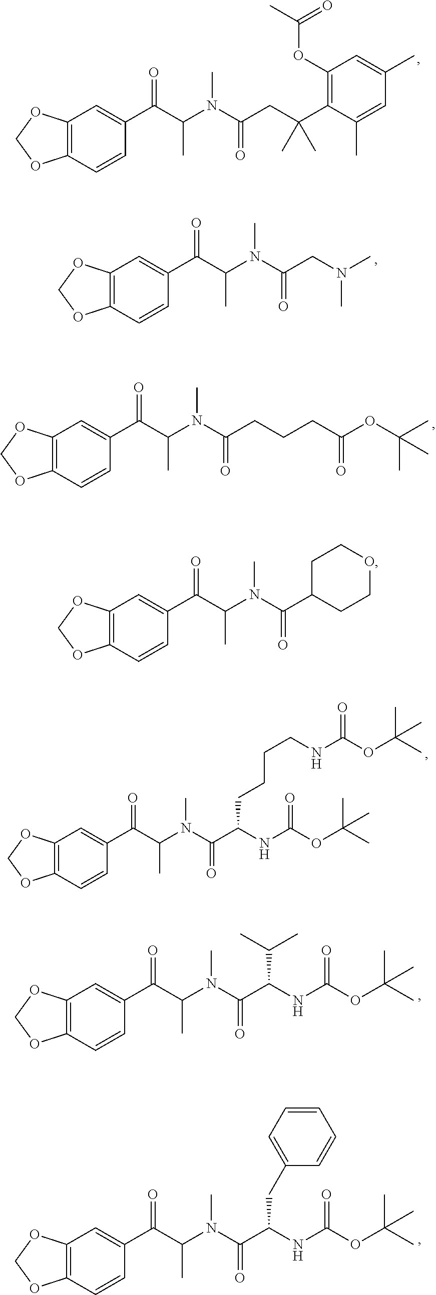 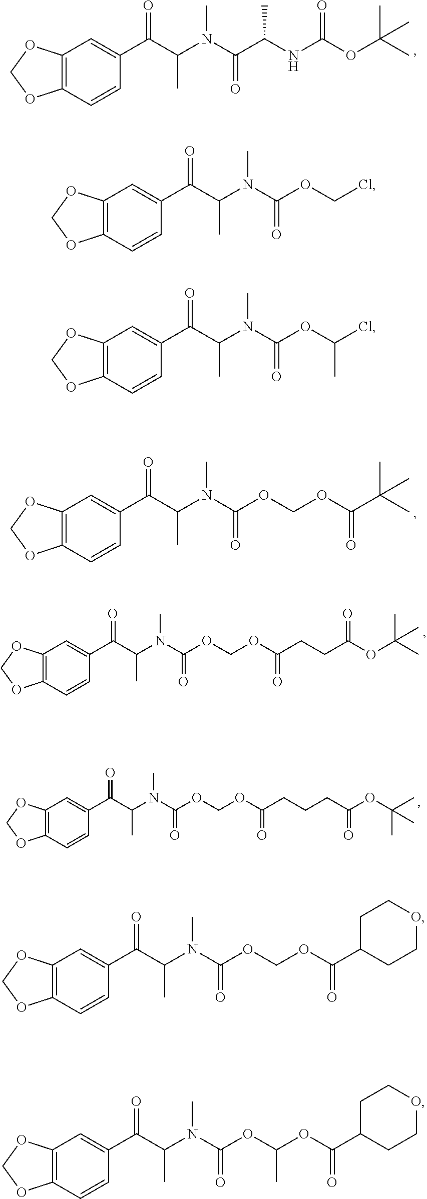 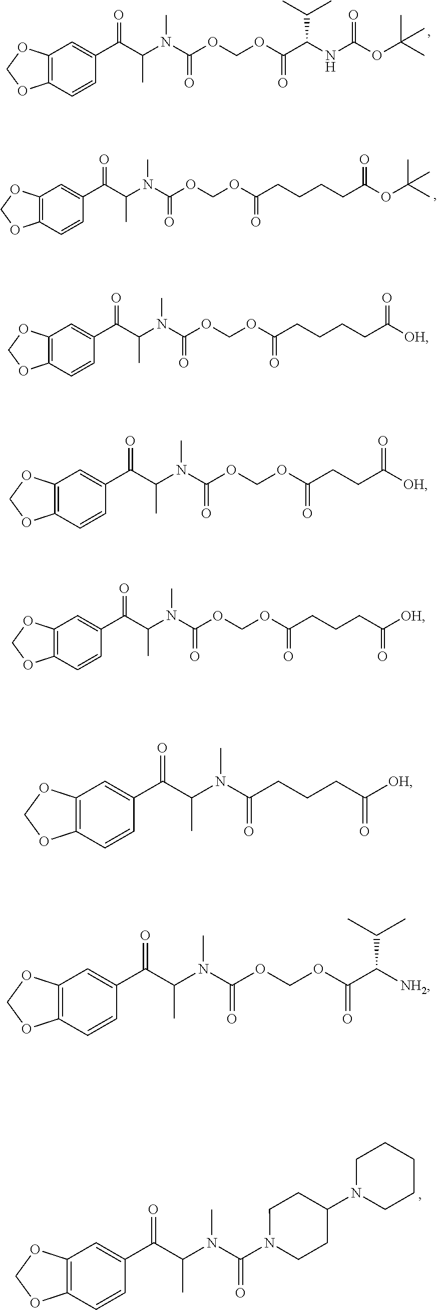 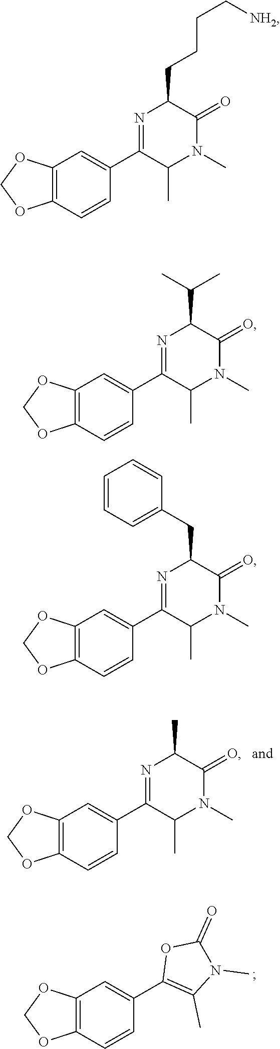
or a pharmaceutically acceptable salt thereof.

23. The compound of any one of claims 1-22, wherein the compound is enriched in deuterium.

24. The compound of any one of claims 1-23, wherein the compound is in the form of a pharmaceutically acceptable salt.

25. A solvate of the compound of any one of claims 1-24, or a pharmaceutically acceptable salt thereof.

26. A pharmaceutical composition comprising a compound of any one of claims 1-24, or a pharmaceutically acceptable salt thereof.

27. A method for method for increasing neuronal plasticity, comprising contacting a neuron with an effective amount of a compound according to any one of claims 1-24 or a pharmaceutical composition according to claim 26.

28. The method of claim 27, wherein contacting comprises administering the compound to a subject.

29. A method for treating a neurological disorder, a psychiatric disorder, or both, comprising contacting a subject having the neurological disorder, psychiatric disorder or both with an effective amount of a compound according to any one of claims 1-24 or a pharmaceutical composition according to claim 26.

30. The method of claim 29, wherein the neurological disorder is a neurodegenerative disorder.

31. The method of claim 29, wherein the neurological disorder, psychiatric disorder, or both, comprises depression, addiction, anxiety, or a post-traumatic stress disorder.

32. The method of claim 29, wherein the neurological disorder, psychiatric disorder, or both, comprises treatment resistant depression, suicidal ideation, major depressive disorder, bipolar disorder, schizophrenia, or substance use disorder.

33. The method of claim 29, wherein the neurological disorder, psychiatric disorder, or both, comprises stroke, traumatic brain injury, or a combination thereof.

34. The method of claim 29, further comprising administering a 5-HT_{2A} antagonist to the subject.

35. The method of claim 34, wherein the 5-HT_{2A} antagonist is selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), olanzapine, risperidone, pimavanserin, nelotanserin and lorcaserin.
